(19) United States
(12) Patent Application Publication
(10) Pub. No.: US 2025/0352625 A1
(43) Pub. Date: Nov. 20, 2025

(54) METHODS AND COMPOSITIONS FOR METHIONINE RESTRICTION

(71) Applicant: PETRI BIO, INC., Torrance, CA (US)

(72) Inventors: Joseph SCHINAMAN, Torrance, CA (US); Shu LI, Torrance, CA (US)

(73) Assignee: PETRI BIO, INC., Torrance, CA (US)

(21) Appl. No.: 18/871,363

(22) PCT Filed: Jun. 2, 2023
(86) PCT No.: PCT/US2023/067840

§ 371 Date: Dec. 3, 2024

Related U.S. Application Data
(60) Provisional application No. 63/348,559, filed on Jun. 3, 2022.

(51) Int. Cl. A61K 38/51; A23L 29/00; A61K 35/741; A61K 38/44; C12N 15/52

(52) U.S. Cl. CPC A61K 38/51; A23L 29/065; A61K 35/741; A61K 38/44; C12N 15/52; C12Y 108/03004; C12Y 404/01011

(57) ABSTRACT

The technology described herein is directed to compositions and methods for reducing levels of methionine. In various aspects described herein are: engineered methionine-reducing probiotic microorganisms; engineered methanethiol-reducing probiotic microorganisms; and engineered taurine-producing probiotic microorganisms. Also described herein are methods of using such engineered microorganisms, such as for reduction of bioavailable methionine or for treatment of a methionine-associated disease or disorder. Also described herein are probiotic dietary supplements, pharmaceutical compositions, and food compositions comprising such engineered microorganisms.

### CROSS-REFERENCE TO RELATED APPLICATIONS

This application claims benefit under 35 U.S.C. § 119(e) of U.S. Provisional Application No. 63/348,559 filed Jun. 3, 2022, the contents of which are incorporated herein by reference in their entirety.

### GOVERNMENT SUPPORT

This invention was made with government support under Grant No. R43HD107885 awarded by the National Institutes of Health. The government has certain rights in the invention.

### SEQUENCE LISTING

The instant application contains a Sequence Listing which has been submitted in XML format via Patent Center and is hereby incorporated by reference in its entirety. Said XML copy, created on Jun. 1, 2023, is named 093451-191710WOPT.xml and is 468,029 bytes in size.

### TECHNICAL FIELD

The technology described herein relates to methods and compositions for methionine restriction, including engineered microorganisms for methionine restriction.

### BACKGROUND

Overconsumption of methionine is linked to fatty liver disease, Alzheimer's, and heart disease. Low levels of methionine extend life and reduce weight in animal models and human cell culture. Reducing methionine in the diet leads to improved outcomes, such as reducing liver adiposity and fat mass in mouse and humans, and increasing efficacy of chemotherapy and radiotherapy in mice. Reduced methionine diets are also the standard of care (SoC) for homocystinuria (HCU), an inherited disorder of methionine metabolism, e.g., due to a deficiency of cystathionine beta synthase or methionine synthase, leading to increased levels of homocysteine (a methionine metabolite) in serum and urine. Furthermore, reduced dietary methionine has an anti-aging impact. Diets with low methionine extended lifespan 55% in an invertebrate model (C. elegans), extended lifespan 40% in a mammalian model (e.g., rat), and extended replicative lifespan 40% in human cells. Overall, dietary restriction of the amino acid methionine has been shown to have health benefits in a variety of model systems, e.g., increasing lifespan in vitro and in vivo and significantly reducing cancer risk and increasing cancer treatment efficacy in mice.

Current approaches to reducing methionine in the diet require very restricted low protein diet and supplementation with Methionine-free amino acid formula. The challenging and costly diet causes low compliance and impact patients' quality of life; these methods are thus unsustainable long term. As such, there is great need for more inexpensive and efficient approaches to decrease methionine levels.

### SUMMARY

The technology described herein is directed to compositions and methods for reducing levels of methionine, e.g., in the mammalian gut. In various aspects described herein are: engineered methionine-reducing probiotic microorganisms; and engineered methanethiol-reducing probiotic microorganisms; engineered taurine-producing probiotic microorganisms. Also described herein are methods of using such engineered microorganisms, such as for reduction of bioavailable methionine or for treatment of a methionine-associated disease or disorder. Also described herein are probiotic dietary supplements, pharmaceutical compositions, and food compositions comprising such engineered microorganisms.

Accordingly, in one aspect, described herein is an engineered probiotic microorganism for reducing bioavailable methionine levels, comprising: (a) at least one exogenous copy of at least one gene encoding an enzyme that catalyzes the degradation of methionine, wherein the gene encoding an enzyme that catalyzes the degradation of methionine encodes a methionine gamma lyase.

In one aspect, described herein is an engineered probiotic microorganism for reducing bioavailable methionine levels, comprising: (a) at least one exogenous copy of at least one gene encoding an enzyme that catalyzes the degradation of methionine, wherein the gene encoding an enzyme that catalyzes the degradation of methionine encodes a methionine gamma lyase; and (b) at least one of the following: (i) at least one exogenous copy of at least one functional methionine importer gene; and/or (ii) at least one endogenous methionine importer gene comprising at least one engineered activating modification.

In one aspect, described herein is an engineered probiotic microorganism for reducing bioavailable methionine levels, comprising: (a) at least one exogenous copy of at least one gene encoding an enzyme that catalyzes the degradation of methionine; (b) at least one exogenous copy of at least one functional methionine importer gene; (c) at least one endogenous methionine importer gene comprising at least one engineered activating modification; (c) at least one endogenous methionine synthesis gene comprising at least one engineered inactivating modification; (d) at least one endogenous methionine regulator gene comprising at least one engineered inactivating or activating modification; or (e) a combination of two or more of (a)-(e).

In some embodiments of any of the aspects, the exogenous gene(s) of (a) and (b), if present, and the endogenous gene(s) of (c), (d), and (e), if present, are expressed by the engineered probiotic microorganism under conditions in the gut.

In some embodiments of any of the aspects, the at least one engineered activating modification comprises: (a) at least one engineered activating mutation in the at least one endogenous methionine importer gene or in the at least one endogenous methionine regulator gene; and/or (b) at least one engineered activating mutation in a promoter operatively linked to the at least one endogenous methionine importer gene or to the at least one endogenous methionine regulator gene.

In some embodiments of any of the aspects, the at least one engineered inactivating modification comprises: (a) at least one engineered inactivating mutation in the at least one endogenous methionine synthesis gene or in the at least one endogenous methionine regulator gene; (b) at least one engineered inactivating mutation in a promoter operatively linked to the at least one endogenous methionine synthesis gene or to the at least one endogenous methionine regulator gene; and/or (c) at least one inhibitory RNA molecule specific to at least one messenger RNA (mRNA) expressed by the at least one endogenous methionine synthesis gene or by the at least one endogenous methionine regulator gene.

In some embodiments of any of the aspects, the enzyme that catalyzes the degradation of methionine generates methanethiol.

In some embodiments of any of the aspects, the gene encoding an enzyme that catalyzes the degradation of methionine encodes a methionine gamma lyase.

In some embodiments of any of the aspects, the engineered probiotic microorganism further comprises and expresses an exogenous gene encoding a methanethiol catabolizing enzyme.

In some embodiments of any of the aspects, the methanethiol-catabolizing enzyme is an esterase or a methanethiol oxidase.

In some embodiments of any of the aspects, the methionine gamma lyase comprises SEQ ID NO: 6 or an amino acid sequence that is at least 90% identical.

In some embodiments of any of the aspects, the methionine gamma lyase comprises one of SEQ ID NOs: 5-6 or an amino acid sequence that is at least 90% identical.

In some embodiments of any of the aspects, the gene encoding an enzyme that catalyzes the degradation of methionine encodes expression of a catalytically-active fragment of a methionine gamma lyase.

In some embodiments of any of the aspects, the gene encoding an enzyme that catalyzes the degradation of methionine encodes expression of a fusion protein comprising a catalytically-active fragment of a methionine gamma lyase.

In some embodiments of any of the aspects, the engineered probiotic microorganism comprises at least one exogenous copy of at least one gene encoding an enzyme that catalyzes the degradation of methionine and at least one exogenous copy of at least one functional methionine importer gene.

In some embodiments of any of the aspects, the engineered probiotic microorganism comprises at least one exogenous copy of at least one gene encoding an enzyme that catalyzes the degradation of methionine and at least one endogenous copy of at least one functional methionine importer gene comprises a mutation that increases the rate of methionine import relative to wild-type of that enzyme.

In some embodiments of any of the aspects, the engineered probiotic microorganism comprises at least one exogenous copy of at least one gene encoding an enzyme that catalyzes the degradation of methionine and at least one endogenous methionine synthesis gene comprising at least one engineered inactivating modification.

In some embodiments of any of the aspects, the engineered probiotic microorganism comprises at least one exogenous copy of at least one gene encoding an enzyme that catalyzes the degradation of methionine and at least one endogenous methionine regulator gene comprising at least one engineered inactivating or activating modification.

In one aspect, described herein is an engineered probiotic microorganism for reducing bioavailable methionine levels, the microorganism comprising: (a) at least one endogenous methionine synthesis gene comprising at least one engineered inactivating modification; (b) at least one copy of an exogenous gene encoding a homocysteine methyltransferase enzyme; (c) at least one copy of an exogenous gene encoding a sulfinoalanine decarboxylase enzyme; and (d) at least one copy of an exogenous gene encoding a Flavin-containing monooxygenase enzyme (FMO); wherein the engineered probiotic microorganism expresses endogenously or exogenously encoded cystathionine β-synthase, cystathionine gamma lyase and cysteine dioxygenase enzymes.

In some embodiments of any of the aspects, the homocysteine methyltransferase enzyme is a YhcE homocysteine methyltransferase enzyme.

In some embodiments of any of the aspects, the at least one engineered inactivating modification comprises: (a) at least one engineered inactivating mutation in the at least one endogenous methionine synthesis gene; (b) at least one engineered inactivating mutation in a promoter operatively linked to the at least one endogenous methionine synthesis gene; and/or (c) at least one silencing RNA molecule specific to at least one messenger RNA (mRNA) expressed by the at least one endogenous methionine synthesis gene.

In one aspect, described herein is an engineered probiotic microorganism for reducing bioavailable methionine levels, the microorganism comprising: (a) at least one endogenous methionine synthesis gene comprising at least one engineered inactivating modification; (b) at least one copy of an exogenous gene encoding a glycine N-methyltransferase (GNMT) enzyme; (c) at least one copy of an exogenous gene encoding a sarcosine N-methyl transferase (SNMT) enzyme; (d) at least one copy of an exogenous gene encoding a sulfinoalanine decarboxylase enzyme; and (e) at least one copy of an exogenous gene encoding a Flavin-containing monooxygenase (FMO) enzyme; wherein the engineered probiotic microorganism expresses endogenously or exogenously encoded methionine adenosyl transferase (MetK), adenosylhomocysteinase (ahcY), cystathionine β-synthase, cystathionine gamma lyase and cysteine dioxygenase enzymes.

In some embodiments of any of the aspects, the FMO enzyme is an FMO1, FMO2 or FMO3 enzyme that catalyzes the catalysis of the conversion of hypotaurine to taurine.

In some embodiments of any of the aspects, the engineered probiotic microorganism metabolizes methionine to taurine.

In some embodiments of any of the aspects, the at least one engineered inactivating modification comprises: (a) at least one engineered inactivating mutation in the at least one endogenous methionine synthesis gene; (b) at least one engineered inactivating mutation in a promoter operatively linked to the at least one endogenous methionine synthesis gene; and/or (c) at least one silencing RNA molecule specific to at least one messenger RNA (mRNA) expressed by the at least one endogenous methionine synthesis gene.

In some embodiments of any of the aspects, the at least one endogenous methionine synthesis gene is MetE and/or MetH.

In one aspect, described herein is a pharmaceutical composition comprising an engineered probiotic microorganism as described herein, and a pharmaceutically acceptable carrier.

In some embodiments of any of the aspects, the purified mixture of live bacteria comprises species present in an amount of at least about 1×10⁸ CFUs/ml.

In some embodiments of any of the aspects, the pharmaceutical composition is formulated for oral administration.

In some embodiments of any of the aspects, the pharmaceutical composition is formulated for delivery to the gut via oral administration.

In some embodiments of any of the aspects, the pharmaceutical composition is enteric coated.

In some embodiments of any of the aspects, the pharmaceutical composition is formulated for injection.

In some embodiments of any of the aspects, the pharmaceutical composition further comprises at least one additional methionine-decreasing or homocysteine-decreasing therapeutic.

In some embodiments of any of the aspects, the pharmaceutical composition is co-administered with at least one additional methionine-decreasing or homocysteine-decreasing therapeutic.

In some embodiments of any of the aspects, the at least one additional methionine-decreasing or homocysteine-decreasing therapeutic is selected from the group consisting of: betaine, taurine, a methionine restriction diet, a methionine-free formula, and combinations thereof.

In one aspect, described herein is a food composition comprising an engineered probiotic microorganism as described herein.

In one aspect, described herein is a probiotic dietary supplement comprising an engineered probiotic microorganism as described herein.

In one aspect, described herein is a method of reducing bioavailable methionine in a mammal in need thereof, the method comprising administering an engineered probiotic microorganism as described herein, a pharmaceutical composition as described herein, a food composition as described herein, or a probiotic dietary supplement as described herein to the mammal.

In some embodiments of any of the aspects, the administering is oral or rectal.

In some embodiments of any of the aspects, the administering is by injection.

In some embodiments of any of the aspects, the administering reduced the level of bioavailable methionine in the gut of the mammal.

In some embodiments of any of the aspects, the method further comprises administering an effective amount of at least one additional methionine-decreasing or homocysteine-decreasing therapeutic.

In some embodiments of any of the aspects, the at least one additional methionine-decreasing or homocysteine-decreasing therapeutic is selected from the group consisting of: betaine, taurine, a methionine restriction diet, a methionine-free formula, and combinations thereof.

In one aspect, described herein is a method of treating a cancer in a subject in need thereof, the method comprising administering an effective amount of an engineered probiotic microorganism as described herein.

In some embodiments of any of the aspects, the cancer is a methionine-dependent cancer.

In some embodiments of any of the aspects, the cancer is selected from the group consisting of: glioma colon cancer, breast cancer, ovarian cancer, prostate cancer, melanoma, and sarcoma.

In some embodiments of any of the aspects, the cancer is a glioma.

In some embodiments of any of the aspects, the method further comprises administering an effective amount of at least one additional methionine-decreasing or homocysteine-decreasing therapeutic.

In some embodiments of any of the aspects, the at least one additional methionine-decreasing or homocysteine-decreasing therapeutic is selected from the group consisting of: betaine, taurine, a methionine restriction diet, a methionine-free formula, and combinations thereof.

In some embodiments of any of the aspects, the method further comprises administering an effective amount of at least one additional cancer therapeutic.

In some embodiments of any of the aspects, the administering is by injection.

In one aspect, described herein is a method of reducing a level of methanethiol, the method comprising contacting methanethiol with a probiotic microorganism that encodes and expresses an exogenous gene encoding a methanethiol catabolizing enzyme.

In some embodiments of any of the aspects, the methanethiol catabolizing enzyme is an esterase.

In some embodiments of any of the aspects, the methanethiol catabolizing enzyme is a methanethiol oxidase.

In some embodiments of any of the aspects, the methanethiol is produced by an engineered probiotic microorganism that comprises and expresses an exogenous gene encoding an enzyme that catalyzes the degradation of methionine to products including methanethiol.

In some embodiments of any of the aspects, the enzyme that catalyzes the degradation of methionine to products including methanethiol comprises a methionine gamma lyase enzyme.

In one aspect described herein is a method of reducing odor produced by a population of gut microbiota that produced methanethiol, the method comprising introducing an engineered probiotic microorganism to the gut microbiota, wherein the engineered probiotic microorganism encodes and expresses an exogenous gene encoding a methanethiol catabolizing enzyme.

In some embodiments of any of the aspects, the methanethiol catabolizing enzyme is an esterase.

In some embodiments of any of the aspects, the methanethiol catabolizing enzyme is a methanethiol oxidase.

In some embodiments of any of the aspects, the methanethiol is produced by an engineered probiotic microorganism that comprises and expresses an exogenous gene encoding an enzyme that catalyzes the degradation of methionine to products including methanethiol.

In some embodiments of any of the aspects, the enzyme that catalyzes the degradation of methionine to products including methanethiol comprises a methionine gamma lyase enzyme.

In one aspect, described herein is a method of generating taurine from methionine in the gut of a mammal, the method comprising introducing an engineered probiotic microorganism as described herein to the gut of the mammal.

In some embodiments of any of the aspects, the microorganism is introduced via oral administration.

### BRIEF DESCRIPTION OF THE DRAWINGS

FIG. 1A-1B is a series of schematics showing engineered bacteria as described herein and methionine-associated pathways. FIG. 1A is a schematic showing five classes of genes encoding methionine-associated proteins that can be engineered as described herein: (1) importer proteins; (2) catabolic enzymes (methionine breakdown); (3) anabolic enzymes (methionine synthesis); and/or (4) methionine metabolic sensors and/or regulatory proteins. FIG. 1B is a schematic showing the demethiolation of methionine by methionine gamma lyase (MGL). A blue circle indicates that the enzyme (e.g., MGL) is an exogenous transgene from another species.

FIG. 2 is a schematic showing a plasmid for expression of methionase-1.

FIG. 3 is an image showing successful transformation of plasmids (see e.g., FIG. 2) into bacteria (E. coli, strain BL21).

FIG. 4A-4B is a series of images confirming integration of methionase DNA in transformed bacteria for 10 candidate enzymes. FIG. 4A shows an electrophoretic gel of colony PCR, using pET28(a) forward and reverse primers (see e.g., FIG. 2). The band of the PCR product indicates presence of the pET28(a) plasmid. FIG. 4B is an image of an agar plate streaked for each engineered bacterium. For FIG. 4A-4B, A1: Methionine Gamma Lyase (MGL) from P. putida; A2: MGL T. denticola; B1: MGL C. freundii; B2: MGL S. thermophilus; C1: MGL B. subtilis; D1: MGL B. linens; E1: MGL Bioreactor Met; F1: CGL-NLV H. sapiens; G1: CGL-Ctrl H. sapiens; H1: MGL B. cereus; “+”: DNA fragment; first “−”: TE buffer; and second “−”: water

FIG. 5 is an image of Coomassie Blue staining of an SDS-PAGE gel, which reveals protein bands at expected sizes of candidate methionase genes (see e.g., boxed bands). For the Coomassie Blue staining, 10 ul of total soluble protein was loaded into each well.

FIG. 6 is an image of Western Blot staining, which confirms expression of candidate methionase proteins via His tag. For the Western blot, 10 ul of total soluble protein was loaded into each well, and the blots were probed with anti-His monoclonal antibody (1:1000).

FIG. 7 is a bar graph showing a screen of relative enzyme efficiency. The indicated purified enzyme was incubated for 30 min with 70 mM L-Met, and the formation of methionine degradation product methanethiol (MeSH; CH₃SH) was detected.

FIG. 8 is a series of bar graphs showing in vitro testing of methionase-expressing E. coli via high-performance liquid chromatography (HPLC). The engineered bacteria reduced methionine levels in the medium, to a level associated with therapeutic benefit in animal models. The methionine reduction was associated with a p value less than 0.0001, determined by one-way ANOVA.

FIG. 9 is a bar graph showing that the engineered bacteria (far right grey bars) reduced methionine level specifically and did not degrade other amino acids, such as glycine (Gly), histidine (His), phenylalanine (Phe), isoleucine (Ile), or alanine (Ala). The methionine reduction was associated with a p value less than 0.0001, determined by one-way ANOVA. The top-down order of the legend is the same as the left-right order of the bars in each group.

FIG. 10 is a schematic showing an advanced demethiolation pathway of methionine, specifically to reduce the levels of the malodorous byproduct, methanethiol, using an exogenous esterase. A blue circle indicates that the enzyme (e.g., MGL; esterase) is encoded by an exogenous transgene from another species.

FIG. 11A-11B are a series of schematics showing advanced or alternative demethiolation pathways of methionine, specifically to reduce the levels of the malodorous byproduct, methanethiol. FIG. 11A is a schematic showing use of exogenous methanethiol oxidase, catalase, and optionally formaldehyde dehydrogenase. FIG. 11B is a schematic showing use of exogenous methanethiol oxidase, catalase, and optionally formaldehyde dehydrogenase, formate acetyltransferase, and/or sulfite reductase. A blue circle indicates that the enzyme (e.g., MGL; methanethiol oxidase; catalase; formaldehyde dehydrogenase; formate acetyltransferase; sulfite reductase) is encoded by an exogenous transgene from another species.

FIG. 12 is a schematic showing a direct pathway for synthesizing taurine from methionine. A blue circle indicates that the enzyme (e.g., YhcE; sulfinoalanine decarboxylase; FMO3) is encoded by an exogenous transgene from another species. An orange circle indicates that the enzyme (e.g., MetE; MetH) is encoded by an endogenous gene comprising at least one engineered inactivating modification. A green circle indicates that the enzyme (e.g., CBS; CGL; cysteine dioxygenase) is encoded by an endogenous gene, and its expression can optionally be optimized by modifications to associated regulatory regions, or its enzymatic kinetics can optionally be modulated by at least one activating modification.

FIG. 13 is a schematic showing an indirect pathway for synthesizing taurine from methionine. A blue circle indicates that the enzyme (e.g., GNMT; SNMT; sulfinoalanine decarboxylase; FMO3) is encoded by an exogenous transgene from another species. An orange circle indicates that the enzyme (e.g., MetE; MetH) is encoded by an endogenous gene comprising at least one engineered inactivating modification. A green circle indicates that the enzyme (e.g., MetK; ahcY; CBS; CGL; cysteine dioxygenase) is encoded by an endogenous gene, and its expression can optionally be optimized by modifications to associated regulatory regions, or its enzymatic kinetics can optionally be modulated by at least one activating modification.

FIG. 14 is a schematic showing the microbe-mediated enzyme substitution approach.

FIG. 15 is a bar graph showing in vitro testing of methionases.

FIG. 16A-16C is a series of schematics showing the cryptic plasmid “pMut1” found naturally in E. coli Nissle 1917. FIG. 16A shows various elements on this natural plasmid that allow it to propagate at high copy number in E. coli Nissle (EcN) without antibiotic selection, making it useful for maintenance in the antibiotic-free environment of the gut. FIG. 16B shows the Gen1 plasmid, which comprises sequences encoding Methionine Gamma Lyase (MGL) and a selection marker (e.g., kanR—a kanamycin resistance gene) inside pMut1. This allows for intracellular expression of methionase inside E. coli Nissle 1917, and rapid selection for prototyping. FIG. 16C shows the Gen2 plasmid, which comprises MGL, a selection marker (e.g., kanR), and a methionine active transporter inside pMut1. Import of methionine into the cell was found to be the rate limiting step for degradation. Therefore, active transport genes specific for methionine were genetically modified for constitutive activity and added to the EcN specific plasmid.

FIG. 17 is a bar graph showing that the bacterial cell wall is the rate limiting step to methionine degradation. Methionine degradation was revealed by colorimetric formation of a methionine metabolite MeSH with the reagent DTNB (Ellman's Reagent, 5,5′-Dithiobis-(2-Nitrobenzoic Acid). E coli Nissle 1917 had minimal methionine degrading activity over the time course studied compared to media alone (EcN CTRL vs. Blank). Transgenic EcN cells expressing enzyme “8” showed significant ability to degrade methionine. Free extracts of extracellular protein (Lysed EcN “8”) showed significantly higher degradation compared to enzyme intracellular in EcN (EcN “8”). These results indicate that the cell wall is the rate limiting step in methionine degradation, and that the engineered bacterium can include methionine importer genes.

FIG. 18 is a bar graph showing that transgenic methionine importer genes improved methionine degrading speed of EcN+MGL Enzyme. First generation intact EcN expressing methionase (EcN “8”) degraded methionine more slowly than the enzymes free in solution (Lysed EcN “8”). The second generation of EcN cells, which are transgenic for MGL “8” and a methionine permease or active importer gene (EcN: 8-A, 8-B, 8-C, 8-D), exhibited improved capacity relative to Gen1 (EcN “8”). Results were significant with p<0.0001, in an unpaired t-test.

FIG. 19 is a bar graph showing a methionine degradation time course for wild type EcN, Gen 1 (MGL), and Gen 2 (MGL+importer). The left-right order of the legend is the same as the left-right order of the bars in each group. The time course shows the ability of three EcN strains to degrade methionine from their surroundings in vitro. Wild type EcN (“EcN”) yielded a minimal amount of the methionine degradation product over the 4-hour time course. EcN expressing a screened methionase enzyme (“EcN+Enzyme”) showed a slow ramp up of degradation over the 4 hours. EcN expressing the screened methionase and importer combination (“EcN+Enzyme+Importer”) showed a rapid onset of methionine degradation, far exceeding the 4-hour mark of EcN expressing enzyme alone after only 1 hour.

FIG. 20 is a bar graph showing methionine concentration assayed via HPLC for bench scale and bioreactor scale doses of strain “8C”. Formulated doses of strain “8C” were frozen (at −80° C.), thawed, and incubated overnight in a simulated gut medium, and supernatant was sent off for HPLC analysis, alongside fresh 8C and control EcN. Both manufacturing methods yielded experimental bacteria exhibiting a >90% reduction in methionine relative to control bacteria. This shows that the food-safe −80° C. storage glycerol buffer functions to preserve bacteria, sufficient for animal testing. Results were significant with p<0.0002, in an unpaired t-test.

FIG. 21 is a schematic showing the dosing schedule for in vivo testing.

FIG. 22 is a bar graph showing that treatment with PTRI-8C reduced plasma homocysteine. Pre-treatment, cystathionine β-synthase knockout (CBS −/−) mice have an average plasma homocysteine of 210 uM, well above the normal upper bound of 15 uM, and symptom-free threshold of ˜100 uM. Within 4 days of treatment with PTRI-8C, plasma homocysteine dropped to 136 uM, a 35% decrease. Results were significant with p<0.0002, in a paired t-test.

FIG. 23 is a bar graph showing Bacillus subtilis expressing MGL. The Gram-positive bacteria expresses the MGL enzyme, and the bacterium indeed cleaved methionine to methanethiol, similar to the E. coli Nissle version.

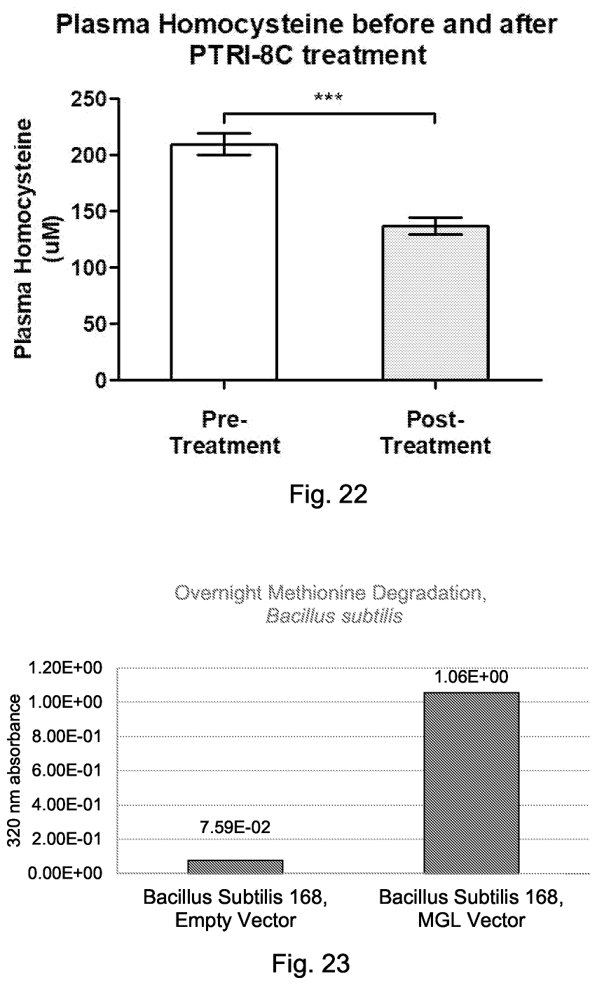

FIG. 24 shows an exemplary HPLC chromatogram for detection of bioavailable methionine in a blood (e.g., plasma or serum) sample.

FIG. 25 is a bar graph showing in vitro testing of the following E. coli bacteria: human cystathionine gamma lyase (hCGL) present but uninduced (-); Bacillus subtilis MGL present but uninduced (-); and E. coli bacteria engineered to express the following exogenous methionine-degrading enzymes, under expression-inducing conditions: Bacillus cereus MGL; hCGL NLV (hCGL-E59N-R119L-E339V variant; see e.g., Yan et al. Biochemistry. 2017 Feb. 14; 56(6): 876-885, the contents of which are incorporated herein by reference in their entirety); Streptococcus thermophilus MGL; Citrobacter freundii MGL; Treponema denticola MGL (see e.g., SEQ ID NO: 6); hCGL; Bioreactor Metagenome MGL (see e.g., SEQ ID NO: 5); Pseudomonas putida MGL; Bacillus subtilis MGL; or Brevibacterium auranticum MGL. Only the Treponema denticola MGL (see e.g., SEQ ID NO: 6) and the Bioreactor Metagenome MGL (see e.g., SEQ ID NO: 5) resulted in significant MGL enzymatic activity above negative control.

### DETAILED DESCRIPTION

The technology described herein is directed to compositions and methods for reducing levels of methionine, e.g., in the mammalian gut. In various aspects described herein are: engineered methionine-reducing probiotic microorganisms; engineered methanethiol-reducing probiotic microorganisms; and engineered taurine-producing probiotic microorganisms. Also described herein are methods of using such engineered microorganisms, such as for reduction of bioavailable methionine or for treatment of a methionine-associated disease or disorder. Also described herein are probiotic dietary supplements, pharmaceutical compositions and food compositions comprising such engineered microorganisms. The methods, supplements, pharmaceutical compositions, or food compositions can comprise any combination of such engineered microorganisms (see e.g., Table 1). Thus, while in some embodiments a single engineered microorganism encodes and expresses the metabolic machinery permitting a reduction in methionine levels, in other embodiments, a consortium of two or more engineered bacteria can be used in which each bacterium performs one or more of the pathway reactions leading to a reduction in methionine and/or methionine catabolic by-product levels. In some embodiments, a consortium of microorganisms that together naturally produce the polypeptides described herein can be used, e.g., to reduce methionine, reduce methanethiol, and/or produce taurine.

TABLE 1
Exemplary combinations of engineered probiotic microorganisms.

| | methanethiol- | |
|---|---|---|
| methionine-reducing | reducing | taurine-producing |
| microorganism | microorganism | microorganism |
| X | | |
| | X | |
| X | X | |
| | | X |
| X | | X |
| | X | X |
| X | X | X |

In some embodiments of any of the aspects, the probiotic microorganism is engineered from a wild-type microorganism selected, for example, from the group consisting of Escherichia coli; Bacillus subtilis; Pseudomonas putida; Treponema denticola; Citrobacter freundii; Bacillus cereus; Streptococcus thermophilus; Saccharomyces cerevisiae; Lactococcus lactis; Lactobacillus plantarum; and Brevibacterium linens, among others. In some embodiments of any of the aspects, the probiotic microorganism is engineered from a wild-type microorganism genus selected, for example, from the group consisting of Escherichia; Bacillus; Pseudomonas; Treponema; Citrobacter; Bacillus; Streptococcus; Saccharomyces; and Brevibacterium. In some embodiments, the probiotic microorganism is a food degree bacteria (e.g., recognized as a “food degree” or “food safe” or “food grade” microorganism by the U.S. Food and Drug Administration or otherwise safe or non-hazardous to be present in a food or beverage); a non-limiting example of such a food degree bacteria is Bacillus subtilis. In some embodiments of any of the aspects, the probiotic microorganism is Escherichia coli (e.g., strain BL21). In some embodiments of any of the aspects, the probiotic microorganism is engineered from lactic acid bacteria.

### Methionine-Reducing Microorganism

In various aspects, described herein are engineered probiotic microorganism for reducing bioavailable methionine levels, and methods of reducing bioavailable methionine levels by administering such an engineered probiotic microorganism. As used herein, the term “bioavailable methionine” refers to methionine that can be absorbed from the gastrointestinal tract and enter circulation to thus have an active effect. In some embodiments, bioavailable methionine is measured using HPLC analysis of blood amino acid content (see e.g., Example 5).

In one aspect, described herein is an engineered probiotic microorganism for reducing bioavailable methionine levels, comprising: (a) at least one exogenous copy of at least one gene encoding an enzyme that catalyzes the degradation of methionine; (b) at least one exogenous copy of at least one functional methionine importer gene; (c) at least one endogenous methionine importer gene comprising at least one engineered activating modification; (d) at least one endogenous methionine synthesis gene comprising at least one engineered inactivating modification; (e) at least one endogenous methionine regulator gene comprising at least one engineered inactivating or activating modification; or (g) a combination of two or more of (a)-(e). Non-limiting examples of such combinations are provided in Table 2.

TABLE 2
Exemplary engineered probiotic microorganisms.

| | | | (d) | |
|---|---|---|---|---|
| (a) | (b) | (c) activated | inactivated | (e) modulated |
| exogenous | exogenous | endogenous | endogenous | endogenous |
| methionase | importer | importer | synthase | regulator |
| X | | | | |
| | X | | | |
| X | X | | | |
| | | X | | |
| X | | X | | |
| | X | X | | |
| X | X | X | | |
| | | | X | |
| X | | | X | |
| | X | | X | |
| X | X | | X | |
| | | X | X | |
| X | | X | X | |
| | X | X | X | |
| X | X | X | X | |
| | | | | X |
| X | | | | X |
| | X | | | X |
| X | X | | | X |
| | | X | | X |
| X | | X | | X |
| | X | X | | X |
| X | X | X | | X |
| | | | X | X |
| X | | | X | X |
| | X | | X | X |
| X | X | | X | X |
| | | X | X | X |
| X | | X | X | X |
| | X | X | X | X |
| X | X | X | X | X |

(“x” indicates inclusion in the microorganism).
(a) indicates at least one exogenous copy of at least one gene encoding an enzyme that catalyzes the degradation of methionine;
(b) indicates at least one exogenous copy of at least one functional methionine importer gene;
(c) indicates at least one endogenous methionine importer gene comprising at least one engineered activating modification;
(d) indicates at least one endogenous methionine synthesis gene comprising at least one engineered inactivating modification; and
(e) indicates at least one endogenous methionine regulator gene comprising at least one engineered inactivating or activating modification.

In some embodiments of any of the aspects, the exogenous gene(s) of (a) and/or (b), if present, and the endogenous gene(s) of (c), (d), (e), and/or (f) are expressed by the engineered probiotic microorganism under conditions in the gut, e.g., physiologically relevant conditions of the mammalian gastrointestinal (GI) tract, including the post-gastric GI tract, including the small intestine (duodenum, jejunum, ileum) and/or colon. “Physiologically relevant condition” of the gastrointestinal tract is understood to mean conditions found in the gastrointestinal tract, e.g., the human GI tract, or relevant portion thereof (e.g., small intestine, colon, etc.). For example, anaerobic conditions and a pH range of about 7-8, 8-9 or a pH of at least 7, at least 7.5, at least 8, at least 8.5, or at least 9 or more. It can also mean conditions such as levels of nutrients or other bacteria and/or their metabolites/proteins as found in the human gut.

In one aspect described herein is a method of reducing bioavailable methionine in a mammal in need thereof. In one aspect, the method comprises administering an engineered probiotic microorganism as described herein, a pharmaceutical composition as described herein, a food composition as described herein, or a probiotic dietary supplement as described herein to the mammal. In some embodiments of any of the aspects, the administering reduces the level of bioavailable methionine in the gut of the mammal. In some embodiments of any of the aspects, the level of bioavailable methionine is reduced in the small intestine, duodenum, jejunum, ileum, cecum, ileocecum, appendix, ascending colon, transverse colon, descending colon, sigmoid colon, rectum, or anus of the mammal. In some embodiments of any of the aspects, the administering is oral or rectal.

In some embodiments of any of the aspects, the level of bioavailable methionine is reduced by at least 5%. In some embodiments of any of the aspects, the level of bioavailable methionine is reduced by at least 50%. In some embodiments of any of the aspects, the level of bioavailable methionine is reduced by at least 95%. In some embodiments of any of the aspects, the level of bioavailable methionine is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or more.

The engineered microorganisms described herein are administered so as to reduce the level of bioavailable methionine to levels that are associated with health benefits. As methionine is an essential amino acid in the mammalian diet, it is contemplated that the reduction of methionine is not so high as to completely or significantly deprive the mammalian body of a healthy level of methionine. Estimates of a healthy methionine level depend on the individual, e.g., their weight, and range from 10-20 mg/kg/day. The amount of methionine intake will depend upon diet and the amount of methionine-containing food consumed; those levels can be modulated to some extent by dietary modification. However, in some embodiments, the administered engineered microorganism as described herein reduces the level of bioavailable methionine in the mammal to the healthy range of 10-20 mg/kg/day, e.g., about 15 mg/kg/day. In some embodiments, the administered engineered microorganism as described herein reduces the level of bioavailable methionine in the mammal to at most 800 mg-1200 mg methionine per day. In some embodiments, the administered engineered microorganism as described herein reduces the level of bioavailable methionine in the mammal to at most 3200 mg methionine per day. In some embodiments, the administered engineered microorganism as described herein reduces the level of bioavailable methionine in the mammal to at most 500 mg, at most 600 mg, at most 700 mg, at most 800 mg, at most 900 mg, at most 1000 mg, at most 1100 mg, at most 1200 mg, at most 1300 mg, at most 1400 mg, at most 1500 mg, at most 1600 mg, at most 1700 mg, at most 1800 mg, at most 1900 mg, at most 2000 mg, at most 2100 mg, at most 2200 mg, at most 2300 mg, at most 2400 mg, at most 2500 mg, at most 2600 mg, at most 2700 mg, at most 2800 mg, at most 2900 mg, at most 3000 mg, at most 3100 mg, at most 3200 mg, at most 3300 mg, at most 3400 mg, or at most 3500 mg methionine per day.

### Methionine Degrading Enzyme

In one aspect, described herein is an engineered probiotic microorganism for reducing bioavailable methionine levels comprising at least one exogenous copy of at least one gene encoding an enzyme that catalyzes the degradation of methionine. An enzyme that catalyzes the degradation of methionine can also be referred to herein as a methionase or a methionine catabolic enzyme.

In some embodiments of any of the aspects, the enzyme that catalyzes the degradation of methionine generates methanethiol. In some embodiments of any of the aspects, the gene encoding an enzyme that catalyzes the degradation of methionine encodes a methionine gamma lyase.

In some embodiments of any of the aspects, the methionine gamma lyase is encoded by one of SEQ ID NO: 1-4 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NO: 1-4, that maintains the same or improved function, or a codon-optimized version thereof. In some embodiments of any of the aspects, the methionine gamma lyase gene is codon optimized according to a specific bacterium, such as Bacillus subtilis (see e.g., SEQ ID NOs: 2, 4).

In some embodiments of any of the aspects, the methionine gamma lyase comprises one of SEQ ID NO: 5-6 or an amino acid sequence that is at least 90% identical. In some embodiments of any of the aspects, the methionine gamma lyase comprises one of SEQ ID NO: 5-6 or an amino acid sequence that is at least 90% similar. In some embodiments of any of the aspects, the methionine gamma lyase comprises one of SEQ ID NO: 5-6 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NO: 5-6, that maintains the same function (e.g. degradation of methionine to methanethiol).

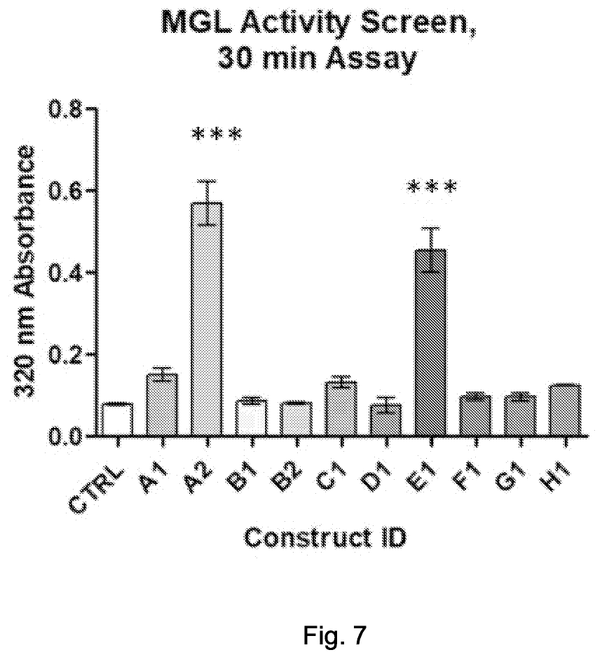

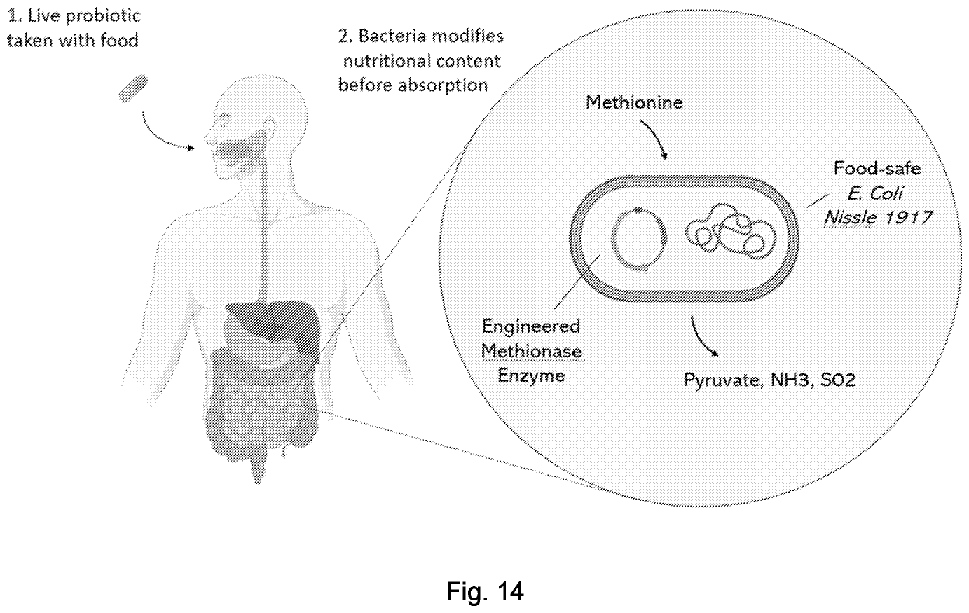

Methionase “BIOREACTOR 1”, 1200 nucleotides (nt)
SEQ ID NO: 1
ATGATGGAAAACGAACAGGAACTGGGCTTTGCGACCCGCCAGATTCATGTGGGCAAAAT
TAAAGAAGCGAGCGGCGCGCTGTGCACCCCGATTTATCAGACCAGCACCTTTGAATTTGA
AACCGTGCAGCAGGGCGGCGCGCGCTTTGCGGGCGAAGAACCGGGCTATATTTATAGCC
GCCTGAGCAACCCGAGCCTGGCGCAGGCGGAAGAAAAACTGGCGAGCCTGGAAAACGG
CGAAGCGGCGCTGGCGGCGGCGAGCGGCATGGGCGCGATTAGCGCGGCGCTGTGGACCA
GCGTGGTGGCGGGCGATGAAATTCTGGCGAGCGATACCCTGTATGGCTGCACCTTTAGCC
TGCTGTGCCATGGCATGACCAAATTTGGCGTGGATACCAAATTTATTGATATGGTGAACC
TGGAAAACTTTCAGAAACATCTGACCCCGAAAACCAAAGTGATTTATTTTGAAACCCCGT
GCAACCCGACCCTGAAAATTCTGGATATTCGCGCGATTGCGGAAGCGGCGCATAAATAT
AACCCGGCGATTCGCGTGATGGTGGATAACACCTTTTGCAGCCCGTATCTGCAGCGCCCG
CTGGAACTGGGCGCGGATGTGGTGGTGCATAGCGCGACCAAATATATTAACGGCCATGG
CGATGTGATTGCGGGCTTTATTGTGGGCACCGCGGAATTTATTGGCCAGTGCCGCACCTT
TGGCCTGAAAGATATGACCGGCGCGGTGATGAGCCCGTTTGATGCGTTTCTGATTGCGCG
CGGCCTGAAAACCCTGGATATTCGCATGGAACGCCATTGCAGCAACGCGCGCAAAGTGG
CGGAATTTCTGCATAGCCATCCGGCGGTGGAAAAAGTGTATTATCCGGGCCTGCCGGATT
TTAAAGGCTATGAAGTGGCGCAGAAACAGATGAAAGATTTTGGCGGCATGCTGAGCATT
GAACTGAAAGCGAGCCGCGAAGAAGTGGCGAACGCGCTGAACAACCTGCGCCTGTGCAC
CATTGCGGTGAGCCTGGGCGATGCGGAAACCCTGGTGGAACATGCGGCGAGCATGACCC
ATAGCACCTATACCCCGGAAGAACTGGCGGCGGCGGGCATTAGCGAAGGCCTGGTGCGC
ATTAGCGTGGGCCTGGAAGATCCGGATGATATTATTGCGGATCTGAAAAGCGTGCTGGA
TACCCTGGTGAGC,
Methionase “BIOREACTOR 1” BACSU, 1200 nucleotides (nt)
SEQ ID NO: 2
ATGATGGAAAATGAACAGGAGCTTGGTTTCGCCACAAGGCAGATACATGTCGGCAAGAT
TAAAGAGGCTTCGGGTGCGTTGTGTACCCCGATCTACCAAACTTCTACGTTTGAATTCGA
AACTGTGCAACAAGGAGGTGCGCGCTTTGCCGGCGAAGAACCGGGCTATATTTATTCAC
GCTTAAGCAACCCTTCCCTTGCCCAGGCAGAGGAGAAACTCGCCTCACTGGAAAACGGA
GAAGCGGCTCTGGCAGCGGCGTCGGGAATGGGCGCAATATCAGCCGCCCTGTGGACCTC
TGTTGTCGCTGGCGATGAAATCCTTGCTTCAGATACCCTGTATGGATGCACGTTTTCATTA
CTATGCCATGGAATGACCAAATTTGGTGTAGATACTAAATTTATTGATATGGTAAACCTT
GAAAACTTTCAGAAGCACCTTACGCCGAAAACGAAAGTGATTTACTTTGAAACGCCTTGC
AACCCGACACTCAAAATTTTGGATATCCGTGCAATCGCAGAAGCTGCTCATAAATATAAT
CCAGCAATTCGAGTTATGGTCGATAATACATTTTGTAGCCCCTATTTGCAACGGCCGCTT
GAACTGGGCGCCGATGTCGTAGTGCACAGCGCCACAAAGTACATCAATGGCCACGGTGA
CGTGATTGCAGGCTTCATTGTTGGGACTGCTGAGTTCATTGGGCAATGCCGGACATTTGG
ACTGAAAGACATGACCGGGGCAGTCATGTCTCCGTTCGATGCTTTTCTAATCGCTCGTGG
CTTAAAAACATTGGACATTCGTATGGAAAGACATTGTAGTAATGCGAGAAAGGTAGCAG
AGTTCCTGCACTCCCATCCAGCGGTTGAAAAAGTCTATTATCCAGGGCTTCCTGACTTTA
AGGGATATGAAGTTGCTCAGAAACAAATGAAAGATTTTGGTGGGATGTTAAGTATCGAA
CTGAAGGCGTCAAGAGAAGAAGTGGCGAATGCATTGAATAACTTAAGACTCTGTACAAT
TGCGGTCAGCCTCGGCGACGCTGAAACGTTAGTAGAGCATGCGGCGAGTATGACACATT
CCACATACACACCTGAGGAGCTGGCAGCCGCCGGAATCAGCGAGGGACTTGTTCGCATA
TCTGTGGGATTAGAAGATCCGGACGACATTATCGCAGATCTTAAATCTGTTTTGGATACG
TTAGTGAGC,
Methionase “SPIROCHETE 1,″ 1203 nt
SEQ ID NO: 3
ATGAATCGCAAAGAGCTGGAAAAACTGGGGTTTGCGTCTAAACAAATCCACGCGGGCAG
CATCAAAAATAAGTACGGTGCTCTGGCTACCCCCATTTACCAGACCTCGACTTTCGCTTTT
GATTCCGCTGAACAAGGTGGCCGTCGTTTCGCACTGGAGGAGGAGGGTTATATCTACAC
ACGTTTGGGGAATCCGACCACAACTGTGGTTGAAGAAAAACTTGCATGTCTTGAAAACG
GCGAGGCTTGTATGAGCGCTTCTTCTGGGATTGGGGCCGTTACGTCGTGCATCTGGAGCA
TCGTTAACGCTGGCGACCATATTGTAGCCGGCAAGACCTTGTACGGGTGTACATTTGCAT
TCCTGAATCATGGGTTGAGCCGCTTTGGAGTTGATGTTACTTTTGTCGATACCCGCGATCC
CGAGAATGTAAAGAAAGCCTTGAAACCCAACACCAAAATCGTTTATCTGGAAACGCCAG
CGAACCCGAACATGTATTTGTGTGATATTGCAGCTGTTTCCAAAATTGCGCATGCCCACA
ACCCGGAGTGCAAGGTCATCGTTGATAACACGTATATGACCCCGTACCTGCAGCGGCCCC
TTGATCTGGGGGCCGATGTGGTGCTGCACAGTGCAACCAAATATCTGAACGGCCATGGC
GATGTCATCGCCGGTTTCGTGGTCGGCAAAAAAGAGTTTATTGATCAGGTGCGGTTTGTA
GGCGTTAAGGACATGACGGGCTCTACACTGGGTCCTTTCGAAGCGTACCTGATCGGCCGC
GGAATGAAAACACTGGACATTCGGATGGAAAAACACTGCGCCAATGCTCAAAAAGTAGC
GGAGTTCTTGGAAAAACACCCAGCGGTTGAGAGCATCGCTTTCCCTGGTCTGAAATCCTT
CCCACAGTATGAACTCGCCAAGAAACAGATGAAGCTCTGTGGTGCGATGATTGCGTTCA
CCGTAAAAGGGGGCCTTGAAGCTGGTAAAACTCTCATCAACTCCGTTAAGTTCGCCACTA
TTGCCGTGTCGCTCGGCGATGCCGAGACCCTGATTCAACATCCGGCAAGCATGACTCATT
CCCCATACACCCCAGAGGAGCGCGCAGCATCCGACATTGCCGAGGGCCTGGTCCGCTTA
AGTGTAGGTCTGGAAGATGCCGAAGATATTATTGCCGATCTGAAACAAGCTCTGGATAA
ACTTGTAAAA,
Methionase “SPIROCHETE 1,″ BACSU 1203 nt
SEQ ID NO: 4
ATGAACCGGAAAGAGCTTGAAAAATTGGGCTTTGCAAGCAAACAAATTCATGCAGGGAG
CATTAAAAATAAATACGGAGCCCTGGCCACACCGATCTACCAGACCAGCACATTCGCGT
TTGATTCAGCTGAGCAAGGCGGCCGGAGATTCGCATTAGAAGAAGAAGGATATATATAT
ACGCGCCTCGGGAATCCTACAACGACAGTAGTTGAGGAGAAACTGGCATGTTTAGAAAA
TGGAGAAGCATGTATGTCCGCATCATCAGGCATTGGCGCAGTGACTAGTTGTATCTGGTC
TATCGTTAATGCGGGTGATCACATTGTCGCCGGTAAAACGTTATATGGCTGCACGTTTGC
TTTTCTGAACCACGGTTTAAGTCGTTTCGGGGTCGATGTAACCTTTGTGGATACAAGGGA
TCCTGAAAATGTAAAGAAGGCCCTTAAGCCGAATACAAAAATTGTCTATTTGGAGACAC
CAGCAAACCCGAACATGTATCTCTGCGATATCGCGGCGGTTAGCAAGATTGCCCATGCCC
ATAATCCTGAGTGTAAAGTGATCGTAGACAACACCTATATGACGCCTTATTTGCAGCGTC
CGTTGGACTTGGGAGCTGATGTGGTGCTCCACTCTGCGACAAAGTACCTCAACGGACATG
GTGATGTCATAGCCGGTTTTGTGGTTGGCAAGAAAGAATTTATTGACCAGGTGAGATTCG
TCGGAGTTAAGGACATGACGGGCTCAACGCTGGGACCGTTTGAAGCATACCTTATCGGA
CGCGGGATGAAAACCCTTGATATTAGAATGGAAAAACATTGCGCAAATGCACAAAAAGT
GGCGGAATTTCTGGAAAAACATCCAGCTGTTGAATCAATTGCTTTCCCTGGGCTAAAATC
GTTTCCGCAGTACGAGCTGGCTAAGAAACAAATGAAACTTTGCGGGGCCATGATCGCGT
TTACAGTAAAAGGAGGCCTTGAAGCGGGCAAAACACTTATAAACTCCGTCAAGTTTGCC
ACTATCGCTGTTTCCCTGGGCGACGCGGAGACTCTGATTCAGCACCCGGCCAGCATGACT
CATTCTCCCTATACCCCAGAAGAGCGAGCTGCTTCTGATATTGCTGAAGGTTTGGTACGC
TTATCGGTTGGATTAGAAGATGCGGAAGATATCATTGCAGACTTAAAACAAGCGCTTGA
CAAACTAGTCAAA,
methionine gamma lyase, Methionase “BIOREACTOR 1,″ MGL “E1″
in FIG. 7, MGL “2″ in FIG. 15, 400 amino acids (aa)
SEQ ID NO: 5
MMENEQELGFATRQIHVGKIKEASGALCTPIYQTSTFEFETVQQGGARFAGEEPGYIYSRLSN
PSLAQAEEKLASLENGEAALAAASGMGAISAALWTSVVAGDEILASDTLYGCTFSLLCHGMT
KFGVDTKFIDMVNLENFQKHLTPKTKVIYFETPCNPTLKILDIRAIAEAAHKYNPAIRVMVDN
TFCSPYLQRPLELGADVVVHSATKYINGHGDVIAGFIVGTAEFIGQCRTFGLKDMTGAVMSPF
DAFLIARGLKTLDIRMERHCSNARKVAEFLHSHPAVEKVYYPGLPDFKGYEVAQKQMKDFG
GMLSIELKASREEVANALNNLRLCTIAVSLGDAETLVEHAASMTHSTYTPEELAAAGISEGLV
RISVGLEDPDDIIADLKSVLDTLVS,
Methionase “SPIROCHETE 1,″ MGL “A2″ in FIG. 7, MGL “8″ in FIG.
15, 401 aa
SEQ ID NO: 6
MNRKELEKLGFASKQIHAGSIKNKYGALATPIYQTSTFAFDSAEQGGRRFALEEEGYIYTRLG
NPTTTVVEEKLACLENGEACMSASSGIGAVTSCIWSIVNAGDHIVAGKTLYGCTFAFLNHGLS
RFGVDVTFVDTRDPENVKKALKPNTKIVYLETPANPNMYLCDIAAVSKIAHAHNPECKVIVD
NTYMTPYLQRPLDLGADVVLHSATKYLNGHGDVIAGFVVGKKEFIDQVRFVGVKDMTGSTL
GPFEAYLIGRGMKTLDIRMEKHCANAQKVAEFLEKHPAVESIAFPGLKSFPQYELAKKQMKL
CGAMIAFTVKGGLEAGKTLINSVKFATIAVSLGDAETLIQHPASMTHSPYTPEERAASDIAEG
LVRLSVGLEDAEDIIADLKQALDKLVK,

In one embodiment, the methionine gamma lyase gene is a methionine gamma lyase gene from Bacillus halodurans. In one embodiment, the methionine gamma lyase is an Entamoeba histolytica methionine gamma lyase gene.

In some embodiments, the methionine gamma lyase gene is a methionine gamma lyase gene from the genus Oscillibacter. In some embodiments, the methionine gamma lyase gene is a methionine gamma lyase gene from Oscillibacter rumenantium (e.g., the closest species found in nature to the bioreactor metagenome SEQ ID NO: 5 is O. rumenantium). In one embodiment, the methionine gamma lyase gene is a methionine gamma lyase gene from the genus Treponema. In some embodiments, the methionine gamma lyase is a methionine gamma lyase gene from Treponema denticola (e.g., SEQ ID NO: 6).

In some embodiments, the methionine gamma lyase gene is not from any of the following genera: Bacillus, Entamoeba, Brevibacterium, Citrobacter, or Porphyromonas. In some embodiments, the methionine gamma lyase gene is not from any of the following species: Bacillus halodurans, Entamoeba histolytica, Brevibacterium aurantiacum, Citrobacter freundii, or Porphyromonas gingivalis.

In some embodiments of any of the aspects, the gene encoding an enzyme that catalyzes the degradation of methionine encodes expression of a catalytically-active fragment of a methionine gamma lyase. In some embodiments of any of the aspects, the enzyme that catalyzes the degradation of methionine comprises a catalytically-active fragment of a methionine gamma lyase. In some embodiments of any of the aspects, the enzyme that catalyzes the degradation of methionine comprises a catalytically-active fragment of one of SEQ ID NO: 5-6.

In some embodiments of any of the aspects, the gene encoding an enzyme that catalyzes the degradation of methionine encodes expression of a fusion protein comprising a catalytically-active fragment of a methionine gamma lyase. In some embodiments of any of the aspects, the fusion protein comprising a catalytically-active fragment of a methionine gamma lyase and a catalytically-active fragment of an esterase. In some embodiments of any of the aspects, the fusion protein comprises a catalytically-active fragment of a methionine gamma lyase and a catalytically-active fragment of a methanethiol oxidase.

In some embodiments of any of the aspects, the gene encoding an enzyme that catalyzes the degradation of methionine further comprises a protein secretion signal sequence. The protein secretion signal sequence allows for extracellular secretion of the enzyme. In some embodiments, the protein secretion signal sequence is derived from a Gram-positive bacterium (see e.g., Tables 10-11). In some embodiments, the protein signal secretion sequence is C-terminal of the enzyme (or 3′ of the enzyme in a nucleic acid encoding it). In some embodiments, the protein secretion signal sequence is N-terminal of the enzyme (or 5′ of the enzyme in a nucleic acid encoding it).

In some embodiments of any of the aspects, the protein secretion signal sequence is encoded by one of SEQ ID NOs: 263-435 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 263-435, that maintains the same function, or a codon-optimized version thereof (see e.g., Table 11, Example 4).

In some embodiments of any of the aspects, the protein secretion signal sequence comprises one of SEQ ID NOs: 90-262 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NOs: 90-262, that maintains the same function (e.g., protein secretion, e.g., in a Gram-positive bacterium; see e.g., Table 10, Example 4).

In some embodiments of any of the aspects, the fusion protein comprises a fusion of a methionine gamma lyase and a methanethiol oxidase (i.e., an MGL-MTO chimera). In some embodiments of any of the aspects, the MGL-MTO fusion protein is encoded by one of SEQ ID NO: 7-8 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NO: 7-8, that maintains the same function, or a codon-optimized version thereof.

In some embodiments of any of the aspects, the MGL-MTO fusion protein comprises one of SEQ ID NO: 9-10 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NO: 9-10, that maintains the same function (e.g., degradation of methionine to methanethiol and/or degradation of methanethiol).

MGL-MTO Chimera 1, 2610 nt
SEQ ID NO: 7
ATGATGGAAAACGAACAGGAACTGGGCTTTGCGACCCGCCAGATTCATGTGGGCAAAAT
TAAAGAAGCGAGCGGCGCGCTGTGCACCCCGATTTATCAGACCAGCACCTTTGAATTTGA
AACCGTGCAGCAGGGCGGCGCGCGCTTTGCGGGCGAAGAACCGGGCTATATTTATAGCC
GCCTGAGCAACCCGAGCCTGGCGCAGGCGGAAGAAAAACTGGCGAGCCTGGAAAACGG
CGAAGCGGCGCTGGCGGCGGCGAGCGGCATGGGCGCGATTAGCGCGGCGCTGTGGACCA
GCGTGGTGGCGGGCGATGAAATTCTGGCGAGCGATACCCTGTATGGCTGCACCTTTAGCC
TGCTGTGCCATGGCATGACCAAATTTGGCGTGGATACCAAATTTATTGATATGGTGAACC
TGGAAAACTTTCAGAAACATCTGACCCCGAAAACCAAAGTGATTTATTTTGAAACCCCGT
GCAACCCGACCCTGAAAATTCTGGATATTCGCGCGATTGCGGAAGCGGCGCATAAATAT
AACCCGGCGATTCGCGTGATGGTGGATAACACCTTTTGCAGCCCGTATCTGCAGCGCCCG
CTGGAACTGGGCGCGGATGTGGTGGTGCATAGCGCGACCAAATATATTAACGGCCATGG
CGATGTGATTGCGGGCTTTATTGTGGGCACCGCGGAATTTATTGGCCAGTGCCGCACCTT
TGGCCTGAAAGATATGACCGGCGCGGTGATGAGCCCGTTTGATGCGTTTCTGATTGCGCG
CGGCCTGAAAACCCTGGATATTCGCATGGAACGCCATTGCAGCAACGCGCGCAAAGTGG
CGGAATTTCTGCATAGCCATCCGGCGGTGGAAAAAGTGTATTATCCGGGCCTGCCGGATT
TTAAAGGCTATGAAGTGGCGCAGAAACAGATGAAAGATTTTGGCGGCATGCTGAGCATT
GAACTGAAAGCGAGCCGCGAAGAAGTGGCGAACGCGCTGAACAACCTGCGCCTGTGCAC
CATTGCGGTGAGCCTGGGCGATGCGGAAACCCTGGTGGAACATGCGGCGAGCATGACCC
ATAGCACCTATACCCCGGAAGAACTGGCGGCGGCGGGCATTAGCGAAGGCCTGGTGCGC
ATTAGCGTGGGCCTGGAAGATCCGGATGATATTATTGCGGATCTGAAAAGCGTGCTGGA
TACCCTGGTGAGCGGCAGCAGCGGCAGCAGCGGCAGCAGCGGCAGCAGCGGCAGCAGC
GGCAGCAGCGGCAGCAGCGGCAGCAGCGGCAGCAGCGGCAGCAGCGGCAGCAGCGGCA
GCAGCATGAAAAAACATCTGCTGGCGGGCGCGTGCGCGCTGGCGATGGGCTTTGCGGTG
ATTCCGGGCACCTTTGCGGATGAAACCTGCAACAGCCCGTTTACCACCGCGCTGATTACC
GGCCAGGAACAGTATCTGCATGTGTGGACCCTGGGCATGCCGGGCGTGGGCGATGAAAG
CGATAAACTGGTGACCATTAGCGTGGATCCGAAAAGCGATAAATATGGCAAAGTGATTA
ACACCCTGAGCGTGGGCGGCCGCGGCGAAGCGCATCATACCGGCTTTACCGATGATCGC
CGCTATCTGTGGGCGGGCCGCCTGGATGATAACAAAATTTTTATTTTTGATCTGATTGATC
CGGCGAACCCGAAACTGATTAAAACCATTACCGATTTTGCGGATCGCACCGGCTATGTGG
GCCCGCATACCTTTTATGCGCTGCCGGGCCGCATGCTGATTCAGGCGCTGAGCAACACCA
AAACCCATGATGGCCAGACCGGCCTGGCGGTGTATAGCAACGCGGGCGAACTGGTGAGC
CTGCATCCGATGCCGGTGACCGATGGCGGCGATGGCTATGGCTATGATATTGGCATTAAC
CCGGCGAAAAACGTGCTGCTGACCAGCAGCTTTACCGGCTGGAACAACTATATGATGGA
TCTGGGCAAAATGGTGAAAGATCCGGAAGCGATGAAACGCTTTGGCAACACCATGGCGA
TTTGGGATCTGAAAAGCATGAAAGCGGAAAAAATTCTGAACGTGCCGGGCGCGCCGCTG
GAAATTCGCTGGAGCCTGAAACCGGAACATAACTGGGCGTATACCGCGACCCTGACCAG
CAAACTGTGGCTGATTAAACAGGATGATAAAGGCGAATGGATTGCGAAAGAAACCGGCA
CCATTGGCGATCCGAGCAAAATTCCGCTGCCGGTGGATATTAGCATTACCGCGGATGCGA
AAGGCCTGTGGGTGAACACCTTTCTGGATGGCACCACCCGCTTTTATGATATTAGCGAAC
CGGAACATCCGAAAGAAGTGTTTAGCAAAAAAATGGGCAACCAGGTGAACATGGTGAG
CCAGAGCTATGATGGCAAACGCGTGTATTTTACCACCAGCCTGATTGCGAACTGGGATAA
AAAAGGCGCGGAAAACGATCAGTGGCTGAAAGCGTATGATTGGGATGGCAAAGAACTG
GTGGAAAAATTTACCGTGGATTTTAACGAACTGAAACTGGGCCGCGCGCATCATATGAA
ATTTAGCAGCAAAACCAACGCGGCGGAACTGGGCACCAACCAGAGCTTTCCGACCCGCC
AG,
MGL-MTO Chimera 2, 2610 nt
SEQ ID NO: 8
ATGAAAAAACATCTGCTGGCGGGCGCGTGCGCGCTGGCGATGGGCTTTGCGGTGATTCC
GGGCACCTTTGCGGATGAAACCTGCAACAGCCCGTTTACCACCGCGCTGATTACCGGCCA
GGAACAGTATCTGCATGTGTGGACCCTGGGCATGCCGGGCGTGGGCGATGAAAGCGATA
AACTGGTGACCATTAGCGTGGATCCGAAAAGCGATAAATATGGCAAAGTGATTAACACC
CTGAGCGTGGGCGGCCGCGGCGAAGCGCATCATACCGGCTTTACCGATGATCGCCGCTA
TCTGTGGGCGGGCCGCCTGGATGATAACAAAATTTTTATTTTTGATCTGATTGATCCGGC
GAACCCGAAACTGATTAAAACCATTACCGATTTTGCGGATCGCACCGGCTATGTGGGCCC
GCATACCTTTTATGCGCTGCCGGGCCGCATGCTGATTCAGGCGCTGAGCAACACCAAAAC
CCATGATGGCCAGACCGGCCTGGCGGTGTATAGCAACGCGGGCGAACTGGTGAGCCTGC
ATCCGATGCCGGTGACCGATGGCGGCGATGGCTATGGCTATGATATTGGCATTAACCCGG
CGAAAAACGTGCTGCTGACCAGCAGCTTTACCGGCTGGAACAACTATATGATGGATCTG
GGCAAAATGGTGAAAGATCCGGAAGCGATGAAACGCTTTGGCAACACCATGGCGATTTG
GGATCTGAAAAGCATGAAAGCGGAAAAAATTCTGAACGTGCCGGGCGCGCCGCTGGAA
ATTCGCTGGAGCCTGAAACCGGAACATAACTGGGCGTATACCGCGACCCTGACCAGCAA
ACTGTGGCTGATTAAACAGGATGATAAAGGCGAATGGATTGCGAAAGAAACCGGCACCA
TTGGCGATCCGAGCAAAATTCCGCTGCCGGTGGATATTAGCATTACCGCGGATGCGAAA
GGCCTGTGGGTGAACACCTTTCTGGATGGCACCACCCGCTTTTATGATATTAGCGAACCG
GAACATCCGAAAGAAGTGTTTAGCAAAAAAATGGGCAACCAGGTGAACATGGTGAGCC
AGAGCTATGATGGCAAACGCGTGTATTTTACCACCAGCCTGATTGCGAACTGGGATAAA
AAAGGCGCGGAAAACGATCAGTGGCTGAAAGCGTATGATTGGGATGGCAAAGAACTGG
TGGAAAAATTTACCGTGGATTTTAACGAACTGAAACTGGGCCGCGCGCATCATATGAAA
TTTAGCAGCAAAACCAACGCGGCGGAACTGGGCACCAACCAGAGCTTTCCGACCCGCCA
GGGCAGCAGCGGCAGCAGCGGCAGCAGCGGCAGCAGCGGCAGCAGCGGCAGCAGCGGC
AGCAGCGGCAGCAGCGGCAGCAGCGGCAGCAGCGGCAGCAGCGGCAGCAGCATGATGG
AAAACGAACAGGAACTGGGCTTTGCGACCCGCCAGATTCATGTGGGCAAAATTAAAGAA
GCGAGCGGCGCGCTGTGCACCCCGATTTATCAGACCAGCACCTTTGAATTTGAAACCGTG
CAGCAGGGCGGCGCGCGCTTTGCGGGCGAAGAACCGGGCTATATTTATAGCCGCCTGAG
CAACCCGAGCCTGGCGCAGGCGGAAGAAAAACTGGCGAGCCTGGAAAACGGCGAAGCG
GCGCTGGCGGCGGCGAGCGGCATGGGCGCGATTAGCGCGGCGCTGTGGACCAGCGTGGT
GGCGGGCGATGAAATTCTGGCGAGCGATACCCTGTATGGCTGCACCTTTAGCCTGCTGTG
CCATGGCATGACCAAATTTGGCGTGGATACCAAATTTATTGATATGGTGAACCTGGAAAA
CTTTCAGAAACATCTGACCCCGAAAACCAAAGTGATTTATTTTGAAACCCCGTGCAACCC
GACCCTGAAAATTCTGGATATTCGCGCGATTGCGGAAGCGGCGCATAAATATAACCCGG
CGATTCGCGTGATGGTGGATAACACCTTTTGCAGCCCGTATCTGCAGCGCCCGCTGGAAC
TGGGCGCGGATGTGGTGGTGCATAGCGCGACCAAATATATTAACGGCCATGGCGATGTG
ATTGCGGGCTTTATTGTGGGCACCGCGGAATTTATTGGCCAGTGCCGCACCTTTGGCCTG
AAAGATATGACCGGCGCGGTGATGAGCCCGTTTGATGCGTTTCTGATTGCGCGCGGCCTG
AAAACCCTGGATATTCGCATGGAACGCCATTGCAGCAACGCGCGCAAAGTGGCGGAATT
TCTGCATAGCCATCCGGCGGTGGAAAAAGTGTATTATCCGGGCCTGCCGGATTTTAAAGG
CTATGAAGTGGCGCAGAAACAGATGAAAGATTTTGGCGGCATGCTGAGCATTGAACTGA
AAGCGAGCCGCGAAGAAGTGGCGAACGCGCTGAACAACCTGCGCCTGTGCACCATTGCG
GTGAGCCTGGGCGATGCGGAAACCCTGGTGGAACATGCGGCGAGCATGACCCATAGCAC
CTATACCCCGGAAGAACTGGCGGCGGCGGGCATTAGCGAAGGCCTGGTGCGCATTAGCG
TGGGCCTGGAAGATCCGGATGATATTATTGCGGATCTGAAAAGCGTGCTGGATACCCTG
GTGAGC,
MGL-MTO Chimera 1, 870 aa
SEQ ID NO: 9
MMENEQELGFATRQIHVGKIKEASGALCTPIYQTSTFEFETVQQGGARFAGEEPGYIYSRLSN
PSLAQAEEKLASLENGEAALAAASGMGAISAALWTSVVAGDEILASDTLYGCTFSLLCHGMT
KFGVDTKFIDMVNLENFQKHLTPKTKVIYFETPCNPTLKILDIRAIAEAAHKYNPAIRVMVDN
TFCSPYLQRPLELGADVVVHSATKYINGHGDVIAGFIVGTAEFIGQCRTFGLKDMTGAVMSPF
DAFLIARGLKTLDIRMERHCSNARKVAEFLHSHPAVEKVYYPGLPDFKGYEVAQKQMKDFG
GMLSIELKASREEVANALNNLRLCTIAVSLGDAETLVEHAASMTHSTYTPEELAAAGISEGLV
RISVGLEDPDDIIADLKSVLDTLVSGSSGSSGSSGSSGSSGSSGSSGSSGSSGSSGSSGSSMKKH
LLAGACALAMGFAVIPGTFADETCNSPFTTALITGQEQYLHVWTLGMPGVGDESDKLVTISV
DPKSDKYGKVINTLSVGGRGEAHHTGFTDDRRYLWAGRLDDNKIFIFDLIDPANPKLIKTITD
FADRTGYVGPHTFYALPGRMLIQALSNTKTHDGQTGLAVYSNAGELVSLHPMPVTDGGDGY
GYDIGINPAKNVLLTSSFTGWNNYMMDLGKMVKDPEAMKRFGNTMAIWDLKSMKAEKILN
VPGAPLEIRWSLKPEHNWAYTATLTSKLWLIKQDDKGEWIAKETGTIGDPSKIPLPVDISITAD
AKGLWVNTFLDGTTRFYDISEPEHPKEVFSKKMGNQVNMVSQSYDGKRVYFTTSLIANWDK
KGAENDQWLKAYDWDGKELVEKFTVDFNELKLGRAHHMKFSSKTNAAELGTNQSFPTRQ,
MGL-MTO Chimera 2, 870 aa
SEQ ID NO: 10
MKKHLLAGACALAMGFAVIPGTFADETCNSPFTTALITGQEQYLHVWTLGMPGVGDESDKL
VTISVDPKSDKYGKVINTLSVGGRGEAHHTGFTDDRRYLWAGRLDDNKIFIFDLIDPANPKLI
KTITDFADRTGYVGPHTFYALPGRMLIQALSNTKTHDGQTGLAVYSNAGELVSLHPMPVTD
GGDGYGYDIGINPAKNVLLTSSFTGWNNYMMDLGKMVKDPEAMKRFGNTMAIWDLKSMK
AEKILNVPGAPLEIRWSLKPEHNWAYTATLTSKLWLIKQDDKGEWIAKETGTIGDPSKIPLPV
DISITADAKGLWVNTFLDGTTRFYDISEPEHPKEVFSKKMGNQVNMVSQSYDGKRVYFTTSL
IANWDKKGAENDQWLKAYDWDGKELVEKFTVDFNELKLGRAHHMKFSSKTNAAELGTNQ
SFPTRQGSSGSSGSSGSSGSSGSSGSSGSSGSSGSSGSSGSSMMENEQELGFATRQIHVGKIKEA
SGALCTPIYQTSTFEFETVQQGGARFAGEEPGYIYSRLSNPSLAQAEEKLASLENGEAALAAA
SGMGAISAALWTSVVAGDEILASDTLYGCTFSLLCHGMTKFGVDTKFIDMVNLENFQKHLTP
KTKVIYFETPCNPTLKILDIRAIAEAAHKYNPAIRVMVDNTFCSPYLQRPLELGADVVVHSAT
KYINGHGDVIAGFIVGTAEFIGQCRTFGLKDMTGAVMSPFDAFLIARGLKTLDIRMERHCSNA
RKVAEFLHSHPAVEKVYYPGLPDFKGYEVAQKQMKDFGGMLSIELKASREEVANALNNLRL
CTIAVSLGDAETLVEHAASMTHSTYTPEELAAAGISEGLVRISVGLEDPDDIIADLKSVLDTLVS,

### Methionine Importer

One way of reducing bioavailable methionine is for bacteria in the gut to take methionine up from their environment to sequester and/or degrade it. In one aspect, described herein is an engineered probiotic microorganism for reducing bioavailable methionine levels comprising at least one exogenous copy of at least one functional methionine importer gene. In some embodiments of any of the aspects, the exogenous methionine importer gene comprises at least one engineered activating modification, e.g., a mutation that increases the rate of methionine import relative to wild-type of that enzyme. In some embodiments of any of the aspects, the engineered probiotic microorganism comprises at least one exogenous copy of at least one gene encoding an enzyme that catalyzes the degradation of methionine and at least one exogenous copy of at least one functional methionine importer gene.

In one aspect, described herein is an engineered probiotic microorganism for reducing bioavailable methionine levels comprising at least one endogenous methionine importer gene comprising at least one engineered activating modification, e.g., a mutation that increases the rate of methionine import relative to wild-type of that enzyme. In some embodiments of any of the aspects, the engineered probiotic microorganism comprises at least one exogenous copy of at least one gene encoding an enzyme that catalyzes the degradation of methionine and at least one exogenous copy of at least one functional methionine importer gene comprises a mutation that increases the rate of methionine import relative to wild-type of that enzyme.

Non-limiting examples of endogenous functional methionine importers include MetN (see e.g., SEQ ID NOs: 11-14, SEQ ID NOs: 23-26, SEQ ID NOs: 80-83); MetI (see e.g., SEQ ID NOs: 15-18. SEQ ID NOs: 84-85); MetP (see e.g., SEQ ID NOs: 27-30); or MetQ (see e.g., SEQ ID NO: 19-22, SEQ ID NOs: 31-34; SEQ ID NOs: 86-89). Non-limiting examples of mutations that increase the rate of methionine import relative to wild-type of that enzyme include N295A MetN (see e.g., SEQ ID NOs: 12, 14, 81, 83), N293A MetN (see e.g., SEQ ID NOs: 24, 26), Y160A MetI (see e.g., SEQ ID NOs: 16, 18), N229A MetQ (see e.g., SEQ ID NOs: 20, 22, 87, 89), and/or N231A MetQ (see e.g., SEQ ID NOs: 32, 34).

In some embodiments of any of the aspects, the methionine importer is encoded by one of SEQ ID NOs: 11, 12, 15, 16, 19, 20, 23, 24, 27, 28, 31, 32 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 11, 12, 15, 16, 19, 20, 23, 24, 27, 28, 31, or 32, that maintains the same function, or a codon-optimized version thereof.

In some embodiments of any of the aspects, the methionine importer comprises one of SEQ ID NOs: 13, 14, 17, 18, 21, 22, 25, 26, 29, 30, 33, 34, 80-89 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NOs: 13, 14, 17, 18, 21, 22, 25, 26, 29, 30, 33, 34, or 80-89 that maintains the same function (e.g., methionine import).

In some embodiments of any of the aspects, the engineered probiotic microorganism comprises at least one engineered activating modification in MetN (see e.g., SEQ ID NOs: 11-14, SEQ ID NOs: 23-26, 80-83), MetI or MetP (see e.g., SEQ ID NOs: 15-18, SEQ ID NOs: 27-30), or MetQ (see e.g., SEQ ID NO: 19-22, SEQ ID NOs: 31-34, 86-89). In some embodiments of any of the aspects, the engineered probiotic microorganism comprises at least one engineered activating modification in MetN (see e.g., SEQ ID NOs: 11-14, SEQ ID NOs: 23-26, 80-83). In some embodiments of any of the aspects, the engineered probiotic microorganism comprises at least one engineered activating modification in MetI or MetP (see e.g., SEQ ID NO: 15-18, SEQ ID NOs: 27-30). In some embodiments of any of the aspects, the engineered probiotic microorganism comprises at least one engineered activating modification in MetQ (see e.g., SEQ ID NO: 19-22, SEQ ID NOs: 31-34, 86-89). In some embodiments of any of the aspects, the engineered probiotic microorganism comprises at least one engineered activating modification in MetN (see e.g., SEQ ID NO: 11-14, SEQ ID NOs: 23-26, 80-83) and MetI or MetP (see e.g., SEQ ID NO: 15-18, SEQ ID NOs: 27-30). In some embodiments of any of the aspects, the engineered probiotic microorganism comprises at least one engineered activating modification in MetN (see e.g., SEQ ID NO: 11-14, SEQ ID NOs: 23-26, 80-83) and or MetQ (see e.g., SEQ ID NO: 19-22, SEQ ID NOs: 31-34, 86-89). In some embodiments of any of the aspects, the engineered probiotic microorganism comprises at least one engineered activating modification in MetI or MetP (see e.g., SEQ ID NO: 15-18, SEQ ID NOs: 27-30) and MetQ (see e.g., SEQ ID NO: 19-22, SEQ ID NOs: 31-34, 86-89). In some embodiments of any of the aspects, the engineered probiotic microorganism comprises at least one engineered activating modification in MetN (see e.g., SEQ ID NO: 11-14, SEQ ID NOs: 23-26, 80-83), MetI or MetP (see e.g., SEQ ID NO: 15-18, SEQ ID NOs: 27-30), and MetQ (see e.g., SEQ ID NO: 19-22, SEQ ID NOs: 31-34, 86-89).

In some embodiments of any of the aspects, the methionine importer is derived from a methionine importer gene or polypeptide of E. coli (see e.g., SEQ ID NOs: 11-22). In some embodiments of any of the aspects, the methionine importer is derived from a methionine importer gene or polypeptide of B. subtilis (see e.g., SEQ ID NOs: 23-34). The operon known as MetNIQ in E. coli is known as MetNPQ in B. subtilis; thus, MetI in E. coli corresponds to MetP in B. subtilis and vice versa. In some embodiments of any of the aspects, the methionine importer is derived from a methionine importer gene or polypeptide of Lactococcus lactis (see e.g., SEQ ID NOs: 80, 81, 84, 86, 87). In some embodiments of any of the aspects, the methionine importer is derived from a methionine importer gene or polypeptide of Lactiplantibacillus plantarum (see e.g., SEQ ID NOs: 82, 83, 85, 88, 89).

In some embodiments of any of the aspects, the at least one engineered activating modification of the endogenous methionine importer gene comprises at least one engineered activating mutation in the at least one endogenous methionine importer gene, e.g., a mutation that increases the rate of methionine import relative to wild-type of that enzyme. In some embodiments of any of the aspects, the at least one engineered activating modification of the endogenous methionine importer gene comprises at least one engineered activating mutation in a promoter operatively linked to the at least one endogenous methionine importer gene.

MetN, E. coli, 1029 nt
SEQ ID NO: 11
ATGATTAAACTGAGCAACATTACCAAAGTGTTTCATCAGGGCACCCGCACCATTCAGGCG
CTGAACAACGTGAGCCTGCATGTGCCGGCGGGCCAGATTTATGGCGTGATTGGCGCGAG
CGGCGCGGGCAAAAGCACCCTGATTCGCTGCGTGAACCTGCTGGAACGCCCGACCGAAG
GCAGCGTGCTGGTGGATGGCCAGGAACTGACCACCCTGAGCGAAAGCGAACTGACCAAA
GCGCGCCGCCAGATTGGCATGATTTTTCAGCATTTTAACCTGCTGAGCAGCCGCACCGTG
TTTGGCAACGTGGCGCTGCCGCTGGAACTGGATAACACCCCGAAAGATGAAGTGAAACG
CCGCGTGACCGAACTGCTGAGCCTGGTGGGCCTGGGCGATAAACATGATAGCTATCCGA
GCAACCTGAGCGGCGGCCAGAAACAGCGCGTGGCGATTGCGCGCGCGCTGGCGAGCAAC
CCGAAAGTGCTGCTGTGCGATGAAGCGACCAGCGCGCTGGATCCGGCGACCACCCGCAG
CATTCTGGAACTGCTGAAAGATATTAACCGCCGCCTGGGCCTGACCATTCTGCTGATTAC
CCATGAAATGGATGTGGTGAAACGCATTTGCGATTGCGTGGCGGTGATTAGCAACGGCG
AACTGATTGAACAGGATACCGTGAGCGAAGTGTTTAGCCATCCGAAAACCCCGCTGGCG
CAGAAATTTATTCAGAGCACCCTGCATCTGGATATTCCGGAAGATTATCAGGAACGCCTG
CAGGCGGAACCGTTTACCGATTGCGTGCCGATGCTGCGCCTGGAATTTACCGGCCAGAGC
GTGGATGCGCCGCTGCTGAGCGAAACCGCGCGCCGCTTTAACGTGAACAACAACATTAT
TAGCGCGCAGATGGATTATGCGGGCGGCGTGAAATTTGGCATTATGCTGACCGAAATGC
ATGGCACCCAGCAGGATACCCAGGCGGCGATTGCGTGGCTGCAGGAACATCATGTGAAA
GTGGAAGTGCTGGGCTATGTG,
MetN, Regulatory Mutant 1029 nt
SEQ ID NO: 12
ATGATTAAACTGAGCAACATTACCAAAGTGTTTCATCAGGGCACCCGCACCATTCAGGCG
CTGAACAACGTGAGCCTGCATGTGCCGGCGGGCCAGATTTATGGCGTGATTGGCGCGAG
CGGCGCGGGCAAAAGCACCCTGATTCGCTGCGTGAACCTGCTGGAACGCCCGACCGAAG
GCAGCGTGCTGGTGGATGGCCAGGAACTGACCACCCTGAGCGAAAGCGAACTGACCAAA
GCGCGCCGCCAGATTGGCATGATTTTTCAGCATTTTAACCTGCTGAGCAGCCGCACCGTG
TTTGGCAACGTGGCGCTGCCGCTGGAACTGGATAACACCCCGAAAGATGAAGTGAAACG
CCGCGTGACCGAACTGCTGAGCCTGGTGGGCCTGGGCGATAAACATGATAGCTATCCGA
GCAACCTGAGCGGCGGCCAGAAACAGCGCGTGGCGATTGCGCGCGCGCTGGCGAGCAAC
CCGAAAGTGCTGCTGTGCGATGAAGCGACCAGCGCGCTGGATCCGGCGACCACCCGCAG
CATTCTGGAACTGCTGAAAGATATTAACCGCCGCCTGGGCCTGACCATTCTGCTGATTAC
CCATGAAATGGATGTGGTGAAACGCATTTGCGATTGCGTGGCGGTGATTAGCAACGGCG
AACTGATTGAACAGGATACCGTGAGCGAAGTGTTTAGCCATCCGAAAACCCCGCTGGCG
CAGAAATTTATTCAGAGCACCCTGCATCTGGATATTCCGGAAGATTATCAGGAACGCCTG
CAGGCGGAACCGTTTACCGATTGCGTGCCGATGCTGCGCCTGGAATTTACCGGCCAGAGC
GTGGATGCGCCGCTGCTGAGCGAAACCGCGCGCCGCTTTAACGTGAACAACGCGATTAT
TAGCGCGCAGATGGATTATGCGGGCGGCGTGAAATTTGGCATTATGCTGACCGAAATGC
ATGGCACCCAGCAGGATACCCAGGCGGCGATTGCGTGGCTGCAGGAACATCATGTGAAA
GTGGAAGTGCTGGGCTATGTG,
MetN, E. coli, 343 aa
SEQ ID NO: 13
MIKLSNITKVFHQGTRTIQALNNVSLHVPAGQIYGVIGASGAGKSTLIRCVNLLERPTEGSVLV
DGQELTTLSESELTKARRQIGMIFQHFNLLSSRTVFGNVALPLELDNTPKDEVKRRVTELLSL
VGLGDKHDSYPSNLSGGQKQRVAIARALASNPKVLLCDEATSALDPATTRSILELLKDINRRL
GLTILLITHEMDVVKRICDCVAVISNGELIEQDTVSEVFSHPKTPLAQKFIQSTLHLDIPEDYQE
RLQAEPFTDCVPMLRLEFTGQSVDAPLLSETARRFNVNNNIISAQMDYAGGVKFGIMLTEMH
GTQQDTQAAIAWLQEHHVKVEVLGYV,
MetN Regulatory Mutant, 343 aa
SEQ ID NO: 14
MIKLSNITKVFHQGTRTIQALNNVSLHVPAGQIYGVIGASGAGKSTLIRCVNLLERPTEGSVLV
DGQELTTLSESELTKARRQIGMIFQHFNLLSSRTVFGNVALPLELDNTPKDEVKRRVTELLSL
VGLGDKHDSYPSNLSGGQKQRVAIARALASNPKVLLCDEATSALDPATTRSILELLKDINRRL
GLTILLITHEMDVVKRICDCVAVISNGELIEQDTVSEVFSHPKTPLAQKFIQSTLHLDIPEDYQE
RLQAEPFTDCVPMLRLEFTGQSVDAPLLSETARRFNVNNAIISAQMDYAGGVKFGIMLTEMH
GTQQDTQAAIAWLQEHHVKVEVLGYV,
MetI E. coli, 651 nt
SEQ ID NO: 15
ATGAGCGAACCGATGATGTGGCTGCTGGTGCGCGGCGTGTGGGAAACCCTGGCGATGAC
CTTTGTGAGCGGCTTTTTTGGCTTTGTGATTGGCCTGCCGGTGGGCGTGCTGCTGTATGTG
ACCCGCCCGGGCCAGATTATTGCGAACGCGAAACTGTATCGCACCGTGAGCGCGATTGT
GAACATTTTTCGCAGCATTCCGTTTATTATTCTGCTGGTGTGGATGATTCCGTTTACCCGC
GTGATTGTGGGCACCAGCATTGGCCTGCAGGCGGCGATTGTGCCGCTGACCGTGGGCGC
GGCGCCGTTTATTGCGCGCATGGTGGAAAACGCGCTGCTGGAAATTCCGACCGGCCTGAT
TGAAGCGAGCCGCGCGATGGGCGCGACCCCGATGCAGATTGTGCGCAAAGTGCTGCTGC
CGGAAGCGCTGCCGGGCCTGGTGAACGCGGCGACCATTACCCTGATTACCCTGGTGGGC
TATAGCGCGATGGGCGGCGCGGTGGGCGCGGGCGGCCTGGGCCAGATTGGCTATCAGTA
TGGCTATATTGGCTATAACGCGACCGTGATGAACACCGTGCTGGTGCTGCTGGTGATTCT
GGTGTATCTGATTCAGTTTGCGGGCGATCGCATTGTGCGCGCGGTGACCCGCAAA,
MetI Regulatory Mutant, 651 nt
SEQ ID NO: 16
ATGAGCGAACCGATGATGTGGCTGCTGGTGCGCGGCGTGTGGGAAACCCTGGCGATGAC
CTTTGTGAGCGGCTTTTTTGGCTTTGTGATTGGCCTGCCGGTGGGCGTGCTGCTGTATGTG
ACCCGCCCGGGCCAGATTATTGCGAACGCGAAACTGTATCGCACCGTGAGCGCGATTGT
GAACATTTTTCGCAGCATTCCGTTTATTATTCTGCTGGTGTGGATGATTCCGTTTACCCGC
GTGATTGTGGGCACCAGCATTGGCCTGCAGGCGGCGATTGTGCCGCTGACCGTGGGCGC
GGCGCCGTTTATTGCGCGCATGGTGGAAAACGCGCTGCTGGAAATTCCGACCGGCCTGAT
TGAAGCGAGCCGCGCGATGGGCGCGACCCCGATGCAGATTGTGCGCAAAGTGCTGCTGC
CGGAAGCGCTGCCGGGCCTGGTGAACGCGGCGACCATTACCCTGATTACCCTGGTGGGC
GCGAGCGCGATGGGCGGCGCGGTGGGCGCGGGCGGCCTGGGCCAGATTGGCTATCAGTA
TGGCTATATTGGCTATAACGCGACCGTGATGAACACCGTGCTGGTGCTGCTGGTGATTCT
GGTGTATCTGATTCAGTTTGCGGGCGATCGCATTGTGCGCGCGGTGACCCGCAAA,
MetI E. coli, 217 aa
SEQ ID NO: 17
MSEPMMWLLVRGVWETLAMTFVSGFFGFVIGLPVGVLLYVTRPGQIIANAKLYRTVSAIVNI
FRSIPFIILLVWMIPFTRVIVGTSIGLQAAIVPLTVGAAPFIARMVENALLEIPTGLIEASRAMGA
TPMQIVRKVLLPEALPGLVNAATITLITLVGYSAMGGAVGAGGLGQIGYQYGYIGYNATVM
NTVLVLLVILVYLIQFAGDRIVRAVTRK,
MetI E. coli, Regulatory Mutant, 217 aa
SEQ ID NO: 18
MSEPMMWLLVRGVWETLAMTFVSGFFGFVIGLPVGVLLYVTRPGQIIANAKLYRTVSAIVNI
FRSIPFIILLVWMIPFTRVIVGTSIGLQAAIVPLTVGAAPFIARMVENALLEIPTGLIEASRAMGA
TPMQIVRKVLLPEALPGLVNAATITLITLVGASAMGGAVGAGGLGQIGYQYGYIGYNATVM
NTVLVLLVILVYLIQFAGDRIVRAVTRK,
MetQ E. coli, 813 nt
SEQ ID NO: 19
ATGGCGTTTAAATTTAAAACCTTTGCGGCGGTGGGCGCGCTGATTGGCAGCCTGGCGCTG
GTGGGCTGCGGCCAGGATGAAAAAGATCCGAACCATATTAAAGTGGGCGTGATTGTGGG
CGCGGAACAGCAGGTGGCGGAAGTGGCGCAGAAAGTGGCGAAAGATAAATATGGCCTG
GATGTGGAACTGGTGACCTTTAACGATTATGTGCTGCCGAACGAAGCGCTGAGCAAAGG
CGATATTGATGCGAACGCGTTTCAGCATAAACCGTATCTGGATCAGCAGCTGAAAGATC
GCGGCTATAAACTGGTGGCGGTGGGCAACACCTTTGTGTATCCGATTGCGGGCTATAGCA
AAAAAATTAAAAGCCTGGATGAACTGCAGGATGGCAGCCAGGTGGCGGTGCCGAACGAT
CCGACCAACCTGGGCCGCAGCCTGCTGCTGCTGCAGAAAGTGGGCCTGATTAAACTGAA
AGATGGCGTGGGCCTGCTGCCGACCGTGCTGGATGTGGTGGAAAACCCGAAAAACCTGA
AAATTGTGGAACTGGAAGCGCCGCAGCTGCCGCGCAGCCTGGATGATGCGCAGATTGCG
CTGGCGGTGATTAACACCACCTATGCGAGCCAGATTGGCCTGACCCCGGCGAAAGATGG
CATTTTTGTGGAAGATAAAGAAAGCCCGTATGTGAACCTGATTGTGACCCGCGAAGATA
ACAAAGATGCGGAAAACGTGAAAAAATTTGTGCAGGCGTATCAGAGCGATGAAGTGTAT
GAAGCGGCGAACAAAGTGTTTAACGGCGGCGCGGTGAAAGGCTGG,
MetQ E. coli Regulatory Mutant, 813 nt
SEQ ID NO: 20
ATGGCGTTTAAATTTAAAACCTTTGCGGCGGTGGGCGCGCTGATTGGCAGCCTGGCGCTG
GTGGGCTGCGGCCAGGATGAAAAAGATCCGAACCATATTAAAGTGGGCGTGATTGTGGG
CGCGGAACAGCAGGTGGCGGAAGTGGCGCAGAAAGTGGCGAAAGATAAATATGGCCTG
GATGTGGAACTGGTGACCTTTAACGATTATGTGCTGCCGAACGAAGCGCTGAGCAAAGG
CGATATTGATGCGAACGCGTTTCAGCATAAACCGTATCTGGATCAGCAGCTGAAAGATC
GCGGCTATAAACTGGTGGCGGTGGGCAACACCTTTGTGTATCCGATTGCGGGCTATAGCA
AAAAAATTAAAAGCCTGGATGAACTGCAGGATGGCAGCCAGGTGGCGGTGCCGAACGAT
CCGACCAACCTGGGCCGCAGCCTGCTGCTGCTGCAGAAAGTGGGCCTGATTAAACTGAA
AGATGGCGTGGGCCTGCTGCCGACCGTGCTGGATGTGGTGGAAAACCCGAAAAACCTGA
AAATTGTGGAACTGGAAGCGCCGCAGCTGCCGCGCAGCCTGGATGATGCGCAGATTGCG
CTGGCGGTGATTAACACCACCTATGCGAGCCAGATTGGCCTGACCCCGGCGAAAGATGG
CATTTTTGTGGAAGATAAAGAAAGCCCGTATGTGGCGCTGATTGTGACCCGCGAAGATA
ACAAAGATGCGGAAAACGTGAAAAAATTTGTGCAGGCGTATCAGAGCGATGAAGTGTAT
GAAGCGGCGAACAAAGTGTTTAACGGCGGCGCGGTGAAAGGCTGG,
MetQ E. coli, 271 aa
SEQ ID NO: 21
MAFKFKTFAAVGALIGSLALVGCGQDEKDPNHIKVGVIVGAEQQVAEVAQKVAKDKYGLD
VELVTFNDYVLPNEALSKGDIDANAFQHKPYLDQQLKDRGYKLVAVGNTFVYPIAGYSKKI
KSLDELQDGSQVAVPNDPTNLGRSLLLLQKVGLIKLKDGVGLLPTVLDVVENPKNLKIVELE
APQLPRSLDDAQIALAVINTTYASQIGLTPAKDGIFVEDKESPYVNLIVTREDNKDAENVKKF
VQAYQSDEVYEAANKVFNGGAVKGW,
MetQ E. coli, Mutant, 271 aa
SEQ ID NO: 22
MAFKFKTFAAVGALIGSLALVGCGQDEKDPNHIKVGVIVGAEQQVAEVAQKVAKDKYGLD
VELVTFNDYVLPNEALSKGDIDANAFQHKPYLDQQLKDRGYKLVAVGNTFVYPIAGYSKKI
KSLDELQDGSQVAVPNDPTNLGRSLLLLQKVGLIKLKDGVGLLPTVLDVVENPKNLKIVELE
APQLPRSLDDAQIALAVINTTYASQIGLTPAKDGIFVEDKESPYVALIVTREDNKDAENVKKF
VQAYQSDEVYEAANKVFNGGAVKGW,
MetN, B. Subtilis, 1026 nt
SEQ ID NO: 23
ATGATCAATCTTCAGGATGTTTCAAAAGTTTACAAGTCGAAACATGGAGATGTCAATGCT
GTCCAAAACGTtTCGCTTTCCATTAAAAAAGGTGAGATTTTTGGAATTATAGGATATAGC
GGAGCTGGTAAGAGTTCCTTAATCCGTCTGCTGAACGGCCTTGAGAAACCAACCTCAGG
AACCGTGGAAGTGGCGGGAACCAAGATTAATGAAGTAAATGGACGCGGTTTAAGAAAA
GCACGCCATGAGATCAGTATGATTTTCCAGCATTTCAATTTGCTTTGGTCGCGGACTGTC
AGAGATAATATCATGTTTCCTTTAGAAATTGCCGGGGTGAAAAAGAGCGAGCGGATCAA
GCGCGCCAATGAACTGATTAAACTGGTAGGTTTAGAAGGAAAAGAAAAATCTTATCCGT
CCCAGCTGAGCGGCGGTCAGAAGCAGCGTGTCGGAATTGCCAGAGCGCTTGCAAACAAT
CCGAAGGTTCTTCTTTGTGACGAAGCGACATCAGCATTAGATCCGCAAACGACAGATTCA
ATTCTGGATCTATTGTCCGATATTAATGAAAGACTCGGTTTGACGATTGTGCTGATTACG
CACGAAATGCATGTCATACGCAAAATCTGCAACAGAGTCGCCGTCATGGAAAACGGCAA
GGTGGTCGAAGAAGGCGAGGTTCTCGATGTgTTCAAAAATCCAAAGGAACAAATGACAA
AACGATTTGTTCAACAGGTGACAGAGCCGGAAGAAACGAAAGAGACtCTTCAGCACCTTC
TTGATGATACAGCATCAGGAAAAATGGTTCAGCTCACATTTGTCGGTGAGTCAGCTGAAC
AGCCTCTGATTACAGAGATGATCAGAAACTTCAATGTCAGCGTCAATATTCTGCAAGGGA
AAATTTCGCAGACGAAGGATGGGGCTTACGGTTCACTGTTCATCCACATTGACGGGGAC
GAGGAAGAAGTGCAAAACGTGATCCGATTCATTAATGACAAACAGGTGAAAGCAGAGG
TGATCACGAATGTTTGA,
MetN, Regulatory Mutant, B. subtilis, 1026 nt
SEQ ID NO: 24
ATGATCAATCTTCAGGATGTTTCAAAAGTTTACAAGTCGAAACATGGAGATGTCAATGCT
GTCCAAAACGTtTCGCTTTCCATTAAAAAAGGTGAGATTTTTGGAATTATAGGATATAGC
GGAGCTGGTAAGAGTTCCTTAATCCGTCTGCTGAACGGCCTTGAGAAACCAACCTCAGG
AACCGTGGAAGTGGCGGGAACCAAGATTAATGAAGTAAATGGACGCGGTTTAAGAAAA
GCACGCCATGAGATCAGTATGATTTTCCAGCATTTCAATTTGCTTTGGTCGCGGACTGTC
AGAGATAATATCATGTTTCCTTTAGAAATTGCCGGGGTGAAAAAGAGCGAGCGGATCAA
GCGCGCCAATGAACTGATTAAACTGGTAGGTTTAGAAGGAAAAGAAAAATCTTATCCGT
CCCAGCTGAGCGGCGGTCAGAAGCAGCGTGTCGGAATTGCCAGAGCGCTTGCAAACAAT
CCGAAGGTTCTTCTTTGTGACGAAGCGACATCAGCATTAGATCCGCAAACGACAGATTCA
ATTCTGGATCTATTGTCCGATATTAATGAAAGACTCGGTTTGACGATTGTGCTGATTACG
CACGAAATGCATGTCATACGCAAAATCTGCAACAGAGTCGCCGTCATGGAAAACGGCAA
GGTGGTCGAAGAAGGCGAGGTTCTCGATGTgTTCAAAAATCCAAAGGAACAAATGACAA
AACGATTTGTTCAACAGGTGACAGAGCCGGAAGAAACGAAAGAGACtCTTCAGCACCTTC
TTGATGATACAGCATCAGGAAAAATGGTTCAGCTCACATTTGTCGGTGAGTCAGCTGAAC
AGCCTCTGATTACAGAGATGATCAGAAACTTCAATGTCAGCGTCgcTATTCTGCAAGGGA
AAATTTCGCAGACGAAGGATGGGGCTTACGGTTCACTGTTCATCCACATTGACGGGGAC
GAGGAAGAAGTGCAAAACGTGATCCGATTCATTAATGACAAACAGGTGAAAGCAGAGG
TGATCACGAATGTTTGA,
MetN, B. subtilis, 341 aa
SEQ ID NO: 25
MINLQDVSKVYKSKHGDVNAVQNVSLSIKKGEIFGIIGYSGAGKSSLIRLLNGLEKPTSGTVE
VAGTKINEVNGRGLRKARHEISMIFQHFNLLWSRTVRDNIMFPLEIAGVKKSERIKRANELIK
LVGLEGKEKSYPSQLSGGQKQRVGIARALANNPKVLLCDEATSALDPQTTDSILDLLSDINER
LGLTIVLITHEMHVIRKICNRVAVMENGKVVEEGEVLDVFKNPKEQMTKRFVQQVTEPEETK
ETLQHLLDDTASGKMVQLTFVGESAEQPLITEMIRNFNVSVNILQGKISQTKDGAYGSLFIHID
GDEEEVQNVIRFINDKQVKAEVITNV,
MetN Regulatory Mutant B. subtilis, 341 aa
SEQ ID NO: 26
MINLQDVSKVYKSKHGDVNAVQNVSLSIKKGEIFGIIGYSGAGKSSLIRLLNGLEKPTSGTVE
VAGTKINEVNGRGLRKARHEISMIFQHFNLLWSRTVRDNIMFPLEIAGVKKSERIKRANELIK
LVGLEGKEKSYPSQLSGGQKQRVGIARALANNPKVLLCDEATSALDPQTTDSILDLLSDINER
LGLTIVLITHEMHVIRKICNRVAVMENGKVVEEGEVLDVFKNPKEQMTKRFVQQVTEPEETK
ETLQHLLDDTASGKMVQLTFVGESAEQPLITEMIRNFNVSVAILQGKISQTKDGAYGSLFIHID
GDEEEVQNVIRFINDKQVKAEVITNV,
MetP B. subtilis, 669 nt
SEQ ID NO: 27
ATGTTTGAAAAATACTTCCCGAACGTTGATTTAACTGAATTATGGAACGCGACATATGAA
ACACTTTATATGACGCTGATTTCTTTACTGTTTGCTTTTGTCATCGGGGTCATCCTCGGCTT
GCTGCTCTTTCTGACGAGCAAAGGAAGCCTCTGGCAGAATAAAGCGGTCAACTCAGTGA
TTGCAGCCGTTGTTAACATATTCAGATCGATTCCGTTCCTTATTTTAATTATTTTACTATTA
GGTTTTACGAAATTTTTAGTCGGCACGATCTTAGGGCCAAACGCCGCATTGCCGGCGCTG
GTAATCGGCTCGGCACCATTTTACGCGCGCCTTGTTGAAATCGCGCTGCGTGAGGTGGAT
AAAGGTGTGATTGAAGCAGCTAAATCAATGGGCGCGAAAACGTCTACGATTATTTTCAA
AGTGCTGATTCCGGAATCAATGCCTGCTCTTATTTCTGGCATTACGGTTACAGCCATCGCT
TTAATCGGTTCAACGGCAATTGCCGGAGCCATTGGTTCAGGAGGCCTTGGAAACCTTGCG
TACGTAGAAGGATATCAGTCTAATAACGCTGATGTTACCTTCGTTGCTACTGTGTTTATTT
TAATCATCGTGTTTATTATTCAAATCATCGGTGATCTAATAACAAATATTATAGACAAAC
GATAA,
MetP Regulatory Mutant B. subtilis, 669 nt
SEQ ID NO: 28
ATGTTTGAAAAATACTTCCCGAACGTTGATTTAACTGAATTATGGAACGCGACATATGAA
ACACTTTATATGACGCTGATTTCTTTACTGTTTGCTTTTGTCATCGGGGTCATCCTCGGCTT
GCTGCTCTTTCTGACGAGCAAAGGAAGCCTCTGGCAGAATAAAGCGGTCAACTCAGTGA
TTGCAGCCGTTGTTAACATATTCAGATCGATTCCGTTCCTTATTTTAATTATTTTACTATTA
GGTTTTACGAAATTTTTAGTCGGCACGATCTTAGGGCCAAACGCCGCATTGCCGGCGCTG
GTAATCGGCTCGGCACCATTTTACGCGCGCCTTGTTGAAATCGCGCTGCGTGAGGTGGAT
AAAGGTGTGATTGAAGCAGCTAAATCAATGGGCGCGAAAACGTCTACGATTATTTTCAA
AGTGCTGATTCCGGAATCAATGCCTGCTCTTATTTCTGGCATTACGGTTACAGCCATCGCT
TTAATCGGTTCAACGGCAATTGCCGGAGCCATTGGTTCAGGAGGCCTTGGAAACCTTGCG
TACGTAGAAGGATATCAGTCTAATAACGCTGATGTTACCTTCGTTGCTACTGTGTTTATTT
TAATCATCGTGTTTATTATTCAAATCATCGGTGATCTAATAACAAATATTATAGACAAAC
GATAA,
MetP B. Subtilis, 222 aa
SEQ ID NO: 29
MFEKYFPNVDLTELWNATYETLYMTLISLLFAFVIGVILGLLLFLTSKGSLWQNKAVNSVIAA
VVNIFRSIPFLILIILLLGFTKFLVGTILGPNAALPALVIGSAPFYARLVEIALREVDKGVIEAAKS
MGAKTSTIIFKVLIPESMPALISGITVTAIALIGSTAIAGAIGSGGLGNLAYVEGYQSNNADVTF
VATVFILIIVFIIQIIGDLITNIIDKR,
MetP B. Subtilis, Regulatory Mutant, 222 aa
SEQ ID NO: 30
MFEKYFPNVDLTELWNATYETLYMTLISLLFAFVIGVILGLLLFLTSKGSLWQNKAVNSVIAA
VVNIFRSIPFLILIILLLGFTKFLVGTILGPNAALPALVIGSAPFYARLVEIALREVDKGVIEAAKS
MGAKTSTIIFKVLIPESMPALISGITVTAIALIGSTAIAGAIGSGGLGNLAYVEGYQSNNADVTF
VATVFILIIVFIIQIIGDLITNIIDKR,
MetQ B. Subtilis, 825 nt
SEQ ID NO: 31
aTGAAAAAGCTATTTTTGGGTGCATTACTGCTTGTATTTGCAGGAGTTATGGCTGCCTGCG
GTTCGAATAACGGCGCTGAATCCGGCAAGAAAGAAATTGTCGTTGCGGCAACAAAAACA
CCGCATGCGGAAATTTTAAAAGAAGCTGAACCATTGCTGAAAGAAAAAGGCTATACGCT
GAAAGTGAAAGTGCTTAGTGATTACAAAATGTACAATAAAGCTTTAGCTGATAAAGAAG
TGGACGCGAACTACTTCCAGCACATTCCTTACCTTGAGCAAGAAATGAAAGAAAACACA
GATTACAAACTTGTGAATGCCGGCGCTGTTCACTTAGAGCCATTCGGTATTTACTCTAAA
ACATACAAATCACTGAAAGACCTTCCAGACGGTGCGACAATCATTCTGACAAACAACGT
TGCTGAACAAGGCCGTATGCTTGCAATGCTTGAAAACGCTGGATTAATCACTCTTGATTC
TAAAGTGGAAACAGTTGACGCAACATTGAAAGACATTAAGAAAAACCCGAAAAACCTTG
AATTCAAAAAAGTAGCGCCTGAATTAACGGCAAAAGCATATGAAAACAAAGAAGGAGAt
GCGGTgTTCATCAATGTAAACTATGCGATCCAAAATAAATTAAATCCTAAAAAAGACGCA
ATTGAAGTAGAATCAACGAAAAACAACCCATACGCTAACATCATCGCAGTAAGAAAAGG
CGAAGAAGATTCTGCAAAAATCAAAGCGCTGATGGAAGTTCTTCACTCTAAAAAGATCA
AAGACTTCATCGAGAAAAAATACGACGGAGCTGTGCTTCCTGTATCTGAATAA,
MetQ B. Subtilis Regulatory Mutant, 825 nt
SEQ ID NO: 32
aTGAAAAAGCTATTTTTGGGTGCATTACTGCTTGTATTTGCAGGAGTTATGGCTGCCTGCG
GTTCGAATAACGGCGCTGAATCCGGCAAGAAAGAAATTGTCGTTGCGGCAACAAAAACA
CCGCATGCGGAAATTTTAAAAGAAGCTGAACCATTGCTGAAAGAAAAAGGCTATACGCT
GAAAGTGAAAGTGCTTAGTGATTACAAAATGTACAATAAAGCTTTAGCTGATAAAGAAG
TGGACGCGAACTACTTCCAGCACATTCCTTACCTTGAGCAAGAAATGAAAGAAAACACA
GATTACAAACTTGTGAATGCCGGCGCTGTTCACTTAGAGCCATTCGGTATTTACTCTAAA
ACATACAAATCACTGAAAGACCTTCCAGACGGTGCGACAATCATTCTGACAAACAACGT
TGCTGAACAAGGCCGTATGCTTGCAATGCTTGAAAACGCTGGATTAATCACTCTTGATTC
TAAAGTGGAAACAGTTGACGCAACATTGAAAGACATTAAGAAAAACCCGAAAAACCTTG
AATTCAAAAAAGTAGCGCCTGAATTAACGGCAAAAGCATATGAAAACAAAGAAGGAGAt
GCGGTgTTCATCAATGTAAACTATGCGATCCAAAATAAATTAAATCCTAAAAAAGACGCA
ATTGAAGTAGAATCAACGAAAAACAACCCATACGCTgcCATCATCGCAGTAAGAAAAGG
CGAAGAAGATTCTGCAAAAATCAAAGCGCTGATGGAAGTTCTTCACTCTAAAAAGATCA
AAGACTTCATCGAGAAAAAATACGACGGAGCTGTGCTTCCTGTATCTGAATAA,
MetQ B. Subtilis, 274 aa
SEQ ID NO: 33
MKKLFLGALLLVFAGVMAACGSNNGAESGKKEIVVAATKTPHAEILKEAEPLLKEKGYTLK
VKVLSDYKMYNKALADKEVDANYFQHIPYLEQEMKENTDYKLVNAGAVHLEPFGIYSKTY
KSLKDLPDGATIILTNNVAEQGRMLAMLENAGLITLDSKVETVDATLKDIKKNPKNLEFKKV
APELTAKAYENKEGDAVFINVNYAIQNKLNPKKDAIEVESTKNNPYANIIAVRKGEEDSAKIK
ALMEVLHSKKIKDFIEKKYDGAVLPVSE,
MetQ B. Subtilis Mutant, 274 aa
SEQ ID NO: 34
MKKLFLGALLLVFAGVMAACGSNNGAESGKKEIVVAATKTPHAEILKEAEPLLKEKGYTLK
VKVLSDYKMYNKALADKEVDANYFQHIPYLEQEMKENTDYKLVNAGAVHLEPFGIYSKTY
KSLKDLPDGATIILTNNVAEQGRMLAMLENAGLITLDSKVETVDATLKDIKKNPKNLEFKKV
APELTAKAYENKEGDAVFINVNYAIQNKLNPKKDAIEVESTKNNPYAAIIAVRKGEEDSAKIK
ALMEVLHSKKIKDFIEKKYDGAVLPVSE,
Lactococcus lactis_MetN_WT
SEQ ID NO: 80
IIELNNLSVQFHQKGRLVTAVKNATLHIEKGDIYGVIGYSGAGKSTLVRTINLLQKPTEGQIVI
NGEKIFDSENPVKFTGAKLREFRQKIGMIFQHFNLLSEKTVFNNVAFALQHSQIEDKNGKKRY
LTKKEKNDKVTELLKLVDLADLSDKYPAQLSGGQKQRVAIARALANDPEILISDEGTSALDP
KTTNQILDLLKSLHEKLGITVVLITHEMQVVKEIANKVAVMQNGEIIEQNSLIDIFAQPKEALT
KQFIETTSSVNRFIASLSKTELLAQLADDEELIHLDYSGSELEDPVVSDITKKFDVTTNIFYGNV
ELLQGQPFGSLVLTLKGSSEHRAAAKAYFVERHLKFEVLGKI,
Lactococcus lactis_MetN N295A
SEQ ID NO: 81
IIELNNLSVQFHQKGRLVTAVKNATLHIEKGDIYGVIGYSGAGKSTLVRTINLLQKPTEGQIVI
NGEKIFDSENPVKFTGAKLREFRQKIGMIFQHFNLLSEKTVFNNVAFALQHSQIEDKNGKKRY
LTKKEKNDKVTELLKLVDLADLSDKYPAQLSGGQKQRVAIARALANDPEILISDEGTSALDP
KTTNQILDLLKSLHEKLGITVVLITHEMQVVKEIANKVAVMQNGEIIEQNSLIDIFAQPKEALT
KQFIETTSSVNRFIASLSKTELLAQLADDEELIHLDYSGSELEDPVVSDITKKFDVTTAIFYGNV
ELLQGQPFGSLVLTLKGSSEHRAAAKAYFVERHLKFEVLGKI,
Lactiplantibacillus plantarum_MetN_WT
SEQ ID NO: 82
MTEAVIDLTKIGVTFKDGQQTIQAVQDVDLKIEAGDIYGIIGYSGAGKSTLVRVINLLQVPTTG
RVVVNGQTLQELSPVALRQARKRVGMIFQHFNLMQSRTVLGNVVYPLLGQKISKSERRAKA
LRLLKLVGLEDYAESYPDKLSGGQKQRVAIARALVTDPQILISDEATSALDPKTTAAILSLLQ
QVNRNLGVTIVLITHEMQVIKSVCHHVAVMENGRIIERGPVAQVFTAPQAPLTVDFVETSTN
VRAAIERITRTIKLSELADDQELIAFKFVGQSTKQGIVSHLSQTLGVDVNILFANIDQIDGQNV
GDMIAIITGNLPAFNQAIAQLADQGVQTHVINEQDVKELVD,
Lactiplantibacillus plantarum_MetN_N295A
SEQ ID NO: 83
MTEAVIDLTKIGVTFKDGQQTIQAVQDVDLKIEAGDIYGIIGYSGAGKSTLVRVINLLQVPTTG
RVVVNGQTLQELSPVALRQARKRVGMIFQHFNLMQSRTVLGNVVYPLLGQKISKSERRAKA
LRLLKLVGLEDYAESYPDKLSGGQKQRVAIARALVTDPQILISDEATSALDPKTTAAILSLLQ
QVNRNLGVTIVLITHEMQVIKSVCHHVAVMENGRIIERGPVAQVFTAPQAPLTVDFVETSTN
VRAAIERITRTIKLSELADDQELIAFKFVGQSTKQGIVSHLSQTLGVDVAILFANIDQIDGQNV
GDMIAIITGNLPAFNQAIAQLADQGVQTHVINEQDVKELVD,
Lactococcus lactis_MetI_WT
SEQ ID NO: 84
MAEWFAHTFPNVVYLGWTGETGWWTAIVQTLYMTFISALIGGLLGLIFGIGVVVTAEDGITP
NRPLFWILDKIVSIGRAFPFIILLAAIAPLTKILVGTQIGVTAALVPLALGVAPFYARQVQASLE
SVDHGKVEAAQTVGADFLDIVFTVYLREELASLVRVSTVTLISLIGLTAMAGAIGAGGLGNT
AISYGYNRFANDVTWFATILILIFVLLVQLVGDFLARRVSHR,
Lactiplantibacillus plantarum_MetI_WT
SEQ ID NO: 85
MLQKLIPNVLQMKGEFVQATWETLYMTFGSAIIAAVLGLLLGVCLVITQPGGILEDVLTYSVL
DKITNLLRSIPFIILLAVISPLTQFLIGTTVGTTASLVPLIVGIFPFYARQVQNALLTVEHGVIEAA
QAMGSSPTEIVFRVYLREGLADILRVSIVTVISLIGLTTMAGAIGSGGLGDVAISIGYARFENDV
TFVAMIIILILVFAVQIIGDLIVKAVEHNE,
Lactococcus lactis_MetQ_WT
SEQ ID NO: 86
MNPRNRNIIIGIIVVVIVAIAAFIGFGQKSQANKTVNKTVKIGIMTGTKEDDSIWQTVSKTAKD
KYGITLKFTHFTDYTQPNTALKNGDIDLNAFQHYAFLKAWNKANNGNLVAIGDTVISPISVY
SKQLKNISDIKEGGTIAVPNDASNESRALYVLKSAGLIKLDVSGQTLATVKDITSNPKNLVIKE
LDASQTARALDSVDAAVINNNYAVTAGLKKSDAIFTEPVNKDSQQWINIIVANKKDENNTVY
KDVVKAYETEATKETIAKAYPDKSTIPAWGLKLK,
Lactococcus lactis_MetQ_N229A
SEQ ID NO: 87
MNPRNRNIIIGIIVVVIVAIAAFIGFGQKSQANKTVNKTVKIGIMTGTKEDDSIWQTVSKTAKD
KYGITLKFTHFTDYTQPNTALKNGDIDLNAFQHYAFLKAWNKANNGNLVAIGDTVISPISVY
SKQLKNISDIKEGGTIAVPNDASNESRALYVLKSAGLIKLDVSGQTLATVKDITSNPKNLVIKE
LDASQTARALDSVDAAVINNNYAVTAGLKKSDAIFTEPVNKDSQQWIAIIVANKKDENNTVY
KDVVKAYETEATKETIAKAYPDKSTIPAWGLKLK,
Lactiplantibacillus plantarum_MetQ_WT
SEQ ID NO: 88
MKKKGILGLLAVAATAFLLVGCGKSSSATKTTTITVGASSVPHAQVLKHVQPELKKEGVNLK
IKAFQDYVLPNKALASKELDANYFQHIPFLDNWNKENNGTLVSAGKVHLEPIGVYSKKVKSL
KDLKDGATVLVSSNVADYGRVLTLFKDAGLITLKPGTKLTSATFNDIKTNKRHLKFKHSYEA
KLMPTFYKNNEGDAVVINANYAVQAGLSPKKDAIALEKSDSPYANIVAVRKGDKNKPAIKK
LMKALRSKSTQQWIEKKYKGAILPVSAD,
Lactiplantibacillus plantarum_MetQ_N229A
SEQ ID NO: 89
MKKKGILGLLAVAATAFLLVGCGKSSSATKTTTITVGASSVPHAQVLKHVQPELKKEGVNLK
IKAFQDYVLPNKALASKELDANYFQHIPFLDNWNKENNGTLVSAGKVHLEPIGVYSKKVKSL
KDLKDGATVLVSSNVADYGRVLTLFKDAGLITLKPGTKLTSATFNDIKTNKRHLKFKHSYEA
KLMPTFYKNNEGDAVVINANYAVQAGLSPKKDAIALEKSDSPYAAIVAVRKGDKNKPAIKK
LMKALRSKSTQQWIEKKYKGAILPVSAD,

### Methionine Synthesis Enzyme

Another approach to reducing bioavailable methionine is to limit the amount of methionine produced by an engineered microorganism. This approach can boost or amplify the efficiency of methionine reduction by bacteria as described herein—coupling mutagenesis to knock out or reduce the activity of methionine biosynthetic pathways with expression of one or more exogenous methionine catabolic pathway enzymes can help to ensure that the engineered microorganism is not fighting itself to reduce bioavailable methionine. As noted, this approach can be combined with any of the other methionine-reducing approaches described herein.

In one aspect, described herein is an engineered probiotic microorganism for reducing bioavailable methionine levels comprising at least one endogenous methionine synthesis gene comprising at least one engineered inactivating modification. The methionine synthesis gene or enzyme can also be referred to herein as a methionine anabolic gene or enzyme. In some embodiments of any of the aspects, at least one endogenous methionine synthesis gene is partially or completely deleted (i.e., knocked out) in the engineered probiotic microorganism. In some embodiments of any of the aspects, the expression of the at least one endogenous methionine synthesis gene is downregulated. In some embodiments of any of the aspects, the engineered probiotic microorganism comprises at least one exogenous copy of at least one gene encoding an enzyme that catalyzes the degradation of methionine and at least one endogenous methionine synthesis gene comprising at least one engineered inactivating modification.

In some embodiments of any of the aspects, the at least one engineered inactivating modification of the endogenous methionine synthesis gene comprises at least one engineered inactivating mutation in the at least one endogenous methionine synthesis gene. In some embodiments of any of the aspects, the at least one engineered inactivating modification of the endogenous methionine synthesis gene comprises at least one engineered inactivating mutation in a promoter operatively linked to the at least one endogenous methionine synthesis gene. In some embodiments of any of the aspects, the at least one engineered inactivating modification of the endogenous methionine synthesis gene comprises at least one inhibitory RNA molecule specific to at least one messenger RNA (mRNA) expressed by the at least one endogenous methionine synthesis gene. Non-limiting examples of inhibitory RNAs include small interfering RNA (siRNA), micro RNA (miRNA), CRISPR RNA (crRNA and associated Cas endonuclease), and the like.

Non-limiting examples of an endogenous methionine synthesis enzyme include MetH (see e.g., SEQ ID NOs: 35-36) or MetE (see e.g., SEQ ID NOs: 37-40). In some embodiments of any of the aspects, the methionine synthesis enzyme is encoded by one of SEQ ID NOs: 35, 37, 39 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 35, 37, or 39, that maintains the same function, or a codon-optimized version thereof.

In some embodiments of any of the aspects, the methionine synthesis enzyme comprises one of SEQ ID NOs: 36, 38, 40, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NOs: 36, 38, or 40 that maintains the same function (e.g., methionine synthesis).

In some embodiments of any of the aspects, the engineered probiotic microorganism comprises at least one engineered inactivating modification in MetH (see e.g., SEQ ID NOs: 35-36) or MetE (see e.g., SEQ ID NOs: 37-40). In some embodiments of any of the aspects, the engineered probiotic microorganism comprises at least one engineered inactivating modification in MetH (see e.g., SEQ ID NOs: 35-36). In some embodiments of any of the aspects, the engineered probiotic microorganism comprises at least one engineered inactivating modification in MetE (see e.g., SEQ ID NOs: 37-40). In some embodiments of any of the aspects, the engineered probiotic microorganism comprises at least one engineered inactivating modification in MetH (see e.g., SEQ ID NOs: 35-36) and MetE (see e.g., SEQ ID NOs: 37-40).

In some embodiments of any of the aspects, the methionine synthesis enzyme is derived from a methionine synthesis gene or polypeptide of E. coli (see e.g., SEQ ID NOs: 35-38). In some embodiments of any of the aspects, the methionine synthesis enzyme is derived from a methionine synthesis gene or polypeptide of B. subtilis (see e.g., SEQ ID NOs: 39-40).

MetH “Methionine Synthase I,” E. coli, 3681 nt,
SEQ ID NO: 35
ATGAGCAGCAAAGTGGAACAGCTGCGCGCGCAGCTGAACGAACGCATTCTGGTGCTGGA
TGGCGGCATGGGCACCATGATTCAGAGCTATCGCCTGAACGAAGCGGATTTTCGCGGCG
AACGCTTTGCGGATTGGCCGTGCGATCTGAAAGGCAACAACGATCTGCTGGTGCTGAGC
AAACCGGAAGTGATTGCGGCGATTCATAACGCGTATTTTGAAGCGGGCGCGGATATTATT
GAAACCAACACCTTTAACAGCACCACCATTGCGATGGCGGATTATCAGATGGAAAGCCT
GAGCGCGGAAATTAACTTTGCGGCGGCGAAACTGGCGCGCGCGTGCGCGGATGAATGGA
CCGCGCGCACCCCGGAAAAACCGCGCTATGTGGCGGGCGTGCTGGGCCCGACCAACCGC
ACCGCGAGCATTAGCCCGGATGTGAACGATCCGGCGTTTCGCAACATTACCTTTGATGGC
CTGGTGGCGGCGTATCGCGAAAGCACCAAAGCGCTGGTGGAAGGCGGCGCGGATCTGAT
TCTGATTGAAACCGTGTTTGATACCCTGAACGCGAAAGCGGCGGTGTTTGCGGTGAAAAC
CGAATTTGAAGCGCTGGGCGTGGAACTGCCGATTATGATTAGCGGCACCATTACCGATGC
GAGCGGCCGCACCCTGAGCGGCCAGACCACCGAAGCGTTTTATAACAGCCTGCGCCATG
CGGAAGCGCTGACCTTTGGCCTGAACTGCGCGCTGGGCCCGGATGAACTGCGCCAGTAT
GTGCAGGAACTGAGCCGCATTGCGGAATGCTATGTGACCGCGCATCCGAACGCGGGCCT
GCCGAACGCGTTTGGCGAATATGATCTGGATGCGGATACCATGGCGAAACAGATTCGCG
AATGGGCGCAGGCGGGCTTTCTGAACATTGTGGGCGGCTGCTGCGGCACCACCCCGCAG
CATATTGCGGCGATGAGCCGCGCGGTGGAAGGCCTGGCGCCGCGCAAACTGCCGGAAAT
TCCGGTGGCGTGCCGCCTGAGCGGCCTGGAACCGCTGAACATTGGCGAAGATAGCCTGT
TTGTGAACGTGGGCGAACGCACCAACGTGACCGGCAGCGCGAAATTTAAACGCCTGATT
AAAGAAGAAAAATATAGCGAAGCGCTGGATGTGGCGCGCCAGCAGGTGGAAAACGGCG
CGCAGATTATTGATATTAACATGGATGAAGGCATGCTGGATGCGGAAGCGGCGATGGTG
CGCTTTCTGAACCTGATTGCGGGCGAACCGGATATTGCGCGCGTGCCGATTATGATTGAT
AGCAGCAAATGGGATGTGATTGAAAAAGGCCTGAAATGCATTCAGGGCAAAGGCATTGT
GAACAGCATTAGCATGAAAGAAGGCGTGGATGCGTTTATTCATCATGCGAAACTGCTGC
GCCGCTATGGCGCGGCGGTGGTGGTGATGGCGTTTGATGAACAGGGCCAGGCGGATACC
CGCGCGCGCAAAATTGAAATTTGCCGCCGCGCGTATAAAATTCTGACCGAAGAAGTGGG
CTTTCCGCCGGAAGATATTATTTTTGATCCGAACATTTTTGCGGTGGCGACCGGCATTGA
AGAACATAACAACTATGCGCAGGATTTTATTGGCGCGTGCGAAGATATTAAACGCGAAC
TGCCGCATGCGCTGATTAGCGGCGGCGTGAGCAACGTGAGCTTTAGCTTTCGCGGCAACG
ATCCGGTGCGCGAAGCGATTCATGCGGTGTTTCTGTATTATGCGATTCGCAACGGCATGG
ATATGGGCATTGTGAACGCGGGCCAGCTGGCGATTTATGATGATCTGCCGGCGGAACTG
CGCGATGCGGTGGAAGATGTGATTCTGAACCGCCGCGATGATGGCACCGAACGCCTGCT
GGAACTGGCGGAAAAATATCGCGGCAGCAAAACCGATGATACCGCGAACGCGCAGCAG
GCGGAATGGCGCAGCTGGGAAGTGAACAAACGCCTGGAATATAGCCTGGTGAAAGGCAT
TACCGAATTTATTGAACAGGATACCGAAGAAGCGCGCCAGCAGGCGACCCGCCCGATTG
AAGTGATTGAAGGCCCGCTGATGGATGGCATGAACGTGGTGGGCGATCTGTTTGGCGAA
GGCAAAATGTTTCTGCCGCAGGTGGTGAAAAGCGCGCGCGTGATGAAACAGGCGGTGGC
GTATCTGGAACCGTTTATTGAAGCGAGCAAAGAACAGGGCAAAACCAACGGCAAAATGG
TGATTGCGACCGTGAAAGGCGATGTGCATGATATTGGCAAAAACATTGTGGGCGTGGTG
CTGCAGTGCAACAACTATGAAATTGTGGATCTGGGCGTGATGGTGCCGGCGGAAAAAAT
TCTGCGCACCGCGAAAGAAGTGAACGCGGATCTGATTGGCCTGAGCGGCCTGATTACCC
CGAGCCTGGATGAAATGGTGAACGTGGCGAAAGAAATGGAACGCCAGGGCTTTACCATT
CCGCTGCTGATTGGCGGCGCGACCACCAGCAAAGCGCATACCGCGGTGAAAATTGAACA
GAACTATAGCGGCCCGACCGTGTATGTGCAGAACGCGAGCCGCACCGTGGGCGTGGTGG
CGGCGCTGCTGAGCGATACCCAGCGCGATGATTTTGTGGCGCGCACCCGCAAAGAATAT
GAAACCGTGCGCATTCAGCATGGCCGCAAAAAACCGCGCACCCCGCCGGTGACCCTGGA
AGCGGCGCGCGATAACGATTTTGCGTTTGATTGGCAGGCGTATACCCCGCCGGTGGCGCA
TCGCCTGGGCGTGCAGGAAGTGGAAGCGAGCATTGAAACCCTGCGCAACTATATTGATT
GGACCCCGTTTTTTATGACCTGGAGCCTGGCGGGCAAATATCCGCGCATTCTGGAAGATG
AAGTGGTGGGCGTGGAAGCGCAGCGCCTGTTTAAAGATGCGAACGATATGCTGGATAAA
CTGAGCGCGGAAAAAACCCTGAACCCGCGCGGCGTGGTGGGCCTGTTTCCGGCGAACCG
CGTGGGCGATGATATTGAAATTTATCGCGATGAAACCCGCACCCATGTGATTAACGTGAG
CCATCATCTGCGCCAGCAGACCGAAAAAACCGGCTTTGCGAACTATTGCCTGGCGGATTT
TGTGGCGCCGAAACTGAGCGGCAAAGCGGATTATATTGGCGCGTTTGCGGTGACCGGCG
GCCTGGAAGAAGATGCGCTGGCGGATGCGTTTGAAGCGCAGCATGATGATTATAACAAA
ATTATGGTGAAAGCGCTGGCGGATCGCCTGGCGGAAGCGTTTGCGGAATATCTGCATGA
ACGCGTGCGCAAAGTGTATTGGGGCTATGCGCCGAACGAAAACCTGAGCAACGAAGAAC
TGATTCGCGAAAACTATCAGGGCATTCGCCCGGCGCCGGGCTATCCGGCGTGCCCGGAA
CATACCGAAAAAGCGACCATTTGGGAACTGCTGGAAGTGGAAAAACATACCGGCATGAA
ACTGACCGAAAGCTTTGCGATGTGGCCGGGCGCGAGCGTGAGCGGCTGGTATTTTAGCC
ATCCGGATAGCAAATATTATGCGGTGGCGCAGATTCAGCGCGATCAGGTGGAAGATTAT
GCGCGCCGCAAAGGCATGAGCGTGACCGAAGTGGAACGCTGGCTGGCGCCGAACCTGGG
CTATGATGCGGAT
MetH “Methionine Synthase I,” E. coli, 1227 aa,
SEQ ID NO: 36
MSSKVEQLRAQLNERILVLDGGMGTMIQSYRLNEADFRGERFADWPCDLKGNNDLLVLSKP
EVIAAIHNAYFEAGADIIETNTFNSTTIAMADYQMESLSAEINFAAAKLARACADEWTARTPE
KPRYVAGVLGPTNRTASISPDVNDPAFRNITFDGLVAAYRESTKALVEGGADLILIETVFDTL
NAKAAVFAVKTEFEALGVELPIMISGTITDASGRTLSGQTTEAFYNSLRHAEALTFGLNCALG
PDELRQYVQELSRIAECYVTAHPNAGLPNAFGEYDLDADTMAKQIREWAQAGFLNIVGGCC
GTTPQHIAAMSRAVEGLAPRKLPEIPVACRLSGLEPLNIGEDSLFVNVGERTNVTGSAKFKRLI
KEEKYSEALDVARQQVENGAQIIDINMDEGMLDAEAAMVRFLNLIAGEPDIARVPIMIDSSK
WDVIEKGLKCIQGKGIVNSISMKEGVDAFIHHAKLLRRYGAAVVVMAFDEQGQADTRARKI
EICRRAYKILTEEVGFPPEDIIFDPNIFAVATGIEEHNNYAQDFIGACEDIKRELPHALISGGVSN
VSFSFRGNDPVREAIHAVFLYYAIRNGMDMGIVNAGQLAIYDDLPAELRDAVEDVILNRRDD
GTERLLELAEKYRGSKTDDTANAQQAEWRSWEVNKRLEYSLVKGITEFIEQDTEEARQQAT
RPIEVIEGPLMDGMNVVGDLFGEGKMFLPQVVKSARVMKQAVAYLEPFIEASKEQGKTNGK
MVIATVKGDVHDIGKNIVGVVLQCNNYEIVDLGVMVPAEKILRTAKEVNADLIGLSGLITPSL
DEMVNVAKEMERQGFTIPLLIGGATTSKAHTAVKIEQNYSGPTVYVQNASRTVGVVAALLS
DTQRDDFVARTRKEYETVRIQHGRKKPRTPPVTLEAARDNDFAFDWQAYTPPVAHRLGVQE
VEASIETLRNYIDWTPFFMTWSLAGKYPRILEDEVVGVEAQRLFKDANDMLDKLSAEKTLNP
RGVVGLFPANRVGDDIEIYRDETRTHVINVSHHLRQQTEKTGFANYCLADFVAPKLSGKADY
IGAFAVTGGLEEDALADAFEAQHDDYNKIMVKALADRLAEAFAEYLHERVRKVYWGYAPN
ENLSNEELIRENYQGIRPAPGYPACPEHTEKATIWELLEVEKHTGMKLTESFAMWPGASVSG
WYFSHPDSKYYAVAQIQRDQVEDYARRKGMSVTEVERWLAPNLGYDAD
MetE “Methionine Synthase II,” E. coli, 2259 nt,
SEQ ID NO: 37
ATGACCATTCTGAACCATACCCTGGGCTTTCCGCGCGTGGGCCTGCGCCGCGAACTGAAA
AAAGCGCAGGAAAGCTATTGGGCGGGCAACAGCACCCGCGAAGAACTGCTGGCGGTGG
GCCGCGAACTGCGCGCGCGCCATTGGGATCAGCAGAAACAGGCGGGCATTGATCTGCTG
CCGGTGGGCGATTTTGCGTGGTATGATCATGTGCTGACCACCAGCCTGCTGCTGGGCAAC
GTGCCGGCGCGCCATCAGAACAAAGATGGCAGCGTGGATATTGATACCCTGTTTCGCATT
GGCCGCGGCCGCGCGCCGACCGGCGAACCGGCGGCGGCGGCGGAAATGACCAAATGGT
TTAACACCAACTATCATTATATGGTGCCGGAATTTGTGAAAGGCCAGCAGTTTAAACTGA
CCTGGACCCAGCTGCTGGATGAAGTGGATGAAGCGCTGGCGCTGGGCCATAAAGTGAAA
CCGGTGCTGCTGGGCCCGGTGACCTGGCTGTGGCTGGGCAAAGTGAAAGGCGAACAGTT
TGATCGCCTGAGCCTGCTGAACGATATTCTGCCGGTGTATCAGCAGGTGCTGGCGGAACT
GGCGAAACGCGGCATTGAATGGGTGCAGATTGATGAACCGGCGCTGGTGCTGGAACTGC
CGCAGGCGTGGCTGGATGCGTATAAACCGGCGTATGATGCGCTGCAGGGCCAGGTGAAA
CTGCTGCTGACCACCTATTTTGAAGGCGTGACCCCGAACCTGGATACCATTACCGCGCTG
CCGGTGCAGGGCCTGCATGTGGATCTGGTGCATGGCAAAGATGATGTGGCGGAACTGCA
TAAACGCCTGCCGAGCGATTGGCTGCTGAGCGCGGGCCTGATTAACGGCCGCAACGTGT
GGCGCGCGGATCTGACCGAAAAATATGCGCAGATTAAAGATATTGTGGGCAAACGCGAT
CTGTGGGTGGCGAGCAGCTGCAGCCTGCTGCATAGCCCGATTGATCTGAGCGTGGAAAC
CCGCCTGGATGCGGAAGTGAAAAGCTGGTTTGCGTTTGCGCTGCAGAAATGCCATGAAC
TGGCGCTGCTGCGCGATGCGCTGAACAGCGGCGATACCGCGGCGCTGGCGGAATGGAGC
GCGCCGATTCAGGCGCGCCGCCATAGCACCCGCGTGCATAACCCGGCGGTGGAAAAACG
CCTGGCGGCGATTACCGCGCAGGATAGCCAGCGCGCGAACGTGTATGAAGTGCGCGCGG
AAGCGCAGCGCGCGCGCTTTAAACTGCCGGCGTGGCCGACCACCACCATTGGCAGCTTTC
CGCAGACCACCGAAATTCGCACCCTGCGCCTGGATTTTAAAAAAGGCAACCTGGATGCG
AACAACTATCGCACCGGCATTGCGGAACATATTAAACAGGCGATTGTGGAACAGGAACG
CCTGGGCCTGGATGTGCTGGTGCATGGCGAAGCGGAACGCAACGATATGGTGGAATATT
TTGGCGAACATCTGGATGGCTTTGTGTTTACCCAGAACGGCTGGGTGCAGAGCTATGGCA
GCCGCTGCGTGAAACCGCCGATTGTGATTGGCGATATTAGCCGCCCGGCGCCGATTACCG
TGGAATGGGCGAAATATGCGCAGAGCCTGACCGATAAACCGGTGAAAGGCATGCTGACC
GGCCCGGTGACCATTCTGTGCTGGAGCTTTCCGCGCGAAGATGTGAGCCGCGAAACCATT
GCGAAACAGATTGCGCTGGCGCTGCGCGATGAAGTGGCGGATCTGGAAGCGGCGGGCAT
TGGCATTATTCAGATTGATGAACCGGCGCTGCGCGAAGGCCTGCCGCTGCGCCGCAGCG
ATTGGGATGCGTATCTGCAGTGGGGCGTGGAAGCGTTTCGCATTAACGCGGCGGTGGCG
AAAGATGATACCCAGATTCATACCCATATGTGCTATTGCGAATTTAACGATATTATGGAT
AGCATTGCGGCGCTGGATGCGGATGTGATTACCATTGAAACCAGCCGCAGCGATATGGA
ACTGCTGGAAAGCTTTGAAGAATTTGATTATCCGAACGAAATTGGCCCGGGCGTGTATGA
TATTCATAGCCCGAACGTGCCGAGCGTGGAATGGATTGAAGCGCTGCTGAAAAAAGCGG
CGAAACGCATTCCGGCGGAACGCCTGTGGGTGAACCCGGATTGCGGCCTGAAAACCCGC
GGCTGGCCGGAAACCCGCGCGGCGCTGGCGAACATGGTGCAGGCGGCGCAGAACCTGCG
CCGCGGC
MetE “Methionine Synthase II” E. coli, 753 aa,
SEQ ID NO: 38
MTILNHTLGFPRVGLRRELKKAQESYWAGNSTREELLAVGRELRARHWDQQKQAGIDLLPV
GDFAWYDHVLTTSLLLGNVPARHQNKDGSVDIDTLFRIGRGRAPTGEPAAAAEMTKWFNTN
YHYMVPEFVKGQQFKLTWTQLLDEVDEALALGHKVKPVLLGPVTWLWLGKVKGEQFDRL
SLLNDILPVYQQVLAELAKRGIEWVQIDEPALVLELPQAWLDAYKPAYDALQGQVKLLLTT
YFEGVTPNLDTITALPVQGLHVDLVHGKDDVAELHKRLPSDWLLSAGLINGRNVWRADLTE
KYAQIKDIVGKRDLWVASSCSLLHSPIDLSVETRLDAEVKSWFAFALQKCHELALLRDALNS
GDTAALAEWSAPIQARRHSTRVHNPAVEKRLAAITAQDSQRANVYEVRAEAQRARFKLPAW
PTTTIGSFPQTTEIRTLRLDFKKGNLDANNYRTGIAEHIKQAIVEQERLGLDVLVHGEAERND
MVEYFGEHLDGFVFTQNGWVQSYGSRCVKPPIVIGDISRPAPITVEWAKYAQSLTDKPVKGM
LTGPVTILCWSFPREDVSRETIAKQIALALRDEVADLEAAGIGIIQIDEPALREGLPLRRSDWD
AYLQWGVEAFRINAAVAKDDTQIHTHMCY CEFNDIMDSIAALDADVITIETSRSDMELLESFE
EFDYPNEIGPGVYDIHSPNVPSVEWIEALLKKAAKRIPAERLWVNPDCGLKTRGWPETRAAL
ANMVQAAQNLRRG
B. subtilis MetE, 2286 nt,
SEQ ID NO: 39
ATGACAACCATCAAAACATCGAATTTAGGATTTCCGAGAATCGGACTGAACCGGGAATG
GAAAAAAGCACTTGAAGCGTATTGGAAAGGCAGTACTGATAAAGATACGTTTTTGAAGC
AAATCGACGAACTATTTTTATCCGCAGTAAAAACACAAATTGACCAGCAGATTGATGTTG
TGCCTGTTTCTGATTTCACACAGTATGACCATGTACTCGACACAGCAGTCAGCTTCAACT
GGATCCCGAAACGGTTCAGACATTTGACTGACGCTACCGATACATACTTCGCTATCGCCC
GCGGAATCAAAGACGCTGTATCTAGTGAAATGACAAAATGGTTTAATACAAATTACCAT
TACATCGTTCCGGAATATGACGAGAGCATTGAGTTCCGTCTGACAAGAAACAAACAACT
CGAAGATTATCGCCGGATCAAACAGGAATACGGTGTGGAAACAAAGCCTGTGATTGTCG
GCCCTTATACGTTCGTTACGCTTGCTAAAGGCTATGAACCGTCTGAAGCAAAAGCGATCC
AAAAACGCCTTGTGCCATTATATGTACAGCTTTTGAAAGAGCTTGAAGAAGAAGGCGTA
AAATGGGTTCAAATCGATGAGCCGGCGCTCGTTACCGCCTCTAGTGAAGATGTACGCGG
CGCAAAAGAATTATTTGAAAGCATTACAAGTGAGCTTTCATCCTTGAATGTGCTTTTGCA
GACGTATTTTGATTCTGTTGATGCTTATGAAGAGCTGATCTCTTACCCGGTTCAGGGAATT
GGCCTTGATTTCGTTCACGACAAAGGCAGAAACCTGGAACAGCTTAAAACACATGGCTT
CCCGACAGATAAAGTGCTGGCAGCCGGCGTTATCGACGGACGCAACATTTGGAAAGCGG
ACCTTGAAGAGCGTCTCGATGCCGTTCTTGATATTCTCAGCATTGCAAAAGTTGATGAAC
TGTGGATTCAGCCTTCCAGCAGCCTGCTGCATGTTCCAGTAGCGAAACACCCTGATGAGC
ATTTGGAAAAAGACCTATTGAACGGATTATCCTACGCAAAAGAAAAGCTGGCCGAGCTG
ACAGCTTTGAAAGAAGGCTTAGTATCAGGAAAAGCGGCGATCAGCGAAGAGATTCAGCA
GGCTAAGGCTGATATCCAGGCGCTTAAACAGTTTGCAACAGGCGCCAATTCTGAACAAA
AGAAAGAGCTTGAGCAATTAACTGATAAAGACTTCAAGCGCCCGATTCCGTTTGAAGAA
CGTTTAGCCCTACAAAATGAATCTCTCGGCCTTCCGCTTTTGCCGACGACAACGATCGGC
AGCTTCCCGCAGTCTGCTGAAGTGCGGAGCGCACGCCAAAAATGGCGGAAAGCTGAGTG
GTCCGATGAACAGTATCAAAACTTTATCAATGCGGAAACAAAAAGATGGATTGATATTC
AGGAAGAATTGGAGCTTGATGTCCTTGTTCACGGCGAATTTGAACGGACAGACATGGTC
GAATACTTCGGTGAAAAGCTGGCCGGTTTCGCCTTCACTAAATATGCCTGGGTTCAATCA
TACGGCTCACGCTGTGTCCGCCCGCCAGTCATTTACGGAGATGTTGAATTTATTGAACCG
ATGACAGTGAAAGACACAGTCTACGCACAGTCATTGACTTCCAAGCATGTGAAAGGAAT
GCTGACGGGCCCGGTTACAATCTTAAACTGGTCTTTCCCTCGAAACGACATCTCGAGGAA
AGAAATCGCCTTCCAAATCGGGCTTGCCCTTCGCAAAGAAGTTAAAGCGCTTGAAGACG
CAGGCATTCAAATCATTCAAGTCGATGAACCAGCGCTGCGTGAAGGCCTTCCATTGAAA
ACCCGCGATTGGGATGAGTATTTGACTTGGGCGGCAGAAGCTTTCAGATTAACCACTTCT
TCCGTGAAAAACGAGACACAAATTCATACACATATGTGCTACAGCAACTTCGAAGATAT
CGTTGATACAATCAATGATCTTGATGCCGATGTGATTACAATCGAACATAGCAGAAGCCA
CGGAGGATTTTTAGATTACTTAAAAAACCACCCGTATTTGAAAGGGCTTGGCCTTGGTGT
ATATGACATTCACAGCCCTCGTGTGCCGTCAACTGAAGAAATGTACAATATTATCGTTGA
TGCGCTTGCCGTCTGTCCGACTGACCGCTTCTGGGTAAATCCAGACTGCGGATTGAAAAC
AAGACAGCAGGAAGAAACGGTTGCAGCATTGAAAAATATGGTTGAAGCCGCAAAACAG
GCAAGAGCACAGCAGACACAGCTAGTA
B. subtilis MetE, 762 aa,
SEQ ID NO: 40
MTTIKTSNLGFPRIGLNREWKKALEAYWKGSTDKDTFLKQIDELFLSAVKTQIDQQIDVVPVS
DFTQYDHVLDTAVSFNWIPKRFRHLTDATDTYFAIARGIKDAVSSEMTKWFNTNYHYIVPEY
DESIEFRLTRNKQLEDYRRIKQEYGVETKPVIVGPYTFVTLAKGYEPSEAKAIQKRLVPLYVQ
LLKELEEEGVKWVQIDEPALVTASSEDVRGAKELFESITSELSSLNVLLQTYFDSVDAYEELIS
YPVQGIGLDFVHDKGRNLEQLKTHGFPTDKVLAAGVIDGRNIWKADLEERLDAVLDILSIAK
VDELWIQPSSSLLHVPVAKHPDEHLEKDLLNGLSYAKEKLAELTALKEGLVSGKAAISEEIQQ
AKADIQALKQFATGANSEQKKELEQLTDKDFKRPIPFEERLALQNESLGLPLLPTTTIGSFPQS
AEVRSARQKWRKAEWSDEQYQNFINAETKRWIDIQEELELDVLVHGEFERTDMVEYFGEKL
AGFAFTKYAWVQSYGSRCVRPPVIYGDVEFIEPMTVKDTVYAQSLTSKHVKGMLTGPVTILN
WSFPRNDISRKEIAFQIGLALRKEVKALEDAGIQIIQVDEPALREGLPLKTRDWDEYLTWAAE
AFRLTTSSVKNETQIHTHMCYSNFEDIVDTINDLDADVITIEHSRSHGGFLDYLKNHPYLKGL
GLGVYDIHSPRVPSTEEMYNIIVDALAVCPTDRFWVNPDCGLKTRQQEETVAALKNMVEAA
KQARAQQTQLV

### Methionine Regulator

Another approach for reducing bioavailable methionine involves manipulation of methionine metabolism by modifying the activity of one or more methionine regulators in a microorganism. In this approach, the microorganism's methionine regulation, which normally involves a balance between methionine import, methionine catabolism, methionine export and methionine synthesis is shifted towards methionine import and/or methionine catabolism. This approach can also be combined with any of the other approaches described herein.

In one aspect, described herein is an engineered probiotic microorganism for reducing bioavailable methionine levels comprising at least one endogenous methionine regulator gene comprising at least one engineered inactivating or activating modification. In some embodiments of any of the aspects, the methionine regulator is a methionine sensor.

In some embodiments of any of the aspects, at least one endogenous methionine regulator gene comprises at least one engineered inactivating modification. In some embodiments of any of the aspects, at least one endogenous methionine regulator gene is partially or completely deleted (i.e., knocked out) in the engineered probiotic microorganism. In some embodiments of any of the aspects, the expression of the at least one endogenous methionine regulator gene is downregulated.

In some embodiments of any of the aspects, at least one endogenous methionine regulator gene comprises at least one engineered activating modification. In some embodiments of any of the aspects, the expression of the at least one endogenous methionine regulator gene is upregulated. In some embodiments of any of the aspects, the at least one engineered activating modification of the endogenous methionine regulator gene comprises at least one engineered activating mutation in the at least one endogenous methionine regulator gene. In some embodiments of any of the aspects, the at least one engineered activating modification of the endogenous methionine regulator gene comprises at least one engineered activating mutation in a promoter operatively linked to the at least one endogenous methionine regulator gene.

In some embodiments of any of the aspects, the at least one engineered inactivating modification of the endogenous methionine regulator gene comprises at least one engineered inactivating mutation in the at least one endogenous methionine regulator gene. In some embodiments of any of the aspects, the at least one engineered inactivating modification of the endogenous methionine regulator gene comprises at least one engineered inactivating mutation in a promoter operatively linked to the at least one endogenous methionine regulator gene. In some embodiments of any of the aspects, the at least one engineered inactivating modification of the endogenous methionine regulator gene comprises at least one inhibitory RNA molecule specific to at least one messenger RNA (mRNA) expressed by the at least one endogenous methionine regulator gene. Non-limiting examples of inhibitory RNAs include small interfering RNA (siRNA), micro RNA (miRNA), CRISPR RNA (crRNA and associated Cas endonuclease), and the like.

In some embodiments of any of the aspects, the engineered probiotic microorganism comprises at least one exogenous copy of at least one gene encoding an enzyme that catalyzes the degradation of methionine and at least one endogenous methionine regulator gene comprising at least one engineered inactivating or activating modification.

Non-limiting examples of an endogenous methionine regulator include MetJ (see e.g., SEQ ID NO: 41-42) or MetR (see e.g., SEQ ID NO: 43-44). MetJ and MetR work in concert to repress synthesis of genes that make and/or import methionine when it is high, and drive synthesis of exporters to help reduce excess methionine. When methionine levels drop, they de-repress methionine importer genes. If methionine levels still fall, MetJ and MetR de-repress methionine synthesis genes to make more methionine from scratch. Modification of these activities can be helpful in shifting the balance of methionine-limiting to methionine-increasing activities towards the methionine-limiting (sequestering/accumulation and/or degradation) side.

In some embodiments of any of the aspects, the methionine regulator is encoded by one of SEQ ID NO: 41 or 43 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NO: 41 or 43, that maintains the same function, or a codon-optimized version thereof.

In some embodiments of any of the aspects, the methionine regulator comprises one of SEQ ID NOs: 42 or 44 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NOs: 42 or 44, that maintains the same function (e.g., sensing of methionine and/or regulation of methionine-associated genes).

In some embodiments of any of the aspects, the engineered probiotic microorganism comprises at least one engineered activating modification in MetJ (see e.g., SEQ ID NO: 41-42) or MetR (see e.g., SEQ ID NO: 43-44). In some embodiments of any of the aspects, the engineered probiotic microorganism comprises at least one engineered activating modification in MetJ (see e.g., SEQ ID NO: 41-42). In some embodiments of any of the aspects, the engineered probiotic microorganism comprises at least one engineered activating modification in MetR (see e.g., SEQ ID NO: 43-44). In some embodiments of any of the aspects, the engineered probiotic microorganism comprises at least one engineered activating modification in MetJ (see e.g., SEQ ID NO: 41-42) and MetR (see e.g., SEQ ID NO: 43-44).

In some embodiments of any of the aspects, the engineered probiotic microorganism comprises at least one engineered inactivating modification in MetJ (see e.g., SEQ ID NO: 41-42) or MetR (see e.g., SEQ ID NO: 43-44). In some embodiments of any of the aspects, the engineered probiotic microorganism comprises at least one engineered inactivating modification in MetJ (see e.g., SEQ ID NO: 41-42). In some embodiments of any of the aspects, the engineered probiotic microorganism comprises at least one engineered inactivating modification in MetR (see e.g., SEQ ID NO: 43-44). In some embodiments of any of the aspects, the engineered probiotic microorganism comprises at least one engineered inactivating modification in MetJ (see e.g., SEQ ID NO: 41-42) and MetR (see e.g., SEQ ID NO: 43-44).

In some embodiments of any of the aspects, the engineered probiotic microorganism comprises at least one engineered activating modification in MetJ (see e.g., SEQ ID NO: 41-42) and at least one engineered inactivating modification in MetR (see e.g., SEQ ID NO: 43-44). In some embodiments of any of the aspects, the engineered probiotic microorganism comprises at least one engineered inactivating modification in MetJ (see e.g., SEQ ID NO: 41-42) and at least one engineered activating modification in MetR (see e.g., SEQ ID NO: 43-44).

In some embodiments of any of the aspects, the methionine regulator is derived from a methionine regulator gene or polypeptide of E. coli (see e.g., SEQ ID NOs: 41-44).

MetJ, E. coli, 315 nt,
SEQ ID NO: 41
ATGGCGGAATGGAGCGGCGAATATATTAGCCCGTATGCGGAACATGGCAAAAAAAGCG
AACAGGTGAAAAAAATTACCGTGAGCATTCCGCTGAAAGTGCTGAAAATTCTGACCGAT
GAACGCACCCGCCGCCAGGTGAACAACCTGCGCCATGCGACCAACAGCGAACTGCTGTG
CGAAGCGTTTCTGCATGCGTTTACCGGCCAGCCGCTGCCGGATGATGCGGATCTGCGCAA
AGAACGCAGCGATGAAATTCCGGAAGCGGCGAAAGAAATTATGCGCGAAATGGGCATT
AACCCGGAAACCTGGGAATAT
MetJ E. coli, 105 aa,
SEQ ID NO: 42
MAEWSGEYISPYAEHGKKSEQVKKITVSIPLKVLKILTDERTRRQVNNLRHATNSELLCEAFL
HAFTGQPLPDDADLRKERSDEIPEAAKEIMREMGINPETWEY
MetR E. coli, 951 nt,
SEQ ID NO: 43
ATGATTGAAGTGAAACATCTGAAAACCCTGCAGGCGCTGCGCAACTGCGGCAGCCTGGC
GGCGGCGGCGGCGACCCTGCATCAGACCCAGAGCGCGCTGAGCCATCAGTTTAGCGATC
TGGAACAGCGCCTGGGCTTTCGCCTGTTTGTGCGCAAAAGCCAGCCGCTGCGCTTTACCC
CGCAGGGCGAAATTCTGCTGCAGCTGGCGAACCAGGTGCTGCCGCAGATTAGCCAGGCG
CTGCAGGCGTGCAACGAACCGCAGCAGACCCGCCTGCGCATTGCGATTGAATGCCATAG
CTGCATTCAGTGGCTGACCCCGGCGCTGGAAAACTTTCATAAAAACTGGCCGCAGGTGG
AAATGGATTTTAAAAGCGGCGTGACCTTTGATCCGCAGCCGGCGCTGCAGCAGGGCGAA
CTGGATCTGGTGATGACCAGCGATATTCTGCCGCGCAGCGGCCTGCATTATAGCCCGATG
TTTGATTATGAAGTGCGCCTGGTGCTGGCGCCGGATCATCCGCTGGCGGCGAAAACCCGC
ATTACCCCGGAAGATCTGGCGAGCGAAACCCTGCTGATTTATCCGGTGCAGCGCAGCCG
CCTGGATGTGTGGCGCCATTTTCTGCAGCCGGCGGGCGTGAGCCCGAGCCTGAAAAGCG
TGGATAACACCCTGCTGCTGATTCAGATGGTGGCGGCGCGCATGGGCATTGCGGCGCTGC
CGCATTGGGTGGTGGAAAGCTTTGAACGCCAGGGCCTGGTGGTGACCAAAACCCTGGGC
GAAGGCCTGTGGAGCCGCCTGTATGCGGCGGTGCGCGATGGCGAACAGCGCCAGCCGGT
GACCGAAGCGTTTATTCGCAGCGCGCGCAACCATGCGTGCGATCATCTGCCGTTTGTGAA
AAGCGCGGAACGCCCGACCTATGATGCGCCGACCGTGCGCCCGGGCAGCCCGGCGCGCC
TG
MetR E. coli, 317 aa,
SEQ ID NO: 44
MIEVKHLKTLQALRNCGSLAAAAATLHQTQSALSHQFSDLEQRLGFRLFVRKSQPLRFTPQG
EILLQLANQVLPQISQALQACNEPQQTRLRIAIECHSCIQWLTPALENFHKNWPQVEMDFKSG
VTFDPQPALQQGELDLVMTSDILPRSGLHYSPMFDYEVRLVLAPDHPLAAKTRITPEDLASET
LLIYPVQRSRLDVWRHFLQPAGVSPSLKSVDNTLLLIQMVAARMGIAALPHWVVESFERQGL
VVTKTLGEGLWSRLYAAVRDGEQRQPVTEAFIRSARNHACDHLPFVKSAERPTYDAPTVRP
GSPARL

### Methanethiol-Reducing Microorganism

Methanethiol is a product of methionine degradation, e.g., by methionine gamma lyase. The exceedingly disagreeable odor of methanethiol is a potential downside of methionine reduction approaches that generate this product. In one aspect, described herein is an engineered probiotic microorganism for reducing methanethiol levels. In some embodiments of any of the aspects, the engineered probiotic microorganism comprises and expresses an exogenous gene encoding a methanethiol catabolizing enzyme. In some embodiments of any of the aspects, the engineered probiotic microorganism comprises and expresses at least one exogenous copy of at least one gene encoding an enzyme that catalyzes the degradation of methionine and an exogenous gene encoding a methanethiol catabolizing enzyme.

In some embodiments of any of the aspects, the engineered probiotic microorganism comprises and expresses an exogenous gene encoding a methanethiol catabolizing enzyme, and one or more of: (a) at least one exogenous copy of at least one gene encoding an enzyme that catalyzes the degradation of methionine; (b) at least one exogenous copy of at least one functional methionine importer gene; (c) at least one endogenous methionine importer gene comprising at least one engineered activating modification; (d) at least one endogenous methionine synthesis gene comprising at least one engineered inactivating modification; and/or (e) at least one endogenous methionine regulator gene comprising at least one engineered inactivating or activating modification. In some embodiments of any of the aspects, the engineered probiotic microorganism comprises and expresses an exogenous gene encoding a methanethiol catabolizing enzyme and one of the exemplary combinations of genes from Table 2.

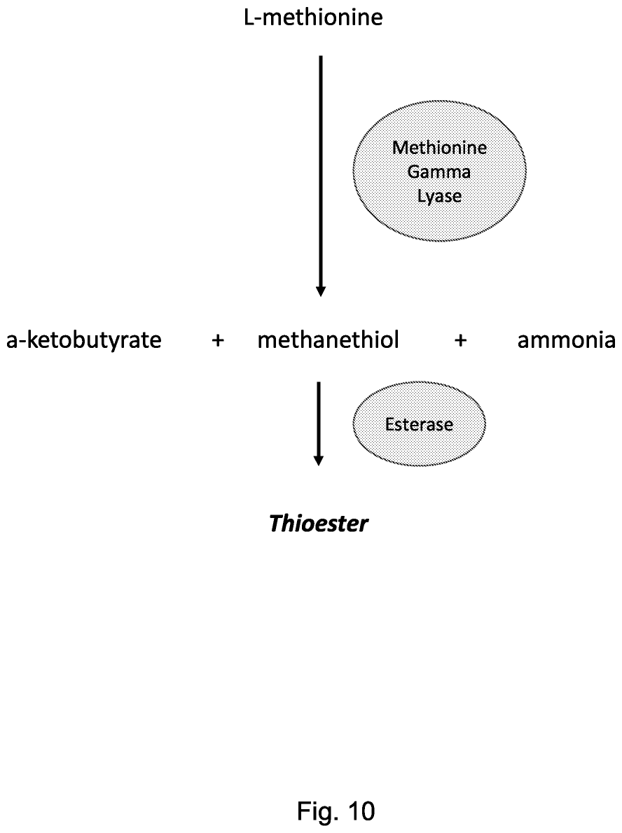

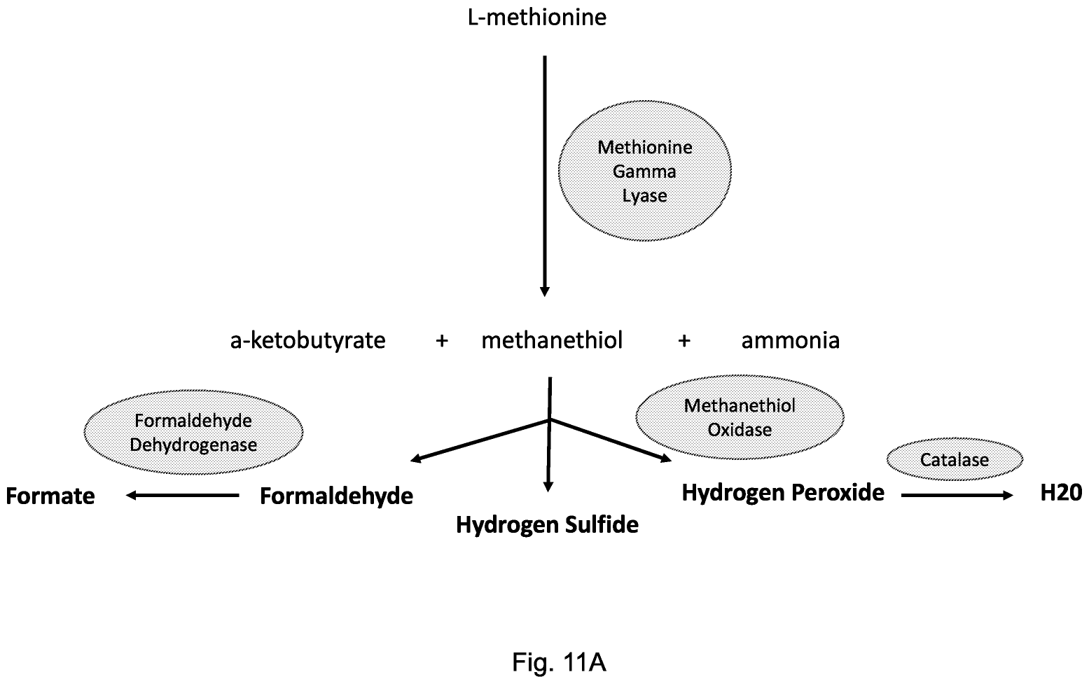

In some embodiments of any of the aspects, the methanethiol-catabolizing enzyme is an esterase or a methanethiol oxidase. In some embodiments of any of the aspects, the methanethiol-catabolizing enzyme is an esterase (see e.g., FIG. 10). In some embodiments of any of the aspects, the methanethiol-catabolizing enzyme is a methanethiol oxidase (see e.g., FIG. 11A-11B). Non-limiting examples of a methanethiol oxidase includes a human methanethiol oxidase (see e.g., SEQ ID NOs: 45-46) or a bacterial methanethiol oxidase (see e.g., SEQ ID NOs: 47-48).

In some embodiments of any of the aspects, the methanethiol oxidase is encoded by one of SEQ ID NOs: 45 or 47 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 45 or 47, that maintains the same function, or a codon-optimized version thereof.

In some embodiments of any of the aspects, the methanethiol oxidase comprises SEQ ID NOs: 46 or 48 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NOs: 46 or 48, that maintains the same function (e.g., methanethiol oxidation; e.g., production of formaldehyde, hydrogen sulfide, and hydrogen peroxide from methanethiol).

In some embodiments of any of the aspects, the methanethiol oxidase is derived from a human methanethiol oxidase gene (see e.g., SEQ ID NOs: 45-46). In some embodiments of any of the aspects, the methionine synthesis enzyme is derived from a bacterial methanethiol oxidase gene (see e.g., SEQ ID NOs: 47-48).

SBP1_HUMAN Methanethiol oxidase, Homo sapiens, 1416 nt,
SEQ ID NO: 45
ATGGCGACCAAATGCGGCAACTGCGGCCCGGGCTATAGCACCCCGCTGGAAGCGATGAA
AGGCCCGCGCGAAGAAATTGTGTATCTGCCGTGCATTTATCGCAACACCGGCACCGAAG
CGCCGGATTATCTGGCGACCGTGGATGTGGATCCGAAAAGCCCGCAGTATTGCCAGGTG
ATTCATCGCCTGCCGATGCCGAACCTGAAAGATGAACTGCATCATAGCGGCTGGAACAC
CTGCAGCAGCTGCTTTGGCGATAGCACCAAAAGCCGCACCAAACTGGTGCTGCCGAGCC
TGATTAGCAGCCGCATTTATGTGGTGGATGTGGGCAGCGAACCGCGCGCGCCGAAACTG
CATAAAGTGATTGAACCGAAAGATATTCATGCGAAATGCGAACTGGCGTTTCTGCATACC
AGCCATTGCCTGGCGAGCGGCGAAGTGATGATTAGCAGCCTGGGCGATGTGAAAGGCAA
CGGCAAAGGCGGCTTTGTGCTGCTGGATGGCGAAACCTTTGAAGTGAAAGGCACCTGGG
AACGCCCGGGCGGCGCGGCGCCGCTGGGCTATGATTTTTGGTATCAGCCGCGCCATAAC
GTGATGATTAGCACCGAATGGGCGGCGCCGAACGTGCTGCGCGATGGCTTTAACCCGGC
GGATGTGGAAGCGGGCCTGTATGGCAGCCATCTGTATGTGTGGGATTGGCAGCGCCATG
AAATTGTGCAGACCCTGAGCCTGAAAGATGGCCTGATTCCGCTGGAAATTCGCTTTCTGC
ATAACCCGGATGCGGCGCAGGGCTTTGTGGGCTGCGCGCTGAGCAGCACCATTCAGCGC
TTTTATAAAAACGAAGGCGGCACCTGGAGCGTGGAAAAAGTGATTCAGGTGCCGCCGAA
AAAAGTGAAAGGCTGGCTGCTGCCGGAAATGCCGGGCCTGATTACCGATATTCTGCTGA
GCCTGGATGATCGCTTTCTGTATTTTAGCAACTGGCTGCATGGCGATCTGCGCCAGTATG
ATATTAGCGATCCGCAGCGCCCGCGCCTGACCGGCCAGCTGTTTCTGGGCGGCAGCATTG
TGAAAGGCGGCCCGGTGCAGGTGCTGGAAGATGAAGAACTGAAAAGCCAGCCGGAACC
GCTGGTGGTGAAAGGCAAACGCGTGGCGGGCGGCCCGCAGATGATTCAGCTGAGCCTGG
ATGGCAAACGCCTGTATATTACCACCAGCCTGTATAGCGCGTGGGATAAACAGTTTTATC
CGGATCTGATTCGCGAAGGCAGCGTGATGCTGCAGGTGGATGTGGATACCGTGAAAGGC
GGCCTGAAACTGAACCCGAACTTTCTGGTGGATTTTGGCAAAGAACCGCTGGGCCCGGC
GCTGGCGCATGAACTGCGCTATCCGGGCGGCGATTGCAGCAGCGATATTTGGATT
Methanethiol oxidase, Homo sapiens, 472 aa,
SEQ ID NO: 46
MATKCGNCGPGYSTPLEAMKGPREEIVYLPCIYRNTGTEAPDYLATVDVDPKSPQYCQVIHR
LPMPNLKDELHHSGWNTCSSCFGDSTKSRTKLVLPSLISSRIYVVDVGSEPRAPKLHKVIEPK
DIHAKCELAFLHTSHCLASGEVMISSLGDVKGNGKGGFVLLDGETFEVKGTWERPGGAAPL
GYDFWYQPRHNVMISTEWAAPNVLRDGFNPADVEAGLYGSHLYVWDWQRHEIVQTLSLKD
GLIPLEIRFLHNPDAAQGFVGCALSSTIQRFYKNEGGTWSVEKVIQVPPKKVKGWLLPEMPGL
ITDILLSLDDRFLYFSNWLHGDLRQYDISDPQRPRLTGQLFLGGSIVKGGPVQVLEDEELKSQP
EPLVVKGKRVAGGPQMIQLSLDGKRLYITTSLYSAWDKQFYPDLIREGSVMLQVDVDTVKG
GLKLNPNFLVDFGKEPLGPALAHELRYPGGDCSSDIWI
MTO_BACTERIA Methanethiol oxidase, 1305 nt,
SEQ ID NO: 47
ATGAAAAAACATCTGCTGGCGGGCGCGTGCGCGCTGGCGATGGGCTTTGCGGTGATTCC
GGGCACCTTTGCGGATGAAACCTGCAACAGCCCGTTTACCACCGCGCTGATTACCGGCCA
GGAACAGTATCTGCATGTGTGGACCCTGGGCATGCCGGGCGTGGGCGATGAAAGCGATA
AACTGGTGACCATTAGCGTGGATCCGAAAAGCGATAAATATGGCAAAGTGATTAACACC
CTGAGCGTGGGCGGCCGCGGCGAAGCGCATCATACCGGCTTTACCGATGATCGCCGCTA
TCTGTGGGCGGGCCGCCTGGATGATAACAAAATTTTTATTTTTGATCTGATTGATCCGGC
GAACCCGAAACTGATTAAAACCATTACCGATTTTGCGGATCGCACCGGCTATGTGGGCCC
GCATACCTTTTATGCGCTGCCGGGCCGCATGCTGATTCAGGCGCTGAGCAACACCAAAAC
CCATGATGGCCAGACCGGCCTGGCGGTGTATAGCAACGCGGGCGAACTGGTGAGCCTGC
ATCCGATGCCGGTGACCGATGGCGGCGATGGCTATGGCTATGATATTGGCATTAACCCGG
CGAAAAACGTGCTGCTGACCAGCAGCTTTACCGGCTGGAACAACTATATGATGGATCTG
GGCAAAATGGTGAAAGATCCGGAAGCGATGAAACGCTTTGGCAACACCATGGCGATTTG
GGATCTGAAAAGCATGAAAGCGGAAAAAATTCTGAACGTGCCGGGCGCGCCGCTGGAA
ATTCGCTGGAGCCTGAAACCGGAACATAACTGGGCGTATACCGCGACCGCGCTGACCAG
CAAACTGTGGCTGATTAAACAGGATGATAAAGGCGAATGGATTGCGAAAGAAACCGGCA
CCATTGGCGATCCGAGCAAAATTCCGCTGCCGGTGGATATTAGCATTACCGCGGATGCGA
AAGGCCTGTGGGTGAACACCTTTCTGGATGGCACCACCCGCTTTTATGATATTAGCGAAC
CGGAACATCCGAAAGAAGTGTTTAGCAAAAAAATGGGCAACCAGGTGAACATGGTGAG
CCAGAGCTATGATGGCAAACGCGTGTATTTTACCACCAGCCTGATTGCGAACTGGGATAA
AAAAGGCGCGGAAAACGATCAGTGGCTGAAAGCGTATGATTGGGATGGCAAAGAACTG
GTGGAAAAATTTACCGTGGATTTTAACGAACTGAAACTGGGCCGCGCGCATCATATGAA
ATTTAGCAGCAAAACCAACGCGGCGGAACTGGGCACCAACCAGAGCTTTCCGACCCGCC
AG
MTO_BACTERIA Methanethiol oxidase, 435 aa,
SEQ ID NO: 48
MKKHLLAGACALAMGFAVIPGTFADETCNSPFTTALITGQEQYLHVWTLGMPGVGDESDKL
VTISVDPKSDKYGKVINTLSVGGRGEAHHTGFTDDRRYLWAGRLDDNKIFIFDLIDPANPKLI
KTITDFADRTGYVGPHTFYALPGRMLIQALSNTKTHDGQTGLAVYSNAGELVSLHPMPVTD
GGDGYGYDIGINPAKNVLLTSSFTGWNNYMMDLGKMVKDPEAMKRFGNTMAIWDLKSMK
AEKILNVPGAPLEIRWSLKPEHNWAYTATALTSKLWLIKQDDKGEWIAKETGTIGDPSKIPLP
VDISITADAKGLWVNTFLDGTTRFYDISEPEHPKEVFSKKMGNQVNMVSQSYDGKRVYFTTS
LIANWDKKGAENDQWLKAYDWDGKELVEKFTVDFNELKLGRAHHMKFSSKTNAAELGTN
QSFPTRQ

In some embodiments of any of the aspects, the engineered probiotic microorganism comprises and expresses an exogenous gene encoding a methanethiol oxidase and at least one exogenous gene selected from the group consisting of a catalase (e.g., katG; see e.g., SEQ ID NO: 49-50); formaldehyde dehydrogenase (e.g., fdhA; see e.g., SEQ ID NO: 51-52); formate acetyltransferase (see e.g., SEQ ID NO: 53-54); and sulfite reductase (e.g., cysJ; see e.g., SEQ ID NO: 55-56); (see e.g., FIG. 11A-11B). In some embodiments of any of the aspects, the catalase is derived from a catalase gene or polypeptide of E. coli (see e.g., SEQ ID NOs: 49-50). In some embodiments of any of the aspects, the formaldehyde dehydrogenase is derived from a formaldehyde dehydrogenase gene or polypeptide of Pseudomonas putida (see e.g., SEQ ID NOs: 51-52). In some embodiments of any of the aspects, the formate acetyltransferase is derived from a formate acetyltransferase gene or polypeptide of Clostridium pasteurianum (see e.g., SEQ ID NOs: 53-54). In some embodiments of any of the aspects, the sulfite reductase is derived from a sulfite reductase gene or polypeptide of E. coli (see e.g., SEQ ID NOs: 55-56). Non-limiting examples of exogenous genes that can be combined with the exogenous methanethiol oxidase are provided in Table 3.

TABLE 3
Exemplary engineered probiotic microorganisms comprising
exogenous methanethiol oxidase and the indicated enzyme.

| | formaldehyde | formate | |
|---|---|---|---|
| catalase | dehydrogenase | acetyltransferase | sulfite reductase |
| X | | | |
| | X | | |
| X | X | | |
| | | X | |
| X | | X | |
| | X | X | |
| X | X | X | |
| | | | X |
| X | | | X |
| | X | | X |
| X | X | | X |
| | | X | X |
| X | | X | X |
| | X | X | X |
| X | X | X | X |

In some embodiments of any of the aspects, the engineered probiotic microorganism comprises and expresses an exogenous gene encoding a methanethiol oxidase, and one of the exemplary combinations of genes from Table 3. In some embodiments of any of the aspects, the engineered probiotic microorganism comprises and expresses an exogenous gene encoding a methanethiol oxidase; one of the exemplary combinations of genes from Table 2; and one of the exemplary combinations of genes from Table 3. The methanethiol-reducing engineered probiotic microorganism can, like the other probiotic microorganisms described herein, be formulated as a dietary supplement, pharmaceutical composition, and/or food composition.

In some embodiments of any of the aspects, the catalase is encoded by SEQ ID NO: 49 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 49, that maintains the same function, or a codon-optimized version thereof.

In some embodiments of any of the aspects, the catalase comprises SEQ ID NO: 50 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 50, that maintains the same function (e.g., production of water from hydrogen peroxide).

KATG, E. coli Catalase-peroxidase, 2178 nt,
SEQ ID NO: 49
ATGAGCACCAGCGATGATATTCATAACACCACCGCGACCGGCAAATGCCCGTTTCATCA
GGGCGGCCATGATCAGAGCGCGGGCGCGGGCACCACCACCCGCGATTGGTGGCCGAACC
AGCTGCGCGTGGATCTGCTGAACCAGCATAGCAACCGCAGCAACCCGCTGGGCGAAGAT
TTTGATTATCGCAAAGAATTTAGCAAACTGGATTATTATGGCCTGAAAAAAGATCTGAAA
GCGCTGCTGACCGAAAGCCAGCCGTGGTGGCCGGCGGATTGGGGCAGCTATGCGGGCCT
GTTTATTCGCATGGCGTGGCATGGCGCGGGCACCTATCGCAGCATTGATGGCCGCGGCGG
CGCGGGCCGCGGCCAGCAGCGCTTTGCGCCGCTGAACAGCTGGCCGGATAACGTGAGCC
TGGATAAAGCGCGCCGCCTGCTGTGGCCGATTAAACAGAAATATGGCCAGAAAATTAGC
TGGGCGGATCTGTTTATTCTGGCGGGCAACGTGGCGCTGGAAAACAGCGGCTTTCGCACC
TTTGGCTTTGGCGCGGGCCGCGAAGATGTGTGGGAACCGGATCTGGATGTGAACTGGGG
CGATGAAAAAGCGTGGCTGACCCATCGCCATCCGGAAGCGCTGGCGAAAGCGCCGCTGG
GCGCGACCGAAATGGGCCTGATTTATGTGAACCCGGAAGGCCCGGATCATAGCGGCGAA
CCGCTGAGCGCGGCGGCGGCGATTCGCGCGACCTTTGGCAACATGGGCATGAACGATGA
AGAAACCGTGGCGCTGATTGCGGGCGGCCATACCCTGGGCAAAACCCATGGCGCGGGCC
CGACCAGCAACGTGGGCCCGGATCCGGAAGCGGCGCCGATTGAAGAACAGGGCCTGGG
CTGGGCGAGCACCTATGGCAGCGGCGTGGGCGCGGATGCGATTACCAGCGGCCTGGAAG
TGGTGTGGACCCAGACCCCGACCCAGTGGAGCAACTATTTTTTTGAAAACCTGTTTAAAT
ATGAATGGGTGCAGACCCGCAGCCCGGCGGGCGCGATTCAGTTTGAAGCGGTGGATGCG
CCGGAAATTATTCCGGATCCGTTTGATCCGAGCAAAAAACGCAAACCGACCATGCTGGT
GACCGATCTGACCCTGCGCTTTGATCCGGAATTTGAAAAAATTAGCCGCCGCTTTCTGAA
CGATCCGCAGGCGTTTAACGAAGCGTTTGCGCGCGCGTGGTTTAAACTGACCCATCGCGA
TATGGGCCCGAAAAGCCGCTATATTGGCCCGGAAGTGCCGAAAGAAGATCTGATTTGGC
AGGATCCGCTGCCGCAGCCGATTTATAACCCGACCGAACAGGATATTATTGATCTGAAAT
TTGCGATTGCGGATAGCGGCCTGAGCGTGAGCGAACTGGTGAGCGTGGCGTGGGCGAGC
GCGAGCACCTTTCGCGGCGGCGATAAACGCGGCGGCGCGAACGGCGCGCGCCTGGCGCT
GATGCCGCAGCGCGATTGGGATGTGAACGCGGCGGCGGTGCGCGCGCTGCCGGTGCTGG
AAAAAATTCAGAAAGAAAGCGGCAAAGCGAGCCTGGCGGATATTATTGTGCTGGCGGGC
GTGGTGGGCGTGGAAAAAGCGGCGAGCGCGGCGGGCCTGAGCATTCATGTGCCGTTTGC
GCCGGGCCGCGTGGATGCGCGCCAGGATCAGACCGATATTGAAATGTTTGAACTGCTGG
AACCGATTGCGGATGGCTTTCGCAACTATCGCGCGCGCCTGGATGTGAGCACCACCGAA
AGCCTGCTGATTGATAAAGCGCAGCAGCTGACCCTGACCGCGCCGGAAATGACCGCGCT
GGTGGGCGGCATGCGCGTGCTGGGCGCGAACTTTGATGGCAGCAAAAACGGCGTGTTTA
CCGATCGCGTGGGCGTGCTGAGCAACGATTTTTTTGTGAACCTGCTGGATATGCGCTATG
AATGGAAAGCGACCGATGAAAGCAAAGAACTGTTTGAAGGCCGCGATCGCGAAACCGG
CGAAGTGAAATTTACCGCGAGCCGCGCGGATCTGGTGTTTGGCAGCAACAGCGTGCTGC
GCGCGGTGGCGGAAGTGTATGCGAGCAGCGATGCGCATGAAAAATTTGTGAAAGATTTT
GTGGCGGCGTGGGTGAAAGTGATGAACCTGGATCGCTTTGATCTGCTG
KATG, E. coli, Catalase-peroxidase, 726 aa,
SEQ ID NO: 50
MSTSDDIHNTTATGKCPFHQGGHDQSAGAGTTTRDWWPNQLRVDLLNQHSNRSNPLGEDF
DYRKEFSKLDYYGLKKDLKALLTESQPWWPADWGSYAGLFIRMAWHGAGTYRSIDGRGGA
GRGQQRFAPLNSWPDNVSLDKARRLLWPIKQKYGQKISWADLFILAGNVALENSGFRTFGFG
AGREDVWEPDLDVNWGDEKAWLTHRHPEALAKAPLGATEMGLIYVNPEGPDHSGEPLSAA
AAIRATFGNMGMNDEETVALIAGGHTLGKTHGAGPTSNVGPDPEAAPIEEQGLGWASTYGS
GVGADAITSGLEVVWTQTPTQWSNYFFENLFKYEWVQTRSPAGAIQFEAVDAPEIIPDPFDPS
KKRKPTMLVTDLTLRFDPEFEKISRRFLNDPQAFNEAFARAWFKLTHRDMGPKSRYIGPEVP
KEDLIWQDPLPQPIYNPTEQDIIDLKFAIADSGLSVSELVSVAWASASTFRGGDKRGGANGAR
LALMPQRDWDVNAAAVRALPVLEKIQKESGKASLADIIVLAGVVGVEKAASAAGLSIHVPF
APGRVDARQDQTDIEMFELLEPIADGFRNYRARLDVSTTESLLIDKAQQLTLTAPEMTALVG
GMRVLGANFDGSKNGVFTDRVGVLSNDFFVNLLDMRYEWKATDESKELFEGRDRETGEVK
FTASRADLVFGSNSVLRAVAEVYASSDAHEKFVKDFVAAWVKVMNLDRFDLL

In some embodiments of any of the aspects, the formaldehyde dehydrogenase is encoded by SEQ ID NO: 51 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 51, that maintains the same function, or a codon-optimized version thereof.

In some embodiments of any of the aspects, the formaldehyde dehydrogenase comprises SEQ ID NO: 52 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 52, that maintains the same function (e.g., production of formate from formaldehyde).

FADH_PSEPU Pseudomonas putida Glutathione-independent formaldehyde
dehydrogenase, 1197 nt,
SEQ ID NO: 51
ATGAGCGGCAACCGCGGCGTGGTGTATCTGGGCAGCGGCAAAGTGGAAGTGCAGAAAAT
TGATTATCCGAAAATGCAGGATCCGCGCGGCAAAAAAATTGAACATGGCGTGATTCTGA
AAGTGGTGAGCACCAACATTTGCGGCAGCGATCAGCATATGGTGCGCGGCCGCACCACC
GCGCAGGTGGGCCTGGTGCTGGGCCATGAAATTACCGGCGAAGTGATTGAAAAAGGCCG
CGATGTGGAAAACCTGCAGATTGGCGATCTGGTGAGCGTGCCGTTTAACGTGGCGTGCG
GCCGCTGCCGCAGCTGCAAAGAAATGCATACCGGCGTGTGCCTGACCGTGAACCCGGCG
CGCGCGGGCGGCGCGTATGGCTATGTGGATATGGGCGATTGGACCGGCGGCCAGGCGGA
ATATCTGCTGGTGCCGTATGCGGATTTTAACCTGCTGAAACTGCCGGATCGCGATAAAGC
GATGGAAAAAATTCGCGATCTGACCTGCCTGAGCGATATTCTGCCGACCGGCTATCATGG
CGCGGTGACCGCGGGCGTGGGCCCGGGCAGCACCGTGTATGTGGCGGGCGCGGGCCCGG
TGGGCCTGGCGGCGGCGGCGAGCGCGCGCCTGCTGGGCGCGGCGGTGGTGATTGTGGGC
GATCTGAACCCGGCGCGCCTGGCGCATGCGAAAGCGCAGGGCTTTGAAATTGCGGATCT
GAGCCTGGATACCCCGCTGCATGAACAGATTGCGGCGCTGCTGGGCGAACCGGAAGTGG
ATTGCGCGGTGGATGCGGTGGGCTTTGAAGCGCGCGGCCATGGCCATGAAGGCGCGAAA
CATGAAGCGCCGGCGACCGTGCTGAACAGCCTGATGCAGGTGACCCGCGTGGCGGGCAA
AATTGGCATTCCGGGCCTGTATGTGACCGAAGATCCGGGCGCGGTGGATGCGGCGGCGA
AAATTGGCAGCCTGAGCATTCGCTTTGGCCTGGGCTGGGCGAAAAGCCATAGCTTTCATA
CCGGCCAGACCCCGGTGATGAAATATAACCGCGCGCTGATGCAGGCGATTATGTGGGAT
CGCATTAACATTGCGGAAGTGGTGGGCGTGCAGGTGATTAGCCTGGATGATGCGCCGCG
CGGCTATGGCGAATTTGATGCGGGCGTGCCGAAAAAATTTGTGATTGATCCGCATAAAA
CCTTTAGCGCGGCG
FADH_PSEPU Pseudomonas putida Glutathione-independent formaldehyde
dehydrogenase, 399 aa,
SEQ ID NO: 52
MSGNRGVVYLGSGKVEVQKIDYPKMQDPRGKKIEHGVILKVVSTNICGSDQHMVRGRTTAQ
VGLVLGHEITGEVIEKGRDVENLQIGDLVSVPFNVACGRCRSCKEMHTGVCLTVNPARAGG
AYGYVDMGDWTGGQAEYLLVPYADFNLLKLPDRDKAMEKIRDLTCLSDILPTGYHGAVTA
GVGPGSTVYVAGAGPVGLAAAASARLLGAAVVIVGDLNPARLAHAKAQGFEIADLSLDTPL
HEQIAALLGEPEVDCAVDAVGFEARGHGHEGAKHEAPATVLNSLMQVTRVAGKIGIPGLYV
TEDPGAVDAAAKIGSLSIRFGLGWAKSHSFHTGQTPVMKYNRALMQAIMWDRINIAEVVGV
QVISLDDAPRGYGEFDAGVPKKFVIDPHKTFSAA

In some embodiments of any of the aspects, the formate acetyltransferase is encoded by SEQ ID NO: 53 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 53, that maintains the same function, or a codon-optimized version thereof.

In some embodiments of any of the aspects, the formate acetyltransferase comprises SEQ ID NO: 54 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 54, that maintains the same function (e.g., production of pyruvate from formate).

PFL_CLOPA Clostridium pasteurianum, Formate acetyltransferase,
2220 nt,
SEQ ID NO: 53
ATGTTTAAACAGTGGGAAGGCTTTCAGGATGGCGAATGGACCAACGATGTGAACGTGCG
CGATTTTATTCAGAAAAACTATAAAGAATATACCGGCGATAAAAGCTTTCTGAAAGGCC
CGACCGAAAAAACCAAAAAAGTGTGGGATAAAGCGGTGAGCCTGATTCTGGAAGAACT
GAAAAAAGGCATTCTGGATGTGGATACCGAAACCATTAGCGGCATTAACAGCTTTAAAC
CGGGCTATCTGGATAAAGATAACGAAGTGATTGTGGGCTTTCAGACCGATGCGCCGCTG
AAACGCATTACCAACCCGTTTGGCGGCATTCGCATGGCGGAACAGAGCCTGAAAGAATA
TGGCTTTAAAATTAGCGATGAAATGCATAACATTTTTACCAACTATCGCAAAACCCATAA
CCAGGGCGTGTTTGATGCGTATAGCGAAGAAACCCGCATTGCGCGCAGCGCGGGCGTGC
TGACCGGCCTGCCGGATGCGTATGGCCGCGGCCGCATTATTGGCGATTATCGCCGCGTGG
CGCTGTATGGCATTGATTTTCTGATTCAGGAAAAAAAAAAAGATCTGAGCAACCTGAAA
GGCGATATGCTGGATGAACTGATTCGCCTGCGCGAAGAAGTGAGCGAACAGATTCGCGC
GCTGGATGAAATTAAAAAAATGGCGCTGAGCTATGGCGTGGATATTAGCCGCCCGGCGG
TGAACGCGAAAGAAGCGGCGCAGTTTCTGTATTTTGGCTATCTGGCGGGCGTGAAAGAA
AACAACGGCGCGGCGATGAGCCTGGGCCGCACCAGCACCTTTCTGGATATTTATATTGAA
CGCGATCTGGAACAGGGCCTGATTACCGAAGATGAAGCGCAGGAAGTGATTGATCAGTT
TATTATTAAACTGCGCCTGGTGCGCCATCTGCGCACCCCGGAATATAACGAACTGTTTGC
GGGCGATCCGACCTGGGTGACCGAAAGCATTGCGGGCGTGGGCATTGATGGCCGCAGCC
TGGTGACCAAAAACAGCTTTCGCTATCTGCATACCCTGATTAACCTGGGCAGCGCGCCGG
AACCGAACATGACCGTGCTGTGGAGCGAAAACCTGCCGGAAAGCTTTAAAAAATTTTGC
GCGGAAATGAGCATTCTGACCGATAGCATTCAGTATGAAAACGATGATATTATGCGCCC
GATTTATGGCGATGATTATGCGATTGCGTGCTGCGTGAGCGCGATGCGCGTGGGCAAAG
ATATGCAGTTTTTTGGCGCGCGCTGCAACCTGGCGAAATGCCTGCTGCTGGCGATTAACG
GCGGCGTGGATGAAAAAAAAGGCATTAAAGTGGTGCCGGATATTGAACCGATTACCGAT
GAAGTGCTGGATTATGAAAAAGTGAAAGAAAACTATTTTAAAGTGCTGGAATATATGGC
GGGCCTGTATGTGAACACCATGAACATTATTCATTTTATGCATGATAAATATGCGTATGA
AGCGAGCCAGATGGCGCTGCATGATACCAAAGTGGGCCGCCTGATGGCGTTTGGCATTG
CGGGCTTTAGCGTGGCGGCGGATAGCCTGAGCGCGATTCGCTATGCGAAAGTGAAACCG
ATTCGCGAAAACGGCATTACCGTGGATTTTGTGAAAGAAGGCGATTTTCCGAAATATGGC
AACGATGATGATCGCGTGGATAGCATTGCGGTGGAAATTGTGGAAAAATTTAGCGATGA
ACTGAAAAAACATCCGACCTATCGCAACGCGAAACATACCCTGAGCGTGCTGACCATTA
CCAGCAACGTGATGTATGGCAAAAAAACCGGCACCACCCCGGATGGCCGCAAAGTGGGC
GAACCGCTGGCGCCGGGCGCGAACCCGATGCATGGCCGCGATATGGAAGGCGCGCTGGC
GAGCCTGAACAGCGTGGCGAAAGTGCCGTATGTGTGCTGCGAAGATGGCGTGAGCAACA
CCTTTAGCATTGTGCCGGATGCGCTGGGCAACGATCATGATGTGCGCATTAACAACCTGG
TGAGCATTATGGGCGGCTATTTTGGCCAGGGCGCGCATCATCTGAACGTGAACGTGCTGA
ACCGCGAAACCCTGATTGATGCGATGAACAACCCGGATAAATATCCGACCCTGACCATT
CGCGTGAGCGGCTATGCGGTGAACTTTAACCGCCTGAGCAAAGATCATCAGAAAGAAGT
GATTAGCCGCACCTTTCATGAAAAACTG
PFL_CLOPA Clostridium pasteurianum, Formate acetyltransferase,
740 aa,
SEQ ID NO: 54
MFKQWEGFQDGEWTNDVNVRDFIQKNYKEYTGDKSFLKGPTEKTKKVWDKAVSLILEELK
KGILDVDTETISGINSFKPGYLDKDNEVIVGFQTDAPLKRITNPFGGIRMAEQSLKEYGFKISDE
MHNIFTNYRKTHNQGVFDAYSEETRIARSAGVLTGLPDAYGRGRIIGDYRRVALYGIDFLIQE
KKKDLSNLKGDMLDELIRLREEVSEQIRALDEIKKMALSYGVDISRPAVNAKEAAQFLYFGY
LAGVKENNGAAMSLGRTSTFLDIYIERDLEQGLITEDEAQEVIDQFIIKLRLVRHLRTPEYNEL
FAGDPTWVTESIAGVGIDGRSLVTKNSFRYLHTLINLGSAPEPNMTVLWSENLPESFKKFCAE
MSILTDSIQYENDDIMRPIYGDDYAIACCVSAMRVGKDMQFFGARCNLAKCLLLAINGGVDE
KKGIKVVPDIEPITDEVLDYEKVKENYFKVLEYMAGLYVNTMNIIHFMHDKYAYEASQMAL
HDTKVGRLMAFGIAGFSVAADSLSAIRYAKVKPIRENGITVDFVKEGDFPKYGNDDDRVDSI
AVEIVEKFSDELKKHPTYRNAKHTLSVLTITSNVMYGKKTGTTPDGRKVGEPLAPGANPMH
GRDMEGALASLNSVAKVPYVCCEDGVSNTFSIVPDALGNDHDVRINNLVSIMGGYFGQGAH
HLNVNVLNRETLIDAMNNPDKYPTLTIRVSGYAVNFNRLSKDHQKEVISRTFHEKL

In some embodiments of any of the aspects, the sulfite reductase is encoded by SEQ ID NO: 55 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 55, that maintains the same function, or a codon-optimized version thereof.

In some embodiments of any of the aspects, the sulfite reductase comprises SEQ ID NO: 56 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 56, that maintains the same function (e.g., production of sulfite from hydrogen sulfide).

CYSJ, E. coli Sulfite reductase [NADPH] flavoprotein alpha-component,
1797 nt,
SEQ ID NO: 55
ATGACCACCCAGGTGCCGCCGAGCGCGCTGCTGCCGCTGAACCCGGAACAGCTGGCGCG
CCTGCAGGCGGCGACCACCGATCTGACCCCGACCCAGCTGGCGTGGGTGAGCGGCTATT
TTTGGGGCGTGCTGAACCAGCAGCCGGCGGCGCTGGCGGCGACCCCGGCGCCGGCGGCG
GAAATGCCGGGCATTACCATTATTAGCGCGAGCCAGACCGGCAACGCGCGCCGCGTGGC
GGAAGCGCTGCGCGATGATCTGCTGGCGGCGAAACTGAACGTGAAACTGGTGAACGCGG
GCGATTATAAATTTAAACAGATTGCGAGCGAAAAACTGCTGATTGTGGTGACCAGCACC
CAGGGCGAAGGCGAACCGCCGGAAGAAGCGGTGGCGCTGCATAAATTTCTGTTTAGCAA
AAAAGCGCCGAAACTGGAAAACACCGCGTTTGCGGTGTTTAGCCTGGGCGATAGCAGCT
ATGAATTTTTTTGCCAGAGCGGCAAAGATTTTGATAGCAAACTGGCGGAACTGGGCGGC
GAACGCCTGCTGGATCGCGTGGATGCGGATGTGGAATATCAGGCGGCGGCGAGCGAATG
GCGCGCGCGCGTGGTGGATGCGCTGAAAAGCCGCGCGCCGGTGGCGGCGCCGAGCCAGA
GCGTGGCGACCGGCGCGGTGAACGAAATTCATACCAGCCCGTATAGCAAAGATGCGCCG
CTGGTGGCGAGCCTGAGCGTGAACCAGAAAATTACCGGCCGCAACAGCGAAAAAGATGT
GCGCCATATTGAAATTGATCTGGGCGATAGCGGCATGCGCTATCAGCCGGGCGATGCGC
TGGGCGTGTGGTATCAGAACGATCCGGCGCTGGTGAAAGAACTGGTGGAACTGCTGTGG
CTGAAAGGCGATGAACCGGTGACCGTGGAAGGCAAAACCCTGCCGCTGAACGAAGCGCT
GCAGTGGCATTTTGAACTGACCGTGAACACCGCGAACATTGTGGAAAACTATGCGACCC
TGACCCGCAGCGAAACCCTGCTGCCGCTGGTGGGCGATAAAGCGAAACTGCAGCATTAT
GCGGCGACCACCCCGATTGTGGATATGGTGCGCTTTAGCCCGGCGCAGCTGGATGCGGA
AGCGCTGATTAACCTGCTGCGCCCGCTGACCCCGCGCCTGTATAGCATTGCGAGCAGCCA
GGCGGAAGTGGAAAACGAAGTGCATGTGACCGTGGGCGTGGTGCGCTATGATGTGGAAG
GCCGCGCGCGCGCGGGCGGCGCGAGCAGCTTTCTGGCGGATCGCGTGGAAGAAGAAGGC
GAAGTGCGCGTGTTTATTGAACATAACGATAACTTTCGCCTGCCGGCGAACCCGGAAACC
CCGGTGATTATGATTGGCCCGGGCACCGGCATTGCGCCGTTTCGCGCGTTTATGCAGCAG
CGCGCGGCGGATGAAGCGCCGGGCAAAAACTGGCTGTTTTTTGGCAACCCGCATTTTACC
GAAGATTTTCTGTATCAGGTGGAATGGCAGCGCTATGTGAAAGATGGCGTGCTGACCCG
CATTGATCTGGCGTGGAGCCGCGATCAGAAAGAAAAAGTGTATGTGCAGGATAAACTGC
GCGAACAGGGCGCGGAACTGTGGCGCTGGATTAACGATGGCGCGCATATTTATGTGTGC
GGCGATGCGAACCGCATGGCGAAAGATGTGGAACAGGCGCTGCTGGAAGTGATTGCGGA
ATTTGGCGGCATGGATACCGAAGCGGCGGATGAATTTCTGAGCGAACTGCGCGTGGAAC
GCCGCTATCAGCGCGATGTGTAT
CYSJ, E. coli Sulfite reductase [NADPH] flavoprotein alpha-
component, 599 aa,
SEQ ID NO: 56
MTTQVPPSALLPLNPEQLARLQAATTDLTPTQLAWVSGYFWGVLNQQPAALAATPAPAAEM
PGITIISASQTGNARRVAEALRDDLLAAKLNVKLVNAGDYKFKQIASEKLLIVVTSTQGEGEP
PEEAVALHKFLFSKKAPKLENTAFAVFSLGDSSYEFFCQSGKDFDSKLAELGGERLLDRVDA
DVEYQAAASEWRARVVDALKSRAPVAAPSQSVATGAVNEIHTSPYSKDAPLVASLSVNQKI
TGRNSEKDVRHIEIDLGDSGMRYQPGDALGVWYQNDPALVKELVELLWLKGDEPVTVEGK
TLPLNEALQWHFELTVNTANIVENYATLTRSETLLPLVGDKAKLQHYAATTPIVDMVRFSPA
QLDAEALINLLRPLTPRLYSIASSQAEVENEVHVTVGVVRYDVEGRARAGGASSFLADRVEE
EGEVRVFIEHNDNFRLPANPETPVIMIGPGTGIAPFRAFMQQRAADEAPGKNWLFFGNPHFTE
DFLYQVEWQRYVKDGVLTRIDLAWSRDQKEKVYVQDKLREQGAELWRWINDGAHIYVCG
DANRMAKDVEQALLEVIAEFGGMDTEAADEFLSELRVERRYQRDVY

In some embodiments of any of the aspects, the exogenous gene of the methanethiol catabolizing enzyme (e.g., esterase or a methanethiol oxidase), catalase, formaldehyde dehydrogenase, formate acetyltransferase, and/or sulfite reductase, if present, are expressed by the engineered probiotic microorganism under conditions in the gut, e.g., physiologically relevant conditions of the mammalian gastrointestinal tract.

In one aspect described herein is a method of reducing a level of methanethiol, e.g., in a mammal or subject in need thereof. In one aspect, the method comprises contacting methanethiol with a probiotic microorganism that encodes and expresses an exogenous gene encoding a methanethiol catabolizing enzyme. In some embodiments of any of the aspects, the methanethiol catabolizing enzyme is an esterase. In some embodiments of any of the aspects, the methanethiol catabolizing enzyme is a methanethiol oxidase. In some embodiments of any of the aspects, the methanethiol is produced by an engineered probiotic microorganism that comprises and expresses an exogenous gene encoding an enzyme that catalyzes the degradation of methionine to products including methanethiol. In some embodiments of any of the aspects, the enzyme that catalyzes the degradation of methionine to products including methanethiol comprises a methionine gamma lyase enzyme.

In some embodiments of any of the aspects, the administering reduces the level of methanethiol in the gut of the mammal. In some embodiments of any of the aspects, the level of methanethiol is reduced in the small intestine, duodenum, jejunum, ileum, cecum, ileocecum, appendix, ascending colon, transverse colon, descending colon, sigmoid colon, rectum, or anus of the mammal. In some embodiments of any of the aspects, the administering is oral or rectal.

In some embodiments of any of the aspects, the level of methanethiol is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or more.

In one aspect described herein is a method of reducing odor produced by a population of gut microbiota that produce methanethiol. In one aspect, the method comprises introducing an engineered probiotic microorganism to the gut microbiota, wherein the engineered probiotic microorganism encodes and expresses an exogenous gene encoding a methanethiol catabolizing enzyme. In some embodiments of any of the aspects, the methanethiol catabolizing enzyme is an esterase. In some embodiments of any of the aspects, the methanethiol catabolizing enzyme is a methanethiol oxidase. In some embodiments of any of the aspects, the methanethiol is produced by an engineered probiotic microorganism that comprises and expresses an exogenous gene encoding an enzyme that catalyzes the degradation of methionine to products including methanethiol. In some embodiments of any of the aspects, the enzyme that catalyzes the degradation of methionine to products including methanethiol comprises a methionine gamma lyase enzyme.

In some embodiments of any of the aspects, the administering reduces the odor in the gut of the mammal. In some embodiments of any of the aspects, the odor is reduced in the small intestine, duodenum, jejunum, ileum, cecum, ileocecum, appendix, ascending colon, transverse colon, descending colon, sigmoid colon, rectum, or anus of the mammal. In some embodiments of any of the aspects, the administering is oral or rectal.

### Taurine-Producing Microorganism

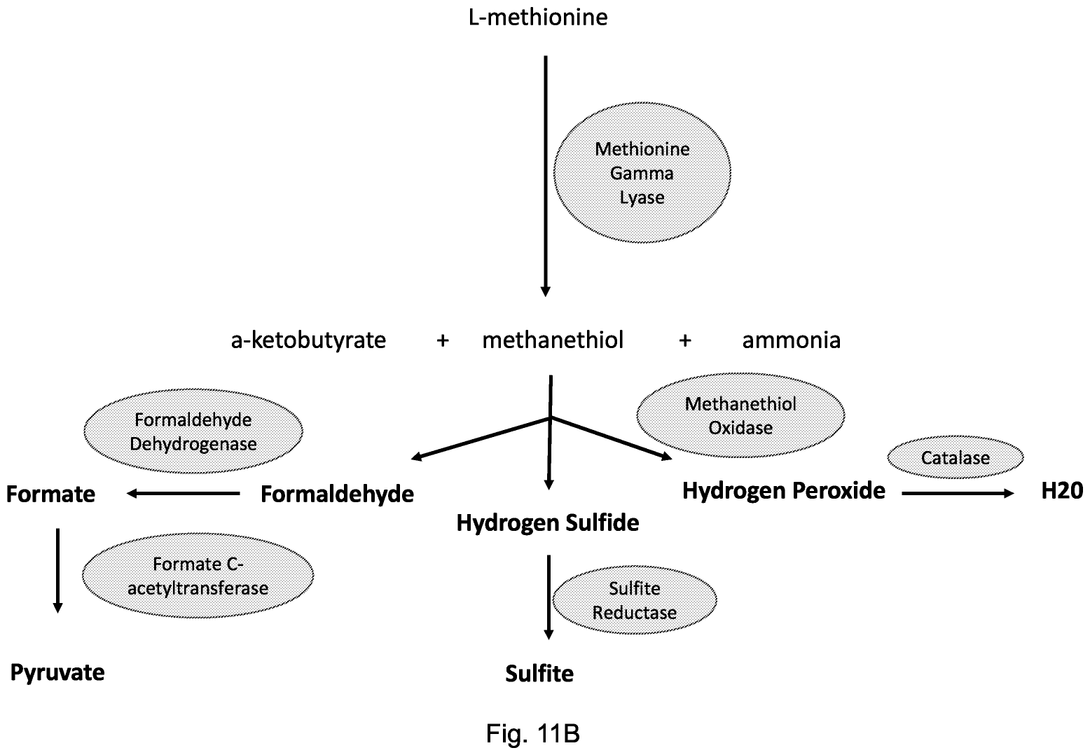

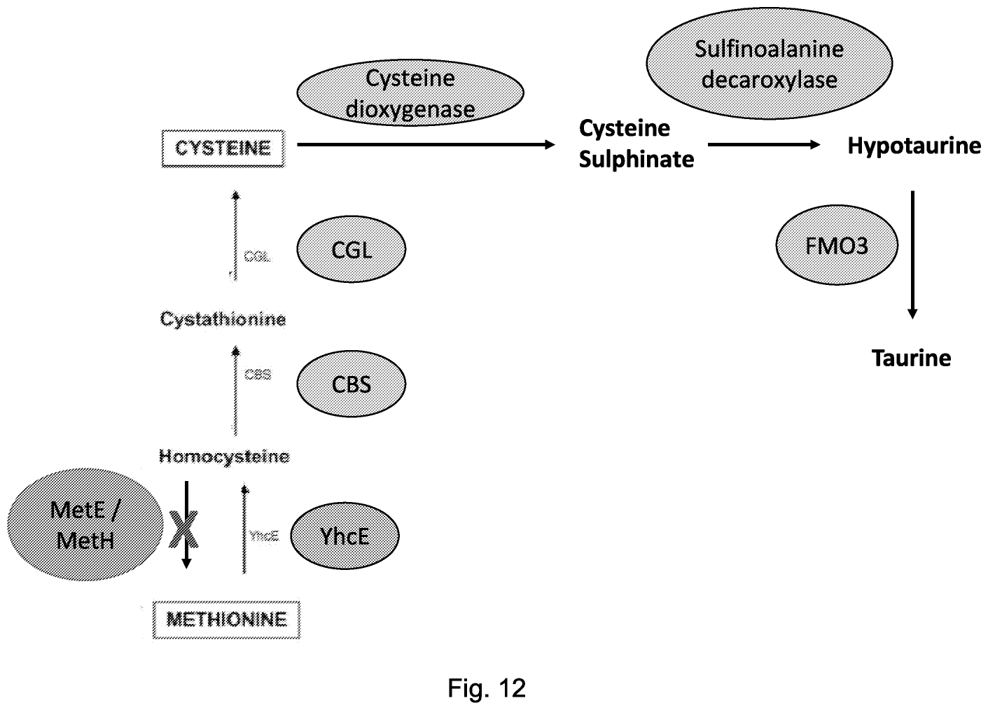

In one aspect, described herein is an engineered probiotic microorganism for reducing bioavailable methionine levels. In some embodiments, the engineered probiotic microorganism metabolizes methionine to taurine (see e.g., FIG. 12-13). The production of taurine, which is not converted back to methionine, can act as a “methionine sink” and thereby reduce the levels of bioavailable methionine. In one aspect, described herein is an engineered probiotic microorganism comprising: (a) at least one endogenous methionine synthesis gene comprising at least one engineered inactivating modification; (b) at least one copy of an exogenous gene encoding a homocysteine methyltransferase enzyme; (c) at least one copy of an exogenous gene encoding a sulfinoalanine decarboxylase enzyme; and (d) at least one copy of an exogenous gene encoding a Flavin-containing monooxygenase enzyme. In some embodiments of any of the aspects, the engineered probiotic microorganism expresses endogenously or exogenously encoded cystathionine β-synthase (CBS), cystathionine gamma lyase (CGL), and cysteine dioxygenase enzymes (see e.g., FIG. 12).

In one aspect, described herein is an engineered probiotic microorganism comprising: (a) at least one endogenous methionine synthesis gene comprising at least one engineered inactivating modification; (b) at least one copy of an exogenous gene encoding a glycine N-methyltransferase (GNMT) enzyme; (c) at least one copy of an exogenous gene encoding a sarcosine N-methyl transferase (SNMT) enzyme; (d) at least one copy of an exogenous gene encoding a sulfinoalanine decarboxylase enzyme; and (e) at least one copy of an exogenous gene encoding a Flavin-containing monooxygenase (FMO) enzyme. In some embodiments of any of the aspects, the engineered probiotic microorganism expresses endogenously or exogenously encoded methionine adenosyl transferase (MetK), adenosylhomocysteinase (ahcY), cystathionine β-synthase, cystathionine gamma lyase and cysteine dioxygenase enzymes (see e.g., FIG. 13).

In one aspect, described herein is an engineered probiotic microorganism comprising any combination of (a) at least one endogenous methionine synthesis gene comprising at least one engineered inactivating modification (e.g., MetE and/or MetH, SEQ ID NO: 35-40); (b) a homocysteine methyltransferase enzyme (e.g., YhcE, SEQ ID NO: 57-58); (c) a glycine N-methyltransferase (e.g., GNMT, SEQ ID NO: 59-61, SEQ ID NO: 78-79); (d) a sarcosine N-methyl transferase enzyme (e.g., SNMT, SEQ ID NOs: 74-77); (e) a sulfinoalanine decarboxylase enzyme (e.g., CSAD, SEQ ID NO: 62-67); and/or (f) a Flavin-containing monooxygenase enzyme (e.g., FMO1, FMO2, FMO3, SEQ ID NO: 68-73). Non-limiting examples of such exogenous gene combinations are provided in Table 4.

TABLE 4
Exemplary engineered probiotic microorganisms for taurine production

| (a) inactivated | (b) exogenous | (c) | (d) | (e) exogenous | (f) exogenous |
|---|---|---|---|---|---|
| methionine | homocysteine | exogenous | exogenous | sulfinoalanine | flavin-containing |
| synthase | methyltransferase | GNMT | SNMT | decarboxylase | monooxygenase |
| X | | | | | |
| | X | | | | |
| X | X | | | | |
| | | X | | | |
| X | | X | | | |
| | X | X | | | |
| X | X | X | | | |
| | | | X | | |
| X | | | X | | |
| | X | | X | | |
| X | X | | X | | |
| | | X | X | | |
| X | | X | X | | |
| | X | X | X | | |
| X | X | X | X | | |
| | | | | X | |
| X | | | | X | |
| | X | | | X | |
| X | X | | | X | |
| | | X | | X | |
| X | | X | | X | |
| | X | X | | X | |
| X | X | X | | X | |
| | | | X | X | |
| X | | | X | X | |
| | X | | X | X | |
| X | X | | X | X | |
| | | X | X | X | |
| X | | X | X | X | |
| | X | X | X | X | |
| X | X | X | X | X | |
| | | | | | X |
| X | | | | | X |
| | X | | | | X |
| X | X | | | | X |
| | | X | | | X |
| X | | X | | | X |
| | X | X | | | X |
| X | X | X | | | X |
| | | | X | | X |
| X | | | X | | X |
| | X | | X | | X |
| X | X | | X | | X |
| | | X | X | | X |
| X | | X | X | | X |
| | X | X | X | | X |
| X | X | X | X | | X |
| | | | | X | X |
| X | | | | X | X |
| | X | | | X | X |
| X | X | | | X | X |
| | | X | | X | X |
| X | | X | | X | X |
| | X | X | | X | X |
| X | X | X | | X | X |
| | | | X | X | X |
| X | | | X | X | X |
| | X | | X | X | X |
| X | X | | X | X | X |
| | | X | X | X | X |
| X | | X | X | X | X |
| | X | X | X | X | X |
| X | X | X | X | X | X |

(“x” indicates inclusion in the microorganism)

In some embodiments of any of the aspects, the engineered probiotic microorganism comprises and expresses one of the exemplary combinations of genes from Table 2; and one of the exemplary combinations of genes from Table 4. A composition comprising an engineered probiotic microorganism from Table 2 and an engineered probiotic microorganism from Table 4 is also specifically contemplated, and can, like the other probiotic microorganisms described herein, be formulated as a dietary supplement, pharmaceutical composition, and/or food composition.

In some embodiments of any of the aspects, the engineered probiotic microorganism comprises one of the exemplary combinations of genes from Table 4 and one or more of: (a) at least one exogenous copy of at least one gene encoding an enzyme that catalyzes the degradation of methionine; (b) at least one exogenous copy of at least one functional methionine importer gene; (c) at least one endogenous methionine importer gene comprising at least one engineered activating modification; (d) at least one endogenous methionine synthesis gene comprising at least one engineered inactivating modification; and/or (e) at least one endogenous methionine regulator gene comprising at least one engineered inactivating or activating modification

In some embodiments of any of the aspects, the at least one engineered inactivating modification of the endogenous methionine synthesis gene comprises at least one engineered inactivating mutation in the at least one endogenous methionine synthesis gene. In some embodiments of any of the aspects, the at least one engineered inactivating modification of the endogenous methionine synthesis gene comprises at least one engineered inactivating mutation in a promoter operatively linked to the at least one endogenous methionine synthesis gene. In some embodiments of any of the aspects, the at least one engineered inactivating modification of the endogenous methionine synthesis gene comprises at least one inhibitory RNA molecule specific to at least one messenger RNA (mRNA) expressed by the at least one endogenous methionine synthesis gene. Non-limiting examples of inhibitory RNAs include small interfering RNA (siRNA), micro RNA (miRNA), CRISPR RNA (crRNA and associated Cas endonuclease), and the like. In some embodiments of any of the aspects, the at least one engineered inactivating modification of the endogenous methionine synthesis gene comprises mutation of MetE and/or MetH genes. In some embodiments of any of the aspects, the methionine synthesis enzyme is MetE (see e.g., SEQ ID NO: 37-40) or MetH (see e.g., SEQ ID NO: 35-36).

A non-limiting example of the homocysteine methyltransferase enzyme is a YhcE homocysteine methyltransferase enzyme. In some embodiments of any of the aspects, the homocysteine methyltransferase is encoded by SEQ ID NO: 57 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 57, that maintains the same function, or a codon-optimized version thereof.

In some embodiments of any of the aspects, the homocysteine methyltransferase enzyme comprises SEQ ID NO: 58 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 58, that maintains the same function (e.g., conversion of methionine to homocysteine). In some embodiments of any of the aspects, the homocysteine methyltransferase is derived from a homocysteine methyltransferase gene or polypeptide of Corynebacterium singulare (see e.g., SEQ ID NOs: 57-58).

yhcE, Corynebacterium singulare Methionine synthase II, 1194 nt,
SEQ ID NO: 57
ATGGTGAACAAAATTCGCACCACCCATGTGGGCAGCCTGCCGCGCACCAAAGAACTGCT
GGAAGCGAACCTGGAACGCAGCGCGGGCACCATTAGCGATGAAAAATTTCATGAAATTC
TGGAACGCAGCGTGGCGGATGTGGTGAAACGCCAGGTGGATCTGGGCGTGGATATTATT
AACGAAGGCGAATATGGCCATATTACCAGCGGCGCGGTGGATTATGGCGCGTGGTGGAA
CTATAGCTTTACCCGCCTGGGCGGCCTGACCATGACCGATAAAGATCGCTGGGAAATTGG
CGATAAAATTCGCAGCGAACCGGGCAAAATTCGCCTGAGCAGCATGAAAGATCGCCGCG
ATCGCGCGCTGTTTAGCGAAGCGTATAACGATCCGGATAGCGGCATTTTTACCGGCCGCA
AAAAAGTGGCGAACCCGGAATTTACCGGCCCGGTGACCTATATTGGCCAGGAACAGGTG
GAAGCGGATGTGAAACTGCTGGCGGATGCGCTGCCGGCGGATACCGAAGGCTTTGTGGC
GGCGCTGAGCCCGGGCGCGGCGGCGCGCCTGCCGAACAAATATTATGAAGATGAAAGCG
AACTGGTGCGCGCGTGCGGCGAAGCGCTGAGCGTGGAATATAAAGCGATTACCGATGCG
GGCCTGACCGTGCAGTTTGATGCGCCGGATCTGGCGGAAGCGTGGGATAGCGTGGTGCC
GGAACCGACCGTGAAAGATTTTCAGGCGTTTCTGCATGAACGCATTGAAATTCTGAACGA
AAGCATTAAAGATATTCCGCGCGAACAGACCCGCCTGCATATTTGCTGGGGCAGCTGGC
ATGGCCCGCATGTGACCGATATTCCGTTTGAAGATATTATTGATGAAATTCTGCAGGCGA
AAGTGGGCGGCTTTAGCTTTGAAGGCGCGAGCCCGCGCCATGCGCATGAATGGCGCGTG
TGGAAAGATCATACCCTGCCGGAAGGCACCGTGATTTATCCGGGCGTGGTGAGCCATAG
CACCAACGCGGTGGAACATCCGCGCCTGGTGGCGGATCGCATTATTCAGTTTGCGGAACT
GGTGGGCCCGGAAAACGTGATTGCGAGCACCGATTGCGGCCTGGGCGGCCGCCTGCATC
ATCAGATTGCGTGGGCGAAACTGGAAAGCCTGGTGGAAGGCGCGGAAATTGCGACCAAA
GAACTGTTT
yhcE, Methionine synthase II (Cobalamin-independent), Corynebacterium
singulare, 398 aa,
SEQ ID NO: 58
MVNKIRTTHVGSLPRTKELLEANLERSAGTISDEKFHEILERSVADVVKRQVDLGVDIINEGE
YGHITSGAVDYGAWWNYSFTRLGGLTMTDKDRWEIGDKIRSEPGKIRLSSMKDRRDRALFS
EAYNDPDSGIFTGRKKVANPEFTGPVTYIGQEQVEADVKLLADALPADTEGFVAALSPGAAA
RLPNKYYEDESELVRACGEALSVEYKAITDAGLTVQFDAPDLAEAWDSVVPEPTVKDFQAFL
HERIEILNESIKDIPREQTRLHICWGSWHGPHVTDIPFEDIIDEILQAKVGGFSFEGASPRHAHE
WRVWKDHTLPEGTVIYPGVVSHSTNAVEHPRLVADRIIQFAELVGPENVIASTDCGLGGRLH
HQIAWAKLESLVEGAEIATKELF

In some embodiments of any of the aspects, the glycine N-methyltransferase (GNMT) is encoded by one of SEQ ID NOs: 59, 60, 78 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NOs: 59, 60 or 78 that maintains the same function, or a codon-optimized version thereof.

In some embodiments of any of the aspects, the glycine N-methyltransferase (GNMT) comprises SEQ ID NOs: 61, 79 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 61 or 79 that maintains the same function (e.g., conversion of S-adenosyl-methionine to S-adenosyl-homocysteine). In some embodiments of any of the aspects, the glycine N-methyltransferase is derived from a human glycine N-methyltransferase gene or polypeptide (see e.g., SEQ ID NOs: 59-61). In some embodiments of any of the aspects, the glycine N-methyltransferase is codon-optimized to be expressed by a specific bacterium, e.g., E. coli (see e.g., SEQ ID NOs: 60). In some embodiments of any of the aspects, the glycine N-methyltransferase is derived from a Halorhodospira halochloris glycine N-methyltransferase gene or polypeptide (see e.g., SEQ ID NOs: 78-79).

GNMT “Glycine-N-Methyltransferase,” H. sapiens, E. coli codon
optimized 885 nt,
SEQ ID NO: 59
ATGGTGGATAGCGTGTATCGCACCCGCAGCCTGGGCGTGGCGGCGGAAGGCCTGCCGGA
TCAGTATGCGGATGGCGAAGCGGCGCGCGTGTGGCAGCTGTATATTGGCGATACCCGCA
GCCGCACCGCGGAATATAAAGCGTGGCTGCTGGGCCTGCTGCGCCAGCATGGCTGCCAG
CGCGTGCTGGATGTGGCGTGCGGCACCGGCGTGGATAGCATTATGCTGGTGGAAGAAGG
CTTTAGCGTGACCAGCGTGGATGCGAGCGATAAAATGCTGAAATATGCGCTGAAAGAAC
GCTGGAACCGCCGCCATGAACCGGCGTTTGATAAATGGGTGATTGAAGAAGCGAACTGG
ATGACCCTGGATAAAGATGTGCCGCAGAGCGCGGAAGGCGGCTTTGATGCGGTGATTTG
CCTGGGCAACAGCTTTGCGCATCTGCCGGATTGCAAAGGCGATCAGAGCGAACATCGCC
TGGCGCTGAAAAACATTGCGAGCATGGTGCGCGCGGGCGGCCTGCTGGTGATTGATCAT
CGCAACTATGATCATATTCTGAGCACCGGCTGCGCGCCGCCGGGCAAAAACATTTATTAT
AAAAGCGATCTGACCAAAGATGTGACCACCAGCGTGCTGATTGTGAACAACAAAGCGCA
TATGGTGACCCTGGATTATACCGTGCAGGTGCCGGGCGCGGGCCAGGATGGCAGCCCGG
GCCTGAGCAAATTTCGCCTGAGCTATTATCCGCATTGCCTGGCGAGCTTTACCGAACTGC
TGCAGGCGGCGTTTGGCGGCAAATGCCAGCATAGCGTGCTGGGCGATTTTAAACCGTAT
AAACCGGGCCAGACCTATATTCCGTGCTATTTTATTCATGTGCTGAAACGCACCGAT
GNMT “Glycine-N-Methyltransferase,” H. sapiens, B. subtilis codon
optimized 885 nt,
SEQ ID NO: 60
ATGGTAGATAGCGTCTATCGGACACGGTCACTGGGGGTTGCTGCTGAGGGATTGCCTGAC
CAGTACGCAGACGGTGAAGCCGCAAGGGTCTGGCAACTCTACATAGGAGATACGAGATC
GCGGACAGCTGAGTATAAAGCATGGCTTCTAGGGCTGCTTAGACAACACGGTTGCCAGA
GAGTGTTAGATGTTGCATGTGGCACTGGCGTAGACTCGATCATGCTGGTGGAAGAAGGA
TTTTCAGTGACCAGCGTCGATGCCTCTGACAAAATGTTGAAATATGCACTGAAAGAACGT
TGGAATCGCCGACATGAGCCCGCGTTCGATAAATGGGTAATCGAAGAGGCCAATTGGAT
GACATTGGATAAAGACGTTCCGCAGAGTGCTGAAGGAGGCTTCGATGCCGTAATATGTC
TTGGAAACTCTTTTGCACACTTACCGGATTGTAAAGGTGACCAATCCGAACACAGACTTG
CTTTAAAGAACATTGCAAGCATGGTGCGCGCGGGAGGCTTGCTTGTCATCGACCATCGTA
ACTATGATCATATTTTAAGTACCGGATGCGCGCCTCCTGGAAAGAATATCTATTACAAAT
CTGATCTCACTAAGGACGTAACCACATCAGTCTTAATTGTTAATAACAAAGCGCATATGG
TGACGCTGGATTATACGGTCCAAGTTCCGGGTGCGGGTCAAGATGGCAGCCCAGGGCTG
TCAAAGTTTCGTTTATCTTATTACCCGCATTGTCTGGCGTCCTTTACAGAATTGCTTCAGG
CCGCTTTCGGGGGCAAATGCCAACATTCCGTGCTTGGCGATTTTAAACCGTACAAGCCTG
GCCAGACATATATTCCATGCTATTTTATTCATGTTCTCAAACGCACGGAT
GNMT “Glycine-N-Methyltransferase,” Homo sapiens, 295 aa,
SEQ ID NO: 61
MVDSVYRTRSLGVAAEGLPDQYADGEAARVWQLYIGDTRSRTAEYKAWLLGLLRQHGCQR
VLDVACGTGVDSIMLVEEGFSVTSVDASDKMLKYALKERWNRRHEPAFDKWVIEEANWMT
LDKDVPQSAEGGFDAVICLGNSFAHLPDCKGDQSEHRLALKNIASMVRAGGLLVIDHRNYD
HILSTGCAPPGKNIYYKSDLTKDVTTSVLIVNNKAHMVTLDYTVQVPGAGQDGSPGLSKFRL
SYYPHCLASFTELLQAAFGGKCQHSVLGDFKPYKPGQTYIPCYFIHVLKRTD
GNMT_HALHR Glycine N-methyltransferase Halorhodospira halochloris,
804 nt,
SEQ ID NO: 78
ATGAATACAACGACGGAACAAGATTTTGGAGCGGACCCTACCAAAGTAAGAGACACAG
ATCATTACACTGAAGAATACGTGGATGGATTCGTTGACAAATGGGATGACTTAATTGATT
GGGATAGCCGGGCTAAGTCCGAAGGGGATTTTTTTATTCAGGAACTTAAAAAGCGCGGG
GCCACGAGAATTCTAGACGCCGCAACAGGCACGGGCTTTCATTCTGTGAGACTTCTCGAA
GCCGGTTTTGATGTCGTCTCCGCGGATGGCTCTGCTGAGATGCTTGCGAAAGCCTTTGAG
AATGGCCGTAAACGTGGACACATCCTCAGGACCGTCCAGGTGGACTGGAGATGGTTGAA
CCGCGATATACACGGTCGGTATGATGCAATCATTTGTCTGGGCAATTCATTTACTCATCT
GTTTAATGAAAAGGATAGACGTAAAACTCTTGCAGAGTTTTACAGCGCATTGAACCCGG
AAGGCGTATTAATCCTGGATCAACGCAACTATGATGGTATACTGGATCATGGCTATGATA
GCAGTCATTCGTATTACTATTGCGGAGAGGGAGTCTCAGTTTATCCGGAACACGTTGACG
ACGGATTAGCGCGATTTAAATATGAATTTAACGACGGATCAACCTACTTCCTGAATATGT
TCCCATTACGTAAAGACTATACACGAAGGTTGATGCATGAAGTAGGGTTCCAAAAGATC
GACACATATGGTGATTTCAAAGCAACATACCGCGATGCTGACCCCGATTTCTTTATTCAT
GTTGCTGAAAAAGAATATCGGGAGGAGGAT
GNMT_HALHR Glycine N-methyltransferase Halorhodospira halochloris,
268 aa,
SEQ ID NO: 79
MNTTTEQDFGADPTKVRDTDHYTEEYVDGFVDKWDDLIDWDSRAKSEGDFFIQELKKRGAT
RILDAATGTGFHSVRLLEAGFDVVSADGSAEMLAKAFENGRKRGHILRTVQVDWRWLNRDI
HGRYDAIICLGNSFTHLFNEKDRRKTLAEFYSALNPEGVLILDQRNYDGILDHGYDSSHSYYY
CGEGVSVYPEHVDDGLARFKYEFNDGSTYFLNMFPLRKDYTRRLMHEVGFQKIDTYGDFKA
TYRDADPDFFIHVAEKEYREED

A non-limiting example of the sulfinoalanine decarboxylase enzyme is cysteine sulfinic acid decarboxylase (CSAD). In some embodiments of any of the aspects, the sulfinoalanine decarboxylase is encoded by SEQ ID NOs: 62, 64, 65, or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NOs: 62, 64, or 65, that maintains the same function, or a codon-optimized version thereof.

In some embodiments of any of the aspects, the sulfinoalanine decarboxylase enzyme comprises SEQ ID NOs: 63, 66, 67, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NOs: 63, 66, or 67, that maintains the same function (e.g., conversion of cysteine sulphinate to hypotaurine).

In some embodiments of any of the aspects, the sulfinoalanine decarboxylase is derived from a human sulfinoalanine decarboxylase gene or polypeptide (see e.g., SEQ ID NOs: 62-63). In some embodiments of any of the aspects, the sulfinoalanine decarboxylase is derived from a prokaryotic sulfinoalanine decarboxylase gene or polypeptide (see e.g., SEQ ID NOs: 64-67). In some embodiments of any of the aspects, the sulfinoalanine decarboxylase comprises at least one regulatory mutation selected from V81L, I250M, and/or D266L (see e.g., SEQ ID NOs: 65, 67).

CSAD Homo sapiens, “Sulfinoalanine Decarboxylase,” 1479 nt,
SEQ ID NO: 62
ATGGCGGATAGCGAAGCGCTGCCGAGCCTGGCGGGCGATCCGGTGGCGGTGGAAGCGCT
GCTGCGCGCGGTGTTTGGCGTGGTGGTGGATGAAGCGATTCAGAAAGGCACCAGCGTGA
GCCAGAAAGTGTGCGAATGGAAAGAACCGGAAGAACTGAAACAGCTGCTGGATCTGGA
ACTGCGCAGCCAGGGCGAAAGCCAGAAACAGATTCTGGAACGCTGCCGCGCGGTGATTC
GCTATAGCGTGAAAACCGGCCATCCGCGCTTTTTTAACCAGCTGTTTAGCGGCCTGGATC
CGCATGCGCTGGCGGGCCGCATTATTACCGAAAGCCTGAACACCAGCCAGTATACCTAT
GAAATTGCGCCGGTGTTTGTGCTGATGGAAGAAGAAGTGCTGCGCAAACTGCGCGCGCT
GGTGGGCTGGAGCAGCGGCGATGGCATTTTTTGCCCGGGCGGCAGCATTAGCAACATGT
ATGCGGTGAACCTGGCGCGCTATCAGCGCTATCCGGATTGCAAACAGCGCGGCCTGCGC
ACCCTGCCGCCGCTGGCGCTGTTTACCAGCAAAGAATGCCATTATAGCATTCAGAAAGGC
GCGGCGTTTCTGGGCCTGGGCACCGATAGCGTGCGCGTGGTGAAAGCGGATGAACGCGG
CAAAATGGTGCCGGAAGATCTGGAACGCCAGATTGGCATGGCGGAAGCGGAAGGCGCG
GTGCCGTTTCTGGTGAGCGCGACCAGCGGCACCACCGTGCTGGGCGCGTTTGATCCGCTG
GAAGCGATTGCGGATGTGTGCCAGCGCCATGGCCTGTGGCTGCATGTGGATGCGGCGTG
GGGCGGCAGCGTGCTGCTGAGCCAGACCCATCGCCATCTGCTGGATGGCATTCAGCGCG
CGGATAGCGTGGCGTGGAACCCGCATAAACTGCTGGCGGCGGGCCTGCAGTGCAGCGCG
CTGCTGCTGCAGGATACCAGCAACCTGCTGAAACGCTGCCATGGCAGCCAGGCGAGCTA
TCTGTTTCAGCAGGATAAATTTTATGATGTGGCGCTGGATACCGGCGATAAAGTGGTGCA
GTGCGGCCGCCGCGTGGATTGCCTGAAACTGTGGCTGATGTGGAAAGCGCAGGGCGATC
AGGGCCTGGAACGCCGCATTGATCAGGCGTTTGTGCTGGCGCGCTATCTGGTGGAAGAA
ATGAAAAAACGCGAAGGCTTTGAACTGGTGATGGAACCGGAATTTGTGAACGTGTGCTT
TTGGTTTGTGCCGCCGAGCCTGCGCGGCAAACAGGAAAGCCCGGATTATCATGAACGCC
TGAGCAAAGTGGCGCCGGTGCTGAAAGAACGCATGGTGAAAGAAGGCAGCATGATGATT
GGCTATCAGCCGCATGGCACCCGCGGCAACTTTTTTCGCGTGGTGGTGGCGAACAGCGCG
CTGACCTGCGCGGATATGGATTTTCTGCTGAACGAACTGGAACGCCTGGGCCAGGATCTG
CSAD “Sulfinoalanine Decarboxylase,” Homo sapiens, 493 aa,
SEQ ID NO: 63
MADSEALPSLAGDPVAVEALLRAVFGVVVDEAIQKGTSVSQKVCEWKEPEELKQLLDLELR
SQGESQKQILERCRAVIRYSVKTGHPRFFNQLFSGLDPHALAGRIITESLNTSQYTYEIAPVFVL
MEEEVLRKLRALVGWSSGDGIFCPGGSISNMYAVNLARYQRYPDCKQRGLRTLPPLALFTSK
ECHYSIQKGAAFLGLGTDSVRVVKADERGKMVPEDLERQIGMAEAEGAVPFLVSATSGTTV
LGAFDPLEAIADVCQRHGLWLHVDAAWGGSVLLSQTHRHLLDGIQRADSVAWNPHKLLAA
GLQCSALLLQDTSNLLKRCHGSQASYLFQQDKFYDVALDTGDKVVQCGRRVDCLKLWLM
WKAQGDQGLERRIDQAFVLARYLVEEMKKREGFELVMEPEFVNVCFWFVPPSLRGKQESPD
YHERLSKVAPVLKERMVKEGSMMIGYQPHGTRGNFFRVVVANSALTCADMDFLLNELERL
GQDL
Prokaryotic CSAD WT 1077 nt,
SEQ ID NO: 64
atgATTACCCCATTAACGCTTGCTACACTCTCGAAAAATCCTATACTGGTTGATTTTTTCGA
TCCTGAAGATGGACGTTGGAATTCACATGTCGATTTAGGCCTCTGGTCAGATCTGTATCT
TATCGCGCCTGCAACGGCGAACACCATCGGAAAAATGGCAGCAGGTATTGCGGACAATC
TTTTATTGACATCTTACTTATCCGCTCGGTGCCCGGTATTTATTGCCCCCGCCATGGATGT
TGATATGTTAATGCATCCGGCAACTCAAAGAAACCTGGGAATCCTTAAATCTTCAGGAAA
CCACATAATTGAGCCGGGTAGCGGGGAGCTTGCCTCTGGTCTAACGGGAAAAGGCCGCA
TGGCAGAACCCGAAGAAATCGTAAGAGAGGTCATTTCGTTTTTCTCAAAAAAGAAAATT
ACCGAAAAACCATTGAATGGACGACGAGTTTTTATTAACGCGGGCCCTACGATTGAACC
GATTGATCCGGTGAGGTTCATATCCAACTATAGCTCCGGGCGGATGGGGATTGCGCTTGC
TGATGCCGCGGCCGCGATGGGAGCTGAGGTGACATTGGTCCTGGGTCCGGTCACTCTGCG
TCCGAGTTCTCAGGACATCAATGTTATCGACGTGAGGAGTGCAGCTGAAATGAAAGAAG
CGTCAGTAGAAGCTTTTAGAGAATGTGACATAGCAATACTTGCCGCCGCTGTCGCAGACT
TTACACCGTTGACCACAAGCGACAAGAAGATTAAACGCGGCTCTGGTGAAATGGTTATC
AATTTAAGACCTACGGAAGATATTGCTGCGGAACTCGGCAAAATGAAAAAGAAGAATCA
ATTGCTGGTTGGGTTTGCTCTGGAGACAGACGATGAAATTACAAATGCGAGCTCAAAACT
GAAACGGAAGAATCTCGATATGATCGTGCTAAATAGCTTAAAGGATCCAGGCGCCGGCT
TTGGACACGAGACTAACCGCATCACAATCATTGATAAAAGTAACAACATCGATAAATTC
GAACTGAAAACGAAAGGCGAGGTGGCAGCAGACATTATTCGTAAGATCTTGACACTTGT
ACAT
Prokaryotic CSAD Regulatory Mutant 1077 nt,
SEQ ID NO: 65
ATGATAACGCCATTAACGCTCGCTACCCTGTCCAAAAATCCGATTTTGGTGGATTTCTTTG
ATCCTGAAGATGGCCGTTGGAATTCACACGTGGATCTTGGTTTATGGTCAGATCTGTACT
TAATTGCCCCTGCGACCGCTAATACAATTGGTAAAATGGCAGCGGGAATTGCAGATAAC
TTATTACTTACGAGCTATCTAAGCGCGCGCTGCCCGGTTTTCATTGCCCCTGCCATGGATC
TTGACATGCTCATGCATCCGGCGACACAACGAAACCTTGGAATACTTAAGTCTAGTGGCA
ATCATATCATCGAACCCGGCTCAGGAGAACTTGCTTCAGGGCTGACAGGTAAAGGGCGG
ATGGCAGAACCGGAGGAGATCGTGAGAGAGGTTATTTCCTTTTTCAGTAAAAAAAAGAT
TACCGAAAAACCGTTGAACGGGCGGCGTGTTTTTATTAATGCCGGTCCAACCATCGAACC
GATCGATCCGGTCCGCTTCATCTCTAATTATAGCAGTGGACGTATGGGAATCGCGTTGGC
AGACGCTGCGGCTGCCATGGGCGCCGAAGTCACATTAGTCTTAGGTCCTGTTACTTTGAG
GCCTTCCTCGCAGGACATTAATGTGATAGACGTGAGATCTGCAGCCGAGATGAAAGAAG
CTTCAGTAGAAGCATTTAGGGAGTGTGACATTGCAATCTTGGCCGCAGCTGTCGCAGACT
CTACTCCGCTGACGACAAGCGATAAAAAGATGAAGCGCGGCAGCGGCGAAATGGTTATA
AACCTTCGACCCACGGAACTGATTGCAGCGGAACTAGGAAAAATGAAGAAAAAAAACC
AACTGCTGGTAGGCTTTGCTCTGGAGACAGATGATGAAATCACAAACGCTTCGTCTAAGC
TCAAGAGAAAAAATCTTGACATGATTGTACTCAATAGCCTTAAGGACCCAGGAGCGGGC
TTTGGGCACGAGACAAACCGGATTACTATCATTGATAAATCAAATAACATTGATAAATTT
GAATTGAAAACAAAAGGAGAAGTCGCGGCGGATATTATCAGAAAAATCCTGACGTTAGT
ACAT
Prokaryotic CSAD WT 359 aa,
SEQ ID NO: 66
MITPLTLATLSKNPILVDFFDPEDGRWNSHVDLGLWSDLYLIAPATANTIGKMAAGIADNLLL
TSYLSARCPVFIAPAMDVDMLMHPATQRNLGILKSSGNHIIEPGSGELASGLTGKGRMAEPEE
IVREVISFFSKKKITEKPLNGRRVFINAGPTIEPIDPVRFISNYSSGRMGIALADAAAAMGAEVT
LVLGPVTLRPSSQDINVIDVRSAAEMKEASVEAFRECDIAILAAAVADFTPLTTSDKKIKRGSG
EMVINLRPTEDIAAELGKMKKKNQLLVGFALETDDEITNASSKLKRKNLDMIVLNSLKDPGA
GFGHETNRITIIDKSNNIDKFELKTKGEVAADIIRKILTLVH
Prokaryotic CSAD Regulatory Mutant 359 aa,
SEQ ID NO: 67
MITPLTLATLSKNPILVDFFDPEDGRWNSHVDLGLWSDLYLIAPATANTIGKMAAGIADNLLL
TSYLSARCPVFIAPAMDLDMLMHPATQRNLGILKSSGNHIIEPGSGELASGLTGKGRMAEPEE
IVREVISFFSKKKITEKPLNGRRVFINAGPTIEPIDPVRFISNYSSGRMGIALADAAAAMGAEVT
LVLGPVTLRPSSQDINVIDVRSAAEMKEASVEAFRECDIAILAAAVADSTPLTTSDKKMKRGS
GEMVINLRPTELIAAELGKMKKKNQLLVGFALETDDEITNASSKLKRKNLDMIVLNSLKDPG
AGFGHETNRITIIDKSNNIDKFELKTKGEVAADIIRKILTLVH

In some embodiments of any of the aspects, the FMO enzyme is FMO1 (see e.g., SEQ ID NO: 68 or 71), FMO2 (see e.g., SEQ ID NO: 69 or 72), or FMO3 (see e.g., SEQ ID NO: 70 or 73). In some embodiments of any of the aspects, the FMO enzyme catalyzes the catalysis of the conversion of hypotaurine to taurine. In some embodiments of any of the aspects, the FMO enzyme is encoded by one of SEQ ID NO: 68-70 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to one of SEQ ID NO: 68-70, that maintains the same function, or a codon-optimized version thereof.

In some embodiments of any of the aspects, the FMO enzyme comprises one of SEQ ID NO: 71-73 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to one of SEQ ID NO: 71-73, that maintains the same function (e.g., the conversion of hypotaurine to taurine). In some embodiments of any of the aspects, the flavin-containing monooxygenase is derived from a human flavin-containing monooxygenase gene or polypeptide (see e.g., SEQ ID NOs: 68-73).

In some embodiments of any of the aspects, the engineered probiotic microorganism comprises FMO1 (see e.g., SEQ ID NO: 68 or 71), FMO2 (see e.g., SEQ ID NO: 69 or 72), or FMO3 (see e.g., SEQ ID NO: 70 or 73). In some embodiments of any of the aspects, the engineered probiotic microorganism comprises FMO1 (see e.g., SEQ ID NO: 68 or 71). In some embodiments of any of the aspects, the engineered probiotic microorganism comprises FMO2 (see e.g., SEQ ID NO: 69 or 72). In some embodiments of any of the aspects, the engineered probiotic microorganism comprises FMO3 (see e.g., SEQ ID NO: 70 or 73).

In some embodiments of any of the aspects, the engineered probiotic microorganism comprises FMO1 (see e.g., SEQ ID NO: 68 or 71) and FMO2 (see e.g., SEQ ID NO: 69 or 72). In some embodiments of any of the aspects, the engineered probiotic microorganism comprises FMO1 and FMO3 (see e.g., SEQ ID NO: 70 or 73). In some embodiments of any of the aspects, the engineered probiotic microorganism comprises FMO2 (see e.g., SEQ ID NO: 69 or 72) and FMO3 (see e.g., SEQ ID NO: 70 or 73). In some embodiments of any of the aspects, the engineered probiotic microorganism comprises FMO1 (see e.g., SEQ ID NO: 68 or 71), FMO2 (see e.g., SEQ ID NO: 69 or 72), and FMO3 (see e.g., SEQ ID NO: 70 or 73).

FMO1 Homo sapiens “Hypotaurine dehydrogenase 1,” 1596 nt,
SEQ ID NO: 68
ATGGCGAAACGCGTGGCGATTGTGGGCGCGGGCGTGAGCGGCCTGGCGAGCATTAAATG
CTGCCTGGAAGAAGGCCTGGAACCGACCTGCTTTGAACGCAGCGATGATCTGGGCGGCC
TGTGGCGCTTTACCGAACATGTGGAAGAAGGCCGCGCGAGCCTGTATAAAAGCGTGGTG
AGCAACAGCTGCAAAGAAATGAGCTGCTATAGCGATTTTCCGTTTCCGGAAGATTATCCG
AACTATGTGCCGAACAGCCAGTTTCTGGAATATCTGAAAATGTATGCGAACCATTTTGAT
CTGCTGAAACATATTCAGTTTAAAACCAAAGTGTGCAGCGTGACCAAATGCAGCGATAG
CGCGGTGAGCGGCCAGTGGGAAGTGGTGACCATGCATGAAGAAAAACAGGAAAGCGCG
ATTTTTGATGCGGTGATGGTGTGCACCGGCTTTCTGACCAACCCGTATCTGCCGCTGGAT
AGCTTTCCGGGCATTAACGCGTTTAAAGGCCAGTATTTTCATAGCCGCCAGTATAAACAT
CCGGATATTTTTAAAGATAAACGCGTGCTGGTGATTGGCATGGGCAACAGCGGCACCGA
TATTGCGGTGGAAGCGAGCCATCTGGCGGAAAAAGTGTTTCTGAGCACCACCGGCGGCG
GCTGGGTGATTAGCCGCATTTTTGATAGCGGCTATCCGTGGGATATGGTGTTTATGACCC
GCTTTCAGAACATGCTGCGCAACAGCCTGCCGACCCCGATTGTGACCTGGCTGATGGAAC
GCAAAATTAACAACTGGCTGAACCATGCGAACTATGGCCTGATTCCGGAAGATCGCACC
CAGCTGAAAGAATTTGTGCTGAACGATGAACTGCCGGGCCGCATTATTACCGGCAAAGT
GTTTATTCGCCCGAGCATTAAAGAAGTGAAAGAAAACAGCGTGATTTTTAACAACACCA
GCAAAGAAGAACCGATTGATATTATTGTGTTTGCGACCGGCTATACCTTTGCGTTTCCGT
TTCTGGATGAAAGCGTGGTGAAAGTGGAAGATGGCCAGGCGAGCCTGTATAAATATATT
TTTCCGGCGCATCTGCAGAAACCGACCCTGGCGATTATTGGCCTGATTAAACCGCTGGGC
AGCATGATTCCGACCGGCGAAACCCAGGCGCGCTGGGCGGTGCGCGTGCTGAAAGGCGT
GAACAAACTGCCGCCGCCGAGCGTGATGATTGAAGAAATTAACGCGCGCAAAGAAAAC
AAACCGAGCTGGTTTGGCCTGTGCTATTGCAAAGCGCTGCAGAGCGATTATATTACCTAT
ATTGATGAACTGCTGACCTATATTAACGCGAAACCGAACCTGTTTAGCATGCTGCTGACC
GATCCGCATCTGGCGCTGACCGTGTTTTTTGGCCCGTGCAGCCCGTATCAGTTTCGCCTGA
CCGGCCCGGGCAAATGGGAAGGCGCGCGCAACGCGATTATGACCCAGTGGGATCGCACC
TTTAAAGTGATTAAAGCGCGCGTGGTGCAGGAAAGCCCGAGCCCGTTTGAAAGCTTTCTG
AAAGTGTTTAGCTTTCTGGCGCTGCTGGTGGCGATTTTTCTGATTTTTCTG
FMO2 Homo sapiens “Hypotaurine dehydrogenase 2,” 1605 nt,
SEQ ID NO: 69
ATGGCGAAAAAAGTGGCGGTGATTGGCGCGGGCGTGAGCGGCCTGATTAGCCTGAAATG
CTGCGTGGATGAAGGCCTGGAACCGACCTGCTTTGAACGCACCGAAGATATTGGCGGCG
TGTGGCGCTTTAAAGAAAACGTGGAAGATGGCCGCGCGAGCATTTATCAGAGCGTGGTG
ACCAACACCAGCAAAGAAATGAGCTGCTTTAGCGATTTTCCGATGCCGGAAGATTTTCCG
AACTTTCTGCATAACAGCAAACTGCTGGAATATTTTCGCATTTTTGCGAAAAAATTTGAT
CTGCTGAAATATATTCAGTTTCAGACCACCGTGCTGAGCGTGCGCAAATGCCCGGATTTT
AGCAGCAGCGGCCAGTGGAAAGTGGTGACCCAGAGCAACGGCAAAGAACAGAGCGCGG
TGTTTGATGCGGTGATGGTGTGCAGCGGCCATCATATTCTGCCGCATATTCCGCTGAAAA
GCTTTCCGGGCATGGAACGCTTTAAAGGCCAGTATTTTCATAGCCGCCAGTATAAACATC
CGGATGGCTTTGAAGGCAAACGCATTCTGGTGATTGGCATGGGCAACAGCGGCAGCGAT
ATTGCGGTGGAACTGAGCAAAAACGCGGCGCAGGTGTTTATTAGCACCCGCCATGGCAC
CTGGGTGATGAGCCGCATTAGCGAAGATGGCTATCCGTGGGATAGCGTGTTTCATACCCG
CTTTCGCAGCATGCTGCGCAACGTGCTGCCGCGCACCGCGGTGAAATGGATGATTGAAC
AGCAGATGAACCGCTGGTTTAACCATGAAAACTATGGCCTGGAACCGCAGAACAAATAT
ATTATGAAAGAACCGGTGCTGAACGATGATGTGCCGAGCCGCCTGCTGTGCGGCGCGAT
TAAAGTGAAAAGCACCGTGAAAGAACTGACCGAAACCAGCGCGATTTTTGAAGATGGCA
CCGTGGAAGAAAACATTGATGTGATTATTTTTGCGACCGGCTATAGCTTTAGCTTTCCGTT
TCTGGAAGATAGCCTGGTGAAAGTGGAAAACAACATGGTGAGCCTGTATAAATATATTT
TTCCGGCGCATCTGGATAAAAGCACCCTGGCGTGCATTGGCCTGATTCAGCCGCTGGGCA
GCATTTTTCCGACCGCGGAACTGCAGGCGCGCTGGGTGACCCGCGTGTTTAAAGGCCTGT
GCAGCCTGCCGAGCGAACGCACCATGATGATGGATATTATTAAACGCAACGAAAAACGC
ATTGATCTGTTTGGCGAAAGCCAGAGCCAGACCCTGCAGACCAACTATGTGGATTATCTG
GATGAACTGGCGCTGGAAATTGGCGCGAAACCGGATTTTTGCAGCCTGCTGTTTAAAGAT
CCGAAACTGGCGGTGCGCCTGTATTTTGGCCCGTGCAACAGCTATCAGTATCGCCTGGTG
GGCCCGGGCCAGTGGGAAGGCGCGCGCAACGCGATTTTTACCCAGAAACAGCGCATTCT
GAAACCGCTGAAAACCCGCGCGCTGAAAGATAGCAGCAACTTTAGCGTGAGCTTTCTGC
TGAAAATTCTGGGCCTGCTGGCGGTGGTGGTGGCGTTTTTTTGCCAGCTGCAGTGGAGC
FMO3 Homo sapiens “Hypotaurine dehydrogenase 3,” 1596 nt,
SEQ ID NO: 70
ATGGGCAAAAAAGTGGCGATTATTGGCGCGGGCGTGAGCGGCCTGGCGAGCATTCGCAG
CTGCCTGGAAGAAGGCCTGGAACCGACCTGCTTTGAAAAAAGCAACGATATTGGCGGCC
TGTGGAAATTTAGCGATCATGCGGAAGAAGGCCGCGCGAGCATTTATAAAAGCGTGTTT
AGCAACAGCAGCAAAGAAATGATGTGCTTTCCGGATTTTCCGTTTCCGGATGATTTTCCG
AACTTTATGCATAACAGCAAAATTCAGGAATATATTATTGCGTTTGCGAAAGAAAAAAA
CCTGCTGAAATATATTCAGTTTAAAACCTTTGTGAGCAGCGTGAACAAACATCCGGATTT
TGCGACCACCGGCCAGTGGGATGTGACCACCGAACGCGATGGCAAAAAAGAAAGCGCG
GTGTTTGATGCGGTGATGGTGTGCAGCGGCCATCATGTGTATCCGAACCTGCCGAAAGAA
AGCTTTCCGGGCCTGAACCATTTTAAAGGCAAATGCTTTCATAGCCGCGATTATAAAGAA
CCGGGCGTGTTTAACGGCAAACGCGTGCTGGTGGTGGGCCTGGGCAACAGCGGCTGCGA
TATTGCGACCGAACTGAGCCGCACCGCGGAACAGGTGATGATTAGCAGCCGCAGCGGCA
GCTGGGTGATGAGCCGCGTGTGGGATAACGGCTATCCGTGGGATATGCTGCTGGTGACC
CGCTTTGGCACCTTTCTGAAAAACAACCTGCCGACCGCGATTAGCGATTGGCTGTATGTG
AAACAGATGAACGCGCGCTTTAAACATGAAAACTATGGCCTGATGCCGCTGAACGGCGT
GCTGCGCAAAGAACCGGTGTTTAACGATGAACTGCCGGCGAGCATTCTGTGCGGCATTGT
GAGCGTGAAACCGAACGTGAAAGAATTTACCGAAACCAGCGCGATTTTTGAAGATGGCA
CCATTTTTGAAGGCATTGATTGCGTGATTTTTGCGACCGGCTATAGCTTTGCGTATCCGTT
TCTGGATGAAAGCATTATTAAAAGCCGCAACAACGAAATTATTCTGTTTAAAGGCGTGTT
TCCGCCGCTGCTGGAAAAAAGCACCATTGCGGTGATTGGCTTTGTGCAGAGCCTGGGCGC
GGCGATTCCGACCGTGGATCTGCAGAGCCGCTGGGCGGCGCAGGTGATTAAAGGCACCT
GCACCCTGCCGAGCATGGAAGATATGATGAACGATATTAACGAAAAAATGGAAAAAAA
ACGCAAATGGTTTGGCAAAAGCGAAACCATTCAGACCGATTATATTGTGTATATGGATG
AACTGAGCAGCTTTATTGGCGCGAAACCGAACATTCCGTGGCTGTTTCTGACCGATCCGA
AACTGGCGATGGAAGTGTATTTTGGCCCGTGCAGCCCGTATCAGTTTCGCCTGGTGGGCC
CGGGCCAGTGGCCGGGCGCGCGCAACGCGATTCTGACCCAGTGGGATCGCAGCCTGAAA
CCGATGCAGACCCGCGTGGTGGGCCGCCTGCAGAAACCGTGCTTTTTTTTTCATTGGCTG
AAACTGTTTGCGATTCCGATTCTGCTGATTGCGGTGTTTCTGGTGCTGACC
FMO1 Homo sapiens “Hypotaurine dehydrogenase 1,” 532 aa,
SEQ ID NO: 71
MAKRVAIVGAGVSGLASIKCCLEEGLEPTCFERSDDLGGLWRFTEHVEEGRASLYKSVVSNS
CKEMSCYSDFPFPEDYPNYVPNSQFLEYLKMYANHFDLLKHIQFKTKVCSVTKCSDSAVSGQ
WEVVTMHEEKQESAIFDAVMVCTGFLTNPYLPLDSFPGINAFKGQYFHSRQYKHPDIFKDKR
VLVIGMGNSGTDIAVEASHLAEKVFLSTTGGGWVISRIFDSGYPWDMVFMTRFQNMLRNSLP
TPIVTWLMERKINNWLNHANYGLIPEDRTQLKEFVLNDELPGRIITGKVFIRPSIKEVKENSVIF
NNTSKEEPIDIIVFATGYTFAFPFLDESVVKVEDGQASLYKYIFPAHLQKPTLAIIGLIKPLGSMI
PTGETQARWAVRVLKGVNKLPPPSVMIEEINARKENKPSWFGLCYCKALQSDYITYIDELLT
YINAKPNLFSMLLTDPHLALTVFFGPCSPYQFRLTGPGKWEGARNAIMTQWDRTFKVIKARV
VQESPSPFESFLKVFSFLALLVAIFLIFL
FMO2 Homo sapiens “Hypotaurine dehydrogenase 2,” 535 aa,
SEQ ID NO: 72
MAKKVAVIGAGVSGLISLKCCVDEGLEPTCFERTEDIGGVWRFKENVEDGRASIYQSVVTNT
SKEMSCFSDFPMPEDFPNFLHNSKLLEYFRIFAKKFDLLKYIQFQTTVLSVRKCPDFSSSGQW
KVVTQSNGKEQSAVFDAVMVCSGHHILPHIPLKSFPGMERFKGQYFHSRQYKHPDGFEGKRI
LVIGMGNSGSDIAVELSKNAAQVFISTRHGTWVMSRISEDGYPWDSVFHTRFRSMLRNVLPR
TAVKWMIEQQMNRWFNHENYGLEPQNKYIMKEPVLNDDVPSRLLCGAIKVKSTVKELTETS
AIFEDGTVEENIDVIIFATGYSFSFPFLEDSLVKVENNMVSLYKYIFPAHLDKSTLACIGLIQPL
GSIFPTAELQARWVTRVFKGLCSLPSERTMMMDIIKRNEKRIDLFGESQSQTLQTNYVDYLDE
LALEIGAKPDFCSLLFKDPKLAVRLYFGPCNSYQYRLVGPGQWEGARNAIFTQKQRILKPLKT
RALKDSSNFSVSFLLKILGLLAVVVAFFCQLQWS
FMO3 “Hypotaurine dehydrogenase 3,” Homo sapiens532 aa,
SEQ ID NO: 73
MGKKVAIIGAGVSGLASIRSCLEEGLEPTCFEKSNDIGGLWKFSDHAEEGRASIYKSVFSNSSK
EMMCFPDFPFPDDFPNFMHNSKIQEYIIAFAKEKNLLKYIQFKTFVSSVNKHPDFATTGQWDV
TTERDGKKESAVFDAVMVCSGHHVYPNLPKESFPGLNHFKGKCFHSRDYKEPGVFNGKRVL
VVGLGNSGCDIATELSRTAEQVMISSRSGSWVMSRVWDNGYPWDMLLVTRFGTFLKNNLPT
AISDWLYVKQMNARFKHENYGLMPLNGVLRKEPVENDELPASILCGIVSVKPNVKEFTETSA
IFEDGTIFEGIDCVIFATGYSFAYPFLDESIIKSRNNEIILFKGVFPPLLEKSTIAVIGFVQSLGAAI
PTVDLQSRWAAQVIKGTCTLPSMEDMMNDINEKMEKKRKWFGKSETIQTDYIVYMDELSSFI
GAKPNIPWLFLTDPKLAMEVYFGPCSPYQFRLVGPGQWPGARNAILTQWDRSLKPMQTRVV
GRLQKPCFFFHWLKLFAIPILLIAVFLVLT

In some embodiments of any of the aspects, the sarcosine N-methyltransferase (SNMT) is encoded by SEQ ID NO: 74 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more identical to SEQ ID NO: 74 that maintains the same function, or a codon-optimized version thereof.

In some embodiments of any of the aspects, the sarcosine N-methyltransferase (SNMT) comprises SEQ ID NO: 75 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, at least 99%, or more similar to SEQ ID NO: 75, that maintains the same function. SNMT catalyzes the methylation of glycine and sarcosine to sarcosine and dimethylglycine or trimethylglycine, respectively, with S-adenosylmethionine (AdoMet) acting as the methyl donor. Specifically, sarcosine N-Methyl Transferase first adds a methyl group to sarcosine to form dimethylglycine. This same SNMT enzyme can then use dimethylglycine as a substrate, adding a methyl group to dimethylglycine to form trimethylglycine, aka betaine. In some embodiments of any of the aspects, the sarcosine N-methyltransferase is derived from a sarcosine N-methyltransferase gene or polypeptide of a Halothece species, e.g., Halothece sp. PCC 7418 (see e.g., SEQ ID NOs: 74-75). In some embodiments of any of the aspects, the sarcosine N-methyltransferase is derived from a Halorhodospira halochloris sarcosine N-methyltransferase gene or polypeptide (see e.g., SEQ ID NOs: 76-77).

Halothece sp. PCC 7418 SNMT 831 nt,
SEQ ID NO: 74
ATGACAAAAGCGGACGCCGTTGCTAAACAAGCCCAAGATTACTATGATTCGGGCAGCGC
TGATGGATTCTACTATAGAATCTGGGGCGGCGAGGATCTGCACATAGGTATATACAATAC
ACCAGATGAACCAATTTACGATGCAAGCGTTCGAACGGTGTCTCGTATCTGCGACAAAAT
CAAAAACTGGCCCGCAGGCACAAAGGTGTTAGACTTAGGTGCGGGGTATGGAGGCAGTG
CCCGCTACATGGCGAAACATCATGGATTCGACGTAGATTGCTTGAACATTTCTTTAGTAC
AAAACGAAAGAAATCGTCAAATGAATCAAGAACAGGGTCTTGCAGATAAAATTAGGGTC
TTTGACGGATCATTTGAGGAATTGCCGTTCGAGAATAAGTCATATGATGTGCTATGGTCC
CAAGACTCCATTCTGCACTCAGGGAATCGCAGAAAAGTTATGGAAGAAGCCGATCGTGT
ACTTAAATCTGGGGGCGATTTTGTCTTTACTGACCCGATGCAAACCGATAACTGTCCTGA
AGGTGTCTTGGAGCCGGTGCTGGCGCGGATTCATCTCGATAGTCTGGGTTCAGTTGGCTT
TTATAGACAAGTGGCAGAGGAATTAGGGTGGGAATTCGTCGAATTTGACGAACAGACGC
ATCAGTTGGTCAATCATTATAGCCGGGTACTTCAGGAGTTAGAAGCTCATTATGATCAGC
TTCAGCCTGAATGTTCGCAGGAATATCTTGATCGCATGAAAGTTGGACTCAATCACTGGA
TTAACGCAGGCAAAAGCGGATATATGGCTTGGGGAATCCTGAAGTTTCATAAGCCG
Halothece sp. PCC 7418SNMT 277 aa,
SEQ ID NO: 75
MTKADAVAKQAQDYYDSGSADGFYYRIWGGEDLHIGIYNTPDEPIYDASVRTVSRICDKIKN
WPAGTKVLDLGAGYGGSARYMAKHHGFDVDCLNISLVQNERNRQMNQEQGLADKIRVFD
GSFEELPFENKSYDVLWSQDSILHSGNRRKVMEEADRVLKSGGDFVFTDPMQTDNCPEGVLE
PVLARIHLDSLGSVGFYRQVAEELGWEFVEFDEQTHQLVNHYSRVLQELEAHYDQLQPECSQ
EYLDRMKVGLNHWINAGKSGYMAWGILKFHKP
SNMT_HALHR Sarcosine N-methyltransferase Halorhodospira
halochloris, 837 nt,
SEQ ID NO: 76
ATGGCCACACGTTACGACGATCAAGCGATTGAGACAGCACGCCAGTACTATAATAGTGA
GGACGCGGATAATTTCTATGCCATTATCTGGGGAGGGGAGGACATTCATATCGGCTTATA
TAACGATGACGAAGAACCTATAGCCGATGCTAGTCGGAGAACTGTTGAACGCATGTCTT
CGTTGTCCAGGCAATTAGGTCCAGACTCTTATGTACTCGATATGGGAGCAGGATACGGGG
GCTCAGCTCGTTATCTTGCACATAAATATGGTTGTAAGGTAGCAGCTTTGAACTTGTCCG
AAAGAGAAAATGAACGAGACCGTCAAATGAACAAAGAACAAGGTGTCGATCATTTAATT
GAAGTCGTTGATGCCGCGTTTGAAGACGTGCCGTATGATGATGGCGTGTTTGATCTCGTC
TGGTCACAAGATTCATTCTTACATAGCCCTGATCGCGAACGTGTACTGAGAGAAGCGAGC
CGTGTTCTGCGGTCTGGAGGAGAGTTCATATTTACAGATCCGATGCAAGCTGACGATTGC
CCGGAGGGAGTTATTCAGCCAATCCTTGATAGAATTCACCTTGAAACGATGGGAACCCC
GAATTTTTATAGACAGACCCTGCGAGACCTAGGATTTGAAGAGATTACGTTCGAAGATCA
TACACACCAGCTTCCCAGGCACTATGGGCGCGTCCGGCGCGAACTGGATAGACGAGAGG
GCGAGCTGCAGGGCCATGTGAGCGCAGAATACATCGAACGGATGAAAAACGGTTTAGAC
CATTGGGTGAATGGCGGCAATAAAGGGTACCTTACGTGGGGTATCTTTTATTTTAGGAAG
GGC
SNMT_HALHR Sarcosine N-methyltransferase Halorhodospira,
halochloris, 279 aa
SEQ ID NO: 77
MATRYDDQAIETARQYYNSEDADNFYAIIWGGEDIHIGLYNDDEEPIADASRRTVERMSSLSR
QLGPDSYVLDMGAGYGGSARYLAHKYGCKVAALNLSERENERDRQMNKEQGVDHLIEVV
DAAFEDVPYDDGVFDLVWSQDSFLHSPDRERVLREASRVLRSGGEFIFTDPMQADDCPEGVI
QPILDRIHLETMGTPNFYRQTLRDLGFEEITFEDHTHQLPRHYGRVRRELDRREGELQGHVSA
EYIERMKNGLDHWVNGGNKGYLTWGIFYFRKG

In one aspect described herein is a method of generating taurine from methionine in the gut of a mammal. In one aspect, the method comprises introducing an engineered taurine-producing probiotic microorganism as described herein to the gut of the mammal. In some embodiments of any of the aspects, the taurine-producing microorganism is introduced via oral administration. In some embodiments of any of the aspects, the taurine-producing microorganism is introduced via rectal administration.

### Administration

In one aspect described herein is a pharmaceutical composition comprising an engineered probiotic microorganism as described herein (e.g., an engineered methionine-reducing probiotic microorganism; an engineered methanethiol-reducing probiotic microorganism; and/or an engineered taurine-producing probiotic microorganism), and a pharmaceutically acceptable carrier. In some embodiments of any of the aspects, the purified mixture of live bacteria comprises species present in an amount of at least about 1×10⁸ CFUs/ml (colony-forming units per milliliter). In some embodiments of any of the aspects, the purified mixture of live bacteria comprises species present in an amount of at least 1×10¹ CFUs/ml, at least 1×10² CFUs/ml, at least 1×10³ CFUs/ml, at least 1×10⁴ CFUs/ml, at least 1×10⁵ CFUs/ml, at least 1×10⁶ CFUs/ml, at least 1×10⁷ CFUs/ml, at least 1×10⁸ CFUs/ml, at least 1×10⁹ CFUs/ml, at least 1×10¹⁰ CFUs/ml, at least 1×10¹¹ CFUs/ml, or at least 1×10¹² CFUs/ml, or more.

In some embodiments of any of the aspects, the pharmaceutical composition is formulated for oral administration. In some embodiments of any of the aspects, the pharmaceutical composition is formulated for delivery to the gut via oral administration. In some embodiments of any of the aspects, the pharmaceutical composition is formulated for delivery to the intestine via oral administration. In some embodiments of any of the aspects, the pharmaceutical composition is enteric coated. In some embodiments of any of the aspects, the pharmaceutical composition is formulated for injection (e.g., into the bloodstream for treatment of cancer). It has been shown in mouse models that intravenously injected bacteria (e.g., E. coli Nissle) selectively colonizes certain tumors while being cleared from healthy tumors.

In some embodiments, the pharmaceutical composition further comprises at least one additional methionine-decreasing or homocysteine-decreasing therapeutic. For example, in some embodiments, the pharmaceutical composition further comprises an effective amount of betaine and/or taurine. In some embodiments, the pharmaceutical composition is co-administered with at least one additional methionine-decreasing or homocysteine-decreasing therapeutic. In some embodiments, the at least one additional methionine-decreasing or homocysteine-decreasing therapeutic is administered before, concurrently, or after the administration of the engineered bacterium describe herein. In some embodiments, the at least one additional methionine-decreasing or homocysteine-decreasing therapeutic is selected from the group consisting of: betaine, taurine, a methionine restriction diet, a methionine-free formula, and combinations thereof. In some embodiments, the at least one additional methionine-decreasing or homocysteine-decreasing therapeutic and the engineered bacterium are all administered orally or rectally. In some embodiments, the at least one additional methionine-decreasing or homocysteine-decreasing therapeutic and the engineered bacterium are all administered by injection. In some embodiments, the at least one additional methionine-decreasing or homocysteine-decreasing therapeutic is injected, and the engineered bacterium is administered orally or rectally. In some embodiments, the at least one additional methionine-decreasing or homocysteine-decreasing therapeutic is administered orally or rectally, and the engineered bacterium is injected.

In one aspect described herein is a dietary supplement comprising an engineered probiotic microorganism as described herein (e.g., an engineered methionine-reducing probiotic microorganism; and/or an engineered methanethiol-reducing probiotic microorganism; an engineered taurine-producing probiotic microorganism). The term “dietary supplement,” which can be used interchangeably with the term “nutritional supplement,” refers to any product that is added to the diet. The primary purpose of the dietary supplement is to promote wellbeing and/or digestive health, as opposed to targeted treatment of a specific disease. In some embodiments, nutritional supplements are taken by mouth and often contain one or more dietary ingredients, including but not limited to vitamins, minerals, herbs, amino acids, enzymes, and cultures of organisms. As used herein, the term “nutraceutical” refers to a food/dietary supplement that is believed and/or taken to provide health benefits.

In one aspect described herein is a food composition comprising an engineered probiotic microorganism as described herein (e.g., an engineered methionine-reducing probiotic microorganism; an engineered methanethiol-reducing probiotic microorganism; and/or an engineered taurine-producing probiotic microorganism). In some embodiments of any of the aspects, the food composition comprises a yogurt. In some embodiments of any of the aspects, the food composition comprises a yogurt a beverage. In some embodiments of any of the aspects, the food composition is a medical food. As used herein, “medical food” is understood to mean a food which is formulated to be consumed or administered enterally under the supervision of a physician and which is intended for the specific dietary management of a disease or condition for which distinctive nutritional requirements, based on recognized scientific principles, are established by medical evaluation.

In some embodiments, the therapeutic composition or dose unit comprises a pharmaceutically acceptable formulation, including an enteric coating or similar to survive the acidity of the stomach and permit delivery into the small or large intestine, a prebiotic (such as, but not limited to, amino acids (e.g., arginine, glutarate, and ornithine), biotin, fructooligosaccharide, galactooligosaccharides, hemi celluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) xylooligosaccharides, polyamines (such as but not limited to spermidine and putrescine), an effective amount of an anti-bacterial agent, anti-fungal agent, anti-viral agent, or anti-parasitic agent, or any combinations of the above.

In some embodiments, the active ingredients of the pharmaceutical composition comprise the engineered probiotic microorganism(s) as described herein. In some embodiments, the active ingredients of the pharmaceutical composition consist essentially of the engineered probiotic microorganism(s) as described herein. In some embodiments, the active ingredients of the pharmaceutical composition consist of the engineered probiotic microorganism(s) as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C2-Cu alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. the engineered probiotic microorganism(s) as described herein.

Pharmaceutical compositions comprising the engineered probiotic microorganism(s) as described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

In some embodiments, the methods described herein relate to treating a subject having or diagnosed as having a methionine-associated disease or disorder with the engineered probiotic microorganism(s) as described herein. In some embodiments, the methods described herein comprise administering an effective amount of compositions described herein, e.g., the engineered probiotic microorganism(s) as described herein, to a subject in order to alleviate a symptom of a methionine-associated disease or disorder. As used herein, “alleviating a symptom of a methionine-associated disease or disorder is ameliorating any condition or symptom associated with the methionine-associated disease or disorder. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to rectal, anal, enteric, or oral administration. Administration can be local or systemic. In some embodiments of any of the aspects, the engineered probiotic microorganism(s) as described herein is administered using a stoma, catheter, oral or nasal tube, enema, suppository, colonoscope, or enteroscope.

The term “effective amount” as used herein refers to the amount of the engineered probiotic microorganism(s) as described herein needed to alleviate at least one or more symptom of the disease or disorder, and relates to a sufficient amount of pharmacological composition to provide the desired effect. The term “therapeutically effective amount” therefore refers to an amount of engineered probiotic microorganism(s) as described herein that is sufficient to provide a particular anti-methionine-associated disease or disorder effect when administered to atypical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals. The dosage can vary depending upon the dosage form employed and the route of administration utilized.

In some embodiments of any of the aspects, the engineered probiotic microorganism(s) as described herein is administered as a monotherapy, e.g., another treatment for the methionine-associated disease or disorder is not administered to the subject.

In some embodiments of any of the aspects, the methods described herein can further comprise administering a second agent and/or treatment for the methionine-associated disease or disorder to the subject, e.g., as part of a combinatorial therapy. In some embodiments, the engineered bacterium described herein is an adjunct therapy that can be used along with other pharmaceutical compositions and/or therapeutics. Without wishing to be bound by theory, it is contemplated that treatments for methionine-associated disease or disorders (such as HCU or methionine-dependent cancers) can achieve high efficacy when combining the engineered bacterium described herein with additional methionine-decreasing or homocysteine-decreasing therapeutics, such as betaine or taurine. In some embodiments of any of the aspects, the engineered bacterium described herein is co-administered with an effective amount of at least one additional methionine-decreasing or homocysteine-decreasing therapeutic. As a non-limiting example, the methionine-decreasing or homocysteine-decreasing therapeutic can be selected from the group consisting of: betaine (e.g., CYSTADANE, betaine anhydrous for oral solution), taurine, a methionine restriction diet, a methionine-free formula (e.g., HOMINEX-2), and combinations thereof. In some embodiments of any of the aspects, the at least one additional methionine-decreasing or homocysteine-decreasing therapeutic is betaine or taurine.

In some embodiments of any of the aspects, the methods described herein can further comprise administering a second agent and/or treatment to the subject, e.g., as part of a combinatorial therapy. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gammalI and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising engineered probiotic microorganism(s) as described herein can be administered to a patient once. In certain embodiments, an effective dose of a composition comprising engineered probiotic microorganism(s) as described herein can be administered to a patient repeatedly. In some embodiments, the administered engineered microorganism colonizes the gut, i.e., establishes a non-transitory residence of the gut. In some embodiments, the administered engineered microorganism does not necessarily colonize the gut and/or is re-administered.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g., a methionine-associated disease or disorder by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the engineered probiotic microorganism(s) as described herein. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. In some embodiments, the engineered microorganism is administered daily, twice daily, three times daily, or more. In some embodiments, the engineered microorganism is administered every two days, every three days, weekly, etc. Further examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising engineered probiotic microorganism(s) as described herein can be administered over a period of time, such as over a 5 minute, 10 minute, 15 minute, 20 minute, or 25 minute period.

In some embodiments, the engineered microorganism is administered with food or a beverage. In some embodiments, the engineered microorganism is administered at meal times, which can be especially beneficial for a meal that comprises a high level of methionine (e.g., a meal comprising turkey, beef, fish, pork, tofu, milk, cheese, nuts, beans, whole grains like quinoa, and other protein-rich foods). In some embodiments, the engineered microorganism is administered at breakfast, brunch, lunch, teatime, dinner, snack time, or another time when food is eaten.

The dosage ranges for the administration of the engineered probiotic microorganism(s) as described herein according to the methods described herein depend upon, for example, the form of the composition, its potency, and the extent to which symptoms, markers, or indicators of a condition described herein are desired to be reduced, for example the percentage reduction desired for methionine-associated disease or disorder. The dosage should not be so large as to cause adverse side effects, such as sepsis, infection, diarrhea, or constipation. Generally, the dosage will vary with the age, condition, and sex of the patient and can be determined by one of skill in the art. The dosage can also be adjusted by the individual physician in the event of any complication. In some embodiments, the dosage can vary with the amount of methionine consumed. As a non-limiting examples, an increased dosage of the engineered methionine-reducing microorganism described herein can be administered with methionine-rich foods, such as turkey, beef, fish, pork, tofu, milk, cheese, nuts, beans, whole grains like quinoa, and other protein-rich foods.

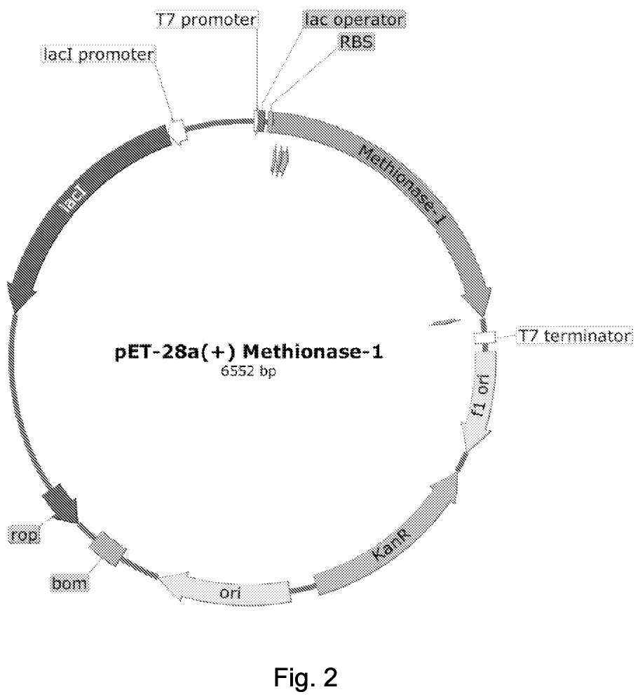

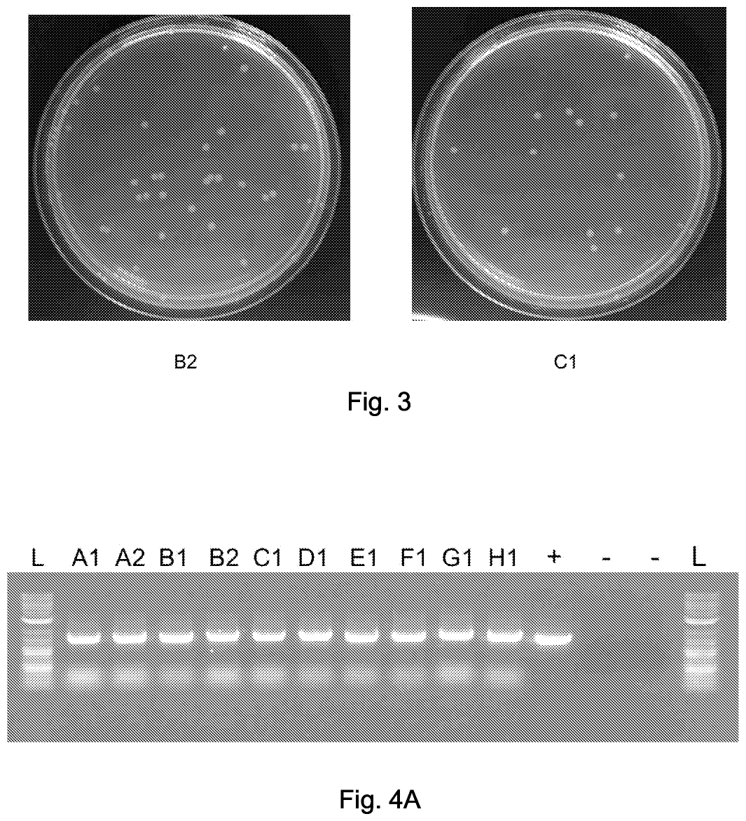

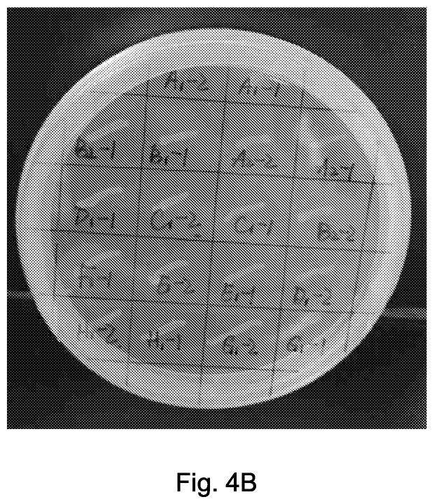

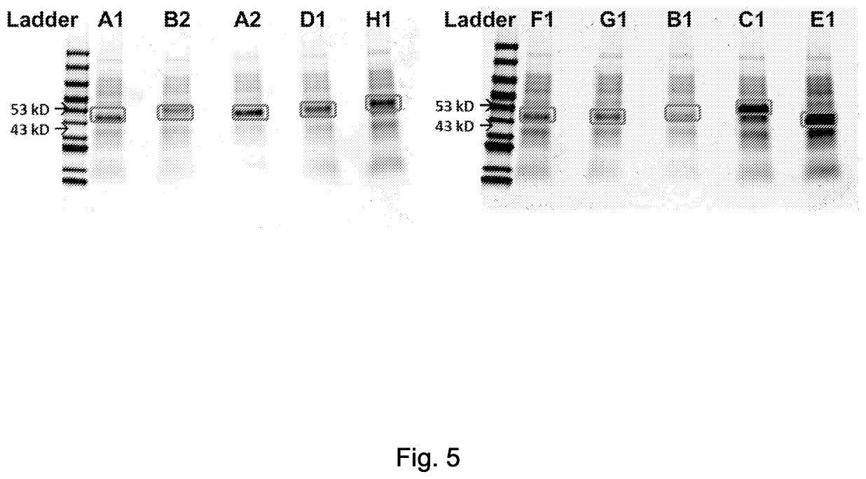

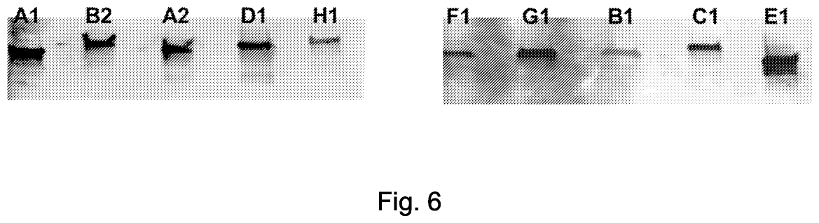

The efficacy of the engineered probiotic microorganism(s) as described herein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. the level of methionine in the gut or blood. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of a methionine-associated disease or disorder. When using an experimental animal model, efficacy of treatment is evidenced when a statistically significant change in a marker is observed, e.g. the level of methionine in the gut or blood. In vitro assays allow the assessment of a given dose of an engineered probiotic microorganism(s) as described herein (see e.g., FIG. 2-9).

### Treatment Methods

The compositions described herein can be administered to a subject in need thereof, for instance for the treatment of a methionine-associated disease or disorder. Non-limiting examples of a methionine-associated disease or disorder include homocystinuria, hypermethioninemia, obesity, and cancer (e.g., a glioma). In some embodiments, the methionine-associated disease or disorder is homocystinuria.

In some embodiments, the method of treatment can comprise first diagnosing a subject or patient who can benefit from treatment by a composition described herein. In some embodiments, such diagnosis comprises detecting or measuring a high level of methionine in a sample from the subject or patient, which is an example of an abnormal level of an analyte. In some embodiments, the method further comprises administering to the patient a composition as described herein.

In some embodiments, the subject has previously been determined to have an abnormal level of an analyte described herein relative to a reference. In some embodiments, the reference level can be the level in a sample of similar cell type, sample type, sample processing, and/or obtained from a subject of similar age, sex and other demographic parameters as the sample/subject. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the step of determining if the subject has an abnormal level of an analyte described herein can comprise i) obtaining or having obtained a sample from the subject and ii) performing or having performed an assay on the sample obtained from the subject to determine/measure the level of the analyte in the subject. In some embodiments of any of the aspects, the step of determining if the subject has an abnormal level of an analyte described herein can comprise performing or having performed an assay on a sample obtained from the subject to determine/measure the level of analyte in the subject. In some embodiments of any of the aspects, the step of determining if the subject has an abnormal level of an analyte described herein can comprise ordering or requesting an assay on a sample obtained from the subject to determine/measure the level of the analyte in the subject. In some embodiments of any of the aspects, the step of determining if the subject has an abnormal level of an analyte described herein can comprise receiving the results of an assay on a sample obtained from the subject to determine/measure the level of the analyte in the subject. In some embodiments of any of the aspects, the step of determining if the subject has an abnormal level of an analyte described herein can comprise receiving a report, results, or other means of identifying the subject as a subject with a decreased level of the analyte.

In one aspect of any of the embodiments, described herein is a method of treating a methionine-associated disease or disorder in a subject in need thereof, the method comprising: a) determining if the subject has an abnormal level of an analyte described herein; and b) instructing or directing that the subject be administered a composition comprising at least one engineered probiotic microorganism as described herein if the level of the analyte is decreased relative to a reference. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In one aspect of any of the embodiments, described herein is a method of treating a cancer in a subject in need thereof, the method comprising administering an effective amount of an engineered probiotic microorganism as described herein. A variety of cancers have been identified using cancer cell lines and xenograft models that are responsive to methionine depletion; see e.g., Wanders et al. “Methionine Restriction and Cancer Biology,” Nutrients. 2020 March; 12(3): 684, the contents of which are incorporated by reference herein in their entirety. In some embodiments, the cancer is a cancer that is responsive to methionine depletion. In some embodiments, the cancer is a methionine-dependent cancer. In some embodiments, the cancer is selected from the group consisting of: glioma (e.g., diffuse midline glioma; see e.g., Example 3), colon cancer, breast cancer (including, but not limited to triple negative breast cancers), ovarian cancer, prostate cancer, melanoma, and sarcoma, which are non-limiting examples of cancers that are methionine-dependent and thus responsive to methionine depletion therapies (see e.g., Table 1 of Wanders). The efficacy of the engineered bacterium described herein can be demonstrated, for example, in a cancer cell line and/or an animal model specific for the cancer (e.g., using procedures described in Examples 1-3).

### Vectors

In some embodiments, one or more of the genes described herein is expressed in a recombinant expression vector or plasmid. As used herein, the term “vector” refers to a polynucleotide molecule suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al., Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., bacteriophage vectors), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined or linked when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATAAT element or Pribnow box, capping sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences or operon sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is pET (see e.g., FIG. 2).

Without limitations, the genes described herein can be included in one vector or two or more separate vectors. For example, the gene encoding an exogenous methionine degrading enzyme (e.g., MGL) or the exogenous methionine importer gene can be included in the same vector. In some embodiments, the gene encoding an exogenous methionine degrading enzyme (e.g., MGL) can be included in a first vector, and the exogenous methionine importer gene can be included in a second vector.

In some embodiments, the gene encoding a methanethiol catabolizing enzyme (e.g., is an esterase or a methanethiol oxidase), or the gene encoding a catalase, or the gene encoding a formaldehyde dehydrogenase, or the gene encoding a formate acetyltransferase, or the gene encoding a sulfite reductase can be included in the same vector. In some embodiments, the gene encoding a methanethiol catabolizing enzyme (e.g., is an esterase or a methanethiol oxidase) can be included in a first vector, or the gene encoding a catalase can be included in a second vector, or the gene encoding a formaldehyde dehydrogenase can be included in a third vector, or the gene encoding a formate acetyltransferase can be included in a fourth vector, or the gene encoding a sulfite reductase can be included in a fifth vector.

In some embodiments, the gene encoding a homocysteine methyltransferase enzyme, or the gene encoding a glycine N-methyltransferase, the gene encoding a sarcosine N-methyl transferase, or the gene encoding a sulfinoalanine decarboxylase enzyme, or the gene encoding a Flavin-containing monooxygenase enzyme can be included in the same vector. In some embodiments, the gene encoding a homocysteine methyltransferase enzyme can be included in a first vector, or the gene encoding a glycine N-methyltransferase can be included in a second vector, or the gene encoding a sarcosine N-methyl transferase can be included in a third vector, or the gene encoding a sulfinoalanine decarboxylase enzyme can be included in a fourth vector, or the gene encoding a Flavin-containing monooxygenase enzyme can be included in a fifth vector.

In some embodiments, one or more of the recombinantly expressed genes can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “probiotic” refers to a live microbial food, supplement, or pharmaceutical ingredient that is beneficial to health.

As used herein, the term “prebiotic” refers to a food ingredient or supplement that is not digestible by the human or other animal ingesting it, but that beneficially affects the human and/or other animal that ingests it by providing a food source for beneficial bacteria. In some embodiments, prebiotics selectively stimulate the growth and/or activity of at least one type of microorganism in the intestinal tract, such that the health of the human and/or other animal is improved.

As used herein, the term “synbiotic” refers to a mixture of prebiotics and probiotics.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its amino acid sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein “inactivating modification” refers to a mutation, including an insertion, deletion, or substitution that decreases or eliminates the expression and/or activity of a relevant gene product. In some embodiments, an inactivating modification refers to the partial or complete deletion of the indicated gene.

As used herein “activating modification” refers to a mutation, including an insertion, deletion, or substitution that increases the expression and/or activity of a relevant gene product.

In some embodiments of any of the aspects, the polypeptides described herein are exogenous. In some embodiments of any of the aspects, the polypeptides described herein is ectopic. In some embodiments of any of the aspects, the polypeptides described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

In some embodiments of any of the aspects, the engineered bacterium comprises at least one functional heterologous gene. As used herein, the term “heterologous” refers to that which is not endogenous to, or naturally occurring in, a referenced sequence, molecule (including e.g., a protein), virus, cell, tissue, or organism. For example, a heterologous sequence of the present disclosure can be derived from a different species, or from the same species but substantially modified from an original form. Also for example, a nucleic acid sequence that is not normally expressed in a cell or a virus is a heterologous nucleic acid sequence with regard to that cell or virus. The term “heterologous” can refer to DNA, RNA, or protein that does not occur naturally as part of the organism in which it is present or which is found in a location or locations in the genome that differ from that in which it occurs in nature. It is DNA, RNA, or protein that is not endogenous to the virus or cell and has been artificially introduced into the virus or cell.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a methionine-associated disease or disorder. A subject can be male or female.

A subject can be one who has been previously diagnosed with or identified as suffering from or having a condition in need of treatment (e.g. a methionine-associated disease or disorder) or one or more complications related to such a condition, and optionally, have already undergone treatment for a methionine-associated disease or disorder or the one or more complications related to a methionine-associated disease or disorder. Alternatively, a subject can also be one who has not been previously diagnosed as having a methionine-associated disease or disorder or one or more complications related to a methionine-associated disease or disorder. For example, a subject can be one who exhibits one or more risk factors for a methionine-associated disease or disorder or one or more complications related to a methionine-associated disease or disorder or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

As used herein, the phrase “maintains the same function”, when used in reference to an enzyme, catalyzes the same reaction as a reference enzyme. When used in reference to an importer, it imports the same molecule, substance, or factor.

In some embodiments, sequencing comprises 16S rRNA gene sequencing, which can also be referred to as “16S ribosomal RNA sequencing”, “16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary by species. 16S rRNA gene sequencing typically comprises using a plurality of universal primers that bind to conserved regions of the 16S rRNA gene, PCR amplifying the bacterial 16S rRNA gene regions (including hypervariable regions), and sequencing the amplified 16S rRNA genes with a next-generation sequencing technology as described herein (see also e.g., U.S. Pat. Nos. 5,654,418; 6,344,316; and 8,889,358; and US Patent Application Numbers US 2013/0157265 and US 2018/0195111, which are incorporated by reference in their entireties).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

“Expression products” include RNA transcribed from a gene, and polypeptides obtained by translation of mRNA transcribed from a gene. The term “gene” refers to the nucleic acid sequence which is transcribed (DNA) to RNA in vitro or in vivo when operably linked to appropriate regulatory sequences. The gene may or may not include regions preceding and following a coding region, e.g. 5′ untranslated (5′UTR) or “leader” sequences and 3′ UTR or “trailer” sequences, as well as intervening sequences (introns) between individual coding segments (exons).

In some embodiments, a nucleic acid encoding a polypeptide as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell. In some embodiments of any of the aspects, the vector and/or nucleic acid sequence described herein is codon-optimized for expression in a yeast or yeast cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art, including numerous forms of bacteriophage vectors.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a methionine-associated disease or disorder. The term “treating” includes reducing or alleviating at least one adverse effect or symptom of a condition, disease or disorder associated with methionine. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. An engineered probiotic microorganism for reducing bioavailable methionine levels, comprising:
   a) at least one exogenous copy of at least one gene encoding an enzyme that catalyzes the degradation of methionine, wherein the gene encoding an enzyme that catalyzes the degradation of methionine encodes a methionine gamma lyase.
2. An engineered probiotic microorganism for reducing bioavailable methionine levels, comprising:
   a) at least one exogenous copy of at least one gene encoding an enzyme that catalyzes the degradation of methionine, wherein the gene encoding an enzyme that catalyzes the degradation of methionine encodes a methionine gamma lyase; and
   b) at least one of the following:
      i) at least one exogenous copy of at least one functional methionine importer gene; and/or
      ii) at least one endogenous methionine importer gene comprising at least one engineered activating modification.
3. An engineered probiotic microorganism for reducing bioavailable methionine levels, comprising:
   a) at least one exogenous copy of at least one gene encoding an enzyme that catalyzes the degradation of methionine;
   b) at least one exogenous copy of at least one functional methionine importer gene;
   c) at least one endogenous methionine importer gene comprising at least one engineered activating modification;
   d) at least one endogenous methionine synthesis gene comprising at least one engineered inactivating modification;
   e) at least one endogenous methionine regulator gene comprising at least one engineered inactivating or activating modification; or
   f) a combination of two or more of (a)-(e).
4. The engineered probiotic microorganism of any one of paragraphs 1-3, wherein the exogenous gene(s) of (a) and (b), if present, and the endogenous gene(s) of (c) (d), and (e), if present, are expressed by the engineered probiotic microorganism under conditions in the gut.
5. The engineered probiotic microorganism of any one of paragraphs 1-3, wherein the at least one engineered activating modification comprises:
   a) at least one engineered activating mutation in the at least one endogenous methionine importer gene or in the at least one endogenous methionine regulator gene; and/or
   b) at least one engineered activating mutation in a promoter operatively linked to the at least one endogenous methionine importer gene or to the at least one endogenous methionine regulator gene.
6. The engineered probiotic microorganism of paragraph 3, wherein the at least one engineered inactivating modification comprises:
   a) at least one engineered inactivating mutation in the at least one endogenous methionine synthesis gene or in the at least one endogenous methionine regulator gene;
   b) at least one engineered inactivating mutation in a promoter operatively linked to the at least one endogenous methionine synthesis gene or to the at least one endogenous methionine regulator gene; and/or
   c) at least one inhibitory RNA molecule specific to at least one messenger RNA (mRNA) expressed by the at least one endogenous methionine synthesis gene or by the at least one endogenous methionine regulator gene.
7. The engineered probiotic microorganism of any one of paragraphs 1-3, wherein the enzyme that catalyzes the degradation of methionine generates methanethiol.
8. The engineered probiotic microorganism of any one of paragraphs 1-3, wherein the gene encoding an enzyme that catalyzes the degradation of methionine encodes a methionine gamma lyase.
9. The engineered probiotic microorganism of any one of paragraphs 1-3, which further comprises and expresses an exogenous gene encoding a methanethiol catabolizing enzyme.
10. The engineered probiotic microorganism of paragraph 7, wherein the methanethiol-catabolizing enzyme is an esterase or a methanethiol oxidase.
11. The engineered probiotic microorganism of any one of any one of paragraphs 1-3, wherein the methionine gamma lyase comprises SEQ ID NO: 6 or an amino acid sequence that is at least 90% identical.
12. The engineered probiotic microorganism of any one of any one of paragraphs 1-3, wherein the methionine gamma lyase comprises one of SEQ ID NOs: 5-6 or an amino acid sequence that is at least 90% identical.
13. The engineered probiotic microorganism of any one of paragraphs 1-3, wherein the gene encoding an enzyme that catalyzes the degradation of methionine encodes expression of a catalytically-active fragment of a methionine gamma lyase.
14. The engineered probiotic microorganism of any one of paragraphs 1-3, wherein the gene encoding an enzyme that catalyzes the degradation of methionine encodes expression of a fusion protein comprising a catalytically-active fragment of a methionine gamma lyase.
15. The engineered probiotic microorganism of any one of paragraphs 1-3, which comprises at least one exogenous copy of at least one gene encoding an enzyme that catalyzes the degradation of methionine and at least one exogenous copy of at least one functional methionine importer gene.
16. The engineered probiotic microorganism of any one of paragraphs 1-3, which comprises at least one exogenous copy of at least one gene encoding an enzyme that catalyzes the degradation of methionine and at least one endogenous copy of at least one functional methionine importer gene comprises a mutation that increases the rate of methionine import relative to wild-type of that enzyme.
17. The engineered probiotic microorganism of any one of paragraphs 1-3, which comprises at least one exogenous copy of at least one gene encoding an enzyme that catalyzes the degradation of methionine and at least one endogenous methionine synthesis gene comprising at least one engineered inactivating modification.
18. The engineered probiotic microorganism of any one of paragraphs 1-3, which comprises at least one exogenous copy of at least one gene encoding an enzyme that catalyzes the degradation of methionine and at least one endogenous methionine regulator gene comprising at least one engineered inactivating or activating modification.
19. An engineered probiotic microorganism for reducing bioavailable methionine levels, the microorganism comprising:
   a) at least one endogenous methionine synthesis gene comprising at least one engineered inactivating modification;
   b) at least one copy of an exogenous gene encoding a homocysteine methyltransferase enzyme;
   c) at least one copy of an exogenous gene encoding a sulfinoalanine decarboxylase enzyme; and
   d) at least one copy of an exogenous gene encoding a Flavin-containing monooxygenase (FMO) enzyme;
   wherein the engineered probiotic microorganism expresses endogenously or exogenously encoded cystathionine β-synthase, cystathionine gamma lyase and cysteine dioxygenase enzymes.
20. The engineered probiotic microorganism of paragraph 19, wherein the homocysteine methyltransferase enzyme is a YhcE homocysteine methyltransferase enzyme.
21. The engineered probiotic microorganism of paragraph 19, wherein the at least one engineered inactivating modification comprises:
   a) at least one engineered inactivating mutation in the at least one endogenous methionine synthesis gene;
   b) at least one engineered inactivating mutation in a promoter operatively linked to the at least one endogenous methionine synthesis gene; and/or
   c) at least one silencing RNA molecule specific to at least one messenger RNA (mRNA) expressed by the at least one endogenous methionine synthesis gene.
22. An engineered probiotic microorganism for reducing bioavailable methionine levels, the microorganism comprising:
   a) at least one endogenous methionine synthesis gene comprising at least one engineered inactivating modification;
   b) at least one copy of an exogenous gene encoding a glycine N-methyltransferase (GNMT) enzyme;
   c) at least one copy of an exogenous gene encoding a sarcosine N-methyl transferase (SNMT) enzyme;
   d) at least one copy of an exogenous gene encoding a sulfinoalanine decarboxylase enzyme; and
   e) at least one copy of an exogenous gene encoding a Flavin-containing monooxygenase (FMO) enzyme;
   wherein the engineered probiotic microorganism expresses endogenously or exogenously encoded methionine adenosyl transferase (MetK), adenosylhomocysteinase (ahcY), cystathionine β-synthase, cystathionine gamma lyase and cysteine dioxygenase enzymes.
23. The engineered probiotic microorganism of paragraph 19 or 22, wherein the FMO enzyme is an FMO1, FMO2 or FMO3 enzyme that catalyzes the catalysis of the conversion of hypotaurine to taurine.
24. The engineered probiotic microorganism of paragraph 19 or 22, which metabolizes methionine to taurine.
25. The engineered probiotic microorganism of paragraph 19 or 22, wherein the at least one engineered inactivating modification comprises:
   a) at least one engineered inactivating mutation in the at least one endogenous methionine synthesis gene;
   b) at least one engineered inactivating mutation in a promoter operatively linked to the at least one endogenous methionine synthesis gene; and/or
   c) at least one silencing RNA molecule specific to at least one messenger RNA (mRNA) expressed by the at least one endogenous methionine synthesis gene.
26. The engineered probiotic microorganism of paragraph 19 or 22, wherein the at least one endogenous methionine synthesis gene is MetE and/or MetH.
27. A pharmaceutical composition comprising an engineered probiotic microorganism of any one of paragraphs 1-3, 19, or 22, and a pharmaceutically acceptable carrier.
28. The pharmaceutical composition of paragraph 27, wherein the purified mixture of live bacteria comprises species present in an amount of at least about 1×10⁸ CFUs/ml.
29. The pharmaceutical composition of paragraph 27, wherein the pharmaceutical composition is formulated for oral administration.
30. The pharmaceutical composition of paragraph 27, wherein the pharmaceutical composition is formulated for delivery to the gut via oral administration.
31. The pharmaceutical composition of paragraph 27, wherein the pharmaceutical composition is enteric coated.
32. The pharmaceutical composition of paragraph 27, wherein the pharmaceutical composition is formulated for injection.
33. The pharmaceutical composition of paragraph 27, wherein the pharmaceutical composition further comprises at least one additional methionine-decreasing or homocysteine-decreasing therapeutic.
34. The pharmaceutical composition of paragraph 27, wherein the pharmaceutical composition is co-administered with at least one additional methionine-decreasing or homocysteine-decreasing therapeutic.
35. The pharmaceutical composition of paragraph 34, wherein the at least one additional methionine-decreasing or homocysteine-decreasing therapeutic is selected from the group consisting of: betaine, taurine, a methionine restriction diet, a methionine-free formula, and combinations thereof.
36. A food composition comprising an engineered probiotic microorganism of any one of paragraphs 1-3, 19, or 22.
37. A probiotic dietary supplement comprising an engineered probiotic microorganism of any one of paragraphs 1-3, 19, or 22.
38. A method of reducing bioavailable methionine in a mammal in need thereof, the method comprising administering an engineered probiotic microorganism of any one of paragraphs 1-3, 19, or 22, or administering a pharmaceutical composition, a food composition, or a probiotic dietary supplement comprising an engineered probiotic microorganism of any one of paragraphs 1-3, 19, or 22, to the mammal.
39. The method of paragraph 38, wherein the administering is oral or rectal.
40. The method of paragraph 38, wherein the administering is by injection.
41. The method of paragraph 38, wherein the administering reduced the level of bioavailable methionine in the gut of the mammal.
42. The method of paragraph 38, wherein the method further comprises administering an effective amount of at least one additional methionine-decreasing or homocysteine-decreasing therapeutic.
43. The method of paragraph 42, wherein the at least one additional methionine-decreasing or homocysteine-decreasing therapeutic is selected from the group consisting of: betaine, taurine, a methionine restriction diet, a methionine-free formula, and combinations thereof.
44. A method of treating a cancer in a subject in need thereof, the method comprising administering an effective amount of an engineered probiotic microorganism of any one of paragraphs 1-3.
45. The method of paragraph 44, wherein the cancer is a methionine-dependent cancer.
46. The method of paragraph 44, wherein the cancer is selected from the group consisting of: glioma colon cancer, breast cancer, ovarian cancer, prostate cancer, melanoma, and sarcoma. 47. The method of paragraph 44, wherein the cancer is a glioma.

48. The method of paragraph 44, wherein the method further comprises administering an effective amount of at least one additional methionine-decreasing or homocysteine-decreasing therapeutic.
49. The method of paragraph 44, wherein the at least one additional methionine-decreasing or homocysteine-decreasing therapeutic is selected from the group consisting of: betaine, taurine, a methionine restriction diet, a methionine-free formula, and combinations thereof.
50. The method of paragraph 44, wherein the method further comprises administering an effective amount of at least one additional cancer therapeutic.
51. The method of paragraph 44, wherein the administering is by injection.
52. A method of reducing a level of methanethiol, the method comprising contacting methanethiol with a probiotic microorganism that encodes and expresses an exogenous gene encoding a methanethiol catabolizing enzyme.
53. The method of paragraph 52, wherein the methanethiol catabolizing enzyme is an esterase.
54. The method of paragraph 52, wherein the methanethiol catabolizing enzyme is a methanethiol oxidase.
55. The method of paragraph 52, wherein the methanethiol is produced by an engineered probiotic microorganism that comprises and expresses an exogenous gene encoding an enzyme that catalyzes the degradation of methionine to products including methanethiol.
56. The method of paragraph 55, wherein the enzyme that catalyzes the degradation of methionine to products including methanethiol comprises a methionine gamma lyase enzyme.
57. A method of reducing odor produced by a population of gut microbiota that produced methanethiol, the method comprising introducing an engineered probiotic microorganism to the gut microbiota, wherein the engineered probiotic microorganism encodes and expresses an exogenous gene encoding a methanethiol catabolizing enzyme.
58. The method of paragraph 57, wherein the methanethiol catabolizing enzyme is an esterase.
59. The method of paragraph 57, wherein the methanethiol catabolizing enzyme is a methanethiol oxidase.
60. The method of paragraph 57, wherein the methanethiol is produced by an engineered probiotic microorganism that comprises and expresses an exogenous gene encoding an enzyme that catalyzes the degradation of methionine to products including methanethiol.
61. The method of paragraph 60, wherein the enzyme that catalyzes the degradation of methionine to products including methanethiol comprises a methionine gamma lyase enzyme.
62. A method of generating taurine from methionine in the gut of a mammal, the method comprising introducing an engineered probiotic microorganism of paragraph 19 or 22 to the gut of the mammal.
63. The method of paragraph 63, wherein the microorganism is introduced via oral administration.

The technology described herein is further illustrated by the following examples which in no way should be construed as being further limiting.

### EXAMPLES

### Example 1: a Probiotic Microorganism that can Reduce Methionine Levels

Poor diets are causing an epidemic of chronic disease, and “dieting” is not effective. Two thirds of Americans are overweight or obese. Half of American adults have a preventable, chronic disease related to poor nutrition. Over 80% of diets fail to result in long term health benefits. After a person eats, approximately 100 trillion gut microbes process and break down food before absorption. Eating habits are hard to break, but nutrition is a function of both diet, and what microbes make from it (e.g., secondary metabolites). Described herein are engineered gut microbes to change what one eats, after one eats it.

Specifically described herein are compositions and methods targeting dietary methionine. Overconsumption of methionine is linked to fatty liver disease, Alzheimer's, and heart disease. Low levels of methionine extend life and reduce weight in animal models and human cell culture. Reducing methionine in the diet leads to improved outcomes, such as reducing liver adiposity and fat mass in mice and humans, and increasing efficacy of chemotherapy and radiotherapy in mice. Reduced methionine diets are also the standard of care (SoC) for homocystinuria (HCU), an inherited disorder of methionine metabolism, e.g., due to a deficiency of cystathionine beta synthase or methionine synthase, leading to increased levels of homocysteine (a methionine metabolite) in serum and urine. Furthermore, reduced dietary methionine has an anti-aging impact. Diets with low methionine extended lifespan 55% in an invertebrate model (C. elegans), extended lifespan 40% in a mammalian model (e.g., rat), and extended replicative lifespan 40% in human cells. Overall, dietary restriction of the amino acid methionine has been shown to have health benefits in a variety of model systems, e.g., increasing lifespan in vitro and in vivo and significantly reducing cancer risk and increasing cancer treatment efficacy in mice.

Current approaches to reducing methionine in the diet require replacing all dietary protein with Met(-) powder mix. As this powder is ˜100 USD/day, these methods are unsustainable long term. As such, there is great need for more inexpensive and efficient approaches to decrease methionine levels.

Accordingly, described herein is a probiotic microorganism that can efficiently consume or convert methionine, e.g., in order to mimic the health benefits of a methionine restricted diet. While natural gut microbiota can break down no more than about 50% of dietary methionine before it can be absorbed, the engineered bacteria described herein push this reduction further to achieve desired low Met levels.

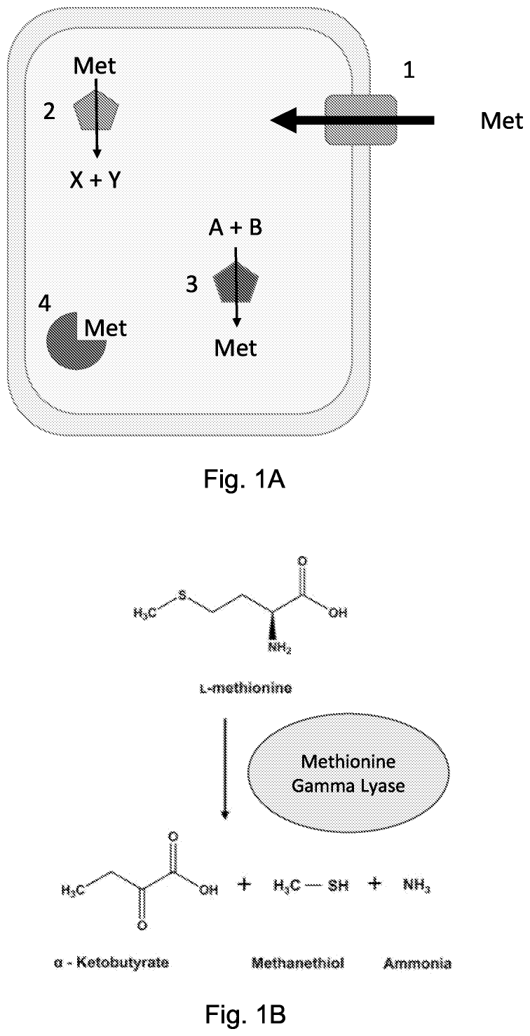

As such, in one aspect described herein is a probiotic microorganism engineered to reduce methionine level in the host environment (see e.g., FIG. 1A-1B). In some embodiments, the probiotic microorganism is selected from the group consisting of E. coli; Bacillus subtilis; Pseudomonas putida; Treponema denticola; Citrobacter freundii; Bacillus cereus; Streptococcus thermophilus; Saccharomyces cerevisiae; Lactococcus lactis; Lactobacillus plantarum; and Brevibacterium linens. In some embodiments, the probiotic microorganism is a food degree bacteria (e.g., recognized as a “food degree” or “food safe” or “food grade” microorganism by the U.S. Food and Drug Administration or otherwise safe or non-hazardous to be present in a food or beverage); a non-limiting example of such a food degree bacteria is Bacillus subtilis. Another non-limiting example of a food-safe Gram-positive organism is Lactococcus lactis or Lactiplantibacillus plantarum. In some embodiments, the microorganism comprises a nucleic acid encoding a methionine gamma lyase (e.g., SEQ ID NOs: 1-6) that can efficiently convert methionine to α-ketobutyrate, ammonia and methyl mercaptan. In some embodiments, the microorganism comprises a nucleic acid encoding an endogenous or exogenous methionine importer, optionally with at least one mutation that increase the rate of methionine import relative to wild-type of that enzyme (see e.g., SEQ ID NOs: 23-34, SEQ ID NOs: 80-89).

In some embodiments, the microorganism is collected from functional screening and directed evolution, e.g., using methionine consumption as a criterion or readout. In some embodiments, no exogenous genetic fragment(s) are inserted in the organism. In some embodiments, the probiotic microorganism can reduce methionine level in surrounding environment by at least 99.9%, at least 99%, at least 98%, at least 95%, at least 94%, at least 93%, at least 92%, at least 91%, at least 90%, at least 80%, at least 70%, at least 60%, at least 50%, at least 40%, at least 30%, at least 20%, at least 10%, at least 5%, or at least 1%.

At least four classes of genes encoding methionine-associated proteins can be engineered (see e.g., FIG. 1A). (1) Methionine importer genes can be mutated to increase the kinetics of methionine import, in order to get methionine out of the gut and into the bacteria as fast as possible. (2) The bacteria can be engineered to express methionine catabolic enzymes. Once methionine is in the cell, it needs somewhere to go, so some sort of breakdown process or sink needs to be there to reduce methionine levels. Such methionine catabolic enzymes can be “mixed and matched” from several organisms, as described further herein. (3) Methionine anabolic enzymes in the bacteria can be knocked out or otherwise inhibited, such that the bacteria cannot produce more methionine (4) Methionine regulatory proteins act as a “thermostat” to the other four methionine-associated proteins described above.

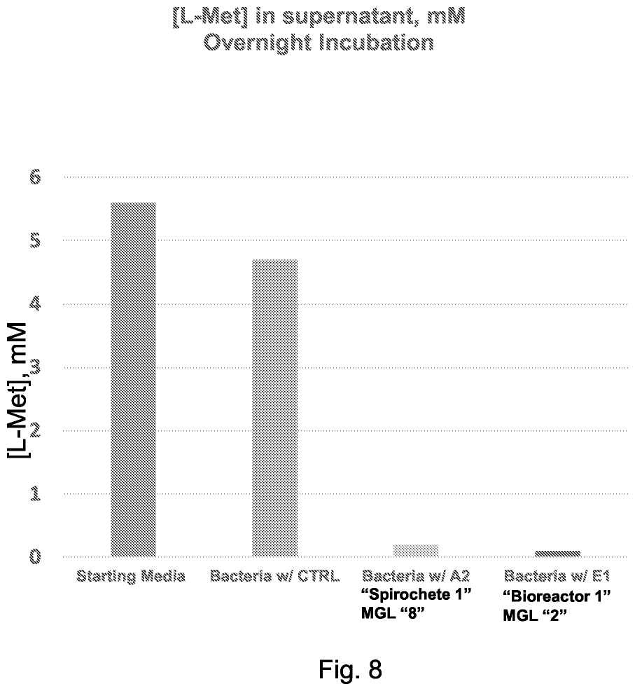

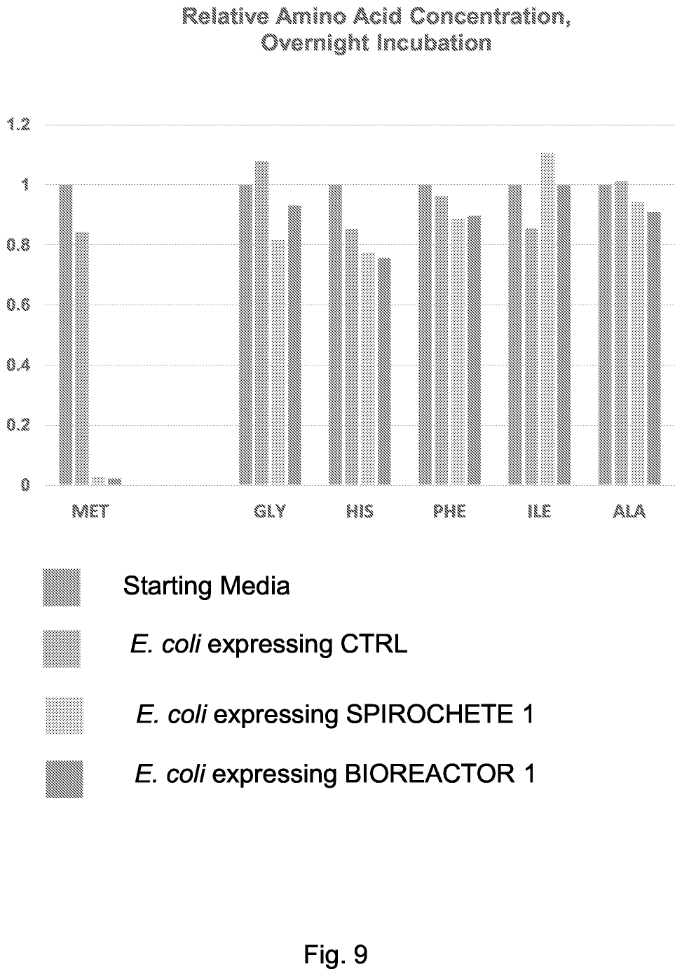

Bacteria were engineered to express heterologous methionine gamma lyase (MGL). FIG. 2-6 show MGL vector construction, transformation, enzyme expression and purification. FIG. 7 shows the screening of candidate methionase enzymes. FIG. 8-9 shows testing of the engineered bacteria for methionase activity. The engineered bacteria reduced methionine levels in media and did not affect levels of other amino acids (see e.g., FIG. 8, FIG. 9).

Microorganisms can also be engineered to reduce levels of methanethiol. Methanethiol is a non-desirable malodorous product created by methionine gamma lyase from methionine. The safety data sheet (SDS) for methanethiol lists it as flammable. Furthermore, the odor of methanethiol has been described as “fish-like” and the odor resembles a 1:1:1 combination of asparagus urine:rotten cabbage:rotten eggs. Probiotic organisms can be engineered to express both a methionine gamma lyase (MGL) and methanethiol-reducing enzyme(s) (see e.g., FIG. 10-11), thus reducing levels of methionine without leading to malodorous products.

Microorganisms can also be engineered to produce taurine from methionine. Methionine is used as a donor of methyl groups to other chemicals, but then it is regenerated by methionine synthase. To increase demand on methyl donation, at least one methionine synthase can be inhibited, thus blocking the ability to regenerate methionine. The methionine product (e.g., homocysteine) is then shunted down a path that ultimately leads to taurine, which cannot be converted back to methionine by bacteria or mammals (see e.g., FIG. 12-13).

### Example 2: Treatment of Homocystinuria (HCU)

Classical Homocystinuria (HCU) is a rare metabolic condition caused by mutations in the cystathionine-b-synthase (CBS) gene. A major barrier to current treatment is adherence to a methionine restricted diet. Described herein is a strategy for producing enzyme therapeutics expressed in bacterial vectors compatible with the gut microbiome. This approach allows excess metabolites (such as methionine in HCU) to be reduced in the gut prior to systemic uptake and thus mimic the effect of a low methionine diet without strictly adhering to one. The bacterial therapeutic for HCU treatment described herein can be scaled up for manufacturing, tested for safety and efficacy, and prepared for human studies and therapeutic use.

Described herein is a synthetic live bacterial therapeutic for homocystinuria (HCU), an inborn metabolic disorder leading to accumulation of homocysteine (Hey), an intermediary of the amino acid methionine. This condition is estimated to occur at an estimated prevalence of 1 in 100,000 to 200,000. The condition can evade detection until specific hallmarks manifest, including lens detachment from the center of the eye or increased incidence of stroke and other thrombotic conditions. Described herein is a probiotic microorganism engineered to break down methionine in the gut to subsequently reduce systemic levels of Hey. Current treatment strategies for pyridoxine non-responsive HCU typically attempt to lower plasma and tissue levels of Hey by a combination of restricting dietary intake of the Hey precursor methionine and dietary supplementation with trimethylglycine, more commonly referred to as betaine. Both strategies are of limited efficacy due to a lack of adherence to the diet, and unpleasant side effects from taking betaine (e.g., diarrhea, nausea, odor). Described herein are prokaryotic strains compatible with the human gut microbiome to serve as expression vectors for therapeutic proteins capable of targeted modulation of metabolic pathways, such as the methionine cycle. In a search for enzymes capable of impacting methionine levels, an in silico screen was carried out for potential methionase enzymes in microbial genomic datasets. Leading candidate enzymes were subsequently cloned, expressed, and tested in vitro for methionine catalysis capabilities. The best performing enzymes were engineered for expression in a bacterial strain which is known to readily engraft into the human gut microbiome. These strains were tested in a murine genetic model system of HCU and mitigated systemic levels of Hey as intended.

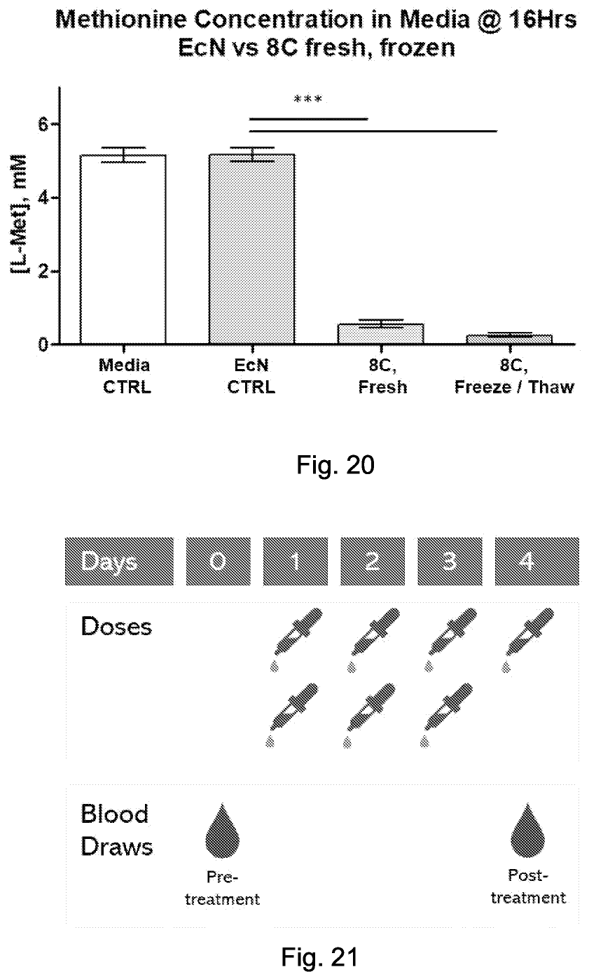

The lead strain can also be tested, for example, in such a murine model of HCU to measure, e.g., mitigation of cognitive deficits due to HCU. The methionase-expressing therapeutic strains decreased levels of systemic methionine and decreased levels of homocysteine (see e.g., plasma homocysteine levels in FIG. 22). In addition, mitigation of other manifestations can be evaluated, including bone density, ophthalmic defects, thrombosis, and cognitive deficits.

### Homocystinuria

HCU is a devastating metabolic disease with a wide range of manifestations, including musculoskeletal, cognitive, ophthalmic effects. The condition requires life-long maintenance via challenging diets and multiple medications. New approaches to treating this condition is urgently needed, and described herein is a strategy to aid in patient adherence of HCU therapies.

HCU heritability and metabolic etiology: Classical homocystinuria (HCU) is caused by deficiency of cystathionine β-synthase (CBS) (EC 4.2.1.22). This enzyme sits at the branch point between the methionine cycle and transsulfuration and catalyzes the condensation of serine and homocysteine (Hcy) into cystathionine which is subsequently converted to cysteine by cystathionine-γ-lyase (CGL) (EC 4.4.1.1). In humans, HCU is characterized by a range of connective tissue disturbances, cognitive deficits and a dramatically increased incidence of vascular disorders, particularly thromboembolic disease. Cardiovascular complications are the major cause of morbidity in HCU, and it has been calculated that an untreated patient with the severest form of this disease has a 27% chance of having a thrombotic event by the age of 15. Furthermore, untreated homocystinuria is associated with a range of both chronic and acute deficits in cognitive function. Patients with untreated B6 responsive HCU have an average IQ score of 79, while untreated B6-nonresponsive patients have an average IQ of 57. Patients diagnosed via newborn screening and kept compliant with dietary therapy had an average IQ of 105, highlighting the urgent need for tools that increase compliance. Acute cognitive problems are associated with HCU as well, such as anxiety and depressive symptoms which can impact working memory tasks. Treatment strategies for pyridoxine non-responsive HCU typically attempt to lower plasma and tissue levels of Hey by a combination of restricting dietary intake of the Hey precursor methionine and dietary supplementation with trimethylglycine, more commonly referred to as betaine. This latter compound serves as a methyl donor in the remethylation of Hey to methionine in a reaction occurring almost exclusively in the liver and catalyzed by betaine-homocysteine S-methyltransferase (BHMT) (EC 2.1.1.5). Early intervention with this treatment can prevent or ameliorate the sequelae of HCU resulting in significantly improved survival and outcome. However, compliance with the methionine-restricted diet is difficult and often poor.

Impacts of HCU: HCU is a genetic condition which indirectly leads to a build-up of homocysteine and subsequently excess methionine, triggering dysregulation of sulfur amino acid metabolism and disease symptoms, leading to elevated risk of stroke and other cardiovascular disease. Estimates indicate that approximately 50% of untreated patients with HCU experience a thromboembolic event before age 30. Current treatments of the disease rely administration of betaine, pyridoxine in patients who have already suffered thrombotic events, and on a highly restrictive methionine-free diet which generally includes fruits, vegetables, plant-based proteins as well as limited types of dairy products and nuts. Animal proteins are especially rich in methionine and are discouraged as part of a methionine-reduced diet. Adherence to dietary treatments is relatively poor given their highly restrictive nature and perhaps more significantly, HCU patients generally “feel fine” on a day-to-day basis. Requiring patient adherence to a challenging diet without additional medical interventions available leads to HCU patients living with a significantly higher risk of stroke and cardiovascular disease. The incidence rate of HCU is also under debate with most historical reports indicating an incidence of approximately 1 in 200,000 globally; however, newborn screening programs have reported higher rates. These tests often rely on methionine levels, however which is an indirect and often inaccurate metric for the condition. Initiatives to improve HCU newborn screening, by directly assaying for homocysteine levels, would likely improve detection rates, early intervention, and a more precise understanding of the condition's incidence.

Current treatment strategies for HCU: Enzyme replacement therapies are under evaluation for HCU. While patients welcome any additional options for treatment, market research indicates that some patients would prefer oral treatments compared to injections such as enzyme replacement therapy (ERT; e.g., TRAVERE). Trials for similar products require 3 injections per day, and have multi-week wash out periods where the intervention cannot be used, to prevent antibody formation. Furthermore, many HCU patients are on blood thinners due to the high risk of thrombotic events. This makes frequent injections even less appealing, due to the increased incidence of painful bruising. In addition, a Cystathionine Beta Synthase (CBS) therapy under development is likely to require IV administration at clinics, presenting adherence challenges for patients and can lead to bruising and bleeding issues given the known link to thrombotic symptoms in HCU.

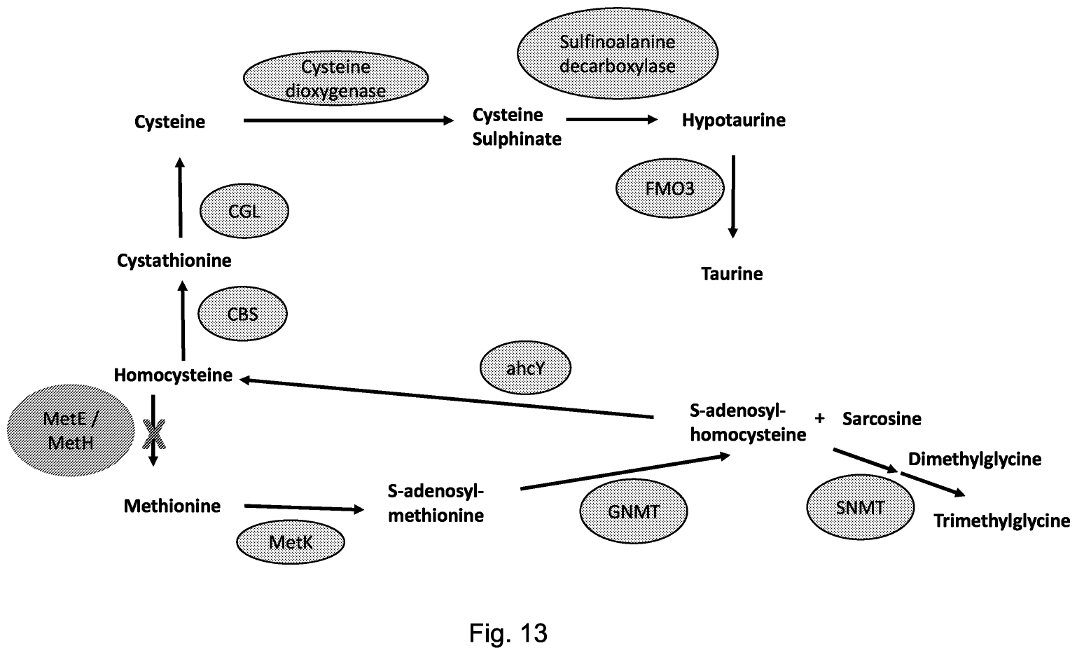

Microbiome therapies: “Additive” microbiome therapeutics describe an approach to treating human disease by administration of bacterial strains, often genetically engineered, to remedy metabolic or microbiome imbalances. These approaches can rely on strains which have intrinsic therapeutic properties against disease, either by producing enzymes or signaling proteins, or by restoring homeostatic microbiome populations among various species. More often, strains are engineered to produce an enzyme which acts on ingested biomass (i.e., food) to modulate the concentration of specific metabolites to redress physiological imbalances. Described herein are microbial strategies with treatment methods related to chronic aging-related disease, obesity, rare metabolic disorders such as HCU (see e.g., FIG. 14).

Specifically, described herein are probiotic microorganisms engineered for methionine metabolism, e.g., for the treatment of HCU, which can include the following features: (1) Dietary methionine restriction by directly metabolizing the amino acid in the gut prior to systemic absorption. Once methionine is absorbed into systemic circulation, treatments are more likely to require intravenous administration, a difficult modality given the bruising and bleeding complications linked to the disease. (2) The use of a bacterially derived methionase which directly reduces methionine levels, permitting homocysteine levels to equilibrate back to normal levels. (3) A gut microbiome compatible bacterial expression vector which allows sustained dosing through protein expression after oral dosing, an easier administration route for improved adherence and fewer bleeding side effects in HCU patients who are known to have thrombotic dysregulation.

Engineering bacterial strains as gut microbiome therapeutics: The number of candidate therapeutics based on bacterial strains compatible with the gut microbiome has grown rapidly in the past 20 years. Strategies for the treatment of infectious disease have been developed including an engineered Lactobacillus jensenii shown to prevent transmission of chimeric simian/human immunodeficiency virus (SHIV) by expressing the antiviral protein cyanovirin-N. Another Lactobacillus species, L. gasseri, has been engineered to express a signaling protein, GLP-1, capable of differentiating intestinal epithelial cells into insulin producing cells mitigating hyperglycemia in rats. While a broad range of bacterial species have been tested for additive microbiome therapeutics, a specific E. coli strain, Nissle 1917, has been used frequently, given its consistently observed safety profile, and intrinsic capabilities to engraft in the human gut microbiome. Nissle 1917 E. coli engineered to produce appetite-suppressing lipids reduced obesity in mice on a high-fat diet and the effect lasted weeks after treatment was suspended. Another group reported the use of Nissle 1917 E. coli strains to express enzymes which convert fructose to mannitol, a means of reducing uptake of dietary sugar and preventing metabolic syndrome. Prevention of cholera virulence with an engineered Nissle strain expressing cholera autoinducer-1 which prevents virulence has also been reported, and adding a transgene for a biofilm degrading enzyme to Nissle 1917 also demonstrated efficacy against gastrointestinal Pseudomonas infections. These data demonstrate that Nissle 1917 can be used as the primary expression vector for the methionine reducing therapeutic for HCU described herein.

Approach to enzyme therapy: bacterial genomes and a gut microbiome compatible vector: An unmet need remains for approaches to repairing complex metabolic pathways disrupted by genetic mutations. Described herein is a strategy for treatment of metabolic disease, leveraging bacterial genomes which often have processing enzymes missing from the human genome, as well as a prokaryotic expression vector which reliably produces active bacterial proteins and is compatible with the human gut microbiome. The bacterial vector allows oral administration of the enzyme therapy, a significant advantage to injected therapies especially in patient populations prone to bleeding and bruising, such as HCU; however, it is contemplated herein that the engineered bacterium can also be injected (e.g., into the bloodstream for treatment of cancer). HCU is a particularly challenging condition, especially since most patients are on antithrombotic therapies, and injection sites become badly bruised and are slow to heal. It should also be noted that in addition to HCU, strategies for reducing systemic methionine levels can be used to treat a range of other conditions. Using in vitro and animal models, methionine restriction has been shown to have efficacy against cancer, and studies are underway to evaluate the efficacy of this strategy in human patients combined with radiotherapy or Akt/ERK inhibitors. Thus, living microbial therapeutic strategies can be used not only for direct modulation of metabolic diseases such as HCU, but many other common life-threatening conditions such as cancer.

Described herein is a methionine-processing, probiotic gut bacteria that modifies plasma homocysteine in a subject with homocystinuria (HCU), without resorting to dietary modifications. Further described herein is: (1) the identification of an enzyme or pathway that significantly and irreversibly degrades methionine in prokaryotes; (2) engineering of the enzyme(s) into a probiotic bacterial chassis and formulating into a storable oral formulation; and (3) testing the formulation on an HCU animal model for efficacy. The results are as follows.

(1) Cloning and Evaluating the Enzymatic Activity of Bacterial Methionases after in Silico Screening.

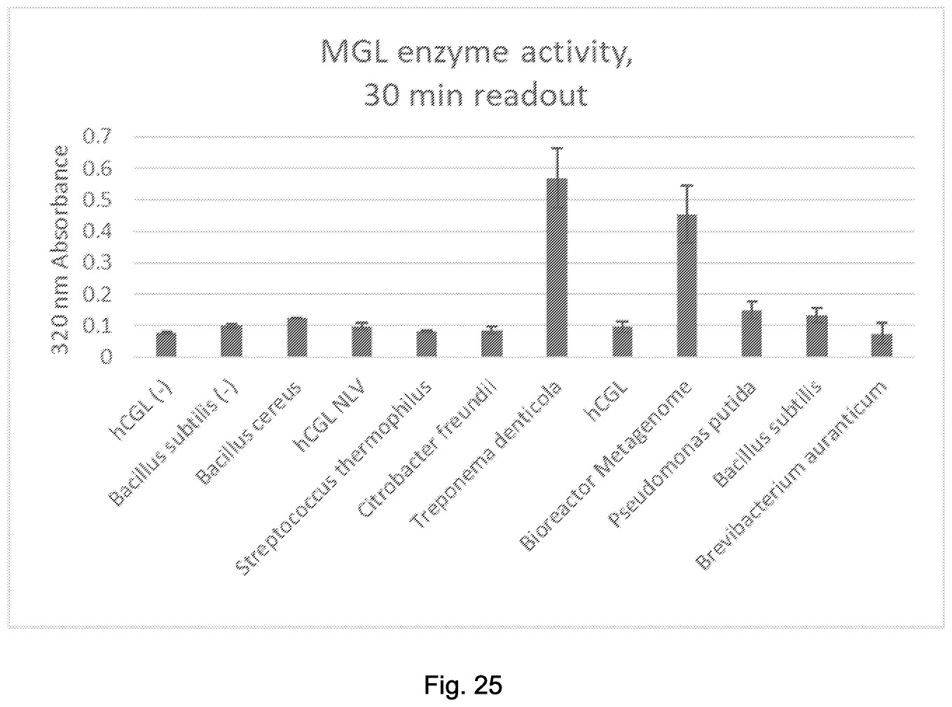

To identify bacterial methionases, bacterial genomes were screened for sequence similarity to the known sequence of methionine-g-lyase (MGL) using BLAST. The class of enzyme is known to free usable ammonia from methionine in bacteria, parasitic protozoa, and plants including Arabidopsis and soybean. Species containing loci with putative MGL activities were then screened against NIAID database PATRIC for known pathogens to eliminate these potentially problematic genes. Remaining loci sequences were then analyzed via VAXIJEN, an open-source, web-based software tool that predicts immunogenic protein sequences from primary sequence data. Using these informatics tools, enzymes in different bacterial species were selected for benchtop analysis. Enzyme sequences were codon optimized, synthesized, and cloned into a standard expression vector under a T7 promoter. The search for usable methionase enzymes began with the top 22 candidates as identified in the bioinformatic pipeline. Positive control (white, “+ Control”) is a previously described methionase; see e.g., Fukumoto et al. “The Role of Amino Acid Residues in the Active Site of 1-Methionine γ-lyase from Pseudomonas putida.” Bioscience, Biotechnology, and Biochemistry. 2012; 76(7):1275-84, the contents of which are incorporated herein by reference in their entirety. The 320 nm absorbance is a result of the reaction of a methionine metabolite MeSH with the reagent DTNB, a way to assay activity level. Of the predicted methionases, five showed greatly enhanced activity against methionine in a 30-minute timeframe. This assay yielded numerous candidates with significantly higher methionine degrading activity than previously described enzymes in the literature. The most active candidates, namely “2” and “8” in FIG. 15, were used for further testing (see also FIG. 25). To confirm that the increase in the MeSH metabolite was a good proxy for reduction in methionine and that the MGL enzyme would work in whole cells as well as purified extracts, E. coli expressing enzymes “2” and “8” were incubated in a simulated gut medium overnight, and the samples were analyzed via HPLC (see e.g., FIG. 8).

Bacteria expressing “2” and “8”, as well as control bacteria were incubated overnight in a simulated gut medium, and supernatant were analyzed by HPLC. Experimental bacteria showed a >90% reduction in methionine relative to control bacteria (see e.g., FIG. 8). Thus, the modified bacteria were capable of depleting methionine from their surrounding environment, and high levels of MeSH/DTNB 320 absorbance (see e.g., FIG. 7, FIG. 15, FIG. 25) indeed corresponded with low levels of methionine in the system. These results confirmed that bacteria expressing MGL enzymes reduced methionine in vitro. Further experiments tested expression of these enzymes in a probiotic bacterial host.

Table 5 includes an overview of Section (1).

TABLE 5
Overview of Section (1).

| Experiment | Evaluation Method | Results |
|---|---|---|
| In silico screening | BLAST search, VAXIJEN | Bacterial methionase genes identified by sequence |
| of bacterial | screening, pathogen cross- | analysis; those with high immunogenicity or |
| methionases | check | pathogenic hosts are eliminated. |
| Cloning and | Cloning in E. coli | PCR confirmation of plasmid transformation; |
| expression of | expression vectors | Coomassie confirmation of protein size; Immunoblot |
| methionases | | for protein size and antibody recognition |
| In vitro test, | Spectrophotometric assay | Significant (e.g., 3-fold above control) increase in 320 |
| isolated enzyme | | nm emission, indicating increased MGL activity in |
| activity | | high throughput screen |
| In vitro test, live | HPLC | Greater than 90% of methionine eliminated from |
| bacteria in | | simulated gut media during incubation with |
| synthetic gut media | | engineered E. coli during 24 hour incubation |

### (2) Generation of Human Microbiome Compatible Bacterial Strains Expressing Methionases

Having found high activity methionases and demonstrated that lab strains expressing these methionase enzymes could deplete methionine in vitro, these enzymes were engineered into bacteria suitable for oral consumption.

Developing an Engineered Bacterial Strain Using E. coli Nissle 1917 (EcN) as a Vector.

Due to its similarity to laboratory strains and 100+ year history of probiotic use in Europe and elsewhere, E. coli Nissle 1917 (EcN) was chosen as the chassis bacterium. It is contemplated herein that any probiotic bacterial strain can be used for the chassis bacterium, including but not limited to non-pathogenic strains of Escherichia coli; Bacillus subtilis; Pseudomonas putida; Treponema denticola; Citrobacter freundii; Bacillus cereus; Streptococcus thermophilus; Saccharomyces cerevisiae; Lactococcus lactis; Lactobacillus plantarum; and Brevibacterium linens, among others. E. coli Nissle 1917 (EcN) is thus used as a non-limiting example of a probiotic bacterium for expression of the MGL methionase.

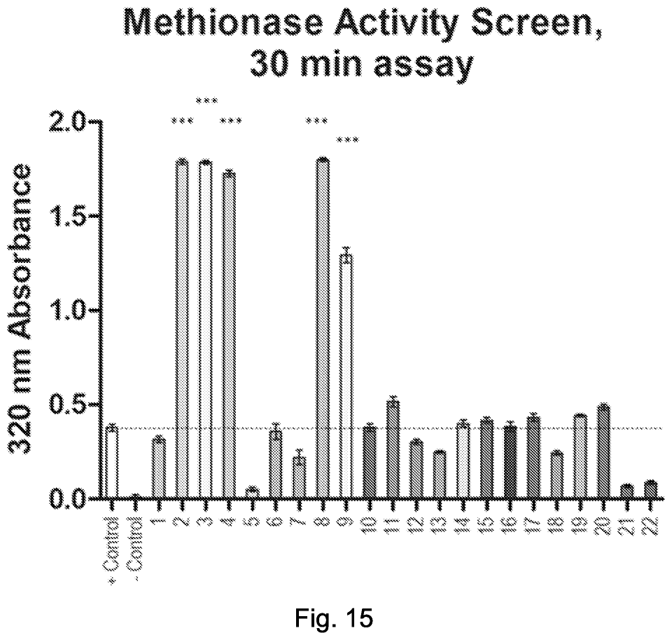

Although EcN is the same species as a laboratory strain, differences in gene regulation between the two prevented the use of the same expression plasmid as used above (e.g., FIG. 15), due to EcN's lack of T7 RNA polymerase (RNAP). Several plasmids were designed, suited specifically for EcN. EcN contains several “cryptic” plasmids, which it maintains without antibiotic selection, one of which is shown in FIG. 16A. This plasmid was used as a backbone for the construct, using gene regulatory elements known to operate in EcN (see e.g., FIG. 16B). The native “cryptic plasmid 1” was deleted from EcN using CRISPR Cas-9, yielding an EcN ready to accept and maintain a transgenic cryptic plasmid 1 (Data not shown). This strain was then made chemically competent, and transformed with the first round of EcN specific, methionase expressing plasmids.

### Characterization of Methionase Expressing Strains.

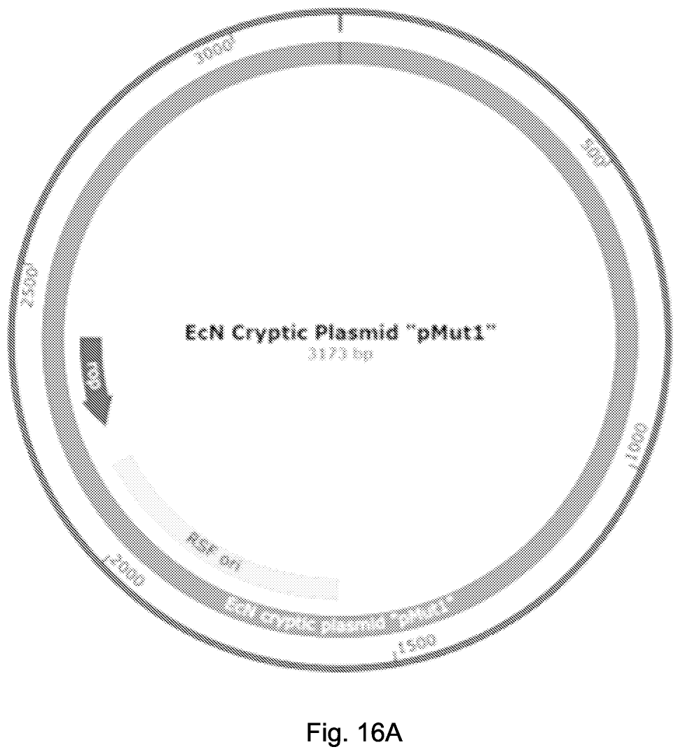

With strains of EcN expressing either MGL “2” or MGL “8”, these strains' ability to degrade methionine was assayed rapidly by colorimetry (see e.g., FIG. 17). While EcN “2” and EcN “8” both significantly yielded more of the methionine degradation marker than control EcN, the activity of intact cells was much lower than by the same quantity of cells lysed, e.g., free floating enzyme. Without wishing to be bound by theory, it was hypothesized that the cell wall would be the rate limiting step of methionine degradation in this system (and concomitantly that a search for marginally improved enzymes would be ineffective for further improving the system).

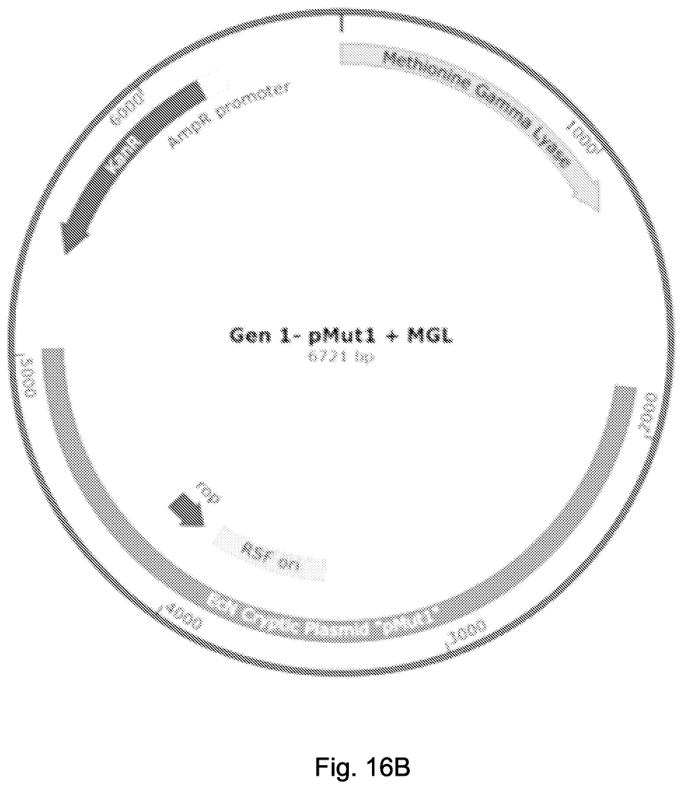

In order to counteract this effect, the first generation of modified cryptic plasmids was further modified to test various different strategies for increasing methionine import (see e.g., FIG. 16C). These strategies included overexpression of the wild type methionine importer operon MetNIQ (8-A), as well as point mutations of the individual genes MetN, MetI, and MetQ (8B-8D) in regions which contribute to feedback inhibition after methionine import; 8-C was engineered with activating mutations in MetN and MetQ. EcN strains expressing enzyme “8” alongside 4 of these combinations is shown in FIG. 18 (EcN: 8-A, 8-B, 8-C, 8-D). While the methionine degrading capacity of these constructs was slightly below that of free enzyme (Lysed EcN “8”), the most effective variant (EcN “8-C”) was a ˜66% improvement over intracellular enzyme alone (EcN “8”).

### Evaluation of Methionine Degradation Time Course.

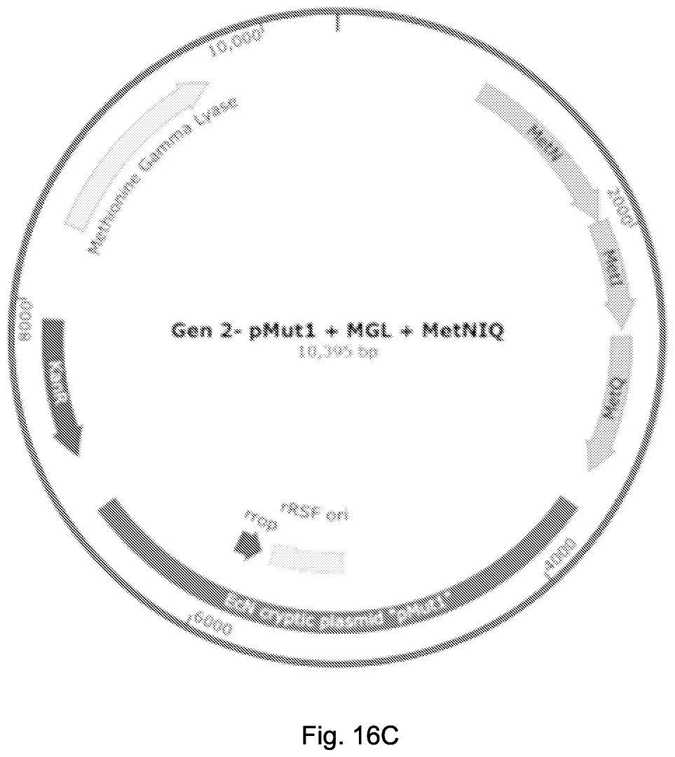

The most improved member of Gen2, “8C”, was used for further analysis. A time course assay was performed to estimate of the speed at which “8C” could degrade methionine from its surroundings (see e.g., FIG. 19). Gen 2 (EcN+MGL enzyme+importer) showed a rapid onset of methionine degradation in 1 hr., as opposed to the gradual ramp up in degradation in Gen1 (EcN+MGL enzyme).

### Evaluation of Methionine Degrading Strains in Simulated Gut Media.

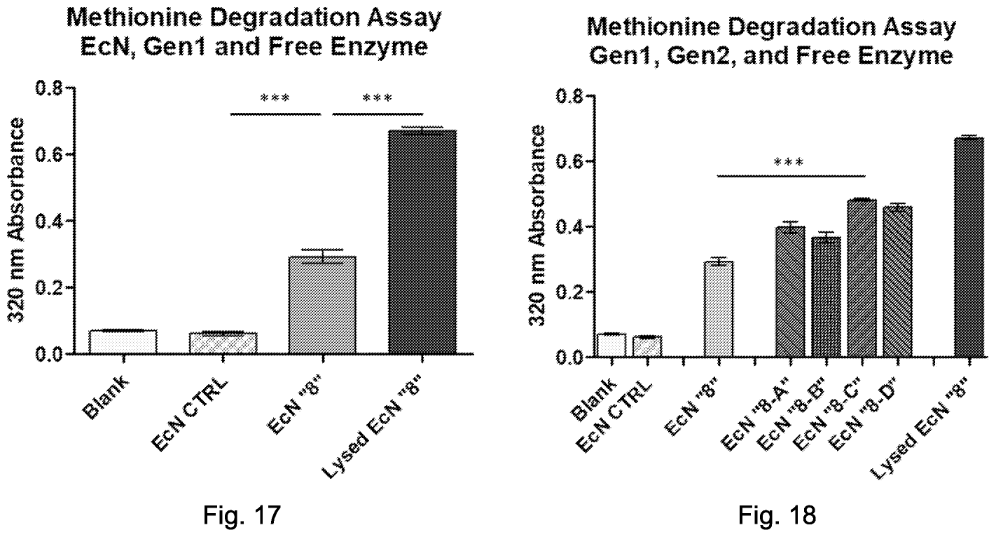

The ability of 8C degrade methionine was tested in simulated gut media, at a volume roughly the size of a mouse's gut (1.4 mL), at a roughly estimated dose used in other mouse studies of EcN (5×10\hat{ }10 colony-forming units (CFU)). Methionine level was analyzed via HPLC, after overnight incubation (see e.g., FIG. 20, “8C, Fresh”). Fresh 8C showed a significant, >90% reduction in methionine from gut media.

In preparation for in vivo studies, a batch of “8C” was grown in a research-grade 10-L bioreactor. The potency of 8C was tested after growth in the 10-L vessel, high speed centrifugation, formulation in a food safe glycerol buffer, more rigorous enumeration, a −80° C. freeze and thaw cycle (see e.g., FIG. 20; “8C, Freeze/Thaw”). This data show that the high density 10-L bioreactor samples were at least equally potent as the 1.4 mL shake flask scale, fresh bacteria. These formulated doses were then tested in an animal model.

Table 6 summarizes Section (2).

TABLE 6
Overview of Section (2).

| Experiment | Evaluation Method | Results |
|---|---|---|
| Cloning of methionases | Cloning in Nissle 1917 E. | PCR confirmation of plasmid |
| into Nissle1917 strain | coli expression vectors | transformation; Coomassie confirmation of |
| | | protein size; Immunoblot for protein size |
| | | and antibody recognition |
| In vitro test, live bacteria | HPLC | 90+% Reduction in methionine level in |
| in synthetic gut media (10 | | simulated gut medium by engineered |
| mL) | | bacteria, sourced from 10 ml shake flask |
| | | scale |
| In vitro test, live bacteria | HPLC | 90+% Reduction in methionine level in |
| in synthetic gut media (10 | | simulated gut medium by engineered |
| L) | | bacteria, sourced from 10 L bioreactor |
| | | scale |

### (3) Testing Bacterial Methionase Vector Strains in a Murine Model of HCU

In a murine model of HCU, engineered bacterial strains were tested for their ability to mitigate homocysteine imbalances, the metabolic hallmark of HCU.

### In Vivo Evaluation of Methionase Expressing Strains in a Murine Model of HCU.

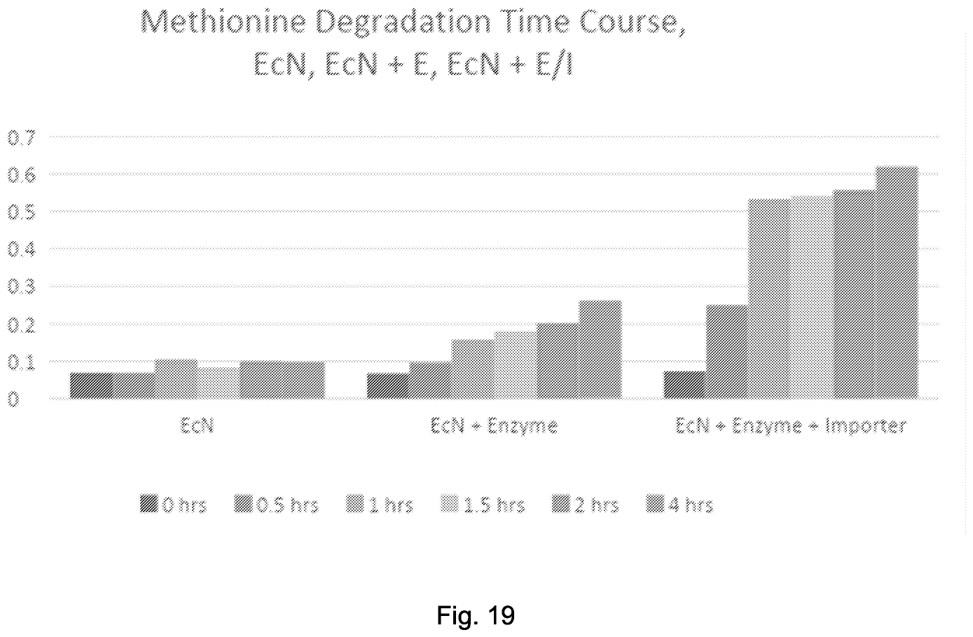

Preformulated bacterial doses were tested in a mouse model of classical homocystinuria, via knockout of the Cystathionine Beta Synthase gene (CBS −/−). To test the efficacy of the probiotic formulation in reducing homocysteine, the in vivo study detailed in FIG. 21 was carried out.

In vivo work was carried out in CBS −/− mice. Mice were allowed to eat a normal diet ad libitum. The first blood draw to check plasma cysteine and homocysteine was taken “Day 0.” Over the next three days, each mouse was gavaged with two doses of PTRI-8C, 5×10\hat{ }10 CFU each, once at 11 AM, and once at 6 PM. On the fourth day, a single dose was given at 11 AM, and at 6 PM a post treatment blood draw was taken. Blood plasma was then analyzed via HPLC.

Despite eating a normal diet ad libitum, an average 35% drop was observed in circulating plasma homocysteine (Hcy) in CBS−/− mice during the 3.5-day course of treatment. The data underlying FIG. 22 are presented in Table 7. All mice studied benefitted from the intervention across the board, with effect sizes ranging from 32% to 43%. Concomitantly, no adverse effects were observed during the trial period. These data surpass the clinical guideline for classical homocystinuria, which states that a 2000 decrease is clinically relevant as an intervention in humans. Given the average homocysteine level of an untreated human HCU patient is 125 uM, and the recommended target level below which there is no increase in thrombotic events is 100 uM, an intervention with this effect size would be sufficient for human patients.

TABLE 7
Homocysteine levels in CBS −/− mice after
dosing with the 8-C engineered microbe (ECN +
E/I: T. denticola MGL and activating mutations
in MetN and Met Q) shown in FIG. 19 and 22.

| | Pre-Treatment | | Post-Treatment | | |
|---|---|---|---|---|---|
| Mouse | Hcy | Cys | Hcy | Cys | % Change Hcy |
| H1373 | 222.2 | 97.9 | 131.6 | 122 | −41% |
| H1375 | 199.5 | 94 | 123.2 | 119.8 | −38% |
| H1383 | 218.3 | 94.4 | 149.7 | 128.1 | −43% |
| H1385 | 230 | 104.1 | 157.5 | 114.9 | −32% |
| H1393 | 178.4 | 106.3 | 122.1 | 132.8 | −32% |
| Mean | 209.7 | 99.4 | 136.8 | 123.5 | −35% |
| Std | 13 | 4.7 | 15.8 | 5.5 | |
| T-test | | | 0.000196 | 0.002907 | |

Table 8 includes an overview of Section (3).

TABLE 8
Overview of Section (3).

| Experiment | Evaluation Method | Results |
|---|---|---|
| Detect plasma levels | Administration of | At least 40% decrease |
| of total homocysteine | Nissle1917 methionase | in homocysteine levels. |
| in HO HCU mice | bacteria to HO HCU | 32-43% reduction was |
| compared to control | mice | observed |
| Toxicity | Empirical observation | No major toxicity |
| | of mice | events observed. |

Sections (1)-(3), described above, were successfully completed by identifying bacterial methionases, testing their ability to be expressed by microbial vectors in engineered bacteria, and revealing their in vivo efficacy at reducing system homocysteine levels.

### (4) Additional In Vivo Testing

Described herein is an in vivo evaluation of the engineered bacterium for efficacy at reducing systemic Hey, as well as measuring a range of other metabolic pathways, proxying thrombotic risk, and evaluating the cognitive function of treated and untreated mice.

Experimental design and power analysis: The HCU model comprises heterozygous mice for a null mutation in the murine CBS gene (cbs^{+/-}) crossed with mice transgenically expressing a low level of the human cbs gene (hCBS^{+/-}). Crossing these mice results in a particular genotype which recapitulates CBS haploinsufficiency (hCBS^{+/-}; cbs^{-/-}) leading to the autosomal recessive disease in humans. Because the hCBS^{+/-}; cbs^{-/-} mice express only the human gene, they are referred to here as “human only” or “HO”. These mice exhibit the metabolic hallmarks of HCU, including severe elevations in both plasma and tissue levels of Hey, methionine, S-adenosylmethionine, and S-adenosylhomocysteine and a concomitant decrease in plasma and hepatic levels of cysteine. This model has been extensively characterized at the biochemical and phenotypic level in both the presence and absence of Hey lowering treatment with betaine and is thus suited to the present studies.

Murine model of HCU using short-term methionase-expressing bacterial treatment: This experiment involves taking a non-lethal blood sample from 8 HO HCU mice before treatment for comparative purposes. Prior analysis of these mice ensures that all have a plasma total homocysteine level >250 μM in order to be included in the trial. All mice can then receive the bacterial treatment via gavage twice each day for 5 days. A non-lethal plasma sample can be taken on day three of the trial. Four hours after the last gavage, the mice are sacrificed by anesthetization and decapitation, and blood and a range of tissues can be taken and snap frozen in liquid nitrogen prior to analysis. Mice in all experimental groups are evenly divided between male and female and aged between 3 and 4 months. All mice are kept on a 12-hour light/-dark cycle at a mean temperature of 22° C. and maintained on standard chow (LabDietNIH5K67, PMI NUTRITION INTERNATIONAL, Brentwood, MO). Mice can be weighed before the commencement and daily during the trial. If weight loss >15% occurs or mice show any visible signs of distress or failure to ambulate, the trial can be terminated at that point.

Cognitive evaluation of HO mice after administration of engineered bacterium: FIG. 22 shows that the engineered bacterium significantly lowered plasma levels of Hey in the presence of a normal protein/methionine diet, a primary endpoint for HCU management. Additional investigation is required to confirm that this metabolic effect has an impact on downstream endpoints of HCU disease. Therefore, in addition to monitoring mice for reduced Hey levels, a series of other physiological parameters, intended to model the manifestations of HCU in humans, can also be evaluated. These parameters include coagulation defects, a range of molecular biomarkers related to inflammation, and changes in baseline cognition. Bone mineral density and ophthalmic defects can also be evaluated. Behavioral deficits in HCU are a mix of acute memory issues due to plasma homocysteine level, as well as developmental delays. Published literature indicates that cognitive issues in HCU patients can be due to memory impairment without underlying neural defects. Given the massive impact of improved cognitive capabilities for HCU patients, these tests can be performed in addition to physiological parameters. Specific methods are presented below for each assay.

Hcy monitoring: Blood plasma can be analyzed for Hey via HPLC. See e.g., Section (3) for representative data of this method (see e.g., FIG. 22).

Coagulation Parameters: The coagulative phenotype of cbs (−/−) mice has previously been assessed by determination of tail bleeding times as a surrogate of hemostasis and thrombosis function. HCU is associated with thrombotic conditions such as stroke. Alterations in the extrinsic coagulation pathway can be investigated using the prothrombin time (PT) assay. Quantitative and qualitative abnormalities in the intrinsic and common pathways of coagulation can be investigated by determining the activated partial thromboplastin time (aPTT). For these analyses, mice can be anesthetized with pentobarbital (50 mg/kg intraperitoneally), and venous blood can be collected via direct right atrial puncture. Plasma samples (20 μl) can then be diluted with 80 μl of water to a final volume of 100 μl and assayed in an electromechanical ST4 coagulation analyzer (DIAGNOSTICA STAGO, Parsippany, NJ) according to the manufacturer's standard protocol.

Molecular biomarker analysis: HCU decreases ApoA-1, ApoA-IV and PON-1 expression in liver and plasma, induces constitutive expression of pro-inflammatory cytokines, and a number of oxidative stress markers in both HO HCU mice and human HCU patients that were either untreated or poorly compliant. These biomarkers can be assessed in mice after the 5-day dosing period via RT-qPCR to detect changes in transcripts and/or multiplex ELISA assays to detect protein.

Cognitive Evaluation in Radial Arm Maze: This test is designed to measure spatial learning and memory in rodents. A testing apparatus consists of eight equidistantly spaced arms, each about 4 feet long, all radiating from a small circular central platform. At the end of each arm there is a food site, the contents of which are not visible from the central platform. Two types of memory that are assessed during the performance in this task are reference memory and working memory. Reference memory is assessed when the mice only visit the arms of the maze which contains the reward. The failure to do so can result in reference memory error. Working memory is assessed when the mice (or rats) enter each arm a single time. Re-entry into the arms can result in a working memory error. Repeated expose of mice to the maze and time taken to find all baited arms are assessed over a 10-day period as an index of memory and learning. Previous experiments have shown that HO HCU mice have profound deficits in learning and memory in this testing and that this cognitive deficit can be significantly improved by aggressive therapeutic lowering of Hey.

Cognitive Evaluation in Conditioned Taste Avoidance (CTA): CTA is a classical conditioning task where mice learn to associate a sense of nausea induced by an injection of lithium chloride (unconditioned stimulus; US) with a novel experience to the taste of chocolate milk (conditioned stimulus; CS) in a single paired trial. Animals that learn the association avoid the CS on a second exposure. The CTA protocol was followed as described in Rachubinski et al., Experimental Gerontology. 2012; 47(9):723-33, the contents of which are incorporated herein by reference in their entirety. Previous experiments have shown that HO HCU mice have profound deficits in learning and memory in this testing paradigm and that this cognitive deficit can be significantly improved by aggressive therapeutic lowering of Hey.

Statistical analysis: All data can be presented as means±standard deviation (SD) and compared using the unpaired Student's t test or ANOVA. Differences between groups can be considered significant at a P value of <0.05. Detailed bioinformatic analysis of metabolomic data can be carried out using METABOANALYST (metaboanalyst.ca). Previous experience and power analysis has shown that the samples sizes described have more than adequate statistical power for the analyses proposed.

Cognitive evaluation can be challenging due to the convolution of acute memory symptoms with systemic developmental delays, and not easily mitigated by acute therapies. Given the prominence of these issues in HCU patients, the engineered probiotic microorganism can be tested to confirm its that it improves cognition for patients. Additional studies are available that also reveal the efficacy and benefits of the engineered bacterium. These tests include bone mineral density scanning by dual-energy x-ray absorption (DEXA or DXA) as well as adiposity, both of which can be reduced in HCU patients. In addition, ophthalmic assays are available to monitor lens physiology, a common concern in HCU patients; this technique involves optical coherence tomography.

Table 9 summarizes Section (4). Briefly, HO mice can be prepared using genetic crosses undertaken to generate enough mice for study (e.g., 8 animals per testing group). The engineered bacterium is administered to the HO mice under a dosing schedule (e.g., twice per day for 2 days, single dose on day 4).

TABLE 9
Overview of Section (4)

| Experiment | Results |
|---|---|
| Hcy plasma levels | Decrease in Hcy blood plasma levels in HO HCU mice administered |
| | the engineered bacterium compared to negative control (see e.g., FIG. |
| | 22) |
| Coagulation parameters | Decrease in coagulation (e.g., increased tail vein bleed time, increased |
| | prothrombin time assay) in HO HCU mice administered the engineered |
| | bacterium compared to negative control |
| Biomarker evaluation of | Upregulation of ApoA-1, ApoA-IV, and PON-1 and remediation to |
| treated mice | normal physiological levels and decrease in proinflammatory cytokines |
| | and oxidative stress markers in HO HCU mice administered the |
| | engineered bacterium compared to negative control |
| Cognitive evaluation of | Improved cognition (e.g., increased scores in radial arm maze and in |
| treated mice | conditioned taste avoidance observed) for HO HCU mice administered |
| | the engineered bacterium compared to negative control |

### Example 3: Cancer Treatment

The engineered probiotic microorganism for reducing bioavailable methionine levels can be used for treating cancer. The engineered probiotic microorganism can express at least one exogenous copy of at least one gene encoding an enzyme that catalyzes the degradation of methionine (e.g., a methionine gamma lyase) and optionally at least one of the following: (i) at least one exogenous copy of at least one functional methionine importer gene; and/or (i) at least one endogenous methionine importer gene comprising at least one engineered activating modification. In order words, the engineered probiotic microorganism comprises a methionase and optionally a methionine importer (see e.g., Examples 1 and 2, FIG. 19).

The effect of the engineered probiotic microorganism in the treatment of cancer can be verified using the methods described, as a non-limiting example, in Golbourn et al. “Loss of MAT2A compromises methionine metabolism and represents a vulnerability in H3K27M mutant glioma by modulating the epigenome,” Nat Cancer. 2022 May, 3(5): 629-648, the contents of which are incorporated herein by reference in their entirety.

Diffuse midline gliomas (DMGs) bearing driver mutations of histone 3 lysine 27 (H3K27M) are incurable brain tumors with unique epigenomes. Golbourn generated a syngeneic H3K27M mouse model to study the amino acid metabolic dependencies of these tumors. H3K27M mutant cells were highly dependent on methionine. Methionine-restricted diets extended survival in multiple models of DMG in vivo.

The H3K27MPP cell line for DMG comprises the three most common alterations observed in human DMG tumors, which cooperate to initiate diffuse intrinsic pontine glioma (DIPG) in human neural stem cells and animal models: mutant histone H3 (H3K27M), overexpression of wild-type platelet-derived growth factor receptor (PDGFRA) and expression of a common hotspot mutation of tumor protein p53 (TP53) (TP53 R237H).

Briefly, 4-week-old non-obese diabetic severe combined immunodeficient male or female mice (Mus musculus NOD-scid IL2Rγ^{null}) are injected with 1×10⁵ H3K27MPP cells (4-6-week-old, in both male and female mice). Cells are resuspended in 2 μl of phosphate buffered saline (PBS) and injected into the pons/midbrain using a stereotactic frame (STOELTING) and automated cell injector (STOELTING) with cells delivered over 4 min. Coordinates can be as follows from the Lambda suture (x=0.8 mm, y=−0.8 mm, z=−5.0 mm).

Mice are injected with tumor and randomized to engineered probiotic microorganism or negative control (e.g., vehicle without the microorganism). The bacteria can be administered as shown in the upper dose schedule of FIG. 21. For example, for the three days following the glioma injection, each mouse can be gavaged with two doses the engineered probiotic microorganism, e.g., 5×10\hat{ }10 CFU each; e.g., once at 11 AM, and once at 6 PM. On the fourth day, a single dose can be given, e.g., at 11 AM. Alternatively, the bacteria can be administered at three days preceding the glioma injection, or three days overlapping the glioma injection, or any other effective administration schedule and dosage, as determined by a skilled person.

A post hoc power analysis can be performed to determine power >0.95. For example, each group can have at least 7 mice (e.g., at least 4 males and 3 females). The animal technician is blinded to experimental condition.

Mice are monitored daily for signs of ill health or overt tumors; once mice display signs of hydrocephalus (domed head) or neurological duress, they are humanely killed. UKCCCR guidelines 1997 recommend limiting solid tumors to 10% of the host's body weight. Brains can be extracted and fixed in 4% PFA. Mice are kept at 73-74° F. with 30% humidity and a dark-light cycle of 14-10 h.

Without wishing to be bound by theory, it is hypothesized herein that the engineered probiotic microorganism can mimic the lifespan prolonging effect of a reduced methionine diet in certain types of gliomas. Specifically, it is hypothesized the engineered probiotic microorganism can significantly prolong the survival of mice in the H3K27MPP DMG model compared to negative control, similar to the results of the low-methionine diet in the Kaplan-Meier survival curve of FIG. 8A of Golbourn.

### Example 4: Secreted Methionine Degrading Enzymes

Strains of Lactococcus lactis and Lactiplantibacillus plantarum can be generated, which secrete methionine-degrading enzymes (e.g., MGLs) into the gut environment, bypassing the need for methionine importers. Gram-positive protein secretion tags (see e.g., Tables 10-11) fused to an MGL enzyme (e.g., SEQ ID NO: 5 or 6) are tested for efficacy. Tables 10-11 include a representative list of the amino acid (AA) and nucleic acid (NT) sequences of gram-positive protein secretion C-terminal fusion tags. The testing of the engineered Lactococcus lactis and/or Lactiplantibacillus plantarum is similar to that described in Examples 1-3.

TABLE 10
Non-Limiting Examples of Gram-positive protein secretion C-terminal tags

| | | SEQ ID |
|---|---|---|
| Enzyme | Amino acid sequence | NO |
| AbnA | MKKKKTWKRFLHFSSAALAAGLIFTSAAPAEA | 90 |
| AmyE | MFAKRFKTSLLPLFAGFLLLFHLVLAGPAAASA | 91 |
| AprE | MRSKKLWISLLFALTLIFTMAFSNMSVQA | 92 |
| AspB | MKLAKRVSALTPSTTLAITAKA | 93 |
| BglC | MKRSISIFITCLLITLLTMGGMIASPASA | 94 |
| BglS | MPYLKRVLLLLVTGLFMSLFAVTATASA | 95 |
| Bpr | MRKKTKNRLISSVLSTVVISSLLFPGAAGA | 96 |
| CccA | MKWNPLIPFLLIAVLGIGLTFFLSVKG | 97 |
| CitH | MGNTRKKVSVIGAGFTGATTAFLIAQKELADV | 98 |
| CotC | MKNRLFILICFCVICLFLSFGQPFFPSMILTVQAAKS | 99 |
| Csn | MKISMQKADFWKKAAISLLVFTMFFTLMMSETVFA | 100 |
| CwlD | MRKKLKWLSFLLGFIILLFLFKYQFSN | 101 |
| DacB | MRIFKKAVFVIMISFLIATVNVNTAHA | 102 |
| DacF | MKRLLSTLLIGIMLLTFAPSAFA | 103 |
| DltD | MKKRFFGPIILAFILFAGAIA | 104 |
| Epr | MKNMSCKLVVSVTLFFSFLTIGPLAHA | 105 |
| FliL | MKKKLMIILLIILIVIGALGAAA | 106 |
| FliZ | MKKSQYFIVFICFFVLFSVHPIAAAAA | 107 |
| GlpQ | MRKNRILALFVLSLGLLSFMVTPVSA | 108 |
| LipA | MKFVKRRIIALVTILMLSVTSLFALQPSAKA | 109 |
| LipB | MKKVLMAFIICLSLILSVLAAPPSGAKA | 110 |
| LytB | MKSCKQLIVCSLAAILLLIPSVSFA | 111 |
| LytC | MRSYIKVLTMCFLGLILFVPTALA | 112 |
| LytD | MKKRLIAPMLLSAASLAFFAMSGSAQA | 113 |
| LytE | MKKQIITATTAVVLGALFA | 114 |
| LytF | MKKKLAAGLTASAIVGTTLVVTPAEA | 115 |
| LytR | MRNERRKKKKTLLLTILTIIGLLVLGTGGYAYYLWHKAA | 116 |
| Mdr | MDTTTAKQASTKFVVLGLLLGILMSAMDNTIVATA | 117 |
| MotB | MARKKKKKHEDEHVDESWLVPYADILTLLLALFIVLYASS | 118 |
| Mpr | MKLVPRFRKQWFAYLTVLCLALAAAVSFGVPAKA | 119 |
| MreC | MPNKRLMLLLLCIIILVAMIGFS | 120 |
| NprB | MRNLTKTSLLLAGLCTAAQMVFVTHASA | 121 |
| NprE | MGLGKKLSVAVAASFMSLSISLPGVQA | 122 |
| NucB | MKKWMAGLFLAAAVLLCLMVPQQIQGASS | 123 |
| Pbp | MKKSIKLYVAVLLLFVVASVPYMHQAALA | 124 |
| PbpB | MIQMPKKNKFMNRGAAILSICFALFFFVILGRMA | 125 |
| PbpD | MTMLRKIIGWILLLCIIPLFAFTVIA | 126 |
| PbpX | MTSPTRRRTAKRRRRKLNKRGKLLFGLLAVMVCITIWNA | 127 |
| Pel | MKKVMLATALFLGLTPAGANA | 128 |
| PelB | MKRLCLWFTVFSLFLVLLPGKALG | 129 |
| PenP | MKLKTKASIKFGICVGLLCLSITGFTPFFNSTHAEA | 130 |
| PhoA | KKMSLFQNMKSKLLPIAAVSVLTAGIFAGA | 131 |
| PhoB | MKKFPKKLLPIAVLSSIAFSSLASGSVPEASA | 132 |
| PhrA | MKSKWMSGLLLVAVGFSFTQVMVHA | 133 |
| PhrC | MLKSKLFVICLAAAAIFTAAGVSANA | 134 |
| PhrF | MKLKSKLLLSCLALSTVFVATTIA | 135 |
| PhrG | MKRFLIGAGVAAVILSGWFIA | 136 |
| PhrK | MKKLVLCVSILAVILSGVA | 137 |
| RpmG | MRKKITLACKTCGNRNYTTMKSSASA | 138 |
| SacB | MNIKKFAKQATVLTFTTALLAGGATQAFA | 139 |
| SacC | MKKRLIQVMIMFTLLLTMAFSADA | 140 |
| SleB | MKSKGSIMACLILFSFTITTFINTETISAFS | 141 |
| SpoIID | MKQFAITLSVLCALILLVPTLLVIPFQHNKEAGA | 142 |
| SpoIIP | MRNKRRNRQIVVAVNGGKAVKAIFLFIVSLIVIFVLSGV | 143 |
| SpoIIQ | MREEEKKTSQVKKLQQFFRKRWVFPAIYLVSAAVILTAVL | 144 |
| SpoIIR | MKKTVIICIYIFLLLSGALV | 145 |
| TasA | MGMKKKLSLGVASAALGLALVGGGTWA | 146 |
| TyrA | MNQMKDTILLAGLGLIGGSIALA | 147 |
| Vpr | MKKGIIRFLLVSFVLFFALSTGITGVQAAPA | 148 |
| WapA | MKKRKRRNFKRFIAAFLVLALMISLVPADVLA | 149 |
| WprA | MKRRKFSSVVAAVLIFALIFSLFSPGTKAAA | 150 |
| XynA | MFKFKKNFLVGLSAALMSISLFSATASA | 151 |
| YbbC | MRKTIFAFLTGLMMFGTITAASA | 152 |
| YbbE | MKTKTLFIFSAILTLSIFAPNETFA | 153 |
| YbbR | MDKFLNNRWAVKIIALLFALLLYVAVNS | 154 |
| YbdG | MKTLWKVLKIVFVSLAALVLLVSVS | 155 |
| YbdN | MVKKWLIQFAVMLSVLSTFTYSASA | 156 |
| YbfO | MKRMIVRMTLPLLIVCLAFSSFSASARA | 157 |
| YbxI | MKKWIYVVLVLSIAGIGGFSVHA | 158 |
| YckD | MKRITINIITMFIAAAVISLTGTAEA | 159 |
| YdbK | MKLFNRKVTLVSLILMAVFQFFMALIIKRIVIS | 160 |
| YddT | MRKKRVITCVMAASLTLGSLLPAGYASA | 161 |
| YdhT | MFKKHTISLLIIFLLASAVLA | 162 |
| YdjM | MLKKVILAAFILVGSTLGAFSFSSDASA | 163 |
| YdiN | MKKRIILLLAVIIAAAAAGVA | 164 |
| YfhK | MKKKQVMLALTAAAGLGLTALHSAPAAKA | 165 |
| YfiS | MKWMCSICCAAVLLAGGAAQA | 166 |
| YfkD | MMKKLFHSTLIVLLFFSFFGVQPIHA | 167 |
| YfkN | MRIQKRRTHVENILRILLPPIMILSLILPTPPIHA | 168 |
| YhaK | MRTWKRIPKTTMLISLVSPFLLITPVLFYAALA | 169 |
| YhcR | MLSVEMISRQNRCHYVYKGGNMMRRILHIVLITALMFLNVMYTFEA | 170 |
| YhdC | MKSLPYTIALLFCGLIIVSMA | 171 |
| YhfM | MKKIVAAIVVIGLVFIAFFYLYSRSGDVYQSVDA | 172 |
| YhjA | MKKAAAVLLSLGLVFGFSYGAGHVAEA | 173 |
| YjcM | MKKELLASLVLCLSLSPLVSTNEVFA | 174 |
| YjcN | MKKKTKIILSLLAALIVILIVLPVLSPVVFTASS | 175 |
| YjdB | MNFKKTVVSALSISALALSVSGVASA | 176 |
| YjfA | MKRLFMKASLVLFAVVFVFAVKGAPAKA | 177 |
| YjiA | MAAQTDYKKQVVGILLSLAFVLFVFS | 178 |
| YknX | MKKVWIGIGIAVIVALFVGINIYRSAAPTSGSA | 179 |
| YkoJ | MLKKKWMVGLLAGCLAAGGFSYNAFA | 180 |
| YkvT | MTTKFTALAVFLLCFMPAAKI | 181 |
| YkvV | MLTKRLLTIYIMLLGLIAWFPGAAQA | 182 |
| YkwD | MKKAFILSAAAAVGLFTFGGVQQASA | 183 |
| YlaE | MKKTFVKKAMLTTAAMTSAALLTFGPDAASA | 184 |
| YlbL | MLRKKHFSWMLVILILIAVLSFIKLPYYITKPGEA | 185 |
| YlqB | MKKIGLLFMLCLAALFTIGFPAQQADA | 186 |
| YlxF | MSGKKKESGKFRSVLLIIILPLMFLLIAGGIVLWAAG | 187 |
| YlxW | MRGKSAVLLSLIMLIAGFLISFSFQMTKENNKSAA | 188 |
| YlxY | MYKKFVPFAVFLFLFFVSFEMMENPHALDYIGA | 189 |
| YncM | MAKPLSKGGILVKKVLIAGAVGTAVLFGTLSSGIPGLPAADA | 190 |
| YndA | MRFTKVVGFLSVLGLAAVFPLTAQA | 191 |
| YnfF | MIPRIKKTICVLLVCFTMLSVMLGPGATEVLA | 192 |
| YngK | MKVCQKSIVRFLVSLIIGTFVISVPFMANA | 193 |
| YnzA | MELSFTKILVILFVGFLVFGPDKLPALG | 194 |
| YoaW | MKKMLMLAFTFLLALTIHVGEASA | 195 |
| YobB | MKIRKILLSSALSFGMLISAVPALA | 196 |
| YobV | MKLERLLAMVVLLISKKQVQA | 197 |
| YocA | MKKKRKGCFAAAGFMMIFVFVIA | 198 |
| YocH | MKKTIMSFVAVAALSTTAFGAHA | 199 |
| YodV | MKVPKTMLLSTAAGLLLSLTATSVSA | 200 |
| YojL | MKKKIVAGLAVSAVVGSSMAAAPAEA | 201 |
| YolA | MKKRITYSLLALLAVVAFAFTDSSKAKA | 202 |
| YolC | MKKRLIGFLVLVPALIMSGITLIEA | 203 |
| YolI | MKKWIVLFLVLIAAAISIFVYVSTGSE | 204 |
| YomL | MRKKRVITCVMAASLTLGSLLPAGYATA | 205 |
| YopL | MKKLIMALVILGALGTSYISA | 206 |
| YogH | MKRFILVLSFLSIIVAYPIQTNA | 207 |
| YogM | MKLRKVLTGSVLSLGLLVSASPAFA | 208 |
| YpbG | KLSVKIAGVLTVAAAAMTAKMYATA | 209 |
| YpcP | MNNNKLLLVDGMALLFRAFFATA | 210 |
| YpjP | MKLWMRKTLVVLFTIVTFGLVSPPAALMA | 211 |
| YpmB | MRKKALIFTVIFGIIFLAVLLVSASIYKSAMA | 212 |
| YpmS | NKWKRLFFILLAINFILAAGFVALVLLPGEQAQV | 213 |
| YpuA | MKKIWIGMLAAAVLLLMVPKVSLADA | 214 |
| YpuD | MGRIKTKITILLVLLLLLAGGYMYINDIELKDVPTAIG | 215 |
| YqfZ | MKRLTLVCSIVFILFILFYDLKIGTIPIQDLPVYEASA | 216 |
| YqgA | MKQGKFSVFLILLLMLTLVVAPKGKAEA | 217 |
| YqxI | MFKKLLLATSALTFSLSLVLPLDGHAKA | 218 |
| YqxM | MFRLFHNQQKAKTKLKVLLIFQLSVIFSLTAAICLQFSDDTSA | 219 |
| YqzC | MTKRGIQAFAGGIILATAVLAAVFYLTDEDQAAA | 220 |
| YqzG | MMIKQCVICLSLLVFGTTAAHA | 221 |
| YraJ | MTLTKLKMLSMLTVMIASLFIFSSQALA | 222 |
| YrrL | MYINQQKKSFFNKKRIILSSIVVLFLIIGGAFL | 223 |
| YrrR | MKISKRMKLAVIAFLIVFFLLLLRLAEI | 224 |
| YrrS | MSNNQSRYENRDKRRKANLVLNILIAIVSILIVVVAA | 225 |
| YrvJ | MNKKYFVLIVCIIFTSALFPTFSSVTA | 226 |
| YuaB | MKRKLLSSLAISALSLGLLVSAPTASFAAE | 227 |
| YunA | MITDIFKPGCRKLCVFNMKGDYFVKVLLSALLLLLFA | 228 |
| YunB | MPRYRGPFRKRGPLPFRYVMLLSVVFFILSTTVSL | 229 |
| YurI | MTKKAWFLPLVCVLLISGWLAPAASASA | 230 |
| YusW | MHLIRAAGAVCLAVVLIAGCRFNEDQHQAEG | 231 |
| YvbX | MKKWLIIAVSLAIAIVLFMYTKGEAKA | 232 |
| YvcE | MRKSLITLGLASVIGTSSFLIPFTSKTASA | 233 |
| YveB | MNYIKAGKWLTVFLTFLGILLFIDL | 234 |
| YvgO | MKRIRIPMTLALGAALTIAPLSFASA | 235 |
| YvgV | MKKKQQSSAKFAVILTVVVVVLLAAIV | 236 |
| YvnB | MRKYTVIASILLSFLSVLSGG | 237 |
| YvpA | MKKIVSILFMFGLVMGFSQFQPSTVFA | 238 |
| YvpB | MKTLRTLCVLMILSGVIFFGLKIDA | 239 |
| YwaD | MKKLLTVMTMAVLTAGTLLLPAQSVTPAAHA | 240 |
| YwcI | MKRLLVSLRVWMVFLMNWVTPDRKTARA | 241 |
| YwdK | MKVFIILGAINALLAVGLGAFG | 242 |
| YweA | MLKRTSFVSSLFISSAVLLSILLPSGQAHA | 243 |
| YwfM | MKGNIYSLFVLIAAFFWGTTGTVQA | 244 |
| YwgB | MKMKSGMEQAVSVLLLLSRLPVQA | 245 |
| YwjE | MKVFIVIMIIVVIFFALILLDIFMGRA | 246 |
| YwmB | MKKKQVSHAIIISVMLSFVIAVFHTIHA | 247 |
| YwmC | MKKRFSLIMMTGLLFGLTSPAFA | 248 |
| YwmD | MKKLLAAGIIGLLTVSIASPSFA | 249 |
| YwoF | MRKWYFILLAGVLTSVILAFVYDKTKA | 250 |
| YwqC | MGESTSLKEILSTLTKRILLIMIVTAAATA | 251 |
| YwqO | MKFLLSVIAGLLILALYLFWKVQPPVWI | 252 |
| YwsB | MNKPTKLFSTLALAAGMTAAAAGGAGTIHA | 253 |
| YwtC | MKFVKAIWPFVAVAIVFMFMSA | 254 |
| YwtD | MNTLANWKKFLLVAVIICFLVPIMTKAEIAEA | 255 |
| YwtF | MEERSQRRKKKRKLKKWVKVVAGLMAFLVIAAGSVGAYA | 256 |
| YxaK | MVKSFRMKALIAGAAVAAAVSAGAVSDVPAAKVLQPTAAYA | 257 |
| YxiA | MFNRLFRVCFLAALIMAFTLPNSVYA | 258 |
| YxiT | MKWNNMLKAAGIAVLLFSVFAYAAPSLKAVQA | 259 |
| YyaB | MVYQTKRDVPVTLMIVFLILLIQADA | 260 |
| YybN | MNKFLKSNFRFLLAAALGISLLASSNFIKA | 261 |
| YycP | MKKWMITIAMLILAGIALFVFISPLKS | 262 |

TABLE 11
Non-Limiting Examples of Gram-positive protein secretion C-terminal fusion tags

| | | SEQ |
|---|---|---|
| Enzyme | Nucleic acid sequence | ID NO |
| AbnA | ATGAAGAAGAAGAAAACCTGGAAGCGTTTCCTTCATTTTTCAAGCGCG | 263 |
| | GCTCTGGCTGCTGGTCTCATTTTCACTTCCGCGGCTCCTGCCGAAGCA | |
| AmyE | ATGTTTGCCAAGCGTTTTAAAACATCTCTGCTTCCGTTGTTTGCGGGAT | 264 |
| | TTCTCTTACTGTTCCATTTGGTGCTGGCCGGACCGGCCGCCGCAAGCG | |
| | CA | |
| AprE | ATGAGATCAAAAAAACTCTGGATTTCCCTGTTATTTGCCCTCACGTTA | 265 |
| | ATCTTCACAATGGCTTTCTCTAATATGAGTGTTCAAGCG | |
| AspB | ATGAAGTTGGCAAAGAGGGTGAGCGCGCTTACTCCAAGCACCACACT | 266 |
| | TGCAATCACAGCAAAAGCA | |
| BglC | ATGAAACGTAGCATCAGCATTTTTATTACATGTTTGCTGATCACATTGT | 267 |
| | TGACCATGGGAGGGATGATCGCCTCCCCGGCCTCTGCC | |
| BglS | ATGCCTTATCTGAAAAGGGTTCTTCTCCTGCTTGTAACGGGCCTGTTTA | 268 |
| | TGTCACTTTTCGCGGTGACAGCAACGGCGTCCGCG | |
| Bpr | ATGAGAAAGAAGACCAAAAACCGCCTTATTTCGTCAGTCCTGAGCAC | 269 |
| | AGTAGTTATCTCTTCCTTACTGTTTCCAGGTGCTGCGGGCGCA | |
| CccA | ATGAAATGGAATCCGTTGATTCCGTTTCTTCTCATAGCAGTACTGGGA | 270 |
| | ATAGGATTAACTTTTTTCTTGTCTGTCAAGGGA | |
| CitH | ATGGGAAACACGCGCAAAAAGGTTTCAGTCATCGGCGCAGGGTTCAC | 271 |
| | AGGAGCTACAACTGCCTTTCTGATTGCCCAAAAAGAGTTAGCAGACGT | |
| | A | |
| CotC | ATGAAGAACAGGTTATTTATCTTGATCTGCTTTTGTGTCATATGCCTCT | 272 |
| | TCTTATCGTTTGGCCAACCGTTTTTTCCTTCTATGATTCTTACGGTACA | |
| | AGCAGCAAAAAGC | |
| Csn | ATGAAGATTAGCATGCAGAAAGCCGACTTCTGGAAGAAAGCGGCTAT | 273 |
| | TTCATTATTAGTTTTTACGATGTTCTTTACTCTGATGATGTCTGAGACT | |
| | GTGTTTGCT | |
| CwID | ATGCGGAAAAAACTGAAATGGCTTAGTTTTCTGTTGGGCTTCATAATT | 274 |
| | CTGCTTTTTTTATTTAAGTATCAATTTTCAAAT | |
| DacB | ATGAGAATTTTTAAAAAAGCGGTTTTTGTCATTATGATCTCCTTTCTGA | 275 |
| | TTGCCACTGTTAACGTCAATACTGCACACGCC | |
| DacF | ATGAAGAGATTATTGTCAACCTTATTAATTGGCATTATGTTACTCACAT | 276 |
| | TTGCACCGAGCGCGTTTGCA | |
| DltD | ATGAAAAAAAGATTCTTCGGGCCAATCATTCTTGCCTTTATTCTGTTTG | 277 |
| | CGGGGGCGATCGCA | |
| Epr | ATGAAAAATATGTCGTGCAAACTTGTGGTATCCGTTACCTTATTTTTTA | 278 |
| | GCTTTCTCACTATCGGCCCTTTAGCGCACGCG | |
| FliL | ATGAAAAAAAAACTTATGATCATTCTTCTTATTATCTTAATCGTGATTG | 279 |
| | GGGCACTGGGAGCAGCTGCG | |
| FliZ | ATGAAAAAGAGCCAGTATTTTATTGTTTTCATCTGCTTTTTTGTACTTT | 280 |
| | TTTCAGTTCATCCTATCGCAGCGGCAGCTGCA | |
| GlpQ | ATGCGGAAAAACCGGATTCTGGCCCTGTTTGTGCTTAGCTTGGGCCTG | 281 |
| | CTGTCTTTCATGGTTACCCCGGTGTCAGCT | |
| LipA | ATGAAATTTGTGAAAAGACGGATTATAGCTCTGGTCACTATTCTTATG | 282 |
| | CTCTCGGTTACGTCGCTGTTCGCTCTTCAACCATCTGCAAAGGCA | |
| LipB | ATGAAGAAGGTGTTGATGGCCTTCATCATCTGCTTATCACTGATACTCT | 283 |
| | CCGTACTCGCCGCACCTCCAAGCGGAGCCAAAGCG | |
| LytB | ATGAAGTCATGCAAACAGTTGATTGTGTGCTCTCTGGCAGCTATTCTC | 284 |
| | CTGTTGATTCCTTCAGTTTCCTTCGCG | |
| LytC | ATGCGTAGCTACATTAAAGTACTCACTATGTGCTTCTTAGGCCTTATTC | 285 |
| | TGTTTGTGCCGACCGCACTGGCC | |
| LytD | ATGAAGAAAAGACTGATTGCACCTATGTTGTTAAGTGCCGCTTCATTG | 286 |
| | GCGTTCTTCGCCATGTCAGGATCAGCCCAGGCC | |
| LytE | ATGAAAAAACAAATCATTACGGCAACTACGGCAGTTGTGCTGGGCGC | 287 |
| | TCTTTTTGCC | |
| LytF | ATGAAAAAAAAACTCGCGGCCGGCCTGACCGCTTCTGCAATCGTCGG | 288 |
| | AACGACATTGGTTGTAACACCGGCCGAAGCT | |
| LytR | ATGCGTAACGAACGGCGAAAAAAAAAAAAAACTCTCCTTCTGACAAT | 289 |
| | TCTTACGATTATTGGACTTTTGGTTCTTGGGACGGGCGGCTACGCGTAC | |
| | TACCTGTGGCATAAAGCAGCG | |
| Mdr | ATGGATACCACCACCGCCAAACAAGCGTCCACAAAATTTGTCGTATTG | 290 |
| | GGACTTCTTCTCGGAATTTTGATGAGCGCGATGGATAACACAATTGTC | |
| | GCGACAGCA | |
| MotB | ATGGCTCGGAAGAAGAAAAAAAAACATGAAGATGAGCATGTCGATGA | 291 |
| | ATCTTGGTTGGTTCCATATGCGGACATCTTAACCCTGCTTCTGGCACTT | |
| | TTTATTGTCCTGTATGCGTCTAGC | |
| Mpr | ATGAAATTAGTTCCGAGGTTCCGCAAACAATGGTTTGCATATTTAACA | 292 |
| | GTGCTGTGCTTGGCACTCGCCGCAGCTGTCAGCTTCGGCGTTCCGGCC | |
| | AAAGCG | |
| MreC | ATGCCTAATAAACGGTTAATGCTGTTGCTTTTATGCATCATTATTTTAG | 293 |
| | TGGCGATGATTGGGTTCTCA | |
| NprB | ATGCGCAATTTGACGAAAACATCATTACTTCTGGCGGGATTGTGCACA | 294 |
| | GCCGCACAAATGGTGTTCGTCACCCACGCCAGTGCG | |
| NprE | ATGGGACTCGGTAAGAAACTCTCCGTAGCAGTTGCGGCAAGCTTTATG | 295 |
| | TCACTTTCAATCTCTTTACCGGGTGTTCAAGCG | |
| NucB | ATGAAAAAGTGGATGGCGGGCCTGTTTCTGGCAGCGGCAGTTCTGCTG | 296 |
| | TGTCTGATGGTCCCGCAACAGATTCAAGGCGCTTCCTCA | |
| Pbp | ATGAAGAAATCAATTAAACTCTATGTAGCTGTTCTGCTCCTTTTTGTGG | 297 |
| | TTGCCAGTGTGCCGTATATGCACCAAGCTGCCCTCGCA | |
| PbpB | ATGATACAGATGCCGAAGAAGAACAAATTTATGAACAGAGGTGCAGC | 298 |
| | CATCTTGAGTATTTGTTTTGCTCTCTTTTTTTTTGTGATTCTTGGACGTA | |
| | TGGCA | |
| PbpD | ATGACGATGCTCCGAAAGATAATTGGGTGGATCTTACTTCTGTGTATT | 299 |
| | ATTCCCCTTTTCGCTTTTACAGTCATCGCT | |
| PbpX | ATGACAAGCCCAACGCGAAGAAGAACGGCAAAAAGGAGGCGTAGAA | 300 |
| | AACTTAACAAAAGAGGCAAATTGTTATTTGGACTTTTAGCGGTTATGG | |
| | TCTGCATAACGATCTGGAACGCC | |
| Pel | ATGAAAAAAGTAATGTTAGCGACAGCACTTTTTCTGGGGCTTACCCCT | 301 |
| | GCAGGAGCGAATGCA | |
| PelB | ATGAAGAGGTTATGTCTGTGGTTCACCGTGTTCTCACTTTTTCTTGTAT | 302 |
| | TACTTCCCGGAAAGGCTCTTGGG | |
| PenP | ATGAAACTGAAAACGAAAGCGAGTATAAAATTTGGGATATGTGTGGG | 303 |
| | ACTTCTTTGTCTGTCCATTACCGGTTTTACACCATTTTTCAATTCTACAC | |
| | ACGCGGAGGCA | |
| PhoA | AAAAAAATGTCTCTGTTCCAAAACATGAAGAGCAAGCTGCTGCCGATC | 304 |
| | GCGGCGGTATCAGTATTAACTGCGGGCATCTTTGCAGGCGCG | |
| PhoB | ATGAAAAAATTTCCAAAGAAACTTTTGCCAATCGCGGTGCTGAGCAGT | 305 |
| | ATTGCATTCTCTTCCTTAGCCTCAGGTTCTGTTCCTGAAGCAAGCGCA | |
| PhrA | ATGAAATCTAAATGGATGAGTGGTCTTTTACTTGTAGCAGTTGGTTTTA | 306 |
| | GTTTTACACAAGTAATGGTTCATGCT | |
| PhrC | ATGCTGAAATCGAAGTTATTTGTTATTTGCCTGGCAGCCGCCGCAATTT | 307 |
| | TTACAGCGGCGGGTGTCTCAGCAAATGCC | |
| PhrF | ATGAAACTGAAATCTAAACTGCTGCTGAGTIGTCTGGCCTTGTCCACA | 308 |
| | GTTTTTGTAGCTACGACCATCGCT | |
| PhrG | ATGAAAAGGTTCCTTATTGGAGCTGGTGTGGCGGCTGTGATACTGTCA | 309 |
| | GGTTGGTTTATCGCA | |
| PhrK | ATGAAAAAACTGGTCTTATGTGTAAGCATTCTCGCAGTCATTCTCTCTG | 310 |
| | GAGTTGCC | |
| RpmG | ATGCGTAAAAAAATTACATTAGCATGTAAAACATGTGGAAACCGCAA | 311 |
| | TTACACAACAATGAAAAGCTCCGCTTCGGCC | |
| SacB | ATGAATATCAAAAAATTCGCTAAGCAAGCGACTGTGTTAACATTTACC | 312 |
| | ACGGCACTCCTGGCAGGAGGTGCTACACAAGCTTTTGCA | |
| SacC | ATGAAAAAGAGATTGATTCAAGTAATGATCATGTTTACGTTGCTTCTG | 313 |
| | ACTATGGCCTTCAGTGCCGATGCT | |
| SleB | ATGAAATCTAAAGGAAGTATCATGGCATGCTTGATTTTGTTTAGCTTT | 314 |
| | ACGATAACGACCTTTATTAACACAGAGACTATTAGCGCCTTCAGT | |
| SpoIID | ATGAAGCAATTTGCTATCACCTTAAGTGTTCTGTGTGCGTTAATATTAC | 315 |
| | TTGTTCCTACGCTCCTCGTGATCCCGTTTCAACATAATAAAGAAGCAG | |
| | GAGCA | |
| SpoIIP | ATGCGTAATAAACGTAGAAACCGGCAGATTGTCGTAGCTGTCAACGG | 316 |
| | AGGTAAAGCAGTAAAAGCTATCTTTCTGTTTATCGTAAGTTTAATTGTT | |
| | ATTTTTGTTTTGTCCGGGGTG | |
| SpoIIQ | ATGAGGGAAGAAGAAAAAAAGACGAGTCAGGTGAAAAAACTCCAAC | 317 |
| | AGTTTTTTAGAAAACGTTGGGTGTTTCCAGCGATCTATCTTGTCTCTGC | |
| | CGCTGTTATCCTGACAGCAGTGTTG | |
| SpoIIR | ATGAAGAAAACAGTTATTATCTGCATTTACATTTTTTTGTTGTTATCAG | 318 |
| | GGGCGCTGGTT | |
| TasA | ATGGGAATGAAAAAAAAACTTTCTCTCGGCGTTGCGTCAGCCGCGTTG | 319 |
| | GGGTTGGCACTTGTAGGAGGTGGAACATGGGCT | |
| TyrA | ATGAATCAGATGAAAGACACGATCCTGCTCGCGGGTTTAGGCTTAATT | 320 |
| | GGCGGCAGCATCGCGTTAGCT | |
| Vpr | ATGAAAAAAGGCATTATCCGGTTCTTGCTCGTTAGCTTCGTATTGTTTT | 321 |
| | TTGCCCTGAGTACTGGTATTACAGGAGTACAAGCGGCCCCCGCT | |
| WapA | ATGAAAAAACGGAAGAGACGGAATTTTAAACGTTTTATTGCGGCTTTT | 322 |
| | CTTGTTCTTGCCTTAATGATTTCTCTTGTGCCTGCAGATGTGTTAGCC | |
| WprA | ATGAAAAGACGTAAATTCTCCAGCGTTGTGGCTGCGGTTTTAATATTT | 323 |
| | GCTTTAATTTTCAGCTTGTTTTCGCCAGGAACTAAAGCGGCCGCG | |
| XynA | ATGTTTAAGTTTAAAAAAAATTTCTTGGTAGGCTTGTCAGCCGCACTG | 324 |
| | ATGTCAATCTCGCTGTTTTCTGCCACCGCGTCCGCC | |
| YbbC | ATGCGCAAGACTATCTTTGCATTCTTAACCGGTTTAATGATGTTTGGCA | 325 |
| | CGATTACCGCTGCATCGGCG | |
| YbbE | ATGAAAACTAAGACCCTGTTTATATTTAGCGCTATTCTGACATTATCTA | 326 |
| | TTTTCGCGCCCAATGAAACATTCGCT | |
| YbbR | ATGGATAAGTTCTTAAATAACCGCTGGGCAGTGAAAATCATCGCATTG | 327 |
| | CTTTTTGCGCTGCTTCTGTATGTAGCCGTTAATAGC | |
| YbdG | ATGAAGACGCTTTGGAAGGTGCTTAAAATCGTTTTCGTGAGTCTTGCG | 328 |
| | GCCTTGGTTTTACTTGTATCTGTAAGC | |
| YbdN | ATGGTTAAGAAATGGCTTATACAGTTCGCAGTTATGTTGAGCGTGCTG | 329 |
| | AGCACCTTCACATATAGCGCGAGCGCA | |
| YbfO | ATGAAACGCATGATTGTCCGAATGACACTGCCGTTGCTGATAGTTTGT | 330 |
| | CTGGCTTTTTCAAGCTTTAGCGCTAGTGCCCGCGCG | |
| YbxI | ATGAAGAAATGGATATATGTTGTACTTGTTTTGTCGATCGCGGGAATC | 331 |
| | GGCGGCTTTTCGGTTCACGCC | |
| YckD | ATGAAAAGAATTACGATTAACATTATTACAATGTTTATTGCTGCAGCC | 332 |
| | GTCATTTCTCTCACGGGCACCGCCGAGGCC | |
| YdbK | ATGAAACTGTTCAATCGGAAAGTCACGTTAGTGAGCTTAATTCTTATG | 333 |
| | GCCGTGTTCCAATTTTTTATGGCCTTAATCATCAAACGAATAGTAATTT | |
| | CC | |
| YddT | ATGAGAAAGAAACGTGTGATCACGTGCGTGATGGCGGCGAGCCTCAC | 334 |
| | ATTAGGAAGCTTACTCCCTGCAGGCTACGCCTCCGCT | |
| YdhT | ATGTTTAAGAAACACACTATATCTTTACTGATCATTTTTCTTCTGGCGT | 335 |
| | CAGCAGTACTTGCA | |
| YdjM | ATGCTGAAAAAGGTAATACTGGCAGCTTTCATCCTGGTGGGAAGCACG | 336 |
| | CTGGGGGCATTTTCTTTCAGCTCTGACGCCTCGGCT | |
| YdjN | ATGAAGAAGCGGATTATCCTCCTTCTGGCGGTTATTATCGCCGCCGCT | 337 |
| | GCAGCGGGGGTTGCC | |
| YfhK | ATGAAAAAAAAGCAGGTGATGCTGGCTCTTACCGCCGCTGCTGGGCTG | 338 |
| | GGACTTACCGCTCTCCATTCTGCTCCTGCCGCGAAAGCA | |
| YfjS | ATGAAATGGATGTGCTCAATCTGCTGTGCTGCAGTATTACTTGCAGGA | 339 |
| | GGAGCTGCTCAAGCT | |
| YfkD | ATGATGAAAAAGCTGTTCCATAGCACACTGATCGTCCTCCTGTTTTTTT | 340 |
| | CTTTTTTTGGGGTACAACCCATACATGCA | |
| YfkN | ATGCGAATTCAGAAACGGAGGACTCATGTAGAAAATATCCTCCGTATT | 341 |
| | TTGTTACCGCCGATAATGATCCTCTCGCTGATTTTGCCTACTCCGCCGA | |
| | TCCATGCA | |
| YhaK | ATGCGCACGTGGAAACGTATTCCGAAAACGACTATGCTCATCTCACTC | 342 |
| | GTGTCACCGTTCCTTTTGATTACACCGGTTCTTTTTTATGCGGCATTAG | |
| | CT | |
| YhcR | ATGCTTTCTGTCGAAATGATTTCCAGGCAGAATCGTTGTCACTACGTCT | 343 |
| | ACAAAGGCGGCAACATGATGAGACGGATTTTACACATCGTACTCATA | |
| | ACCGCGTTGATGTTTCTTAATGTTATGTATACTTTTGAAGCG | |
| YhdC | ATGAAATCTCTCCCGTATACGATCGCCCTGCTGTTTTGTGGCTTAATTA | 344 |
| | TTGTCTCCATGGCA | |
| YhfM | ATGAAAAAAATTGTCGCAGCGATCGTAGTGATCGGATTGGTTTTCATT | 345 |
| | GCTTTTTTTTATCTGTATTCACGATCTGGAGACGTTTATCAATCCGTTG | |
| | ATGCA | |
| YhjA | ATGAAAAAGGCCGCGGCAGTGCTGCTTTCTCTGGGACTGGTGTTCGGG | 346 |
| | TTCAGCTACGGAGCAGGTCACGTGGCTGAAGCC | |
| YjcM | ATGAAAAAAGAGCTGCTCGCGAGTCTGGTCCTGTGCTTATCCCTGAGC | 347 |
| | CCTTTGGTATCTACAAACGAGGTGTTCGCA | |
| YjcN | ATGAAAAAAAAAACAAAGATTATCCTCTCTCTTCTTGCGGCCTTGATC | 348 |
| | GTCATTTTGATCGTGTTGCCGGTCCTTTCCCCGGTTGTATTTACAGCAA | |
| | GTTCG | |
| YjdB | ATGAATTTTAAAAAAACAGTTGTATCAGCGTTGTCAATCTCTGCACTT | 349 |
| | GCGCTTTCAGTGTCAGGTGTCGCAAGCGCT | |
| YjfA | ATGAAAAGATTGTTTATGAAAGCAAGTTTAGTTTTGTTTGCGGTTGTTT | 350 |
| | TCGTGTTCGCGGTAAAAGGCGCGCCTGCCAAAGCC | |
| YjiA | ATGGCTGCTCAGACCGATTATAAAAAGCAAGTCGTCGGAATACTTCTC | 351 |
| | AGCCTTGCCTTTGTGTTGTTTGTGTTTTCT | |
| YknX | ATGAAAAAAGTGTGGATCGGAATCGGCATTGCCGTAATCGTTGCTTTA | 352 |
| | TTTGTAGGCATTAATATTTATAGGTCGGCCGCACCGACATCTGGGTCG | |
| | GCA | |
| YkoJ | ATGCTGAAAAAAAAGTGGATGGTTGGACTTCTGGCTGGCTGCCTGGCG | 353 |
| | GCTGGAGGATTTTCTTACAATGCATTTGCG | |
| YkvT | ATGACCACAAAATTCACCGCTCTGGCGGTGTTCTTGTTATGTTTCATGC | 354 |
| | CGGCTGCAAAAATC | |
| YkvV | ATGCTCACAAAACGTCTTTTAACGATCTATATCATGCTTTTAGGTCTGA | 355 |
| | TAGCGTGGTTTCCTGGGGCTGCCCAAGCG | |
| YkwD | ATGAAAAAGGCATTTATCTTATCGGCAGCGGCGGCAGTAGGATTGTTT | 356 |
| | ACATTTGGTGGGGTCCAACAGGCCTCCGCA | |
| YlaE | ATGAAGAAAACCTTTGTGAAAAAGGCAATGCTGACAACGGCGGCGAT | 357 |
| | GACCTCGGCCGCACTGCTTACATTCGGCCCGGACGCAGCTTCAGCC | |
| YlbL | ATGTTGAGAAAAAAACATTTTTCGTGGATGCTTGTGATTCTTATCTTAA | 358 |
| | TTGCAGTGCTTTCATTCATTAAATTGCCGTACTATATCACAAAACCCGG | |
| | AGAAGCA | |
| YlqB | ATGAAGAAGATCGGCTTGCTTTTTATGTTGTGTCTGGCTGCTTTGTTCA | 359 |
| | CAATTGGCTTTCCAGCTCAGCAAGCTGATGCG | |
| Y1xF | ATGTCTGGAAAAAAGAAAGAGTCCGGGAAATTTCGCTCAGTTTTGCTG | 360 |
| | ATCATTATTTTGCCGCTTATGTTTCTGTTGATTGCTGGGGGTATCGTCT | |
| | TATGGGCGGCGGGA | |
| YlxW | ATGCGGGGCAAAAGTGCTGTATTACTTAGTCTCATCATGTTAATCGCA | 361 |
| | GGATTTCTCATTAGCTTCTCGTTTCAGATGACCAAGGAGAATAATAAA | |
| | TCGGCAGCA | |
| YlxY | ATGTACAAAAAATTTGTCCCATTTGCGGTGTTTTTGTTTCTTTTTTTTGT | 362 |
| | GTCTTTTGAAATGATGGAGAATCCACATGCACTTGATTACATCGGCGC | |
| | A | |
| YncM | ATGGCAAAACCGCTTTCCAAAGGAGGAATATTGGTGAAAAAAGTTCT | 363 |
| | GATCGCCGGTGCGGTAGGTACAGCGGTATTATTTGGGACTTTATCATC | |
| | CGGAATCCCTGGTTTACCAGCGGCAGATGCT | |
| YndA | ATGAGATTTACAAAGGTAGTCGGCTTTTTATCTGTCCTTGGATTGGCG | 364 |
| | GCGGTTTTCCCGCTGACGGCGCAGGCG | |
| YnfF | ATGATACCGAGGATTAAAAAGACGATTTGCGTGCTGCTGGTATGTTTC | 365 |
| | ACGATGTTGAGTGTCATGCTTGGTCCAGGAGCGACAGAAGTATTGGCG | |
| YngK | ATGAAAGTCTGTCAAAAGAGTATAGTCAGATTTCTTGTATCTCTTATTA | 366 |
| | TTGGTACCTTTGTTATTTCAGTCCCATTTATGGCAAACGCG | |
| YnzA | ATGGAATTATCCTTTACGAAAATCCTGGTTATTCTTTTTGTCGGGTTTT | 367 |
| | TGGTTTTTGGCCCGGATAAACTTCCAGCTCTTGGA | |
| YoaW | ATGAAAAAAATGTTGATGCTGGCCTTCACTTTCCTGCTTGCCCTCACA | 368 |
| | ATCCATGTTGGTGAAGCGTCGGCA | |
| YobB | ATGAAAATCAGAAAAATTCTGTTGAGCTCAGCTCTTTCATTTGGTATG | 369 |
| | TTGATCAGCGCTGTACCTGCACTTGCA | |
| YobV | ATGAAACTCGAACGGTTGTTAGCAATGGTAGTCCTCTTAATAAGTAAA | 370 |
| | AAACAGGTTCAAGCG | |
| YocA | ATGAAAAAAAAAAGAAAGGGGTGCTTTGCGGCAGCCGGGTTCATGAT | 371 |
| | GATCTTTGTTTTCGTCATTGCG | |
| YocH | ATGAAAAAGACAATCATGTCATTTGTGGCAGTTGCAGCACTTAGTACA | 372 |
| | ACAGCGTTCGGCGCACATGCA | |
| YodV | ATGAAGGTCCCCAAAACGATGCTGTTGTCTACAGCGGCGGGACTCTTA | 373 |
| | CTCTCACTGACGGCGACGTCCGTGTCAGCC | |
| YojL | ATGAAAAAGAAAATTGTCGCGGGGCTGGCCGTTTCAGCGGTAGTCGG | 374 |
| | TTCTAGTATGGCCGCAGCTCCGGCTGAAGCC | |
| YolA | ATGAAGAAGCGCATTACTTATTCTCTTTTGGCACTGCTTGCAGTTGTTG | 375 |
| | CATTCGCTTTTACCGACTCGTCTAAGGCGAAAGCA | |
| YolC | ATGAAAAAGCGTCTGATTGGCTTTCTGGTGTTAGTACCGGCATTAATT | 376 |
| | ATGTCTGGAATTACCTTAATTGAAGCA | |
| YolI | ATGAAAAAATGGATTGTCCTGTTTTTAGTACTTATCGCAGCTGCGATTT | 377 |
| | CTATATTTGTCTATGTGTCCACAGGGTCAGAG | |
| YomL | ATGAGGAAGAAACGGGTGATTACCTGCGTAATGGCAGCTAGTCTGAC | 378 |
| | TCTGGGCTCGCTTCTTCCAGCAGGATATGCCACAGCT | |
| YopL | ATGAAAAAACTGATTATGGCGCTTGTTATCTTGGGGGCACTTGGAACC | 379 |
| | TCTTACATTTCAGCC | |
| YoqH | ATGAAACGCTTTATACTCGTGTTATCTTTTCTCTCGATTATAGTAGCGT | 380 |
| | ATCCGATTCAAACTAACGCA | |
| YoqM | ATGAAATTACGCAAAGTACTCACCGGATCCGTCTTAAGCTTAGGATTG | 381 |
| | TTAGTCAGCGCGAGTCCGGCTTTCGCC | |
| YpbG | AAATTGTCTGTAAAGATTGCAGGCGTGCTCACCGTTGCAGCTGCTGCC | 382 |
| | ATGACGGCGAAAATGTACGCAACAGCC | |
| YpcP | ATGAACAATAACAAGCTTCTCCTTGTCGATGGCATGGCACTGCTGTTT | 383 |
| | CGTGCCTTTTTTGCGACCGCA | |
| YpjP | ATGAAGCTGTGGATGCGTAAAACGCTTGTCGTGCTTTTTACCATCGTA | 384 |
| | ACTTTTGGCCTGGTATCTCCTCCAGCTGCTTTGATGGCG | |
| YpmB | ATGCGGAAGAAGGCATTAATTTTTACGGTTATTTTTGGTATCATTTTCC | 385 |
| | TGGCAGTGTTGTTGGTCTCTGCAAGCATATATAAATCAGCAATGGCC | |
| YpmS | AACAAATGGAAGCGTCTGTTTTTTATTCTGCTTGCTATCAATTTTATCC | 386 |
| | TGGCTGCCGGCTTTGTCGCGTTAGTGCTTTTACCGGGAGAACAAGCTC | |
| | AAGTC | |
| YpuA | ATGAAGAAGATCTGGATCGGCATGCTTGCCGCGGCAGTATTGTTGCTT | 387 |
| | ATGGTGCCAAAGGTTAGTCTGGCCGATGCT | |
| YpuD | ATGGGACGTATCAAGACGAAGATTACGATATTATTGGTTCTGTTACTC | 388 |
| | TTGTTAGCGGGAGGTTACATGTATATCAATGATATCGAACTTAAAGAT | |
| | GTCCCTACAGCTATAGGA | |
| YqfZ | ATGAAACGCCTGACACTGGTCTGTTCTATCGTTTTTATCCTCTTTATTTT | 389 |
| | GTTTTATGATCTTAAAATCGGTACAATTCCCATCCAAGACCTTCCGGTA | |
| | TACGAGGCCTCGGCT | |
| YqgA | ATGAAACAAGGTAAATTTTCGGTCTTTTTGATTCTTCTGCTTATGCTCA | 390 |
| | CGCTTGTTGTGGCACCAAAAGGAAAAGCGGAAGCC | |
| YqxI | ATGTTCAAGAAACTTCTGCTCGCTACAAGCGCCCTTACATTTTCTCTTT | 391 |
| | CACTGGTATTGCCTTTGGACGGACACGCCAAAGCG | |
| YqxM | ATGTTCAGGCTGTTTCATAACCAGCAGAAAGCTAAAACAAAATTAAA | 392 |
| | GGTTTTATTGATATTTCAACTGAGCGTCATTTTTAGTTTAACCGCCGCC | |
| | ATTTGTTTACAATTTAGCGATGATACATCCGCA | |
| YqzC | ATGACGAAGAGAGGCATCCAGGCCTTCGCGGGGGGCATCATTTTGGC | 393 |
| | GACAGCAGTGCTTGCCGCTGTATTCTATCTTACAGACGAGGATCAAGC | |
| | AGCTGCT | |
| YqzG | ATGATGATCAAACAGTGTGTTATTTGCCTTAGCTTACTTGTCTTCGGAA | 394 |
| | CAACAGCCGCCCACGCC | |
| YraJ | ATGACACTCACGAAATTGAAAATGCTCAGTATGCTGACAGTAATGATT | 395 |
| | GCATCATTGTTTATCTTTAGCTCACAGGCTCTTGCG | |
| YrrL | ATGTATATTAACCAGCAAAAAAAATCATTTTTTAACAAAAAACGAATT | 396 |
| | ATCTTGTCAAGCATCGTCGTATTGTTTTTAATCATCGGAGGCGCTTTCC | |
| | TC | |
| YrrR | ATGAAAATCAGCAAACGCATGAAACTTGCCGTTATTGCTTTTTTGATT | 397 |
| | GTCTTTTTTCTGCTTTTGCTCAGACTTGCTGAGATC | |
| YrrS | ATGTCTAATAACCAATCCCGGTACGAAAATCGCGACAAACGCAGAAA | 398 |
| | AGCAAACCTGGTATTAAACATTTTAATTGCAATCGTCAGCATCCTCAT | |
| | TGTGGTGGTGGCTGCT | |
| YrvJ | ATGAATAAAAAGTACTTTGTCCTGATCGTATGTATTATTTTCACATCCG | 399 |
| | CCTTATTTCCGACGTTCTCGTCCGTGACAGCT | |
| YuaB | ATGAAACGGAAACTGCTGTCTTCTCTTGCCATTTCAGCTTTGTCATTAG | 400 |
| | GATTACTTGTTAGCGCGCCGACGGCGTCTTTTGCAGCTGAG | |
| YunA | ATGATCACAGACATTTTCAAACCAGGTTGCAGAAAATTATGCGTATTT | 401 |
| | AATATGAAGGGCGATTACTTTGTTAAAGTCCTCCTGTCTGCTTTACTTC | |
| | TGCTCCTTTTTGCA | |
| YunB | ATGCCCAGGTACCGGGGACCGTTTCGCAAAAGAGGACCTTTACCTTTT | 402 |
| | CGGTATGTCATGTTACTTAGCGTAGTTTTTTTCATTTTAAGCACAACAG | |
| | TTTCACTT | |
| YurI | ATGACGAAAAAAGCATGGTTTCTCCCGCTCGTTTGTGTTCTCTTAATCA | 403 |
| | GTGGATGGTTAGCCCCTGCAGCGTCTGCCAGTGCG | |
| YusW | ATGCATTTAATAAGGGCGGCTGGCGCGGTGTGCCTTGCAGTTGTTTTA | 404 |
| | ATCGCTGGATGTAGATTTAATGAGGATCAACACCAAGCAGAAGGT | |
| YvbX | ATGAAGAAGTGGCTGATCATTGCGGTCAGTCTCGCCATTGCGATTGTG | 405 |
| | CTGTTTATGTATACAAAAGGCGAGGCGAAAGCT | |
| YvcE | ATGCGGAAAAGCCTGATCACGCTGGGCCTGGCATCTGTCATTGGCACA | 406 |
| | AGCTCTTTTCTCATTCCGTTTACTTCAAAGACAGCGTCTGCT | |
| YveB | ATGAATTATATTAAGGCCGGCAAATGGCTCACGGTCTTCTTAACATTT | 407 |
| | CTGGGAATTTTACTGTTCATTGATCTG | |
| YvgO | ATGAAGCGCATCAGGATCCCTATGACGCTCGCACTGGGTGCGGCACTT | 408 |
| | ACCATCGCGCCGTTGTCCTTTGCAAGCGCG | |
| YvgV | ATGAAGAAAAAGCAACAAAGTTCAGCTAAATTTGCTGTTATCTTAACA | 409 |
| | GTAGTTGTGGTCGTGTTGTTGGCAGCGATTGTC | |
| YvnB | ATGCGTAAATACACAGTAATTGCCTCGATCCTGCTTTCATTTCTGTCGG | 410 |
| | TGCTGTCTGGTGGC | |
| YvpA | ATGAAAAAAATCGTATCAATTCTGTTTATGTTTGGATTGGTCATGGGA | 411 |
| | TTTAGTCAGTTCCAGCCGTCGACAGTATTTGCA | |
| YvpB | ATGAAAACATTGCGCACGCTTTGTGTACTTATGATCTTGAGCGGTGTT | 412 |
| | ATATTTTTTGGCCTGAAAATTGACGCC | |
| YwaD | ATGAAGAAGCTGCTCACAGTAATGACAATGGCCGTTTTGACGGCAGG | 413 |
| | AACCTTGCTCTTGCCGGCGCAGTCTGTGACCCCGGCCGCTCACGCA | |
| YwcI | ATGAAACGCTTGCTCGTAAGCTTGAGAGTTTGGATGGTGTTCTTGATG | 414 |
| | AACTGGGTTACACCGGACCGCAAAACGGCTCGTGCC | |
| YwdK | ATGAAAGTTTTTATCATACTTGGCGCTATCAATGCGTTGTTAGCAGTG | 415 |
| | GGACTCGGGGCCTTTGGT | |
| YweA | ATGTTGAAGCGCACTTCATTCGTAAGCTCTCTTTTCATATCAAGTGCGG | 416 |
| | TTCTGCTTAGCATTCTTCTGCCGAGCGGACAGGCGCACGCC | |
| YwfM | ATGAAAGGCAACATTTATTCATTGTTTGTGTTAATTGCTGCCTTCTTCT | 417 |
| | GGGGTACAACAGGCACAGTCCAAGCG | |
| YwgB | ATGAAAATGAAATCAGGTATGGAGCAGGCGGTATCAGTCCTTCTTCTT | 418 |
| | TTGTCGCGTTTGCCAGTTCAGGCC | |
| YwjE | ATGAAAGTTTTTATCGTCATAATGATTATCGTGGTTATTTTTTTCGCGC | 419 |
| | TTATTCTTTTAGATATCTTCATGGGACGTGCG | |
| YwmB | ATGAAGAAAAAGCAGGTCTCACACGCCATTATTATTAGCGTTATGTTA | 420 |
| | TCTTTTGTAATCGCAGTTTTCCACACGATTCATGCG | |
| YwmC | ATGAAAAAGCGGTTTTCTTTAATCATGATGACCGGATTGTTATTCGGT | 421 |
| | CTTACCTCTCCCGCATTCGCT | |
| YwmD | ATGAAAAAATTACTTGCAGCCGGCATTATTGGGTTGCTTACGGTGTCC | 422 |
| | ATTGCCAGCCCGTCTTTTGCC | |
| YwoF | ATGCGTAAATGGTATTTTATCTTATTAGCTGGCGTCCTTACCTCCGTTA | 423 |
| | TCCTTGCGTTTGTCTATGATAAAACGAAAGCG | |
| YwqC | ATGGGCGAATCTACATCCCTCAAAGAAATTTTAAGCACCTTGACTAAA | 424 |
| | CGCATCCTTCTGATCATGATAGTGACGGCCGCTGCCACCGCA | |
| YwqO | ATGAAATTTTTGCTGTCGGTGATTGCTGGCCTTTTAATCCTGGCATTAT | 425 |
| | ATTTGTTTTGGAAAGTCCAACCACCTGTCTGGATT | |
| YwsB | ATGAACAAACCTACAAAGCTCTTTTCAACGCTTGCATTAGCTGCTGGA | 426 |
| | ATGACAGCAGCAGCCGCCGGAGGAGCCGGGACAATCCATGCC | |
| YwtC | ATGAAATTCGTTAAAGCTATTTGGCCGTTTGTTGCAGTAGCAATCGTCT | 427 |
| | TTATGTTTATGTCGGCA | |
| YwtD | ATGAATACACTTGCAAACTGGAAGAAATTTCTGTTAGTCGCTGTGATT | 428 |
| | ATTTGCTTTCTGGTACCTATTATGACAAAGGCCGAAATTGCAGAAGCA | |
| YwtF | ATGGAAGAAAGAAGCCAGCGCCGGAAAAAAAAGAGAAAGCTCAAAA | 429 |
| | AATGGGTAAAAGTTGTAGCTGGACTTATGGCATTTCTTGTCATTGCGG | |
| | CCGGATCAGTTGGCGCCTATGCT | |
| YxaK | ATGGTCAAATCGTTCAGAATGAAGGCATTGATCGCAGGCGCAGCCGT | 430 |
| | AGCGGCGGCCGTGTCCGCAGGCGCCGTGTCCGATGTCCCGGCTGCTAA | |
| | AGTACTCCAACCCACAGCGGCCTACGCT | |
| YxiA | ATGTTCAACCGTTTGTTTCGGGTGTGTTTTCTTGCGGCGCTGATTATGG | 431 |
| | CATTCACGTTGCCTAACTCTGTTTATGCC | |
| YxiT | ATGAAGTGGAACAATATGCTGAAAGCGGCGGGCATTGCTGTCTTGTTA | 432 |
| | TTTAGCGTATTCGCATATGCCGCACCGTCATTAAAAGCAGTACAGGCC | |
| YyaB | ATGGTATATCAGACAAAAAGAGATGTTCCTGTCACACTTATGATTGTC | 433 |
| | TTCTTAATCCTGCTGATTCAGGCCGATGCA | |
| YybN | ATGAATAAGTTTTTAAAATCGAACTTCCGTTTCCTGTTAGCTGCCGCTC | 434 |
| | TTGGAATATCTCTTCTTGCGTCATCAAACTTCATCAAAGCG | |
| YycP | ATGAAAAAATGGATGATTACAATCGCCATGCTCATTTTGGCCGGAATC | 435 |
| | GCATTATTTGTTTTCATTTCTCCTCTGAAAAGC | |

### Example 5: Hcy and Cys Determination by HPLC

Scope: This procedure defines an exemplary method of quantitative determination of total homocysteine in plasma (serum) by liquid. The method can detect four thiol amino acids, Hcy, Cys, Cys-gly, and GSH. Use here can advantageously detect Hcy and Cys.

Principle: Aminothiols in plasma (serum) are modified by using SBD-F and subsequently separated and quantified by HPLC with fluorometric detection. Oxidized forms of aminothiols can be reduced by using trialkylphosphin (TCEP) prior modification.

Pre-analytical phase sample: Determination of total homocysteine concentration is performed in the plasma (serum). Blood is taken into EDTA (tube partially immersed in water with ice). Within 1 h after sampling, plasma should be separated (centrifugation 5 min, min. 2000 g) and frozen. The amount of sample for analysis is 5 ul to 50 ul of plasma (serum). K2 (K3) EDTA, heparin plasma—is stable 1 year at least at −18° C.

Apparatuses can include the following: Liquid chromatograph with fluorimetric detector; HPLC separation column (e.g.: NUCLEOSIL 100-3 C18 (100×3.2 mm) from WATREX); thermoblock; automatic pipette; analytical balance; table microcentrifuges; pH meter; glassware.

Chemicals can include the following: (1) L-homocysteine (HCYin) M.W.=268.40 g/mol, SIGMA-ALDRICH (H6010) [L-4, 4′-Dithiobis[2-aminobutanoic]acid]; (2) L-cysteine (CYS) >99.5% (m/m), M.W.=121.16 g/mol, FLUKA (30089) [(R)-2-Amino-3-mercaptopropionic acid]; (3) optionally cysteinyl-glycine (CYS-GLY) >85% (m/m), M.W.=178.21 g/mol, SIGMA-ALDRICH (C 0166); (4) optionally Glutathione (GSH red.) 98-100% (m/m), M.W.=307.30 g/mol, SIGMA-ALDRICH (G6529), Glutathione Fragment Reduced Form Free acid; (5) Mercaptopropionylglycine (I.S.) 98-100% (m/m), M.W.=163.20 g/mol, SIGMA-ALDRICH (M6635) N (2-mercaptopropionyl)-glycine cristalline; (6) Acetonitrile (ACN) gradient grade for HPLC, M.W.=40.05 g/mol; (7) tris(carboxyethyl)phosphine hydrochloride (TCEP) M.W.=286.65 g/mol, SIGMA-ALDRICH (C4706); (8) Trichloro acetic acid (TCA) >99% (m/m), M.W.=163.39 g/mol; (9) ethylendiaminetetraacetic acid disodium salt dihydrate (EDTA) 99% (m/m), M.W.=372.24 g/mol; (10) Sodium tetraborate decahydrate (BORAX) 99.5-105% (m/m), M.W.=381.40 g/mol; (11) 7-fluorobenzofurazane-4-sulfonic acid ammonium salt (SBD-F) >98.5% (m/m) FLUKA (46640), M.W.=235.19 g/mol; (12) Potassium dihydrogen phosphate=(KH2PO4) >99.5% (m/m), M.W.=136.09 g/mol; (13) Orto-phosphoric acid (H3PO4) >85% (m/m), M.W.=98.00 g/mol; (14) phosphate buffered saline (PBS).

Reagents: can include the following (1) TCEP (reducing agent for)—Reagent B, 8 mg TCEP dissolved in 0.15 ml PBS (Phosphate buffered saline), always prepare fresh; (2) TCA-EDTA (reagent deproteination)—Reagent C, 10% TCA in 1 mM EDTA, dissolve 5 g TCA and 19 mg EDTA in 50 ml of demineralized water reagent is stable for 1 month at 2-8° C.; (3) BORAX-EDTA (alkaline reagent for SBD-F solubilization), 125 mmol/L BORAX in 4 mmol/L EDTA (pH 9.5), dissolve 2.4 g borax and 74 mg EDTA in 50 ml of demineralized water, reagent is stable for 1 month at room temperature (can crystallize in the refrigerator); (4) SBD-F (derivatization reagent) Stock SBD-F, dissolve 1 mg of SBD-F in 1 ml of borax-EDTA, better to prepare a fresh solution, if possible, protected from light. but it can be stored at 4° C. protected from light for one or two months; (5) Working solution of SBD-F for 1 sample—Reagent D: to 30 ul stock SBD-F, add 70 ul of borax-EDTA, always prepare a fresh solution, if possible, protected from light; (6) I.S. mercaptopropionylglycine (internal standard)—Reagent A, 8.16 mg dissolved in 500 ml of demineralized water, the concentration of a solution is 100 umol/l, then the solution is diluted 1:3 with demineralized water, to create, solution with a concentration of 25 umol/l, or 200 uM can be prepared to dilute 1:6, always prepare fresh for dilution; (7) PBS phosphate buffered saline (solvent for TCEP), dissolve 8.0 g NaCl, 0.2 g KCl, 1.15 g Na₂HPO 4×12H₂O in 1000 ml of deionized water.

Preparation of calibration standards. Stock solutions for calibration: HCY in 6.00 mmol/l in water (aliquots in tubes, kept at −80° C.); CYS 80.00 mmol/l in water (aliquots tubes, kept at −80° C.). Note: the test is customized for HO mice or Meada mice.

There are two standard curves for HO mice and meada mice, respectively. One is designed as High standards and one as Low standards.

TABLE 12
High standard curve:

| | High 6 | High 5 | High 4 | High 3 | High 2 | High 1 |
|---|---|---|---|---|---|---|
| Hcy (uM) | 600 | 300 | 150 | 75 | 37.5 | 18.75 |
| Cys (uM) | 400 | 200 | 100 | 50 | 25 | 12.5 |

Solution of High 6 is aliquoted into tubes with 100 ul each and kept at −80° C. One tube can be pulled out for each HPLC run, and the serials of High standard curve can be diluted with water freshly for use.

TABLE 13
Low standard curve:

| | Low 6 | Low 5 | Low 4 | Low 3 | Low 2 | Low 1 |
|---|---|---|---|---|---|---|
| Hcy (uM) | 40 | 20 | 16 | 8 | 4 | 2 |
| Cys (uM) | 400 | 200 | 100 | 50 | 25 | 12.5 |

Solution of Low 6 is aliquoted into clear tubes with 50 ul each and kept at −80° C. One tube can be pulled out for each HPLC run and the serials of Low standard curve can be diluted with water freshly for use.

Preparation of mobile phase for HPLC. Mobile phase A=50 mM KH₂PO₄ pH ˜1.9 (adjusted with 85% HPO₄). Mobile phase B=30% ACN: 70% mobile phase A (150 ml ACN: 350 ml of mobile phase A)

SAMPLE PROCESSING: All samples and standards were prepared in the same procedure described below. The whole procedure is carried out in EPPENDORF tubes after adding SBD-F.

Table 14: Sample processing steps. Thaw and mix thoroughly all samples, standards and chemicals. Work on ice.

TABLE 14

| | | |
|---|---|---|
| Plasma (serum) or Calibrator | 50 | ul |
| I.S. (reagent A) | 40 | ul |
| TCEP (reagent B) | 12.5 | ul |

Mix and let stand 30 minutes at RT. Transfer back to ice.

| | | |
|---|---|---|
| TCA - EDTA (reagent C) | 50 | ul |

Mix and let stand for 10 minutes on ice
Centrifuge 4 minutes at 10000 g (then use the supernatant **)
EPPENDORF tubes

| | | |
|---|---|---|
| SBD-F - Borax - EDTA (reagent D) 100 ml | 100 | ul |
| Supernatant ** | 25 | ul |

Protect from light. Derivatization for 30 minutes at 60° C.
Immediately cool on ice and mix.
Always spin down samples in bench microcentrifuge prior to transfer into the vials.
Transfer samples into amber vials and place them into HPLC autosampler.

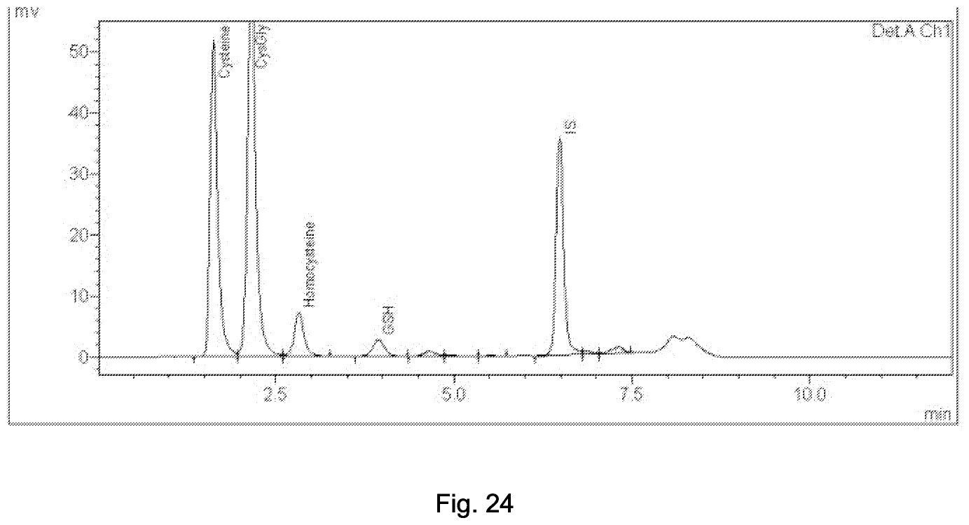

Sample processing: All samples are analyzed by HPLC with fluorometric detection: Column: WATREX 100×3.2 mm, NUCLEOSIL 100-3 C18. Flow rate: 0.7 ml/min. Gradient elution can be performed with mobile phases A and B. Injection volume can be 10 ul. The approximate retention times are as follows: CYS 1.7 min; Hey 3.0 min; I.S. 6.0 min (retention time depends on the strength of the column and mobile phase−% ACN). See e.g., FIG. 24 for an exemplary HPLC readout.

The resulting values of plasma (serum) aminothiols are calculated from the calibration curves and internal standard (e.g., using software from SHIMADZU, this system is common to use for HPLC analysis). The resulting value of aminothiols in a sample is expressed in umole/l.

Table 15 shows the biological reference interval in the plasma (serum) of homocysteine, depending on age or pregnancy state. The reference range was obtained using published data.

TABLE 15

| | |
|---|---|
| age | Homocysteine [mg/dL] |

| | | |
|---|---|---|
| <15 | years | ≤10 |
| 15-65 | years | ≤15 |
| >65 | years | ≤20 |

| | |
|---|---|
| pregnancy | ≤10 |

In some embodiments, the volume of plasma can be lowered to 5 ul if the sample is very limited. Such a 5 uL volume can produce enough solution for running on HPLC.

Hey is not very stable; do not freeze-thaw the Hey standards.

What is claimed herein is:

1. An engineered probiotic microorganism for reducing bioavailable methionine levels, comprising:
a) at least one exogenous copy of at least one gene encoding an enzyme that catalyzes the degradation of methionine, wherein the gene encoding an enzyme that catalyzes the degradation of methionine encodes a methionine gamma lyase.

2. An engineered probiotic microorganism for reducing bioavailable methionine levels, comprising:
a) at least one exogenous copy of at least one gene encoding an enzyme that catalyzes the degradation of methionine, wherein the gene encoding an enzyme that catalyzes the degradation of methionine encodes a methionine gamma lyase; and
b) at least one of the following:
i) at least one exogenous copy of at least one functional methionine importer gene; and/or
ii) at least one endogenous methionine importer gene comprising at least one engineered activating modification.

3. An engineered probiotic microorganism for reducing bioavailable methionine levels, comprising:
a) at least one exogenous copy of at least one gene encoding an enzyme that catalyzes the degradation of methionine;
b) at least one exogenous copy of at least one functional methionine importer gene;
c) at least one endogenous methionine importer gene comprising at least one engineered activating modification;
d) at least one endogenous methionine synthesis gene comprising at least one engineered inactivating modification;
e) at least one endogenous methionine regulator gene comprising at least one engineered inactivating or activating modification; or
f) a combination of two or more of (a)-(e).

4. The engineered probiotic microorganism of any one of claims 1-3, wherein the exogenous gene(s) of (a) and (b), if present, and the endogenous gene(s) of (c) (d), and (e), if present, are expressed by the engineered probiotic microorganism under conditions in the gut.

5. The engineered probiotic microorganism of any one of claims 1-3, wherein the at least one engineered activating modification comprises:
a) at least one engineered activating mutation in the at least one endogenous methionine importer gene or in the at least one endogenous methionine regulator gene; and/or
b) at least one engineered activating mutation in a promoter operatively linked to the at least one endogenous methionine importer gene or to the at least one endogenous methionine regulator gene.

6. The engineered probiotic microorganism of claim 3, wherein the at least one engineered inactivating modification comprises:
a) at least one engineered inactivating mutation in the at least one endogenous methionine synthesis gene or in the at least one endogenous methionine regulator gene;
b) at least one engineered inactivating mutation in a promoter operatively linked to the at least one endogenous methionine synthesis gene or to the at least one endogenous methionine regulator gene; and/or
c) at least one inhibitory RNA molecule specific to at least one messenger RNA (mRNA) expressed by the at least one endogenous methionine synthesis gene or by the at least one endogenous methionine regulator gene.

7. The engineered probiotic microorganism of any one of claims 1-3, wherein the enzyme that catalyzes the degradation of methionine generates methanethiol.

8. The engineered probiotic microorganism of any one of claims 1-3, wherein the gene encoding an enzyme that catalyzes the degradation of methionine encodes a methionine gamma lyase.

9. The engineered probiotic microorganism of any one of claims 1-3, which further comprises and expresses an exogenous gene encoding a methanethiol catabolizing enzyme.

10. The engineered probiotic microorganism of claim 7, wherein the methanethiol-catabolizing enzyme is an esterase or a methanethiol oxidase.

11. The engineered probiotic microorganism of any one of any one of claims 1-3, wherein the methionine gamma lyase comprises SEQ ID NO: 6 or an amino acid sequence that is at least 90% identical.

12. The engineered probiotic microorganism of any one of any one of claims 1-3, wherein the methionine gamma lyase comprises one of SEQ ID NOs: 5-6 or an amino acid sequence that is at least 90% identical.

13. The engineered probiotic microorganism of any one of claims 1-3, wherein the gene encoding an enzyme that catalyzes the degradation of methionine encodes expression of a catalytically-active fragment of a methionine gamma lyase.

14. The engineered probiotic microorganism of any one of claims 1-3, wherein the gene encoding an enzyme that catalyzes the degradation of methionine encodes expression of a fusion protein comprising a catalytically-active fragment of a methionine gamma lyase.

15. The engineered probiotic microorganism of any one of claims 1-3, which comprises at least one exogenous copy of at least one gene encoding an enzyme that catalyzes the degradation of methionine and at least one exogenous copy of at least one functional methionine importer gene.

16. The engineered probiotic microorganism of any one of claims 1-3, which comprises at least one exogenous copy of at least one gene encoding an enzyme that catalyzes the degradation of methionine and at least one endogenous copy of at least one functional methionine importer gene comprises a mutation that increases the rate of methionine import relative to wild-type of that enzyme.

17. The engineered probiotic microorganism of any one of claims 1-3, which comprises at least one exogenous copy of at least one gene encoding an enzyme that catalyzes the degradation of methionine and at least one endogenous methionine synthesis gene comprising at least one engineered inactivating modification.

18. The engineered probiotic microorganism of any one of claims 1-3, which comprises at least one exogenous copy of at least one gene encoding an enzyme that catalyzes the degradation of methionine and at least one endogenous methionine regulator gene comprising at least one engineered inactivating or activating modification.

19. An engineered probiotic microorganism for reducing bioavailable methionine levels, the microorganism comprising:
a) at least one endogenous methionine synthesis gene comprising at least one engineered inactivating modification;
b) at least one copy of an exogenous gene encoding a homocysteine methyltransferase enzyme;
c) at least one copy of an exogenous gene encoding a sulfinoalanine decarboxylase enzyme; and
d) at least one copy of an exogenous gene encoding a Flavin-containing monooxygenase (FMO) enzyme;
wherein the engineered probiotic microorganism expresses endogenously or exogenously encoded cystathionine β-synthase, cystathionine gamma lyase and cysteine dioxygenase enzymes.

20. The engineered probiotic microorganism of claim 19, wherein the homocysteine methyltransferase enzyme is a YhcE homocysteine methyltransferase enzyme.

21. The engineered probiotic microorganism of claim 19, wherein the at least one engineered inactivating modification comprises:
a) at least one engineered inactivating mutation in the at least one endogenous methionine synthesis gene;
b) at least one engineered inactivating mutation in a promoter operatively linked to the at least one endogenous methionine synthesis gene; and/or
c) at least one silencing RNA molecule specific to at least one messenger RNA (mRNA) expressed by the at least one endogenous methionine synthesis gene.

22. An engineered probiotic microorganism for reducing bioavailable methionine levels, the microorganism comprising:
a) at least one endogenous methionine synthesis gene comprising at least one engineered inactivating modification;
b) at least one copy of an exogenous gene encoding a glycine N-methyltransferase (GNMT) enzyme;
c) at least one copy of an exogenous gene encoding a sarcosine N-methyl transferase (SNMT) enzyme;
d) at least one copy of an exogenous gene encoding a sulfinoalanine decarboxylase enzyme; and
e) at least one copy of an exogenous gene encoding a Flavin-containing monooxygenase (FMO) enzyme;
wherein the engineered probiotic microorganism expresses endogenously or exogenously encoded methionine adenosyl transferase (MetK), adenosylhomocysteinase (ahcY), cystathionine β-synthase, cystathionine gamma lyase and cysteine dioxygenase enzymes.

23. The engineered probiotic microorganism of claim 19 or 22, wherein the FMO enzyme is an FMO1, FMO2 or FMO3 enzyme that catalyzes the catalysis of the conversion of hypotaurine to taurine.

24. The engineered probiotic microorganism of claim 19 or 22, which metabolizes methionine to taurine.

25. The engineered probiotic microorganism of claim 19 or 22, wherein the at least one engineered inactivating modification comprises:
a) at least one engineered inactivating mutation in the at least one endogenous methionine synthesis gene;
b) at least one engineered inactivating mutation in a promoter operatively linked to the at least one endogenous methionine synthesis gene; and/or
c) at least one silencing RNA molecule specific to at least one messenger RNA (mRNA) expressed by the at least one endogenous methionine synthesis gene.

26. The engineered probiotic microorganism of claim 19 or 22, wherein the at least one endogenous methionine synthesis gene is MetE and/or MetH.

27. A pharmaceutical composition comprising an engineered probiotic microorganism of any one of claims 1-3, 19, or 22, and a pharmaceutically acceptable carrier.

28. The pharmaceutical composition of claim 27, wherein the purified mixture of live bacteria comprises species present in an amount of at least about 1×10⁸ CFUs/ml.

29. The pharmaceutical composition of claim 27, wherein the pharmaceutical composition is formulated for oral administration.

30. The pharmaceutical composition of claim 27, wherein the pharmaceutical composition is formulated for delivery to the gut via oral administration.

31. The pharmaceutical composition of claim 27, wherein the pharmaceutical composition is enteric coated.

32. The pharmaceutical composition of claim 27, wherein the pharmaceutical composition is formulated for injection.

33. The pharmaceutical composition of claim 27, wherein the pharmaceutical composition further comprises at least one additional methionine-decreasing or homocysteine-decreasing therapeutic.

34. The pharmaceutical composition of claim 27, wherein the pharmaceutical composition is co-administered with at least one additional methionine-decreasing or homocysteine-decreasing therapeutic.

35. The pharmaceutical composition of claim 34, wherein the at least one additional methionine-decreasing or homocysteine-decreasing therapeutic is selected from the group consisting of: betaine, taurine, a methionine restriction diet, a methionine-free formula, and combinations thereof.

36. A food composition comprising an engineered probiotic microorganism of any one of claims 1-3, 19, or 22.

37. A probiotic dietary supplement comprising an engineered probiotic microorganism of any one of claims 1-3, 19, or 22.

38. A method of reducing bioavailable methionine in a mammal in need thereof, the method comprising administering an engineered probiotic microorganism of any one of claims 1-3, 19, or 22, or administering a pharmaceutical composition, a food composition, or a probiotic dietary supplement comprising an engineered probiotic microorganism of any one of claims 1-3, 19, or 22, to the mammal.

39. The method of claim 38, wherein the administering is oral or rectal.

40. The method of claim 38, wherein the administering is by injection.

41. The method of claim 38, wherein the administering reduced the level of bioavailable methionine in the gut of the mammal.

42. The method of claim 38, wherein the method further comprises administering an effective amount of at least one additional methionine-decreasing or homocysteine-decreasing therapeutic.

43. The method of claim 42, wherein the at least one additional methionine-decreasing or homocysteine-decreasing therapeutic is selected from the group consisting of: betaine, taurine, a methionine restriction diet, a methionine-free formula, and combinations thereof.

44. A method of treating a cancer in a subject in need thereof, the method comprising administering an effective amount of an engineered probiotic microorganism of any one of claims 1-3.

45. The method of claim 44, wherein the cancer is a methionine-dependent cancer.

46. The method of claim 44, wherein the cancer is selected from the group consisting of: glioma colon cancer, breast cancer, ovarian cancer, prostate cancer, melanoma, and sarcoma.

47. The method of claim 44, wherein the cancer is a glioma.

48. The method of claim 44, wherein the method further comprises administering an effective amount of at least one additional methionine-decreasing or homocysteine-decreasing therapeutic.

49. The method of claim 44, wherein the at least one additional methionine-decreasing or homocysteine-decreasing therapeutic is selected from the group consisting of: betaine, taurine, a methionine restriction diet, a methionine-free formula, and combinations thereof.

50. The method of claim 44, wherein the method further comprises administering an effective amount of at least one additional cancer therapeutic.

51. The method of claim 44, wherein the administering is by injection.

52. A method of reducing a level of methanethiol, the method comprising contacting methanethiol with a probiotic microorganism that encodes and expresses an exogenous gene encoding a methanethiol catabolizing enzyme.

53. The method of claim 52, wherein the methanethiol catabolizing enzyme is an esterase.

54. The method of claim 52, wherein the methanethiol catabolizing enzyme is a methanethiol oxidase.

55. The method of claim 52, wherein the methanethiol is produced by an engineered probiotic microorganism that comprises and expresses an exogenous gene encoding an enzyme that catalyzes the degradation of methionine to products including methanethiol.

56. The method of claim 55, wherein the enzyme that catalyzes the degradation of methionine to products including methanethiol comprises a methionine gamma lyase enzyme.

57. A method of reducing odor produced by a population of gut microbiota that produced methanethiol, the method comprising introducing an engineered probiotic microorganism to the gut microbiota, wherein the engineered probiotic microorganism encodes and expresses an exogenous gene encoding a methanethiol catabolizing enzyme.

58. The method of claim 57, wherein the methanethiol catabolizing enzyme is an esterase.

59. The method of claim 57, wherein the methanethiol catabolizing enzyme is a methanethiol oxidase.

60. The method of claim 57, wherein the methanethiol is produced by an engineered probiotic microorganism that comprises and expresses an exogenous gene encoding an enzyme that catalyzes the degradation of methionine to products including methanethiol.

61. The method of claim 60, wherein the enzyme that catalyzes the degradation of methionine to products including methanethiol comprises a methionine gamma lyase enzyme.

62. A method of generating taurine from methionine in the gut of a mammal, the method comprising introducing an engineered probiotic microorganism of claim 19 or 22 to the gut of the mammal.

63. The method of claim 63, wherein the microorganism is introduced via oral administration.


SEQUENCE LISTING (435 sequences)

SEQ ID NO: 1 - DNA, 1200 bp, Oscillibacter rumenantium
  source 1..1200: mol_type = unassigned DNA
atgatggaaaacgaacaggaactgggctttgcgacccgccagattcatgtgggcaaaattaaagaagcgagcggcgcgctgtgcaccccgatttatcagaccagcacctttgaatttgaaaccgtgcagcagggcggcgcgcgctttgcgggcgaagaaccgggctatatttatagccgcctgagcaacccgagcctggcgcaggcggaagaaaaactggcgagcctggaaaacggcgaagcggcgctggcggcggcgagcggcatgggcgcgattagcgcggcgctgtggaccagcgtggtggcgggcgatgaaattctggcgagcgataccctgtatggctgcacctttagcctgctgtgccatggcatgaccaaatttggcgtggataccaaatttattgatatggtgaacctggaaaactttcagaaacatctgaccccgaaaaccaaagtgatttattttgaaaccccgtgcaacccgaccctgaaaattctggatattcgcgcgattgcggaagcggcgcataaatataacccggcgattcgcgtgatggtggataacaccttttgcagcccgtatctgcagcgcccgctggaactgggcgcggatgtggtggtgcatagcgcgaccaaatatattaacggccatggcgatgtgattgcgggctttattgtgggcaccgcggaatttattggccagtgccgcacctttggcctgaaagatatgaccggcgcggtgatgagcccgtttgatgcgtttctgattgcgcgcggcctgaaaaccctggatattcgcatggaacgccattgcagcaacgcgcgcaaagtggcggaatttctgcatagccatccggcggtggaaaaagtgtattatccgggcctgccggattttaaaggctatgaagtggcgcagaaacagatgaaagattttggcggcatgctgagcattgaactgaaagcgagccgcgaagaagtggcgaacgcgctgaacaacctgcgcctgtgcaccattgcggtgagcctgggcgatgcggaaaccctggtggaacatgcggcgagcatgacccatagcacctataccccggaagaactggcggcggcgggcattagcgaaggcctggtgcgcattagcgtgggcctggaagatccggatgatattattgcggatctgaaaagcgtgctggataccctggtgagc

SEQ ID NO: 2 - DNA, 1200 bp, synthetic construct
  source 1..1200: mol_type = other DNA
atgatggaaaatgaacaggagcttggtttcgccacaaggcagatacatgtcggcaagattaaagaggcttcgggtgcgttgtgtaccccgatctaccaaacttctacgtttgaattcgaaactgtgcaacaaggaggtgcgcgctttgccggcgaagaaccgggctatatttattcacgcttaagcaacccttcccttgcccaggcagaggagaaactcgcctcactggaaaacggagaagcggctctggcagcggcgtcgggaatgggcgcaatatcagccgccctgtggacctctgttgtcgctggcgatgaaatccttgcttcagataccctgtatggatgcacgttttcattactatgccatggaatgaccaaatttggtgtagatactaaatttattgatatggtaaaccttgaaaactttcagaagcaccttacgccgaaaacgaaagtgatttactttgaaacgccttgcaacccgacactcaaaattttggatatccgtgcaatcgcagaagctgctcataaatataatccagcaattcgagttatggtcgataatacattttgtagcccctatttgcaacggccgcttgaactgggcgccgatgtcgtagtgcacagcgccacaaagtacatcaatggccacggtgacgtgattgcaggcttcattgttgggactgctgagttcattgggcaatgccggacatttggactgaaagacatgaccggggcagtcatgtctccgttcgatgcttttctaatcgctcgtggcttaaaaacattggacattcgtatggaaagacattgtagtaatgcgagaaaggtagcagagttcctgcactcccatccagcggttgaaaaagtctattatccagggcttcctgactttaagggatatgaagttgctcagaaacaaatgaaagattttggtgggatgttaagtatcgaactgaaggcgtcaagagaagaagtggcgaatgcattgaataacttaagactctgtacaattgcggtcagcctcggcgacgctgaaacgttagtagagcatgcggcgagtatgacacattccacatacacacctgaggagctggcagccgccggaatcagcgagggacttgttcgcatatctgtgggattagaagatccggacgacattatcgcagatcttaaatctgttttggatacgttagtgagc

SEQ ID NO: 3 - DNA, 1203 bp, Treponema denticola
  source 1..1203: mol_type = unassigned DNA
atgaatcgcaaagagctggaaaaactggggtttgcgtctaaacaaatccacgcgggcagcatcaaaaataagtacggtgctctggctacccccatttaccagacctcgactttcgcttttgattccgctgaacaaggtggccgtcgtttcgcactggaggaggagggttatatctacacacgtttggggaatccgaccacaactgtggttgaagaaaaacttgcatgtcttgaaaacggcgaggcttgtatgagcgcttcttctgggattggggccgttacgtcgtgcatctggagcatcgttaacgctggcgaccatattgtagccggcaagaccttgtacgggtgtacatttgcattcctgaatcatgggttgagccgctttggagttgatgttacttttgtcgatacccgcgatcccgagaatgtaaagaaagccttgaaacccaacaccaaaatcgtttatctggaaacgccagcgaacccgaacatgtatttgtgtgatattgcagctgtttccaaaattgcgcatgcccacaacccggagtgcaaggtcatcgttgataacacgtatatgaccccgtacctgcagcggccccttgatctgggggccgatgtggtgctgcacagtgcaaccaaatatctgaacggccatggcgatgtcatcgccggtttcgtggtcggcaaaaaagagtttattgatcaggtgcggtttgtaggcgttaaggacatgacgggctctacactgggtcctttcgaagcgtacctgatcggccgcggaatgaaaacactggacattcggatggaaaaacactgcgccaatgctcaaaaagtagcggagttcttggaaaaacacccagcggttgagagcatcgctttccctggtctgaaatccttcccacagtatgaactcgccaagaaacagatgaagctctgtggtgcgatgattgcgttcaccgtaaaagggggccttgaagctggtaaaactctcatcaactccgttaagttcgccactattgccgtgtcgctcggcgatgccgagaccctgattcaacatccggcaagcatgactcattccccatacaccccagaggagcgcgcagcatccgacattgccgagggcctggtccgcttaagtgtaggtctggaagatgccgaagatattattgccgatctgaaacaagctctggataaacttgtaaaa

SEQ ID NO: 4 - DNA, 1203 bp, synthetic construct
  source 1..1203: mol_type = other DNA
atgaaccggaaagagcttgaaaaattgggctttgcaagcaaacaaattcatgcagggagcattaaaaataaatacggagccctggccacaccgatctaccagaccagcacattcgcgtttgattcagctgagcaaggcggccggagattcgcattagaagaagaaggatatatatatacgcgcctcgggaatcctacaacgacagtagttgaggagaaactggcatgtttagaaaatggagaagcatgtatgtccgcatcatcaggcattggcgcagtgactagttgtatctggtctatcgttaatgcgggtgatcacattgtcgccggtaaaacgttatatggctgcacgtttgcttttctgaaccacggtttaagtcgtttcggggtcgatgtaacctttgtggatacaagggatcctgaaaatgtaaagaaggcccttaagccgaatacaaaaattgtctatttggagacaccagcaaacccgaacatgtatctctgcgatatcgcggcggttagcaagattgcccatgcccataatcctgagtgtaaagtgatcgtagacaacacctatatgacgccttatttgcagcgtccgttggacttgggagctgatgtggtgctccactctgcgacaaagtacctcaacggacatggtgatgtcatagccggttttgtggttggcaagaaagaatttattgaccaggtgagattcgtcggagttaaggacatgacgggctcaacgctgggaccgtttgaagcataccttatcggacgcgggatgaaaacccttgatattagaatggaaaaacattgcgcaaatgcacaaaaagtggcggaatttctggaaaaacatccagctgttgaatcaattgctttccctgggctaaaatcgtttccgcagtacgagctggctaagaaacaaatgaaactttgcggggccatgatcgcgtttacagtaaaaggaggccttgaagcgggcaaaacacttataaactccgtcaagtttgccactatcgctgtttccctgggcgacgcggagactctgattcagcacccggccagcatgactcattctccctataccccagaagagcgagctgcttctgatattgctgaaggtttggtacgcttatcggttggattagaagatgcggaagatatcattgcagacttaaaacaagcgcttgacaaactagtcaaa

SEQ ID NO: 5 - protein, 400 aa, Oscillibacter rumenantium
MMENEQELGFATRQIHVGKIKEASGALCTPIYQTSTFEFETVQQGGARFAGEEPGYIYSRLSNPSLAQAEEKLASLENGEAALAAASGMGAISAALWTSVVAGDEILASDTLYGCTFSLLCHGMTKFGVDTKFIDMVNLENFQKHLTPKTKVIYFETPCNPTLKILDIRAIAEAAHKYNPAIRVMVDNTFCSPYLQRPLELGADVVVHSATKYINGHGDVIAGFIVGTAEFIGQCRTFGLKDMTGAVMSPFDAFLIARGLKTLDIRMERHCSNARKVAEFLHSHPAVEKVYYPGLPDFKGYEVAQKQMKDFGGMLSIELKASREEVANALNNLRLCTIAVSLGDAETLVEHAASMTHSTYTPEELAAAGISEGLVRISVGLEDPDDIIADLKSVLDTLVS

SEQ ID NO: 6 - protein, 401 aa, Treponema denticola
MNRKELEKLGFASKQIHAGSIKNKYGALATPIYQTSTFAFDSAEQGGRRFALEEEGYIYTRLGNPTTTVVEEKLACLENGEACMSASSGIGAVTSCIWSIVNAGDHIVAGKTLYGCTFAFLNHGLSRFGVDVTFVDTRDPENVKKALKPNTKIVYLETPANPNMYLCDIAAVSKIAHAHNPECKVIVDNTYMTPYLQRPLDLGADVVLHSATKYLNGHGDVIAGFVVGKKEFIDQVRFVGVKDMTGSTLGPFEAYLIGRGMKTLDIRMEKHCANAQKVAEFLEKHPAVESIAFPGLKSFPQYELAKKQMKLCGAMIAFTVKGGLEAGKTLINSVKFATIAVSLGDAETLIQHPASMTHSPYTPEERAASDIAEGLVRLSVGLEDAEDIIADLKQALDKLVK

SEQ ID NO: 7 - DNA, 2610 bp, synthetic construct
  source 1..2610: mol_type = other DNA
atgatggaaaacgaacaggaactgggctttgcgacccgccagattcatgtgggcaaaattaaagaagcgagcggcgcgctgtgcaccccgatttatcagaccagcacctttgaatttgaaaccgtgcagcagggcggcgcgcgctttgcgggcgaagaaccgggctatatttatagccgcctgagcaacccgagcctggcgcaggcggaagaaaaactggcgagcctggaaaacggcgaagcggcgctggcggcggcgagcggcatgggcgcgattagcgcggcgctgtggaccagcgtggtggcgggcgatgaaattctggcgagcgataccctgtatggctgcacctttagcctgctgtgccatggcatgaccaaatttggcgtggataccaaatttattgatatggtgaacctggaaaactttcagaaacatctgaccccgaaaaccaaagtgatttattttgaaaccccgtgcaacccgaccctgaaaattctggatattcgcgcgattgcggaagcggcgcataaatataacccggcgattcgcgtgatggtggataacaccttttgcagcccgtatctgcagcgcccgctggaactgggcgcggatgtggtggtgcatagcgcgaccaaatatattaacggccatggcgatgtgattgcgggctttattgtgggcaccgcggaatttattggccagtgccgcacctttggcctgaaagatatgaccggcgcggtgatgagcccgtttgatgcgtttctgattgcgcgcggcctgaaaaccctggatattcgcatggaacgccattgcagcaacgcgcgcaaagtggcggaatttctgcatagccatccggcggtggaaaaagtgtattatccgggcctgccggattttaaaggctatgaagtggcgcagaaacagatgaaagattttggcggcatgctgagcattgaactgaaagcgagccgcgaagaagtggcgaacgcgctgaacaacctgcgcctgtgcaccattgcggtgagcctgggcgatgcggaaaccctggtggaacatgcggcgagcatgacccatagcacctataccccggaagaactggcggcggcgggcattagcgaaggcctggtgcgcattagcgtgggcctggaagatccggatgatattattgcggatctgaaaagcgtgctggataccctggtgagcggcagcagcggcagcagcggcagcagcggcagcagcggcagcagcggcagcagcggcagcagcggcagcagcggcagcagcggcagcagcggcagcagcggcagcagcatgaaaaaacatctgctggcgggcgcgtgcgcgctggcgatgggctttgcggtgattccgggcacctttgcggatgaaacctgcaacagcccgtttaccaccgcgctgattaccggccaggaacagtatctgcatgtgtggaccctgggcatgccgggcgtgggcgatgaaagcgataaactggtgaccattagcgtggatccgaaaagcgataaatatggcaaagtgattaacaccctgagcgtgggcggccgcggcgaagcgcatcataccggctttaccgatgatcgccgctatctgtgggcgggccgcctggatgataacaaaatttttatttttgatctgattgatccggcgaacccgaaactgattaaaaccattaccgattttgcggatcgcaccggctatgtgggcccgcataccttttatgcgctgccgggccgcatgctgattcaggcgctgagcaacaccaaaacccatgatggccagaccggcctggcggtgtatagcaacgcgggcgaactggtgagcctgcatccgatgccggtgaccgatggcggcgatggctatggctatgatattggcattaacccggcgaaaaacgtgctgctgaccagcagctttaccggctggaacaactatatgatggatctgggcaaaatggtgaaagatccggaagcgatgaaacgctttggcaacaccatggcgatttgggatctgaaaagcatgaaagcggaaaaaattctgaacgtgccgggcgcgccgctggaaattcgctggagcctgaaaccggaacataactgggcgtataccgcgaccctgaccagcaaactgtggctgattaaacaggatgataaaggcgaatggattgcgaaagaaaccggcaccattggcgatccgagcaaaattccgctgccggtggatattagcattaccgcggatgcgaaaggcctgtgggtgaacacctttctggatggcaccacccgcttttatgatattagcgaaccggaacatccgaaagaagtgtttagcaaaaaaatgggcaaccaggtgaacatggtgagccagagctatgatggcaaacgcgtgtattttaccaccagcctgattgcgaactgggataaaaaaggcgcggaaaacgatcagtggctgaaagcgtatgattgggatggcaaagaactggtggaaaaatttaccgtggattttaacgaactgaaactgggccgcgcgcatcatatgaaatttagcagcaaaaccaacgcggcggaactgggcaccaaccagagctttccgacccgccag

SEQ ID NO: 8 - DNA, 2610 bp, synthetic construct
  source 1..2610: mol_type = other DNA
atgaaaaaacatctgctggcgggcgcgtgcgcgctggcgatgggctttgcggtgattccgggcacctttgcggatgaaacctgcaacagcccgtttaccaccgcgctgattaccggccaggaacagtatctgcatgtgtggaccctgggcatgccgggcgtgggcgatgaaagcgataaactggtgaccattagcgtggatccgaaaagcgataaatatggcaaagtgattaacaccctgagcgtgggcggccgcggcgaagcgcatcataccggctttaccgatgatcgccgctatctgtgggcgggccgcctggatgataacaaaatttttatttttgatctgattgatccggcgaacccgaaactgattaaaaccattaccgattttgcggatcgcaccggctatgtgggcccgcataccttttatgcgctgccgggccgcatgctgattcaggcgctgagcaacaccaaaacccatgatggccagaccggcctggcggtgtatagcaacgcgggcgaactggtgagcctgcatccgatgccggtgaccgatggcggcgatggctatggctatgatattggcattaacccggcgaaaaacgtgctgctgaccagcagctttaccggctggaacaactatatgatggatctgggcaaaatggtgaaagatccggaagcgatgaaacgctttggcaacaccatggcgatttgggatctgaaaagcatgaaagcggaaaaaattctgaacgtgccgggcgcgccgctggaaattcgctggagcctgaaaccggaacataactgggcgtataccgcgaccctgaccagcaaactgtggctgattaaacaggatgataaaggcgaatggattgcgaaagaaaccggcaccattggcgatccgagcaaaattccgctgccggtggatattagcattaccgcggatgcgaaaggcctgtgggtgaacacctttctggatggcaccacccgcttttatgatattagcgaaccggaacatccgaaagaagtgtttagcaaaaaaatgggcaaccaggtgaacatggtgagccagagctatgatggcaaacgcgtgtattttaccaccagcctgattgcgaactgggataaaaaaggcgcggaaaacgatcagtggctgaaagcgtatgattgggatggcaaagaactggtggaaaaatttaccgtggattttaacgaactgaaactgggccgcgcgcatcatatgaaatttagcagcaaaaccaacgcggcggaactgggcaccaaccagagctttccgacccgccagggcagcagcggcagcagcggcagcagcggcagcagcggcagcagcggcagcagcggcagcagcggcagcagcggcagcagcggcagcagcggcagcagcggcagcagcatgatggaaaacgaacaggaactgggctttgcgacccgccagattcatgtgggcaaaattaaagaagcgagcggcgcgctgtgcaccccgatttatcagaccagcacctttgaatttgaaaccgtgcagcagggcggcgcgcgctttgcgggcgaagaaccgggctatatttatagccgcctgagcaacccgagcctggcgcaggcggaagaaaaactggcgagcctggaaaacggcgaagcggcgctggcggcggcgagcggcatgggcgcgattagcgcggcgctgtggaccagcgtggtggcgggcgatgaaattctggcgagcgataccctgtatggctgcacctttagcctgctgtgccatggcatgaccaaatttggcgtggataccaaatttattgatatggtgaacctggaaaactttcagaaacatctgaccccgaaaaccaaagtgatttattttgaaaccccgtgcaacccgaccctgaaaattctggatattcgcgcgattgcggaagcggcgcataaatataacccggcgattcgcgtgatggtggataacaccttttgcagcccgtatctgcagcgcccgctggaactgggcgcggatgtggtggtgcatagcgcgaccaaatatattaacggccatggcgatgtgattgcgggctttattgtgggcaccgcggaatttattggccagtgccgcacctttggcctgaaagatatgaccggcgcggtgatgagcccgtttgatgcgtttctgattgcgcgcggcctgaaaaccctggatattcgcatggaacgccattgcagcaacgcgcgcaaagtggcggaatttctgcatagccatccggcggtggaaaaagtgtattatccgggcctgccggattttaaaggctatgaagtggcgcagaaacagatgaaagattttggcggcatgctgagcattgaactgaaagcgagccgcgaagaagtggcgaacgcgctgaacaacctgcgcctgtgcaccattgcggtgagcctgggcgatgcggaaaccctggtggaacatgcggcgagcatgacccatagcacctataccccggaagaactggcggcggcgggcattagcgaaggcctggtgcgcattagcgtgggcctggaagatccggatgatattattgcggatctgaaaagcgtgctggataccctggtgagc

SEQ ID NO: 9 - protein, 870 aa, synthetic construct
MMENEQELGFATRQIHVGKIKEASGALCTPIYQTSTFEFETVQQGGARFAGEEPGYIYSRLSNPSLAQAEEKLASLENGEAALAAASGMGAISAALWTSVVAGDEILASDTLYGCTFSLLCHGMTKFGVDTKFIDMVNLENFQKHLTPKTKVIYFETPCNPTLKILDIRAIAEAAHKYNPAIRVMVDNTFCSPYLQRPLELGADVVVHSATKYINGHGDVIAGFIVGTAEFIGQCRTFGLKDMTGAVMSPFDAFLIARGLKTLDIRMERHCSNARKVAEFLHSHPAVEKVYYPGLPDFKGYEVAQKQMKDFGGMLSIELKASREEVANALNNLRLCTIAVSLGDAETLVEHAASMTHSTYTPEELAAAGISEGLVRISVGLEDPDDIIADLKSVLDTLVSGSSGSSGSSGSSGSSGSSGSSGSSGSSGSSGSSGSSMKKHLLAGACALAMGFAVIPGTFADETCNSPFTTALITGQEQYLHVWTLGMPGVGDESDKLVTISVDPKSDKYGKVINTLSVGGRGEAHHTGFTDDRRYLWAGRLDDNKIFIFDLIDPANPKLIKTITDFADRTGYVGPHTFYALPGRMLIQALSNTKTHDGQTGLAVYSNAGELVSLHPMPVTDGGDGYGYDIGINPAKNVLLTSSFTGWNNYMMDLGKMVKDPEAMKRFGNTMAIWDLKSMKAEKILNVPGAPLEIRWSLKPEHNWAYTATLTSKLWLIKQDDKGEWIAKETGTIGDPSKIPLPVDISITADAKGLWVNTFLDGTTRFYDISEPEHPKEVFSKKMGNQVNMVSQSYDGKRVYFTTSLIANWDKKGAENDQWLKAYDWDGKELVEKFTVDFNELKLGRAHHMKFSSKTNAAELGTNQSFPTRQ

SEQ ID NO: 10 - protein, 870 aa, synthetic construct
MKKHLLAGACALAMGFAVIPGTFADETCNSPFTTALITGQEQYLHVWTLGMPGVGDESDKLVTISVDPKSDKYGKVINTLSVGGRGEAHHTGFTDDRRYLWAGRLDDNKIFIFDLIDPANPKLIKTITDFADRTGYVGPHTFYALPGRMLIQALSNTKTHDGQTGLAVYSNAGELVSLHPMPVTDGGDGYGYDIGINPAKNVLLTSSFTGWNNYMMDLGKMVKDPEAMKRFGNTMAIWDLKSMKAEKILNVPGAPLEIRWSLKPEHNWAYTATLTSKLWLIKQDDKGEWIAKETGTIGDPSKIPLPVDISITADAKGLWVNTFLDGTTRFYDISEPEHPKEVFSKKMGNQVNMVSQSYDGKRVYFTTSLIANWDKKGAENDQWLKAYDWDGKELVEKFTVDFNELKLGRAHHMKFSSKTNAAELGTNQSFPTRQGSSGSSGSSGSSGSSGSSGSSGSSGSSGSSGSSGSSMMENEQELGFATRQIHVGKIKEASGALCTPIYQTSTFEFETVQQGGARFAGEEPGYIYSRLSNPSLAQAEEKLASLENGEAALAAASGMGAISAALWTSVVAGDEILASDTLYGCTFSLLCHGMTKFGVDTKFIDMVNLENFQKHLTPKTKVIYFETPCNPTLKILDIRAIAEAAHKYNPAIRVMVDNTFCSPYLQRPLELGADVVVHSATKYINGHGDVIAGFIVGTAEFIGQCRTFGLKDMTGAVMSPFDAFLIARGLKTLDIRMERHCSNARKVAEFLHSHPAVEKVYYPGLPDFKGYEVAQKQMKDFGGMLSIELKASREEVANALNNLRLCTIAVSLGDAETLVEHAASMTHSTYTPEELAAAGISEGLVRISVGLEDPDDIIADLKSVLDTLVS

SEQ ID NO: 11 - DNA, 1029 bp, Escherichia coli
  source 1..1029: mol_type = unassigned DNA
atgattaaactgagcaacattaccaaagtgtttcatcagggcacccgcaccattcaggcgctgaacaacgtgagcctgcatgtgccggcgggccagatttatggcgtgattggcgcgagcggcgcgggcaaaagcaccctgattcgctgcgtgaacctgctggaacgcccgaccgaaggcagcgtgctggtggatggccaggaactgaccaccctgagcgaaagcgaactgaccaaagcgcgccgccagattggcatgatttttcagcattttaacctgctgagcagccgcaccgtgtttggcaacgtggcgctgccgctggaactggataacaccccgaaagatgaagtgaaacgccgcgtgaccgaactgctgagcctggtgggcctgggcgataaacatgatagctatccgagcaacctgagcggcggccagaaacagcgcgtggcgattgcgcgcgcgctggcgagcaacccgaaagtgctgctgtgcgatgaagcgaccagcgcgctggatccggcgaccacccgcagcattctggaactgctgaaagatattaaccgccgcctgggcctgaccattctgctgattacccatgaaatggatgtggtgaaacgcatttgcgattgcgtggcggtgattagcaacggcgaactgattgaacaggataccgtgagcgaagtgtttagccatccgaaaaccccgctggcgcagaaatttattcagagcaccctgcatctggatattccggaagattatcaggaacgcctgcaggcggaaccgtttaccgattgcgtgccgatgctgcgcctggaatttaccggccagagcgtggatgcgccgctgctgagcgaaaccgcgcgccgctttaacgtgaacaacaacattattagcgcgcagatggattatgcgggcggcgtgaaatttggcattatgctgaccgaaatgcatggcacccagcaggatacccaggcggcgattgcgtggctgcaggaacatcatgtgaaagtggaagtgctgggctatgtg

SEQ ID NO: 12 - DNA, 1029 bp, synthetic construct
  source 1..1029: mol_type = other DNA
atgattaaactgagcaacattaccaaagtgtttcatcagggcacccgcaccattcaggcgctgaacaacgtgagcctgcatgtgccggcgggccagatttatggcgtgattggcgcgagcggcgcgggcaaaagcaccctgattcgctgcgtgaacctgctggaacgcccgaccgaaggcagcgtgctggtggatggccaggaactgaccaccctgagcgaaagcgaactgaccaaagcgcgccgccagattggcatgatttttcagcattttaacctgctgagcagccgcaccgtgtttggcaacgtggcgctgccgctggaactggataacaccccgaaagatgaagtgaaacgccgcgtgaccgaactgctgagcctggtgggcctgggcgataaacatgatagctatccgagcaacctgagcggcggccagaaacagcgcgtggcgattgcgcgcgcgctggcgagcaacccgaaagtgctgctgtgcgatgaagcgaccagcgcgctggatccggcgaccacccgcagcattctggaactgctgaaagatattaaccgccgcctgggcctgaccattctgctgattacccatgaaatggatgtggtgaaacgcatttgcgattgcgtggcggtgattagcaacggcgaactgattgaacaggataccgtgagcgaagtgtttagccatccgaaaaccccgctggcgcagaaatttattcagagcaccctgcatctggatattccggaagattatcaggaacgcctgcaggcggaaccgtttaccgattgcgtgccgatgctgcgcctggaatttaccggccagagcgtggatgcgccgctgctgagcgaaaccgcgcgccgctttaacgtgaacaacgcgattattagcgcgcagatggattatgcgggcggcgtgaaatttggcattatgctgaccgaaatgcatggcacccagcaggatacccaggcggcgattgcgtggctgcaggaacatcatgtgaaagtggaagtgctgggctatgtg

SEQ ID NO: 13 - protein, 343 aa, Escherichia coli
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEVKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAPLLSETARRFNVNNNIISAQMDYAGGVKFGIMLTEMHGTQQDTQAAIAWLQEHHVKVEVLGYV

SEQ ID NO: 14 - protein, 343 aa, Synthetic construct
MIKLSNITKVFHQGTRTIQALNNVSLHVPAGQIYGVIGASGAGKSTLIRCVNLLERPTEGSVLVDGQELTTLSESELTKARRQIGMIFQHFNLLSSRTVFGNVALPLELDNTPKDEVKRRVTELLSLVGLGDKHDSYPSNLSGGQKQRVAIARALASNPKVLLCDEATSALDPATTRSILELLKDINRRLGLTILLITHEMDVVKRICDCVAVISNGELIEQDTVSEVFSHPKTPLAQKFIQSTLHLDIPEDYQERLQAEPFTDCVPMLRLEFTGQSVDAPLLSETARRFNVNNAIISAQMDYAGGVKFGIMLTEMHGTQQDTQAAIAWLQEHHVKVEVLGYV

SEQ ID NO: 15 - DNA, 651 bp, Escherichia coli
  source 1..651: mol_type = unassigned DNA
atgagcgaaccgatgatgtggctgctggtgcgcggcgtgtgggaaaccctggcgatgacctttgtgagcggcttttttggctttgtgattggcctgccggtgggcgtgctgctgtatgtgacccgcccgggccagattattgcgaacgcgaaactgtatcgcaccgtgagcgcgattgtgaacatttttcgcagcattccgtttattattctgctggtgtggatgattccgtttacccgcgtgattgtgggcaccagcattggcctgcaggcggcgattgtgccgctgaccgtgggcgcggcgccgtttattgcgcgcatggtggaaaacgcgctgctggaaattccgaccggcctgattgaagcgagccgcgcgatgggcgcgaccccgatgcagattgtgcgcaaagtgctgctgccggaagcgctgccgggcctggtgaacgcggcgaccattaccctgattaccctggtgggctatagcgcgatgggcggcgcggtgggcgcgggcggcctgggccagattggctatcagtatggctatattggctataacgcgaccgtgatgaacaccgtgctggtgctgctggtgattctggtgtatctgattcagtttgcgggcgatcgcattgtgcgcgcggtgacccgcaaa

SEQ ID NO: 16 - DNA, 651 bp, Synthetic construct
  source 1..651: mol_type = other DNA
atgagcgaaccgatgatgtggctgctggtgcgcggcgtgtgggaaaccctggcgatgacctttgtgagcggcttttttggctttgtgattggcctgccggtgggcgtgctgctgtatgtgacccgcccgggccagattattgcgaacgcgaaactgtatcgcaccgtgagcgcgattgtgaacatttttcgcagcattccgtttattattctgctggtgtggatgattccgtttacccgcgtgattgtgggcaccagcattggcctgcaggcggcgattgtgccgctgaccgtgggcgcggcgccgtttattgcgcgcatggtggaaaacgcgctgctggaaattccgaccggcctgattgaagcgagccgcgcgatgggcgcgaccccgatgcagattgtgcgcaaagtgctgctgccggaagcgctgccgggcctggtgaacgcggcgaccattaccctgattaccctggtgggcgcgagcgcgatgggcggcgcggtgggcgcgggcggcctgggccagattggctatcagtatggctatattggctataacgcgaccgtgatgaacaccgtgctggtgctgctggtgattctggtgtatctgattcagtttgcgggcgatcgcattgtgcgcgcggtgacccgcaaa

SEQ ID NO: 17 - protein, 217 aa, Escherichia coli
MSEPMMWLLVRGVWETLAMTFVSGFFGFVIGLPVGVLLYVTRPGQIIANAKLYRTVSAIVNIFRSIPFIILLVWMIPFTRVIVGTSIGLQAAIVPLTVGAAPFIARMVENALLEIPTGLIEASRAMGATPMQIVRKVLLPEALPGLVNAATITLITLVGYSAMGGAVGAGGLGQIGYQYGYIGYNATVMNTVLVLLVILVYLIQFAGDRIVRAVTRK

SEQ ID NO: 18 - protein, 217 aa, synthetic construct
MSEPMMWLLVRGVWETLAMTFVSGFFGFVIGLPVGVLLYVTRPGQIIANAKLYRTVSAIVNIFRSIPFIILLVWMIPFTRVIVGTSIGLQAAIVPLTVGAAPFIARMVENALLEIPTGLIEASRAMGATPMQIVRKVLLPEALPGLVNAATITLITLVGASAMGGAVGAGGLGQIGYQYGYIGYNATVMNTVLVLLVILVYLIQFAGDRIVRAVTRK

SEQ ID NO: 19 - DNA, 813 bp, Escherichia coli
  source 1..813: mol_type = unassigned DNA
atggcgtttaaatttaaaacctttgcggcggtgggcgcgctgattggcagcctggcgctggtgggctgcggccaggatgaaaaagatccgaaccatattaaagtgggcgtgattgtgggcgcggaacagcaggtggcggaagtggcgcagaaagtggcgaaagataaatatggcctggatgtggaactggtgacctttaacgattatgtgctgccgaacgaagcgctgagcaaaggcgatattgatgcgaacgcgtttcagcataaaccgtatctggatcagcagctgaaagatcgcggctataaactggtggcggtgggcaacacctttgtgtatccgattgcgggctatagcaaaaaaattaaaagcctggatgaactgcaggatggcagccaggtggcggtgccgaacgatccgaccaacctgggccgcagcctgctgctgctgcagaaagtgggcctgattaaactgaaagatggcgtgggcctgctgccgaccgtgctggatgtggtggaaaacccgaaaaacctgaaaattgtggaactggaagcgccgcagctgccgcgcagcctggatgatgcgcagattgcgctggcggtgattaacaccacctatgcgagccagattggcctgaccccggcgaaagatggcatttttgtggaagataaagaaagcccgtatgtgaacctgattgtgacccgcgaagataacaaagatgcggaaaacgtgaaaaaatttgtgcaggcgtatcagagcgatgaagtgtatgaagcggcgaacaaagtgtttaacggcggcgcggtgaaaggctgg

SEQ ID NO: 20 - DNA, 813 bp, synthetic construct
  source 1..813: mol_type = other DNA
atggcgtttaaatttaaaacctttgcggcggtgggcgcgctgattggcagcctggcgctggtgggctgcggccaggatgaaaaagatccgaaccatattaaagtgggcgtgattgtgggcgcggaacagcaggtggcggaagtggcgcagaaagtggcgaaagataaatatggcctggatgtggaactggtgacctttaacgattatgtgctgccgaacgaagcgctgagcaaaggcgatattgatgcgaacgcgtttcagcataaaccgtatctggatcagcagctgaaagatcgcggctataaactggtggcggtgggcaacacctttgtgtatccgattgcgggctatagcaaaaaaattaaaagcctggatgaactgcaggatggcagccaggtggcggtgccgaacgatccgaccaacctgggccgcagcctgctgctgctgcagaaagtgggcctgattaaactgaaagatggcgtgggcctgctgccgaccgtgctggatgtggtggaaaacccgaaaaacctgaaaattgtggaactggaagcgccgcagctgccgcgcagcctggatgatgcgcagattgcgctggcggtgattaacaccacctatgcgagccagattggcctgaccccggcgaaagatggcatttttgtggaagataaagaaagcccgtatgtggcgctgattgtgacccgcgaagataacaaagatgcggaaaacgtgaaaaaatttgtgcaggcgtatcagagcgatgaagtgtatgaagcggcgaacaaagtgtttaacggcggcgcggtgaaaggctgg

SEQ ID NO: 21 - protein, 271 aa, Escherichia coli
MAFKFKTFAAVGALIGSLALVGCGQDEKDPNHIKVGVIVGAEQQVAEVAQKVAKDKYGLDVELVTFNDYVLPNEALSKGDIDANAFQHKPYLDQQLKDRGYKLVAVGNTFVYPIAGYSKKIKSLDELQDGSQVAVPNDPTNLGRSLLLLQKVGLIKLKDGVGLLPTVLDVVENPKNLKIVELEAPQLPRSLDDAQIALAVINTTYASQIGLTPAKDGIFVEDKESPYVNLIVTREDNKDAENVKKFVQAYQSDEVYEAANKVFNGGAVKGW

SEQ ID NO: 22 - protein, 271 aa, synthetic construct
MAFKFKTFAAVGALIGSLALVGCGQDEKDPNHIKVGVIVGAEQQVAEVAQKVAKDKYGLDVELVTFNDYVLPNEALSKGDIDANAFQHKPYLDQQLKDRGYKLVAVGNTFVYPIAGYSKKIKSLDELQDGSQVAVPNDPTNLGRSLLLLQKVGLIKLKDGVGLLPTVLDVVENPKNLKIVELEAPQLPRSLDDAQIALAVINTTYASQIGLTPAKDGIFVEDKESPYVALIVTREDNKDAENVKKFVQAYQSDEVYEAANKVFNGGAVKGW

SEQ ID NO: 23 - DNA, 1026 bp, Bacillus subtilis
  source 1..1026: mol_type = unassigned DNA
atgatcaatcttcaggatgtttcaaaagtttacaagtcgaaacatggagatgtcaatgctgtccaaaacgtttcgctttccattaaaaaaggtgagatttttggaattataggatatagcggagctggtaagagttccttaatccgtctgctgaacggccttgagaaaccaacctcaggaaccgtggaagtggcgggaaccaagattaatgaagtaaatggacgcggtttaagaaaagcacgccatgagatcagtatgattttccagcatttcaatttgctttggtcgcggactgtcagagataatatcatgtttcctttagaaattgccggggtgaaaaagagcgagcggatcaagcgcgccaatgaactgattaaactggtaggtttagaaggaaaagaaaaatcttatccgtcccagctgagcggcggtcagaagcagcgtgtcggaattgccagagcgcttgcaaacaatccgaaggttcttctttgtgacgaagcgacatcagcattagatccgcaaacgacagattcaattctggatctattgtccgatattaatgaaagactcggtttgacgattgtgctgattacgcacgaaatgcatgtcatacgcaaaatctgcaacagagtcgccgtcatggaaaacggcaaggtggtcgaagaaggcgaggttctcgatgtgttcaaaaatccaaaggaacaaatgacaaaacgatttgttcaacaggtgacagagccggaagaaacgaaagagactcttcagcaccttcttgatgatacagcatcaggaaaaatggttcagctcacatttgtcggtgagtcagctgaacagcctctgattacagagatgatcagaaacttcaatgtcagcgtcaatattctgcaagggaaaatttcgcagacgaaggatggggcttacggttcactgttcatccacattgacggggacgaggaagaagtgcaaaacgtgatccgattcattaatgacaaacaggtgaaagcagaggtgatcacgaatgtttga

SEQ ID NO: 24 - DNA, 1026 bp, synthetic construct
  source 1..1026: mol_type = other DNA
atgatcaatcttcaggatgtttcaaaagtttacaagtcgaaacatggagatgtcaatgctgtccaaaacgtttcgctttccattaaaaaaggtgagatttttggaattataggatatagcggagctggtaagagttccttaatccgtctgctgaacggccttgagaaaccaacctcaggaaccgtggaagtggcgggaaccaagattaatgaagtaaatggacgcggtttaagaaaagcacgccatgagatcagtatgattttccagcatttcaatttgctttggtcgcggactgtcagagataatatcatgtttcctttagaaattgccggggtgaaaaagagcgagcggatcaagcgcgccaatgaactgattaaactggtaggtttagaaggaaaagaaaaatcttatccgtcccagctgagcggcggtcagaagcagcgtgtcggaattgccagagcgcttgcaaacaatccgaaggttcttctttgtgacgaagcgacatcagcattagatccgcaaacgacagattcaattctggatctattgtccgatattaatgaaagactcggtttgacgattgtgctgattacgcacgaaatgcatgtcatacgcaaaatctgcaacagagtcgccgtcatggaaaacggcaaggtggtcgaagaaggcgaggttctcgatgtgttcaaaaatccaaaggaacaaatgacaaaacgatttgttcaacaggtgacagagccggaagaaacgaaagagactcttcagcaccttcttgatgatacagcatcaggaaaaatggttcagctcacatttgtcggtgagtcagctgaacagcctctgattacagagatgatcagaaacttcaatgtcagcgtcgctattctgcaagggaaaatttcgcagacgaaggatggggcttacggttcactgttcatccacattgacggggacgaggaagaagtgcaaaacgtgatccgattcattaatgacaaacaggtgaaagcagaggtgatcacgaatgtttga

SEQ ID NO: 25 - protein, 341 aa, Bacillus subtilis
MINLQDVSKVYKSKHGDVNAVQNVSLSIKKGEIFGIIGYSGAGKSSLIRLLNGLEKPTSGTVEVAGTKINEVNGRGLRKARHEISMIFQHFNLLWSRTVRDNIMFPLEIAGVKKSERIKRANELIKLVGLEGKEKSYPSQLSGGQKQRVGIARALANNPKVLLCDEATSALDPQTTDSILDLLSDINERLGLTIVLITHEMHVIRKICNRVAVMENGKVVEEGEVLDVFKNPKEQMTKRFVQQVTEPEETKETLQHLLDDTASGKMVQLTFVGESAEQPLITEMIRNFNVSVNILQGKISQTKDGAYGSLFIHIDGDEEEVQNVIRFINDKQVKAEVITNV

SEQ ID NO: 26 - protein, 341 aa, synthetic construct
MINLQDVSKVYKSKHGDVNAVQNVSLSIKKGEIFGIIGYSGAGKSSLIRLLNGLEKPTSGTVEVAGTKINEVNGRGLRKARHEISMIFQHFNLLWSRTVRDNIMFPLEIAGVKKSERIKRANELIKLVGLEGKEKSYPSQLSGGQKQRVGIARALANNPKVLLCDEATSALDPQTTDSILDLLSDINERLGLTIVLITHEMHVIRKICNRVAVMENGKVVEEGEVLDVFKNPKEQMTKRFVQQVTEPEETKETLQHLLDDTASGKMVQLTFVGESAEQPLITEMIRNFNVSVAILQGKISQTKDGAYGSLFIHIDGDEEEVQNVIRFINDKQVKAEVITNV

SEQ ID NO: 27 - DNA, 669 bp, Bacillus subtilis
  source 1..669: mol_type = unassigned DNA
atgtttgaaaaatacttcccgaacgttgatttaactgaattatggaacgcgacatatgaaacactttatatgacgctgatttctttactgtttgcttttgtcatcggggtcatcctcggcttgctgctctttctgacgagcaaaggaagcctctggcagaataaagcggtcaactcagtgattgcagccgttgttaacatattcagatcgattccgttccttattttaattattttactattaggttttacgaaatttttagtcggcacgatcttagggccaaacgccgcattgccggcgctggtaatcggctcggcaccattttacgcgcgccttgttgaaatcgcgctgcgtgaggtggataaaggtgtgattgaagcagctaaatcaatgggcgcgaaaacgtctacgattattttcaaagtgctgattccggaatcaatgcctgctcttatttctggcattacggttacagccatcgctttaatcggttcaacggcaattgccggagccattggttcaggaggccttggaaaccttgcgtacgtagaaggatatcagtctaataacgctgatgttaccttcgttgctactgtgtttattttaatcatcgtgtttattattcaaatcatcggtgatctaataacaaatattatagacaaacgataa

SEQ ID NO: 28 - DNA, 669 bp, synthetic construct; identical to SEQ ID NO: 27
  source 1..669: mol_type = other DNA

SEQ ID NO: 29 - protein, 222 aa, Bacillus subtilis
MFEKYFPNVDLTELWNATYETLYMTLISLLFAFVIGVILGLLLFLTSKGSLWQNKAVNSVIAAVVNIFRSIPFLILIILLLGFTKFLVGTILGPNAALPALVIGSAPFYARLVEIALREVDKGVIEAAKSMGAKTSTIIFKVLIPESMPALISGITVTAIALIGSTAIAGAIGSGGLGNLAYVEGYQSNNADVTFVATVFILIIVFIIQIIGDLITNIIDKR

SEQ ID NO: 30 - protein, 222 aa, synthetic construct; identical to SEQ ID NO: 29

SEQ ID NO: 31 - DNA, 825 bp, Bacillus subtilis
  source 1..825: mol_type = unassigned DNA
atgaaaaagctatttttgggtgcattactgcttgtatttgcaggagttatggctgcctgcggttcgaataacggcgctgaatccggcaagaaagaaattgtcgttgcggcaacaaaaacaccgcatgcggaaattttaaaagaagctgaaccattgctgaaagaaaaaggctatacgctgaaagtgaaagtgcttagtgattacaaaatgtacaataaagctttagctgataaagaagtggacgcgaactacttccagcacattccttaccttgagcaagaaatgaaagaaaacacagattacaaacttgtgaatgccggcgctgttcacttagagccattcggtatttactctaaaacatacaaatcactgaaagaccttccagacggtgcgacaatcattctgacaaacaacgttgctgaacaaggccgtatgcttgcaatgcttgaaaacgctggattaatcactcttgattctaaagtggaaacagttgacgcaacattgaaagacattaagaaaaacccgaaaaaccttgaattcaaaaaagtagcgcctgaattaacggcaaaagcatatgaaaacaaagaaggagatgcggtgttcatcaatgtaaactatgcgatccaaaataaattaaatcctaaaaaagacgcaattgaagtagaatcaacgaaaaacaacccatacgctaacatcatcgcagtaagaaaaggcgaagaagattctgcaaaaatcaaagcgctgatggaagttcttcactctaaaaagatcaaagacttcatcgagaaaaaatacgacggagctgtgcttcctgtatctgaataa

SEQ ID NO: 32 - DNA, 825 bp, synthetic construct
  source 1..825: mol_type = other DNA
atgaaaaagctatttttgggtgcattactgcttgtatttgcaggagttatggctgcctgcggttcgaataacggcgctgaatccggcaagaaagaaattgtcgttgcggcaacaaaaacaccgcatgcggaaattttaaaagaagctgaaccattgctgaaagaaaaaggctatacgctgaaagtgaaagtgcttagtgattacaaaatgtacaataaagctttagctgataaagaagtggacgcgaactacttccagcacattccttaccttgagcaagaaatgaaagaaaacacagattacaaacttgtgaatgccggcgctgttcacttagagccattcggtatttactctaaaacatacaaatcactgaaagaccttccagacggtgcgacaatcattctgacaaacaacgttgctgaacaaggccgtatgcttgcaatgcttgaaaacgctggattaatcactcttgattctaaagtggaaacagttgacgcaacattgaaagacattaagaaaaacccgaaaaaccttgaattcaaaaaagtagcgcctgaattaacggcaaaagcatatgaaaacaaagaaggagatgcggtgttcatcaatgtaaactatgcgatccaaaataaattaaatcctaaaaaagacgcaattgaagtagaatcaacgaaaaacaacccatacgctgccatcatcgcagtaagaaaaggcgaagaagattctgcaaaaatcaaagcgctgatggaagttcttcactctaaaaagatcaaagacttcatcgagaaaaaatacgacggagctgtgcttcctgtatctgaataa

SEQ ID NO: 33 - protein, 274 aa, Bacillus subtilis
MKKLFLGALLLVFAGVMAACGSNNGAESGKKEIVVAATKTPHAEILKEAEPLLKEKGYTLKVKVLSDYKMYNKALADKEVDANYFQHIPYLEQEMKENTDYKLVNAGAVHLEPFGIYSKTYKSLKDLPDGATIILTNNVAEQGRMLAMLENAGLITLDSKVETVDATLKDIKKNPKNLEFKKVAPELTAKAYENKEGDAVFINVNYAIQNKLNPKKDAIEVESTKNNPYANIIAVRKGEEDSAKIKALMEVLHSKKIKDFIEKKYDGAVLPVSE

SEQ ID NO: 34 - protein, 274 aa, synthetic construct
MKKLFLGALLLVFAGVMAACGSNNGAESGKKEIVVAATKTPHAEILKEAEPLLKEKGYTLKVKVLSDYKMYNKALADKEVDANYFQHIPYLEQEMKENTDYKLVNAGAVHLEPFGIYSKTYKSLKDLPDGATIILTNNVAEQGRMLAMLENAGLITLDSKVETVDATLKDIKKNPKNLEFKKVAPELTAKAYENKEGDAVFINVNYAIQNKLNPKKDAIEVESTKNNPYAAIIAVRKGEEDSAKIKALMEVLHSKKIKDFIEKKYDGAVLPVSE

SEQ ID NO: 35 - DNA, 3681 bp, Escherichia coli
  source 1..3681: mol_type = unassigned DNA
atgagcagcaaagtggaacagctgcgcgcgcagctgaacgaacgcattctggtgctggatggcggcatgggcaccatgattcagagctatcgcctgaacgaagcggattttcgcggcgaacgctttgcggattggccgtgcgatctgaaaggcaacaacgatctgctggtgctgagcaaaccggaagtgattgcggcgattcataacgcgtattttgaagcgggcgcggatattattgaaaccaacacctttaacagcaccaccattgcgatggcggattatcagatggaaagcctgagcgcggaaattaactttgcggcggcgaaactggcgcgcgcgtgcgcggatgaatggaccgcgcgcaccccggaaaaaccgcgctatgtggcgggcgtgctgggcccgaccaaccgcaccgcgagcattagcccggatgtgaacgatccggcgtttcgcaacattacctttgatggcctggtggcggcgtatcgcgaaagcaccaaagcgctggtggaaggcggcgcggatctgattctgattgaaaccgtgtttgataccctgaacgcgaaagcggcggtgtttgcggtgaaaaccgaatttgaagcgctgggcgtggaactgccgattatgattagcggcaccattaccgatgcgagcggccgcaccctgagcggccagaccaccgaagcgttttataacagcctgcgccatgcggaagcgctgacctttggcctgaactgcgcgctgggcccggatgaactgcgccagtatgtgcaggaactgagccgcattgcggaatgctatgtgaccgcgcatccgaacgcgggcctgccgaacgcgtttggcgaatatgatctggatgcggataccatggcgaaacagattcgcgaatgggcgcaggcgggctttctgaacattgtgggcggctgctgcggcaccaccccgcagcatattgcggcgatgagccgcgcggtggaaggcctggcgccgcgcaaactgccggaaattccggtggcgtgccgcctgagcggcctggaaccgctgaacattggcgaagatagcctgtttgtgaacgtgggcgaacgcaccaacgtgaccggcagcgcgaaatttaaacgcctgattaaagaagaaaaatatagcgaagcgctggatgtggcgcgccagcaggtggaaaacggcgcgcagattattgatattaacatggatgaaggcatgctggatgcggaagcggcgatggtgcgctttctgaacctgattgcgggcgaaccggatattgcgcgcgtgccgattatgattgatagcagcaaatgggatgtgattgaaaaaggcctgaaatgcattcagggcaaaggcattgtgaacagcattagcatgaaagaaggcgtggatgcgtttattcatcatgcgaaactgctgcgccgctatggcgcggcggtggtggtgatggcgtttgatgaacagggccaggcggatacccgcgcgcgcaaaattgaaatttgccgccgcgcgtataaaattctgaccgaagaagtgggctttccgccggaagatattatttttgatccgaacatttttgcggtggcgaccggcattgaagaacataacaactatgcgcaggattttattggcgcgtgcgaagatattaaacgcgaactgccgcatgcgctgattagcggcggcgtgagcaacgtgagctttagctttcgcggcaacgatccggtgcgcgaagcgattcatgcggtgtttctgtattatgcgattcgcaacggcatggatatgggcattgtgaacgcgggccagctggcgatttatgatgatctgccggcggaactgcgcgatgcggtggaagatgtgattctgaaccgccgcgatgatggcaccgaacgcctgctggaactggcggaaaaatatcgcggcagcaaaaccgatgataccgcgaacgcgcagcaggcggaatggcgcagctgggaagtgaacaaacgcctggaatatagcctggtgaaaggcattaccgaatttattgaacaggataccgaagaagcgcgccagcaggcgacccgcccgattgaagtgattgaaggcccgctgatggatggcatgaacgtggtgggcgatctgtttggcgaaggcaaaatgtttctgccgcaggtggtgaaaagcgcgcgcgtgatgaaacaggcggtggcgtatctggaaccgtttattgaagcgagcaaagaacagggcaaaaccaacggcaaaatggtgattgcgaccgtgaaaggcgatgtgcatgatattggcaaaaacattgtgggcgtggtgctgcagtgcaacaactatgaaattgtggatctgggcgtgatggtgccggcggaaaaaattctgcgcaccgcgaaagaagtgaacgcggatctgattggcctgagcggcctgattaccccgagcctggatgaaatggtgaacgtggcgaaagaaatggaacgccagggctttaccattccgctgctgattggcggcgcgaccaccagcaaagcgcataccgcggtgaaaattgaacagaactatagcggcccgaccgtgtatgtgcagaacgcgagccgcaccgtgggcgtggtggcggcgctgctgagcgatacccagcgcgatgattttgtggcgcgcacccgcaaagaatatgaaaccgtgcgcattcagcatggccgcaaaaaaccgcgcaccccgccggtgaccctggaagcggcgcgcgataacgattttgcgtttgattggcaggcgtataccccgccggtggcgcatcgcctgggcgtgcaggaagtggaagcgagcattgaaaccctgcgcaactatattgattggaccccgttttttatgacctggagcctggcgggcaaatatccgcgcattctggaagatgaagtggtgggcgtggaagcgcagcgcctgtttaaagatgcgaacgatatgctggataaactgagcgcggaaaaaaccctgaacccgcgcggcgtggtgggcctgtttccggcgaaccgcgtgggcgatgatattgaaatttatcgcgatgaaacccgcacccatgtgattaacgtgagccatcatctgcgccagcagaccgaaaaaaccggctttgcgaactattgcctggcggattttgtggcgccgaaactgagcggcaaagcggattatattggcgcgtttgcggtgaccggcggcctggaagaagatgcgctggcggatgcgtttgaagcgcagcatgatgattataacaaaattatggtgaaagcgctggcggatcgcctggcggaagcgtttgcggaatatctgcatgaacgcgtgcgcaaagtgtattggggctatgcgccgaacgaaaacctgagcaacgaagaactgattcgcgaaaactatcagggcattcgcccggcgccgggctatccggcgtgcccggaacataccgaaaaagcgaccatttgggaactgctggaagtggaaaaacataccggcatgaaactgaccgaaagctttgcgatgtggccgggcgcgagcgtgagcggctggtattttagccatccggatagcaaatattatgcggtggcgcagattcagcgcgatcaggtggaagattatgcgcgccgcaaaggcatgagcgtgaccgaagtggaacgctggctggcgccgaacctgggctatgatgcggat

SEQ ID NO: 36 - protein, 1227 aa, Escherichia coli
MSSKVEQLRAQLNERILVLDGGMGTMIQSYRLNEADFRGERFADWPCDLKGNNDLLVLSKPEVIAAIHNAYFEAGADIIETNTFNSTTIAMADYQMESLSAEINFAAAKLARACADEWTARTPEKPRYVAGVLGPTNRTASISPDVNDPAFRNITFDGLVAAYRESTKALVEGGADLILIETVFDTLNAKAAVFAVKTEFEALGVELPIMISGTITDASGRTLSGQTTEAFYNSLRHAEALTFGLNCALGPDELRQYVQELSRIAECYVTAHPNAGLPNAFGEYDLDADTMAKQIREWAQAGFLNIVGGCCGTTPQHIAAMSRAVEGLAPRKLPEIPVACRLSGLEPLNIGEDSLFVNVGERTNVTGSAKFKRLIKEEKYSEALDVARQQVENGAQIIDINMDEGMLDAEAAMVRFLNLIAGEPDIARVPIMIDSSKWDVIEKGLKCIQGKGIVNSISMKEGVDAFIHHAKLLRRYGAAVVVMAFDEQGQADTRARKIEICRRAYKILTEEVGFPPEDIIFDPNIFAVATGIEEHNNYAQDFIGACEDIKRELPHALISGGVSNVSFSFRGNDPVREAIHAVFLYYAIRNGMDMGIVNAGQLAIYDDLPAELRDAVEDVILNRRDDGTERLLELAEKYRGSKTDDTANAQQAEWRSWEVNKRLEYSLVKGITEFIEQDTEEARQQATRPIEVIEGPLMDGMNVVGDLFGEGKMFLPQVVKSARVMKQAVAYLEPFIEASKEQGKTNGKMVIATVKGDVHDIGKNIVGVVLQCNNYEIVDLGVMVPAEKILRTAKEVNADLIGLSGLITPSLDEMVNVAKEMERQGFTIPLLIGGATTSKAHTAVKIEQNYSGPTVYVQNASRTVGVVAALLSDTQRDDFVARTRKEYETVRIQHGRKKPRTPPVTLEAARDNDFAFDWQAYTPPVAHRLGVQEVEASIETLRNYIDWTPFFMTWSLAGKYPRILEDEVVGVEAQRLFKDANDMLDKLSAEKTLNPRGVVGLFPANRVGDDIEIYRDETRTHVINVSHHLRQQTEKTGFANYCLADFVAPKLSGKADYIGAFAVTGGLEEDALADAFEAQHDDYNKIMVKALADRLAEAFAEYLHERVRKVYWGYAPNENLSNEELIRENYQGIRPAPGYPACPEHTEKATIWELLEVEKHTGMKLTESFAMWPGASVSGWYFSHPDSKYYAVAQIQRDQVEDYARRKGMSVTEVERWLAPNLGYDAD

SEQ ID NO: 37 - DNA, 2259 bp, Escherichia coli
  source 1..2259: mol_type = unassigned DNA
atgaccattctgaaccataccctgggctttccgcgcgtgggcctgcgccgcgaactgaaaaaagcgcaggaaagctattgggcgggcaacagcacccgcgaagaactgctggcggtgggccgcgaactgcgcgcgcgccattgggatcagcagaaacaggcgggcattgatctgctgccggtgggcgattttgcgtggtatgatcatgtgctgaccaccagcctgctgctgggcaacgtgccggcgcgccatcagaacaaagatggcagcgtggatattgataccctgtttcgcattggccgcggccgcgcgccgaccggcgaaccggcggcggcggcggaaatgaccaaatggtttaacaccaactatcattatatggtgccggaatttgtgaaaggccagcagtttaaactgacctggacccagctgctggatgaagtggatgaagcgctggcgctgggccataaagtgaaaccggtgctgctgggcccggtgacctggctgtggctgggcaaagtgaaaggcgaacagtttgatcgcctgagcctgctgaacgatattctgccggtgtatcagcaggtgctggcggaactggcgaaacgcggcattgaatgggtgcagattgatgaaccggcgctggtgctggaactgccgcaggcgtggctggatgcgtataaaccggcgtatgatgcgctgcagggccaggtgaaactgctgctgaccacctattttgaaggcgtgaccccgaacctggataccattaccgcgctgccggtgcagggcctgcatgtggatctggtgcatggcaaagatgatgtggcggaactgcataaacgcctgccgagcgattggctgctgagcgcgggcctgattaacggccgcaacgtgtggcgcgcggatctgaccgaaaaatatgcgcagattaaagatattgtgggcaaacgcgatctgtgggtggcgagcagctgcagcctgctgcatagcccgattgatctgagcgtggaaacccgcctggatgcggaagtgaaaagctggtttgcgtttgcgctgcagaaatgccatgaactggcgctgctgcgcgatgcgctgaacagcggcgataccgcggcgctggcggaatggagcgcgccgattcaggcgcgccgccatagcacccgcgtgcataacccggcggtggaaaaacgcctggcggcgattaccgcgcaggatagccagcgcgcgaacgtgtatgaagtgcgcgcggaagcgcagcgcgcgcgctttaaactgccggcgtggccgaccaccaccattggcagctttccgcagaccaccgaaattcgcaccctgcgcctggattttaaaaaaggcaacctggatgcgaacaactatcgcaccggcattgcggaacatattaaacaggcgattgtggaacaggaacgcctgggcctggatgtgctggtgcatggcgaagcggaacgcaacgatatggtggaatattttggcgaacatctggatggctttgtgtttacccagaacggctgggtgcagagctatggcagccgctgcgtgaaaccgccgattgtgattggcgatattagccgcccggcgccgattaccgtggaatgggcgaaatatgcgcagagcctgaccgataaaccggtgaaaggcatgctgaccggcccggtgaccattctgtgctggagctttccgcgcgaagatgtgagccgcgaaaccattgcgaaacagattgcgctggcgctgcgcgatgaagtggcggatctggaagcggcgggcattggcattattcagattgatgaaccggcgctgcgcgaaggcctgccgctgcgccgcagcgattgggatgcgtatctgcagtggggcgtggaagcgtttcgcattaacgcggcggtggcgaaagatgatacccagattcatacccatatgtgctattgcgaatttaacgatattatggatagcattgcggcgctggatgcggatgtgattaccattgaaaccagccgcagcgatatggaactgctggaaagctttgaagaatttgattatccgaacgaaattggcccgggcgtgtatgatattcatagcccgaacgtgccgagcgtggaatggattgaagcgctgctgaaaaaagcggcgaaacgcattccggcggaacgcctgtgggtgaacccggattgcggcctgaaaacccgcggctggccggaaacccgcgcggcgctggcgaacatggtgcaggcggcgcagaacctgcgccgcggc

SEQ ID NO: 38 - protein, 753 aa, Escherichia coli
MTILNHTLGFPRVGLRRELKKAQESYWAGNSTREELLAVGRELRARHWDQQKQAGIDLLPVGDFAWYDHVLTTSLLLGNVPARHQNKDGSVDIDTLFRIGRGRAPTGEPAAAAEMTKWFNTNYHYMVPEFVKGQQFKLTWTQLLDEVDEALALGHKVKPVLLGPVTWLWLGKVKGEQFDRLSLLNDILPVYQQVLAELAKRGIEWVQIDEPALVLELPQAWLDAYKPAYDALQGQVKLLLTTYFEGVTPNLDTITALPVQGLHVDLVHGKDDVAELHKRLPSDWLLSAGLINGRNVWRADLTEKYAQIKDIVGKRDLWVASSCSLLHSPIDLSVETRLDAEVKSWFAFALQKCHELALLRDALNSGDTAALAEWSAPIQARRHSTRVHNPAVEKRLAAITAQDSQRANVYEVRAEAQRARFKLPAWPTTTIGSFPQTTEIRTLRLDFKKGNLDANNYRTGIAEHIKQAIVEQERLGLDVLVHGEAERNDMVEYFGEHLDGFVFTQNGWVQSYGSRCVKPPIVIGDISRPAPITVEWAKYAQSLTDKPVKGMLTGPVTILCWSFPREDVSRETIAKQIALALRDEVADLEAAGIGIIQIDEPALREGLPLRRSDWDAYLQWGVEAFRINAAVAKDDTQIHTHMCYCEFNDIMDSIAALDADVITIETSRSDMELLESFEEFDYPNEIGPGVYDIHSPNVPSVEWIEALLKKAAKRIPAERLWVNPDCGLKTRGWPETRAALANMVQAAQNLRRG

SEQ ID NO: 39 - DNA, 2286 bp, Bacillus subtilis
  source 1..2286: mol_type = unassigned DNA
atgacaaccatcaaaacatcgaatttaggatttccgagaatcggactgaaccgggaatggaaaaaagcacttgaagcgtattggaaaggcagtactgataaagatacgtttttgaagcaaatcgacgaactatttttatccgcagtaaaaacacaaattgaccagcagattgatgttgtgcctgtttctgatttcacacagtatgaccatgtactcgacacagcagtcagcttcaactggatcccgaaacggttcagacatttgactgacgctaccgatacatacttcgctatcgcccgcggaatcaaagacgctgtatctagtgaaatgacaaaatggtttaatacaaattaccattacatcgttccggaatatgacgagagcattgagttccgtctgacaagaaacaaacaactcgaagattatcgccggatcaaacaggaatacggtgtggaaacaaagcctgtgattgtcggcccttatacgttcgttacgcttgctaaaggctatgaaccgtctgaagcaaaagcgatccaaaaacgccttgtgccattatatgtacagcttttgaaagagcttgaagaagaaggcgtaaaatgggttcaaatcgatgagccggcgctcgttaccgcctctagtgaagatgtacgcggcgcaaaagaattatttgaaagcattacaagtgagctttcatccttgaatgtgcttttgcagacgtattttgattctgttgatgcttatgaagagctgatctcttacccggttcagggaattggccttgatttcgttcacgacaaaggcagaaacctggaacagcttaaaacacatggcttcccgacagataaagtgctggcagccggcgttatcgacggacgcaacatttggaaagcggaccttgaagagcgtctcgatgccgttcttgatattctcagcattgcaaaagttgatgaactgtggattcagccttccagcagcctgctgcatgttccagtagcgaaacaccctgatgagcatttggaaaaagacctattgaacggattatcctacgcaaaagaaaagctggccgagctgacagctttgaaagaaggcttagtatcaggaaaagcggcgatcagcgaagagattcagcaggctaaggctgatatccaggcgcttaaacagtttgcaacaggcgccaattctgaacaaaagaaagagcttgagcaattaactgataaagacttcaagcgcccgattccgtttgaagaacgtttagccctacaaaatgaatctctcggccttccgcttttgccgacgacaacgatcggcagcttcccgcagtctgctgaagtgcggagcgcacgccaaaaatggcggaaagctgagtggtccgatgaacagtatcaaaactttatcaatgcggaaacaaaaagatggattgatattcaggaagaattggagcttgatgtccttgttcacggcgaatttgaacggacagacatggtcgaatacttcggtgaaaagctggccggtttcgccttcactaaatatgcctgggttcaatcatacggctcacgctgtgtccgcccgccagtcatttacggagatgttgaatttattgaaccgatgacagtgaaagacacagtctacgcacagtcattgacttccaagcatgtgaaaggaatgctgacgggcccggttacaatcttaaactggtctttccctcgaaacgacatctcgaggaaagaaatcgccttccaaatcgggcttgcccttcgcaaagaagttaaagcgcttgaagacgcaggcattcaaatcattcaagtcgatgaaccagcgctgcgtgaaggccttccattgaaaacccgcgattgggatgagtatttgacttgggcggcagaagctttcagattaaccacttcttccgtgaaaaacgagacacaaattcatacacatatgtgctacagcaacttcgaagatatcgttgatacaatcaatgatcttgatgccgatgtgattacaatcgaacatagcagaagccacggaggatttttagattacttaaaaaaccacccgtatttgaaagggcttggccttggtgtatatgacattcacagccctcgtgtgccgtcaactgaagaaatgtacaatattatcgttgatgcgcttgccgtctgtccgactgaccgcttctgggtaaatccagactgcggattgaaaacaagacagcaggaagaaacggttgcagcattgaaaaatatggttgaagccgcaaaacaggcaagagcacagcagacacagctagta

SEQ ID NO: 40 - protein, 762 aa, Bacillus subtilis
MTTIKTSNLGFPRIGLNREWKKALEAYWKGSTDKDTFLKQIDELFLSAVKTQIDQQIDVVPVSDFTQYDHVLDTAVSFNWIPKRFRHLTDATDTYFAIARGIKDAVSSEMTKWFNTNYHYIVPEYDESIEFRLTRNKQLEDYRRIKQEYGVETKPVIVGPYTFVTLAKGYEPSEAKAIQKRLVPLYVQLLKELEEEGVKWVQIDEPALVTASSEDVRGAKELFESITSELSSLNVLLQTYFDSVDAYEELISYPVQGIGLDFVHDKGRNLEQLKTHGFPTDKVLAAGVIDGRNIWKADLEERLDAVLDILSIAKVDELWIQPSSSLLHVPVAKHPDEHLEKDLLNGLSYAKEKLAELTALKEGLVSGKAAISEEIQQAKADIQALKQFATGANSEQKKELEQLTDKDFKRPIPFEERLALQNESLGLPLLPTTTIGSFPQSAEVRSARQKWRKAEWSDEQYQNFINAETKRWIDIQEELELDVLVHGEFERTDMVEYFGEKLAGFAFTKYAWVQSYGSRCVRPPVIYGDVEFIEPMTVKDTVYAQSLTSKHVKGMLTGPVTILNWSFPRNDISRKEIAFQIGLALRKEVKALEDAGIQIIQVDEPALREGLPLKTRDWDEYLTWAAEAFRLTTSSVKNETQIHTHMCYSNFEDIVDTINDLDADVITIEHSRSHGGFLDYLKNHPYLKGLGLGVYDIHSPRVPSTEEMYNIIVDALAVCPTDRFWVNPDCGLKTRQQEETVAALKNMVEAAKQARAQQTQLV

SEQ ID NO: 41 - DNA, 315 bp, Escherichia coli
  source 1..315: mol_type = unassigned DNA
atggcggaatggagcggcgaatatattagcccgtatgcggaacatggcaaaaaaagcgaacaggtgaaaaaaattaccgtgagcattccgctgaaagtgctgaaaattctgaccgatgaacgcacccgccgccaggtgaacaacctgcgccatgcgaccaacagcgaactgctgtgcgaagcgtttctgcatgcgtttaccggccagccgctgccggatgatgcggatctgcgcaaagaacgcagcgatgaaattccggaagcggcgaaagaaattatgcgcgaaatgggcattaacccggaaacctgggaatat

SEQ ID NO: 42 - protein, 105 aa, Escherichia coli
MAEWSGEYISPYAEHGKKSEQVKKITVSIPLKVLKILTDERTRRQVNNLRHATNSELLCEAFLHAFTGQPLPDDADLRKERSDEIPEAAKEIMREMGINPETWEY

SEQ ID NO: 43 - DNA, 951 bp, Escherichia coli
  source 1..951: mol_type = unassigned DNA
atgattgaagtgaaacatctgaaaaccctgcaggcgctgcgcaactgcggcagcctggcggcggcggcggcgaccctgcatcagacccagagcgcgctgagccatcagtttagcgatctggaacagcgcctgggctttcgcctgtttgtgcgcaaaagccagccgctgcgctttaccccgcagggcgaaattctgctgcagctggcgaaccaggtgctgccgcagattagccaggcgctgcaggcgtgcaacgaaccgcagcagacccgcctgcgcattgcgattgaatgccatagctgcattcagtggctgaccccggcgctggaaaactttcataaaaactggccgcaggtggaaatggattttaaaagcggcgtgacctttgatccgcagccggcgctgcagcagggcgaactggatctggtgatgaccagcgatattctgccgcgcagcggcctgcattatagcccgatgtttgattatgaagtgcgcctggtgctggcgccggatcatccgctggcggcgaaaacccgcattaccccggaagatctggcgagcgaaaccctgctgatttatccggtgcagcgcagccgcctggatgtgtggcgccattttctgcagccggcgggcgtgagcccgagcctgaaaagcgtggataacaccctgctgctgattcagatggtggcggcgcgcatgggcattgcggcgctgccgcattgggtggtggaaagctttgaacgccagggcctggtggtgaccaaaaccctgggcgaaggcctgtggagccgcctgtatgcggcggtgcgcgatggcgaacagcgccagccggtgaccgaagcgtttattcgcagcgcgcgcaaccatgcgtgcgatcatctgccgtttgtgaaaagcgcggaacgcccgacctatgatgcgccgaccgtgcgcccgggcagcccggcgcgcctg

SEQ ID NO: 44 - protein, 317 aa, Escherichia coli
MIEVKHLKTLQALRNCGSLAAAAATLHQTQSALSHQFSDLEQRLGFRLFVRKSQPLRFTPQGEILLQLANQVLPQISQALQACNEPQQTRLRIAIECHSCIQWLTPALENFHKNWPQVEMDFKSGVTFDPQPALQQGELDLVMTSDILPRSGLHYSPMFDYEVRLVLAPDHPLAAKTRITPEDLASETLLIYPVQRSRLDVWRHFLQPAGVSPSLKSVDNTLLLIQMVAARMGIAALPHWVVESFERQGLVVTKTLGEGLWSRLYAAVRDGEQRQPVTEAFIRSARNHACDHLPFVKSAERPTYDAPTVRPGSPARL

SEQ ID NO: 45 - DNA, 1416 bp, Homo sapiens
  source 1..1416: mol_type = unassigned DNA
atggcgaccaaatgcggcaactgcggcccgggctatagcaccccgctggaagcgatgaaaggcccgcgcgaagaaattgtgtatctgccgtgcatttatcgcaacaccggcaccgaagcgccggattatctggcgaccgtggatgtggatccgaaaagcccgcagtattgccaggtgattcatcgcctgccgatgccgaacctgaaagatgaactgcatcatagcggctggaacacctgcagcagctgctttggcgatagcaccaaaagccgcaccaaactggtgctgccgagcctgattagcagccgcatttatgtggtggatgtgggcagcgaaccgcgcgcgccgaaactgcataaagtgattgaaccgaaagatattcatgcgaaatgcgaactggcgtttctgcataccagccattgcctggcgagcggcgaagtgatgattagcagcctgggcgatgtgaaaggcaacggcaaaggcggctttgtgctgctggatggcgaaacctttgaagtgaaaggcacctgggaacgcccgggcggcgcggcgccgctgggctatgatttttggtatcagccgcgccataacgtgatgattagcaccgaatgggcggcgccgaacgtgctgcgcgatggctttaacccggcggatgtggaagcgggcctgtatggcagccatctgtatgtgtgggattggcagcgccatgaaattgtgcagaccctgagcctgaaagatggcctgattccgctggaaattcgctttctgcataacccggatgcggcgcagggctttgtgggctgcgcgctgagcagcaccattcagcgcttttataaaaacgaaggcggcacctggagcgtggaaaaagtgattcaggtgccgccgaaaaaagtgaaaggctggctgctgccggaaatgccgggcctgattaccgatattctgctgagcctggatgatcgctttctgtattttagcaactggctgcatggcgatctgcgccagtatgatattagcgatccgcagcgcccgcgcctgaccggccagctgtttctgggcggcagcattgtgaaaggcggcccggtgcaggtgctggaagatgaagaactgaaaagccagccggaaccgctggtggtgaaaggcaaacgcgtggcgggcggcccgcagatgattcagctgagcctggatggcaaacgcctgtatattaccaccagcctgtatagcgcgtgggataaacagttttatccggatctgattcgcgaaggcagcgtgatgctgcaggtggatgtggataccgtgaaaggcggcctgaaactgaacccgaactttctggtggattttggcaaagaaccgctgggcccggcgctggcgcatgaactgcgctatccgggcggcgattgcagcagcgatatttggatt

SEQ ID NO: 46 - protein, 472 aa, Homo sapiens
MATKCGNCGPGYSTPLEAMKGPREEIVYLPCIYRNTGTEAPDYLATVDVDPKSPQYCQVIHRLPMPNLKDELHHSGWNTCSSCFGDSTKSRTKLVLPSLISSRIYVVDVGSEPRAPKLHKVIEPKDIHAKCELAFLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPDAAQGFVGCALSSTIQRFYKNEGGTWSVEKVIQVPPKKVKGWLLPEMPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWDKQFYPDLIREGSVMLQVDVDTVKGGLKLNPNFLVDFGKEPLGPALAHELRYPGGDCSSDIWI

SEQ ID NO: 47 - DNA, 1305 bp, unidentified
  source 1..1305: mol_type = unassigned DNA
atgaaaaaacatctgctggcgggcgcgtgcgcgctggcgatgggctttgcggtgattccgggcacctttgcggatgaaacctgcaacagcccgtttaccaccgcgctgattaccggccaggaacagtatctgcatgtgtggaccctgggcatgccgggcgtgggcgatgaaagcgataaactggtgaccattagcgtggatccgaaaagcgataaatatggcaaagtgattaacaccctgagcgtgggcggccgcggcgaagcgcatcataccggctttaccgatgatcgccgctatctgtgggcgggccgcctggatgataacaaaatttttatttttgatctgattgatccggcgaacccgaaactgattaaaaccattaccgattttgcggatcgcaccggctatgtgggcccgcataccttttatgcgctgccgggccgcatgctgattcaggcgctgagcaacaccaaaacccatgatggccagaccggcctggcggtgtatagcaacgcgggcgaactggtgagcctgcatccgatgccggtgaccgatggcggcgatggctatggctatgatattggcattaacccggcgaaaaacgtgctgctgaccagcagctttaccggctggaacaactatatgatggatctgggcaaaatggtgaaagatccggaagcgatgaaacgctttggcaacaccatggcgatttgggatctgaaaagcatgaaagcggaaaaaattctgaacgtgccgggcgcgccgctggaaattcgctggagcctgaaaccggaacataactgggcgtataccgcgaccgcgctgaccagcaaactgtggctgattaaacaggatgataaaggcgaatggattgcgaaagaaaccggcaccattggcgatccgagcaaaattccgctgccggtggatattagcattaccgcggatgcgaaaggcctgtgggtgaacacctttctggatggcaccacccgcttttatgatattagcgaaccggaacatccgaaagaagtgtttagcaaaaaaatgggcaaccaggtgaacatggtgagccagagctatgatggcaaacgcgtgtattttaccaccagcctgattgcgaactgggataaaaaaggcgcggaaaacgatcagtggctgaaagcgtatgattgggatggcaaagaactggtggaaaaatttaccgtggattttaacgaactgaaactgggccgcgcgcatcatatgaaatttagcagcaaaaccaacgcggcggaactgggcaccaaccagagctttccgacccgccag

SEQ ID NO: 48 - protein, 435 aa, unidentified
MKKHLLAGACALAMGFAVIPGTFADETCNSPFTTALITGQEQYLHVWTLGMPGVGDESDKLVTISVDPKSDKYGKVINTLSVGGRGEAHHTGFTDDRRYLWAGRLDDNKIFIFDLIDPANPKLIKTITDFADRTGYVGPHTFYALPGRMLIQALSNTKTHDGQTGLAVYSNAGELVSLHPMPVTDGGDGYGYDIGINPAKNVLLTSSFTGWNNYMMDLGKMVKDPEAMKRFGNTMAIWDLKSMKAEKILNVPGAPLEIRWSLKPEHNWAYTATALTSKLWLIKQDDKGEWIAKETGTIGDPSKIPLPVDISITADAKGLWVNTFLDGTTRFYDISEPEHPKEVFSKKMGNQVNMVSQSYDGKRVYFTTSLIANWDKKGAENDQWLKAYDWDGKELVEKFTVDFNELKLGRAHHMKFSSKTNAAELGTNQSFPTRQ

SEQ ID NO: 49 - DNA, 2178 bp, Escherichia coli
  source 1..2178: mol_type = unassigned DNA
atgagcaccagcgatgatattcataacaccaccgcgaccggcaaatgcccgtttcatcagggcggccatgatcagagcgcgggcgcgggcaccaccacccgcgattggtggccgaaccagctgcgcgtggatctgctgaaccagcatagcaaccgcagcaacccgctgggcgaagattttgattatcgcaaagaatttagcaaactggattattatggcctgaaaaaagatctgaaagcgctgctgaccgaaagccagccgtggtggccggcggattggggcagctatgcgggcctgtttattcgcatggcgtggcatggcgcgggcacctatcgcagcattgatggccgcggcggcgcgggccgcggccagcagcgctttgcgccgctgaacagctggccggataacgtgagcctggataaagcgcgccgcctgctgtggccgattaaacagaaatatggccagaaaattagctgggcggatctgtttattctggcgggcaacgtggcgctggaaaacagcggctttcgcacctttggctttggcgcgggccgcgaagatgtgtgggaaccggatctggatgtgaactggggcgatgaaaaagcgtggctgacccatcgccatccggaagcgctggcgaaagcgccgctgggcgcgaccgaaatgggcctgatttatgtgaacccggaaggcccggatcatagcggcgaaccgctgagcgcggcggcggcgattcgcgcgacctttggcaacatgggcatgaacgatgaagaaaccgtggcgctgattgcgggcggccataccctgggcaaaacccatggcgcgggcccgaccagcaacgtgggcccggatccggaagcggcgccgattgaagaacagggcctgggctgggcgagcacctatggcagcggcgtgggcgcggatgcgattaccagcggcctggaagtggtgtggacccagaccccgacccagtggagcaactatttttttgaaaacctgtttaaatatgaatgggtgcagacccgcagcccggcgggcgcgattcagtttgaagcggtggatgcgccggaaattattccggatccgtttgatccgagcaaaaaacgcaaaccgaccatgctggtgaccgatctgaccctgcgctttgatccggaatttgaaaaaattagccgccgctttctgaacgatccgcaggcgtttaacgaagcgtttgcgcgcgcgtggtttaaactgacccatcgcgatatgggcccgaaaagccgctatattggcccggaagtgccgaaagaagatctgatttggcaggatccgctgccgcagccgatttataacccgaccgaacaggatattattgatctgaaatttgcgattgcggatagcggcctgagcgtgagcgaactggtgagcgtggcgtgggcgagcgcgagcacctttcgcggcggcgataaacgcggcggcgcgaacggcgcgcgcctggcgctgatgccgcagcgcgattgggatgtgaacgcggcggcggtgcgcgcgctgccggtgctggaaaaaattcagaaagaaagcggcaaagcgagcctggcggatattattgtgctggcgggcgtggtgggcgtggaaaaagcggcgagcgcggcgggcctgagcattcatgtgccgtttgcgccgggccgcgtggatgcgcgccaggatcagaccgatattgaaatgtttgaactgctggaaccgattgcggatggctttcgcaactatcgcgcgcgcctggatgtgagcaccaccgaaagcctgctgattgataaagcgcagcagctgaccctgaccgcgccggaaatgaccgcgctggtgggcggcatgcgcgtgctgggcgcgaactttgatggcagcaaaaacggcgtgtttaccgatcgcgtgggcgtgctgagcaacgatttttttgtgaacctgctggatatgcgctatgaatggaaagcgaccgatgaaagcaaagaactgtttgaaggccgcgatcgcgaaaccggcgaagtgaaatttaccgcgagccgcgcggatctggtgtttggcagcaacagcgtgctgcgcgcggtggcggaagtgtatgcgagcagcgatgcgcatgaaaaatttgtgaaagattttgtggcggcgtgggtgaaagtgatgaacctggatcgctttgatctgctg

SEQ ID NO: 50 - protein, 726 aa, Escherichia coli
MSTSDDIHNTTATGKCPFHQGGHDQSAGAGTTTRDWWPNQLRVDLLNQHSNRSNPLGEDFDYRKEFSKLDYYGLKKDLKALLTESQPWWPADWGSYAGLFIRMAWHGAGTYRSIDGRGGAGRGQQRFAPLNSWPDNVSLDKARRLLWPIKQKYGQKISWADLFILAGNVALENSGFRTFGFGAGREDVWEPDLDVNWGDEKAWLTHRHPEALAKAPLGATEMGLIYVNPEGPDHSGEPLSAAAAIRATFGNMGMNDEETVALIAGGHTLGKTHGAGPTSNVGPDPEAAPIEEQGLGWASTYGSGVGADAITSGLEVVWTQTPTQWSNYFFENLFKYEWVQTRSPAGAIQFEAVDAPEIIPDPFDPSKKRKPTMLVTDLTLRFDPEFEKISRRFLNDPQAFNEAFARAWFKLTHRDMGPKSRYIGPEVPKEDLIWQDPLPQPIYNPTEQDIIDLKFAIADSGLSVSELVSVAWASASTFRGGDKRGGANGARLALMPQRDWDVNAAAVRALPVLEKIQKESGKASLADIIVLAGVVGVEKAASAAGLSIHVPFAPGRVDARQDQTDIEMFELLEPIADGFRNYRARLDVSTTESLLIDKAQQLTLTAPEMTALVGGMRVLGANFDGSKNGVFTDRVGVLSNDFFVNLLDMRYEWKATDESKELFEGRDRETGEVKFTASRADLVFGSNSVLRAVAEVYASSDAHEKFVKDFVAAWVKVMNLDRFDLL

SEQ ID NO: 51 - DNA, 1197 bp, Pseudomonas putida
  source 1..1197: mol_type = unassigned DNA
atgagcggcaaccgcggcgtggtgtatctgggcagcggcaaagtggaagtgcagaaaattgattatccgaaaatgcaggatccgcgcggcaaaaaaattgaacatggcgtgattctgaaagtggtgagcaccaacatttgcggcagcgatcagcatatggtgcgcggccgcaccaccgcgcaggtgggcctggtgctgggccatgaaattaccggcgaagtgattgaaaaaggccgcgatgtggaaaacctgcagattggcgatctggtgagcgtgccgtttaacgtggcgtgcggccgctgccgcagctgcaaagaaatgcataccggcgtgtgcctgaccgtgaacccggcgcgcgcgggcggcgcgtatggctatgtggatatgggcgattggaccggcggccaggcggaatatctgctggtgccgtatgcggattttaacctgctgaaactgccggatcgcgataaagcgatggaaaaaattcgcgatctgacctgcctgagcgatattctgccgaccggctatcatggcgcggtgaccgcgggcgtgggcccgggcagcaccgtgtatgtggcgggcgcgggcccggtgggcctggcggcggcggcgagcgcgcgcctgctgggcgcggcggtggtgattgtgggcgatctgaacccggcgcgcctggcgcatgcgaaagcgcagggctttgaaattgcggatctgagcctggataccccgctgcatgaacagattgcggcgctgctgggcgaaccggaagtggattgcgcggtggatgcggtgggctttgaagcgcgcggccatggccatgaaggcgcgaaacatgaagcgccggcgaccgtgctgaacagcctgatgcaggtgacccgcgtggcgggcaaaattggcattccgggcctgtatgtgaccgaagatccgggcgcggtggatgcggcggcgaaaattggcagcctgagcattcgctttggcctgggctgggcgaaaagccatagctttcataccggccagaccccggtgatgaaatataaccgcgcgctgatgcaggcgattatgtgggatcgcattaacattgcggaagtggtgggcgtgcaggtgattagcctggatgatgcgccgcgcggctatggcgaatttgatgcgggcgtgccgaaaaaatttgtgattgatccgcataaaacctttagcgcggcg

SEQ ID NO: 52 - protein, 399 aa, Pseudomonas putida
MSGNRGVVYLGSGKVEVQKIDYPKMQDPRGKKIEHGVILKVVSTNICGSDQHMVRGRTTAQVGLVLGHEITGEVIEKGRDVENLQIGDLVSVPFNVACGRCRSCKEMHTGVCLTVNPARAGGAYGYVDMGDWTGGQAEYLLVPYADFNLLKLPDRDKAMEKIRDLTCLSDILPTGYHGAVTAGVGPGSTVYVAGAGPVGLAAAASARLLGAAVVIVGDLNPARLAHAKAQGFEIADLSLDTPLHEQIAALLGEPEVDCAVDAVGFEARGHGHEGAKHEAPATVLNSLMQVTRVAGKIGIPGLYVTEDPGAVDAAAKIGSLSIRFGLGWAKSHSFHTGQTPVMKYNRALMQAIMWDRINIAEVVGVQVISLDDAPRGYGEFDAGVPKKFVIDPHKTFSAA

SEQ ID NO: 53 - DNA, 2220 bp, Clostridium pasteurianum
  source 1..2220: mol_type = unassigned DNA
atgtttaaacagtgggaaggctttcaggatggcgaatggaccaacgatgtgaacgtgcgcgattttattcagaaaaactataaagaatataccggcgataaaagctttctgaaaggcccgaccgaaaaaaccaaaaaagtgtgggataaagcggtgagcctgattctggaagaactgaaaaaaggcattctggatgtggataccgaaaccattagcggcattaacagctttaaaccgggctatctggataaagataacgaagtgattgtgggctttcagaccgatgcgccgctgaaacgcattaccaacccgtttggcggcattcgcatggcggaacagagcctgaaagaatatggctttaaaattagcgatgaaatgcataacatttttaccaactatcgcaaaacccataaccagggcgtgtttgatgcgtatagcgaagaaacccgcattgcgcgcagcgcgggcgtgctgaccggcctgccggatgcgtatggccgcggccgcattattggcgattatcgccgcgtggcgctgtatggcattgattttctgattcaggaaaaaaaaaaagatctgagcaacctgaaaggcgatatgctggatgaactgattcgcctgcgcgaagaagtgagcgaacagattcgcgcgctggatgaaattaaaaaaatggcgctgagctatggcgtggatattagccgcccggcggtgaacgcgaaagaagcggcgcagtttctgtattttggctatctggcgggcgtgaaagaaaacaacggcgcggcgatgagcctgggccgcaccagcacctttctggatatttatattgaacgcgatctggaacagggcctgattaccgaagatgaagcgcaggaagtgattgatcagtttattattaaactgcgcctggtgcgccatctgcgcaccccggaatataacgaactgtttgcgggcgatccgacctgggtgaccgaaagcattgcgggcgtgggcattgatggccgcagcctggtgaccaaaaacagctttcgctatctgcataccctgattaacctgggcagcgcgccggaaccgaacatgaccgtgctgtggagcgaaaacctgccggaaagctttaaaaaattttgcgcggaaatgagcattctgaccgatagcattcagtatgaaaacgatgatattatgcgcccgatttatggcgatgattatgcgattgcgtgctgcgtgagcgcgatgcgcgtgggcaaagatatgcagttttttggcgcgcgctgcaacctggcgaaatgcctgctgctggcgattaacggcggcgtggatgaaaaaaaaggcattaaagtggtgccggatattgaaccgattaccgatgaagtgctggattatgaaaaagtgaaagaaaactattttaaagtgctggaatatatggcgggcctgtatgtgaacaccatgaacattattcattttatgcatgataaatatgcgtatgaagcgagccagatggcgctgcatgataccaaagtgggccgcctgatggcgtttggcattgcgggctttagcgtggcggcggatagcctgagcgcgattcgctatgcgaaagtgaaaccgattcgcgaaaacggcattaccgtggattttgtgaaagaaggcgattttccgaaatatggcaacgatgatgatcgcgtggatagcattgcggtggaaattgtggaaaaatttagcgatgaactgaaaaaacatccgacctatcgcaacgcgaaacataccctgagcgtgctgaccattaccagcaacgtgatgtatggcaaaaaaaccggcaccaccccggatggccgcaaagtgggcgaaccgctggcgccgggcgcgaacccgatgcatggccgcgatatggaaggcgcgctggcgagcctgaacagcgtggcgaaagtgccgtatgtgtgctgcgaagatggcgtgagcaacacctttagcattgtgccggatgcgctgggcaacgatcatgatgtgcgcattaacaacctggtgagcattatgggcggctattttggccagggcgcgcatcatctgaacgtgaacgtgctgaaccgcgaaaccctgattgatgcgatgaacaacccggataaatatccgaccctgaccattcgcgtgagcggctatgcggtgaactttaaccgcctgagcaaagatcatcagaaagaagtgattagccgcacctttcatgaaaaactg

SEQ ID NO: 54 - protein, 740 aa, Clostridium pasteurianum
MFKQWEGFQDGEWTNDVNVRDFIQKNYKEYTGDKSFLKGPTEKTKKVWDKAVSLILEELKKGILDVDTETISGINSFKPGYLDKDNEVIVGFQTDAPLKRITNPFGGIRMAEQSLKEYGFKISDEMHNIFTNYRKTHNQGVFDAYSEETRIARSAGVLTGLPDAYGRGRIIGDYRRVALYGIDFLIQEKKKDLSNLKGDMLDELIRLREEVSEQIRALDEIKKMALSYGVDISRPAVNAKEAAQFLYFGYLAGVKENNGAAMSLGRTSTFLDIYIERDLEQGLITEDEAQEVIDQFIIKLRLVRHLRTPEYNELFAGDPTWVTESIAGVGIDGRSLVTKNSFRYLHTLINLGSAPEPNMTVLWSENLPESFKKFCAEMSILTDSIQYENDDIMRPIYGDDYAIACCVSAMRVGKDMQFFGARCNLAKCLLLAINGGVDEKKGIKVVPDIEPITDEVLDYEKVKENYFKVLEYMAGLYVNTMNIIHFMHDKYAYEASQMALHDTKVGRLMAFGIAGFSVAADSLSAIRYAKVKPIRENGITVDFVKEGDFPKYGNDDDRVDSIAVEIVEKFSDELKKHPTYRNAKHTLSVLTITSNVMYGKKTGTTPDGRKVGEPLAPGANPMHGRDMEGALASLNSVAKVPYVCCEDGVSNTFSIVPDALGNDHDVRINNLVSIMGGYFGQGAHHLNVNVLNRETLIDAMNNPDKYPTLTIRVSGYAVNFNRLSKDHQKEVISRTFHEKL

SEQ ID NO: 55 - DNA, 1797 bp, Escherichia coli
  source 1..1797: mol_type = unassigned DNA
atgaccacccaggtgccgccgagcgcgctgctgccgctgaacccggaacagctggcgcgcctgcaggcggcgaccaccgatctgaccccgacccagctggcgtgggtgagcggctatttttggggcgtgctgaaccagcagccggcggcgctggcggcgaccccggcgccggcggcggaaatgccgggcattaccattattagcgcgagccagaccggcaacgcgcgccgcgtggcggaagcgctgcgcgatgatctgctggcggcgaaactgaacgtgaaactggtgaacgcgggcgattataaatttaaacagattgcgagcgaaaaactgctgattgtggtgaccagcacccagggcgaaggcgaaccgccggaagaagcggtggcgctgcataaatttctgtttagcaaaaaagcgccgaaactggaaaacaccgcgtttgcggtgtttagcctgggcgatagcagctatgaatttttttgccagagcggcaaagattttgatagcaaactggcggaactgggcggcgaacgcctgctggatcgcgtggatgcggatgtggaatatcaggcggcggcgagcgaatggcgcgcgcgcgtggtggatgcgctgaaaagccgcgcgccggtggcggcgccgagccagagcgtggcgaccggcgcggtgaacgaaattcataccagcccgtatagcaaagatgcgccgctggtggcgagcctgagcgtgaaccagaaaattaccggccgcaacagcgaaaaagatgtgcgccatattgaaattgatctgggcgatagcggcatgcgctatcagccgggcgatgcgctgggcgtgtggtatcagaacgatccggcgctggtgaaagaactggtggaactgctgtggctgaaaggcgatgaaccggtgaccgtggaaggcaaaaccctgccgctgaacgaagcgctgcagtggcattttgaactgaccgtgaacaccgcgaacattgtggaaaactatgcgaccctgacccgcagcgaaaccctgctgccgctggtgggcgataaagcgaaactgcagcattatgcggcgaccaccccgattgtggatatggtgcgctttagcccggcgcagctggatgcggaagcgctgattaacctgctgcgcccgctgaccccgcgcctgtatagcattgcgagcagccaggcggaagtggaaaacgaagtgcatgtgaccgtgggcgtggtgcgctatgatgtggaaggccgcgcgcgcgcgggcggcgcgagcagctttctggcggatcgcgtggaagaagaaggcgaagtgcgcgtgtttattgaacataacgataactttcgcctgccggcgaacccggaaaccccggtgattatgattggcccgggcaccggcattgcgccgtttcgcgcgtttatgcagcagcgcgcggcggatgaagcgccgggcaaaaactggctgttttttggcaacccgcattttaccgaagattttctgtatcaggtggaatggcagcgctatgtgaaagatggcgtgctgacccgcattgatctggcgtggagccgcgatcagaaagaaaaagtgtatgtgcaggataaactgcgcgaacagggcgcggaactgtggcgctggattaacgatggcgcgcatatttatgtgtgcggcgatgcgaaccgcatggcgaaagatgtggaacaggcgctgctggaagtgattgcggaatttggcggcatggataccgaagcggcggatgaatttctgagcgaactgcgcgtggaacgccgctatcagcgcgatgtgtat

SEQ ID NO: 56 - protein, 599 aa, Escherichia coli
MTTQVPPSALLPLNPEQLARLQAATTDLTPTQLAWVSGYFWGVLNQQPAALAATPAPAAEMPGITIISASQTGNARRVAEALRDDLLAAKLNVKLVNAGDYKFKQIASEKLLIVVTSTQGEGEPPEEAVALHKFLFSKKAPKLENTAFAVFSLGDSSYEFFCQSGKDFDSKLAELGGERLLDRVDADVEYQAAASEWRARVVDALKSRAPVAAPSQSVATGAVNEIHTSPYSKDAPLVASLSVNQKITGRNSEKDVRHIEIDLGDSGMRYQPGDALGVWYQNDPALVKELVELLWLKGDEPVTVEGKTLPLNEALQWHFELTVNTANIVENYATLTRSETLLPLVGDKAKLQHYAATTPIVDMVRFSPAQLDAEALINLLRPLTPRLYSIASSQAEVENEVHVTVGVVRYDVEGRARAGGASSFLADRVEEEGEVRVFIEHNDNFRLPANPETPVIMIGPGTGIAPFRAFMQQRAADEAPGKNWLFFGNPHFTEDFLYQVEWQRYVKDGVLTRIDLAWSRDQKEKVYVQDKLREQGAELWRWINDGAHIYVCGDANRMAKDVEQALLEVIAEFGGMDTEAADEFLSELRVERRYQRDVY

SEQ ID NO: 57 - DNA, 1194 bp, Corynebacterium singulare
  source 1..1194: mol_type = unassigned DNA
atggtgaacaaaattcgcaccacccatgtgggcagcctgccgcgcaccaaagaactgctggaagcgaacctggaacgcagcgcgggcaccattagcgatgaaaaatttcatgaaattctggaacgcagcgtggcggatgtggtgaaacgccaggtggatctgggcgtggatattattaacgaaggcgaatatggccatattaccagcggcgcggtggattatggcgcgtggtggaactatagctttacccgcctgggcggcctgaccatgaccgataaagatcgctgggaaattggcgataaaattcgcagcgaaccgggcaaaattcgcctgagcagcatgaaagatcgccgcgatcgcgcgctgtttagcgaagcgtataacgatccggatagcggcatttttaccggccgcaaaaaagtggcgaacccggaatttaccggcccggtgacctatattggccaggaacaggtggaagcggatgtgaaactgctggcggatgcgctgccggcggataccgaaggctttgtggcggcgctgagcccgggcgcggcggcgcgcctgccgaacaaatattatgaagatgaaagcgaactggtgcgcgcgtgcggcgaagcgctgagcgtggaatataaagcgattaccgatgcgggcctgaccgtgcagtttgatgcgccggatctggcggaagcgtgggatagcgtggtgccggaaccgaccgtgaaagattttcaggcgtttctgcatgaacgcattgaaattctgaacgaaagcattaaagatattccgcgcgaacagacccgcctgcatatttgctggggcagctggcatggcccgcatgtgaccgatattccgtttgaagatattattgatgaaattctgcaggcgaaagtgggcggctttagctttgaaggcgcgagcccgcgccatgcgcatgaatggcgcgtgtggaaagatcataccctgccggaaggcaccgtgatttatccgggcgtggtgagccatagcaccaacgcggtggaacatccgcgcctggtggcggatcgcattattcagtttgcggaactggtgggcccggaaaacgtgattgcgagcaccgattgcggcctgggcggccgcctgcatcatcagattgcgtgggcgaaactggaaagcctggtggaaggcgcggaaattgcgaccaaagaactgttt

SEQ ID NO: 58 - protein, 398 aa, Corynebacterium singulare
MVNKIRTTHVGSLPRTKELLEANLERSAGTISDEKFHEILERSVADVVKRQVDLGVDIINEGEYGHITSGAVDYGAWWNYSFTRLGGLTMTDKDRWEIGDKIRSEPGKIRLSSMKDRRDRALFSEAYNDPDSGIFTGRKKVANPEFTGPVTYIGQEQVEADVKLLADALPADTEGFVAALSPGAAARLPNKYYEDESELVRACGEALSVEYKAITDAGLTVQFDAPDLAEAWDSVVPEPTVKDFQAFLHERIEILNESIKDIPREQTRLHICWGSWHGPHVTDIPFEDIIDEILQAKVGGFSFEGASPRHAHEWRVWKDHTLPEGTVIYPGVVSHSTNAVEHPRLVADRIIQFAELVGPENVIASTDCGLGGRLHHQIAWAKLESLVEGAEIATKELF

SEQ ID NO: 59 - DNA, 885 bp, synthetic construct
  source 1..885: mol_type = other DNA
atggtggatagcgtgtatcgcacccgcagcctgggcgtggcggcggaaggcctgccggatcagtatgcggatggcgaagcggcgcgcgtgtggcagctgtatattggcgatacccgcagccgcaccgcggaatataaagcgtggctgctgggcctgctgcgccagcatggctgccagcgcgtgctggatgtggcgtgcggcaccggcgtggatagcattatgctggtggaagaaggctttagcgtgaccagcgtggatgcgagcgataaaatgctgaaatatgcgctgaaagaacgctggaaccgccgccatgaaccggcgtttgataaatgggtgattgaagaagcgaactggatgaccctggataaagatgtgccgcagagcgcggaaggcggctttgatgcggtgatttgcctgggcaacagctttgcgcatctgccggattgcaaaggcgatcagagcgaacatcgcctggcgctgaaaaacattgcgagcatggtgcgcgcgggcggcctgctggtgattgatcatcgcaactatgatcatattctgagcaccggctgcgcgccgccgggcaaaaacatttattataaaagcgatctgaccaaagatgtgaccaccagcgtgctgattgtgaacaacaaagcgcatatggtgaccctggattataccgtgcaggtgccgggcgcgggccaggatggcagcccgggcctgagcaaatttcgcctgagctattatccgcattgcctggcgagctttaccgaactgctgcaggcggcgtttggcggcaaatgccagcatagcgtgctgggcgattttaaaccgtataaaccgggccagacctatattccgtgctattttattcatgtgctgaaacgcaccgat

SEQ ID NO: 60 - DNA, 885 bp, synthetic construct
  source 1..885: mol_type = other DNA
atggtagatagcgtctatcggacacggtcactgggggttgctgctgagggattgcctgaccagtacgcagacggtgaagccgcaagggtctggcaactctacataggagatacgagatcgcggacagctgagtataaagcatggcttctagggctgcttagacaacacggttgccagagagtgttagatgttgcatgtggcactggcgtagactcgatcatgctggtggaagaaggattttcagtgaccagcgtcgatgcctctgacaaaatgttgaaatatgcactgaaagaacgttggaatcgccgacatgagcccgcgttcgataaatgggtaatcgaagaggccaattggatgacattggataaagacgttccgcagagtgctgaaggaggcttcgatgccgtaatatgtcttggaaactcttttgcacacttaccggattgtaaaggtgaccaatccgaacacagacttgctttaaagaacattgcaagcatggtgcgcgcgggaggcttgcttgtcatcgaccatcgtaactatgatcatattttaagtaccggatgcgcgcctcctggaaagaatatctattacaaatctgatctcactaaggacgtaaccacatcagtcttaattgttaataacaaagcgcatatggtgacgctggattatacggtccaagttccgggtgcgggtcaagatggcagcccagggctgtcaaagtttcgtttatcttattacccgcattgtctggcgtcctttacagaattgcttcaggccgctttcgggggcaaatgccaacattccgtgcttggcgattttaaaccgtacaagcctggccagacatatattccatgctattttattcatgttctcaaacgcacggat

SEQ ID NO: 61 - protein, 295 aa, Homo sapiens
MVDSVYRTRSLGVAAEGLPDQYADGEAARVWQLYIGDTRSRTAEYKAWLLGLLRQHGCQRVLDVACGTGVDSIMLVEEGFSVTSVDASDKMLKYALKERWNRRHEPAFDKWVIEEANWMTLDKDVPQSAEGGFDAVICLGNSFAHLPDCKGDQSEHRLALKNIASMVRAGGLLVIDHRNYDHILSTGCAPPGKNIYYKSDLTKDVTTSVLIVNNKAHMVTLDYTVQVPGAGQDGSPGLSKFRLSYYPHCLASFTELLQAAFGGKCQHSVLGDFKPYKPGQTYIPCYFIHVLKRTD

SEQ ID NO: 62 - DNA, 1479 bp, Homo sapiens
  source 1..1479: mol_type = unassigned DNA
atggcggatagcgaagcgctgccgagcctggcgggcgatccggtggcggtggaagcgctgctgcgcgcggtgtttggcgtggtggtggatgaagcgattcagaaaggcaccagcgtgagccagaaagtgtgcgaatggaaagaaccggaagaactgaaacagctgctggatctggaactgcgcagccagggcgaaagccagaaacagattctggaacgctgccgcgcggtgattcgctatagcgtgaaaaccggccatccgcgcttttttaaccagctgtttagcggcctggatccgcatgcgctggcgggccgcattattaccgaaagcctgaacaccagccagtatacctatgaaattgcgccggtgtttgtgctgatggaagaagaagtgctgcgcaaactgcgcgcgctggtgggctggagcagcggcgatggcattttttgcccgggcggcagcattagcaacatgtatgcggtgaacctggcgcgctatcagcgctatccggattgcaaacagcgcggcctgcgcaccctgccgccgctggcgctgtttaccagcaaagaatgccattatagcattcagaaaggcgcggcgtttctgggcctgggcaccgatagcgtgcgcgtggtgaaagcggatgaacgcggcaaaatggtgccggaagatctggaacgccagattggcatggcggaagcggaaggcgcggtgccgtttctggtgagcgcgaccagcggcaccaccgtgctgggcgcgtttgatccgctggaagcgattgcggatgtgtgccagcgccatggcctgtggctgcatgtggatgcggcgtggggcggcagcgtgctgctgagccagacccatcgccatctgctggatggcattcagcgcgcggatagcgtggcgtggaacccgcataaactgctggcggcgggcctgcagtgcagcgcgctgctgctgcaggataccagcaacctgctgaaacgctgccatggcagccaggcgagctatctgtttcagcaggataaattttatgatgtggcgctggataccggcgataaagtggtgcagtgcggccgccgcgtggattgcctgaaactgtggctgatgtggaaagcgcagggcgatcagggcctggaacgccgcattgatcaggcgtttgtgctggcgcgctatctggtggaagaaatgaaaaaacgcgaaggctttgaactggtgatggaaccggaatttgtgaacgtgtgcttttggtttgtgccgccgagcctgcgcggcaaacaggaaagcccggattatcatgaacgcctgagcaaagtggcgccggtgctgaaagaacgcatggtgaaagaaggcagcatgatgattggctatcagccgcatggcacccgcggcaacttttttcgcgtggtggtggcgaacagcgcgctgacctgcgcggatatggattttctgctgaacgaactggaacgcctgggccaggatctg

SEQ ID NO: 63 - protein, 493 aa, Homo sapiens
MADSEALPSLAGDPVAVEALLRAVFGVVVDEAIQKGTSVSQKVCEWKEPEELKQLLDLELRSQGESQKQILERCRAVIRYSVKTGHPRFFNQLFSGLDPHALAGRIITESLNTSQYTYEIAPVFVLMEEEVLRKLRALVGWSSGDGIFCPGGSISNMYAVNLARYQRYPDCKQRGLRTLPPLALFTSKECHYSIQKGAAFLGLGTDSVRVVKADERGKMVPEDLERQIGMAEAEGAVPFLVSATSGTTVLGAFDPLEAIADVCQRHGLWLHVDAAWGGSVLLSQTHRHLLDGIQRADSVAWNPHKLLAAGLQCSALLLQDTSNLLKRCHGSQASYLFQQDKFYDVALDTGDKVVQCGRRVDCLKLWLMWKAQGDQGLERRIDQAFVLARYLVEEMKKREGFELVMEPEFVNVCFWFVPPSLRGKQESPDYHERLSKVAPVLKERMVKEGSMMIGYQPHGTRGNFFRVVVANSALTCADMDFLLNELERLGQDL

SEQ ID NO: 64 - DNA, 1077 bp, unidentified
  source 1..1077: mol_type = unassigned DNA
atgattaccccattaacgcttgctacactctcgaaaaatcctatactggttgattttttcgatcctgaagatggacgttggaattcacatgtcgatttaggcctctggtcagatctgtatcttatcgcgcctgcaacggcgaacaccatcggaaaaatggcagcaggtattgcggacaatcttttattgacatcttacttatccgctcggtgcccggtatttattgcccccgccatggatgttgatatgttaatgcatccggcaactcaaagaaacctgggaatccttaaatcttcaggaaaccacataattgagccgggtagcggggagcttgcctctggtctaacgggaaaaggccgcatggcagaacccgaagaaatcgtaagagaggtcatttcgtttttctcaaaaaagaaaattaccgaaaaaccattgaatggacgacgagtttttattaacgcgggccctacgattgaaccgattgatccggtgaggttcatatccaactatagctccgggcggatggggattgcgcttgctgatgccgcggccgcgatgggagctgaggtgacattggtcctgggtccggtcactctgcgtccgagttctcaggacatcaatgttatcgacgtgaggagtgcagctgaaatgaaagaagcgtcagtagaagcttttagagaatgtgacatagcaatacttgccgccgctgtcgcagactttacaccgttgaccacaagcgacaagaagattaaacgcggctctggtgaaatggttatcaatttaagacctacggaagatattgctgcggaactcggcaaaatgaaaaagaagaatcaattgctggttgggtttgctctggagacagacgatgaaattacaaatgcgagctcaaaactgaaacggaagaatctcgatatgatcgtgctaaatagcttaaaggatccaggcgccggctttggacacgagactaaccgcatcacaatcattgataaaagtaacaacatcgataaattcgaactgaaaacgaaaggcgaggtggcagcagacattattcgtaagatcttgacacttgtacat

SEQ ID NO: 65 - DNA, 1077 bp, synthetic construct
  source 1..1077: mol_type = other DNA
atgataacgccattaacgctcgctaccctgtccaaaaatccgattttggtggatttctttgatcctgaagatggccgttggaattcacacgtggatcttggtttatggtcagatctgtacttaattgcccctgcgaccgctaatacaattggtaaaatggcagcgggaattgcagataacttattacttacgagctatctaagcgcgcgctgcccggttttcattgcccctgccatggatcttgacatgctcatgcatccggcgacacaacgaaaccttggaatacttaagtctagtggcaatcatatcatcgaacccggctcaggagaacttgcttcagggctgacaggtaaagggcggatggcagaaccggaggagatcgtgagagaggttatttcctttttcagtaaaaaaaagattaccgaaaaaccgttgaacgggcggcgtgtttttattaatgccggtccaaccatcgaaccgatcgatccggtccgcttcatctctaattatagcagtggacgtatgggaatcgcgttggcagacgctgcggctgccatgggcgccgaagtcacattagtcttaggtcctgttactttgaggccttcctcgcaggacattaatgtgatagacgtgagatctgcagccgagatgaaagaagcttcagtagaagcatttagggagtgtgacattgcaatcttggccgcagctgtcgcagactctactccgctgacgacaagcgataaaaagatgaagcgcggcagcggcgaaatggttataaaccttcgacccacggaactgattgcagcggaactaggaaaaatgaagaaaaaaaaccaactgctggtaggctttgctctggagacagatgatgaaatcacaaacgcttcgtctaagctcaagagaaaaaatcttgacatgattgtactcaatagccttaaggacccaggagcgggctttgggcacgagacaaaccggattactatcattgataaatcaaataacattgataaatttgaattgaaaacaaaaggagaagtcgcggcggatattatcagaaaaatcctgacgttagtacat

SEQ ID NO: 66 - protein, 359 aa, unidentified
MITPLTLATLSKNPILVDFFDPEDGRWNSHVDLGLWSDLYLIAPATANTIGKMAAGIADNLLLTSYLSARCPVFIAPAMDVDMLMHPATQRNLGILKSSGNHIIEPGSGELASGLTGKGRMAEPEEIVREVISFFSKKKITEKPLNGRRVFINAGPTIEPIDPVRFISNYSSGRMGIALADAAAAMGAEVTLVLGPVTLRPSSQDINVIDVRSAAEMKEASVEAFRECDIAILAAAVADFTPLTTSDKKIKRGSGEMVINLRPTEDIAAELGKMKKKNQLLVGFALETDDEITNASSKLKRKNLDMIVLNSLKDPGAGFGHETNRITIIDKSNNIDKFELKTKGEVAADIIRKILTLVH

SEQ ID NO: 67 - protein, 359 aa, synthetic construct
MITPLTLATLSKNPILVDFFDPEDGRWNSHVDLGLWSDLYLIAPATANTIGKMAAGIADNLLLTSYLSARCPVFIAPAMDLDMLMHPATQRNLGILKSSGNHIIEPGSGELASGLTGKGRMAEPEEIVREVISFFSKKKITEKPLNGRRVFINAGPTIEPIDPVRFISNYSSGRMGIALADAAAAMGAEVTLVLGPVTLRPSSQDINVIDVRSAAEMKEASVEAFRECDIAILAAAVADSTPLTTSDKKMKRGSGEMVINLRPTELIAAELGKMKKKNQLLVGFALETDDEITNASSKLKRKNLDMIVLNSLKDPGAGFGHETNRITIIDKSNNIDKFELKTKGEVAADIIRKILTLVH

SEQ ID NO: 68 - DNA, 1596 bp, Homo sapiens
  source 1..1596: mol_type = unassigned DNA
atggcgaaacgcgtggcgattgtgggcgcgggcgtgagcggcctggcgagcattaaatgctgcctggaagaaggcctggaaccgacctgctttgaacgcagcgatgatctgggcggcctgtggcgctttaccgaacatgtggaagaaggccgcgcgagcctgtataaaagcgtggtgagcaacagctgcaaagaaatgagctgctatagcgattttccgtttccggaagattatccgaactatgtgccgaacagccagtttctggaatatctgaaaatgtatgcgaaccattttgatctgctgaaacatattcagtttaaaaccaaagtgtgcagcgtgaccaaatgcagcgatagcgcggtgagcggccagtgggaagtggtgaccatgcatgaagaaaaacaggaaagcgcgatttttgatgcggtgatggtgtgcaccggctttctgaccaacccgtatctgccgctggatagctttccgggcattaacgcgtttaaaggccagtattttcatagccgccagtataaacatccggatatttttaaagataaacgcgtgctggtgattggcatgggcaacagcggcaccgatattgcggtggaagcgagccatctggcggaaaaagtgtttctgagcaccaccggcggcggctgggtgattagccgcatttttgatagcggctatccgtgggatatggtgtttatgacccgctttcagaacatgctgcgcaacagcctgccgaccccgattgtgacctggctgatggaacgcaaaattaacaactggctgaaccatgcgaactatggcctgattccggaagatcgcacccagctgaaagaatttgtgctgaacgatgaactgccgggccgcattattaccggcaaagtgtttattcgcccgagcattaaagaagtgaaagaaaacagcgtgatttttaacaacaccagcaaagaagaaccgattgatattattgtgtttgcgaccggctatacctttgcgtttccgtttctggatgaaagcgtggtgaaagtggaagatggccaggcgagcctgtataaatatatttttccggcgcatctgcagaaaccgaccctggcgattattggcctgattaaaccgctgggcagcatgattccgaccggcgaaacccaggcgcgctgggcggtgcgcgtgctgaaaggcgtgaacaaactgccgccgccgagcgtgatgattgaagaaattaacgcgcgcaaagaaaacaaaccgagctggtttggcctgtgctattgcaaagcgctgcagagcgattatattacctatattgatgaactgctgacctatattaacgcgaaaccgaacctgtttagcatgctgctgaccgatccgcatctggcgctgaccgtgttttttggcccgtgcagcccgtatcagtttcgcctgaccggcccgggcaaatgggaaggcgcgcgcaacgcgattatgacccagtgggatcgcacctttaaagtgattaaagcgcgcgtggtgcaggaaagcccgagcccgtttgaaagctttctgaaagtgtttagctttctggcgctgctggtggcgatttttctgatttttctg

SEQ ID NO: 69 - DNA, 1605 bp, Homo sapiens
  source 1..1605: mol_type = unassigned DNA
atggcgaaaaaagtggcggtgattggcgcgggcgtgagcggcctgattagcctgaaatgctgcgtggatgaaggcctggaaccgacctgctttgaacgcaccgaagatattggcggcgtgtggcgctttaaagaaaacgtggaagatggccgcgcgagcatttatcagagcgtggtgaccaacaccagcaaagaaatgagctgctttagcgattttccgatgccggaagattttccgaactttctgcataacagcaaactgctggaatattttcgcatttttgcgaaaaaatttgatctgctgaaatatattcagtttcagaccaccgtgctgagcgtgcgcaaatgcccggattttagcagcagcggccagtggaaagtggtgacccagagcaacggcaaagaacagagcgcggtgtttgatgcggtgatggtgtgcagcggccatcatattctgccgcatattccgctgaaaagctttccgggcatggaacgctttaaaggccagtattttcatagccgccagtataaacatccggatggctttgaaggcaaacgcattctggtgattggcatgggcaacagcggcagcgatattgcggtggaactgagcaaaaacgcggcgcaggtgtttattagcacccgccatggcacctgggtgatgagccgcattagcgaagatggctatccgtgggatagcgtgtttcatacccgctttcgcagcatgctgcgcaacgtgctgccgcgcaccgcggtgaaatggatgattgaacagcagatgaaccgctggtttaaccatgaaaactatggcctggaaccgcagaacaaatatattatgaaagaaccggtgctgaacgatgatgtgccgagccgcctgctgtgcggcgcgattaaagtgaaaagcaccgtgaaagaactgaccgaaaccagcgcgatttttgaagatggcaccgtggaagaaaacattgatgtgattatttttgcgaccggctatagctttagctttccgtttctggaagatagcctggtgaaagtggaaaacaacatggtgagcctgtataaatatatttttccggcgcatctggataaaagcaccctggcgtgcattggcctgattcagccgctgggcagcatttttccgaccgcggaactgcaggcgcgctgggtgacccgcgtgtttaaaggcctgtgcagcctgccgagcgaacgcaccatgatgatggatattattaaacgcaacgaaaaacgcattgatctgtttggcgaaagccagagccagaccctgcagaccaactatgtggattatctggatgaactggcgctggaaattggcgcgaaaccggatttttgcagcctgctgtttaaagatccgaaactggcggtgcgcctgtattttggcccgtgcaacagctatcagtatcgcctggtgggcccgggccagtgggaaggcgcgcgcaacgcgatttttacccagaaacagcgcattctgaaaccgctgaaaacccgcgcgctgaaagatagcagcaactttagcgtgagctttctgctgaaaattctgggcctgctggcggtggtggtggcgtttttttgccagctgcagtggagc

SEQ ID NO: 70 - DNA, 1596 bp, Homo sapiens
  source 1..1596: mol_type = unassigned DNA
atgggcaaaaaagtggcgattattggcgcgggcgtgagcggcctggcgagcattcgcagctgcctggaagaaggcctggaaccgacctgctttgaaaaaagcaacgatattggcggcctgtggaaatttagcgatcatgcggaagaaggccgcgcgagcatttataaaagcgtgtttagcaacagcagcaaagaaatgatgtgctttccggattttccgtttccggatgattttccgaactttatgcataacagcaaaattcaggaatatattattgcgtttgcgaaagaaaaaaacctgctgaaatatattcagtttaaaacctttgtgagcagcgtgaacaaacatccggattttgcgaccaccggccagtgggatgtgaccaccgaacgcgatggcaaaaaagaaagcgcggtgtttgatgcggtgatggtgtgcagcggccatcatgtgtatccgaacctgccgaaagaaagctttccgggcctgaaccattttaaaggcaaatgctttcatagccgcgattataaagaaccgggcgtgtttaacggcaaacgcgtgctggtggtgggcctgggcaacagcggctgcgatattgcgaccgaactgagccgcaccgcggaacaggtgatgattagcagccgcagcggcagctgggtgatgagccgcgtgtgggataacggctatccgtgggatatgctgctggtgacccgctttggcacctttctgaaaaacaacctgccgaccgcgattagcgattggctgtatgtgaaacagatgaacgcgcgctttaaacatgaaaactatggcctgatgccgctgaacggcgtgctgcgcaaagaaccggtgtttaacgatgaactgccggcgagcattctgtgcggcattgtgagcgtgaaaccgaacgtgaaagaatttaccgaaaccagcgcgatttttgaagatggcaccatttttgaaggcattgattgcgtgatttttgcgaccggctatagctttgcgtatccgtttctggatgaaagcattattaaaagccgcaacaacgaaattattctgtttaaaggcgtgtttccgccgctgctggaaaaaagcaccattgcggtgattggctttgtgcagagcctgggcgcggcgattccgaccgtggatctgcagagccgctgggcggcgcaggtgattaaaggcacctgcaccctgccgagcatggaagatatgatgaacgatattaacgaaaaaatggaaaaaaaacgcaaatggtttggcaaaagcgaaaccattcagaccgattatattgtgtatatggatgaactgagcagctttattggcgcgaaaccgaacattccgtggctgtttctgaccgatccgaaactggcgatggaagtgtattttggcccgtgcagcccgtatcagtttcgcctggtgggcccgggccagtggccgggcgcgcgcaacgcgattctgacccagtgggatcgcagcctgaaaccgatgcagacccgcgtggtgggccgcctgcagaaaccgtgctttttttttcattggctgaaactgtttgcgattccgattctgctgattgcggtgtttctggtgctgacc

SEQ ID NO: 71 - protein, 532 aa, Homo sapiens
MAKRVAIVGAGVSGLASIKCCLEEGLEPTCFERSDDLGGLWRFTEHVEEGRASLYKSVVSNSCKEMSCYSDFPFPEDYPNYVPNSQFLEYLKMYANHFDLLKHIQFKTKVCSVTKCSDSAVSGQWEVVTMHEEKQESAIFDAVMVCTGFLTNPYLPLDSFPGINAFKGQYFHSRQYKHPDIFKDKRVLVIGMGNSGTDIAVEASHLAEKVFLSTTGGGWVISRIFDSGYPWDMVFMTRFQNMLRNSLPTPIVTWLMERKINNWLNHANYGLIPEDRTQLKEFVLNDELPGRIITGKVFIRPSIKEVKENSVIFNNTSKEEPIDIIVFATGYTFAFPFLDESVVKVEDGQASLYKYIFPAHLQKPTLAIIGLIKPLGSMIPTGETQARWAVRVLKGVNKLPPPSVMIEEINARKENKPSWFGLCYCKALQSDYITYIDELLTYINAKPNLFSMLLTDPHLALTVFFGPCSPYQFRLTGPGKWEGARNAIMTQWDRTFKVIKARVVQESPSPFESFLKVFSFLALLVAIFLIFL

SEQ ID NO: 72 - protein, 535 aa, Homo sapiens [sequence omitted]

SEQ ID NO: 73 - protein, 532 aa, Homo sapiens
MGKKVAIIGAGVSGLASIRSCLEEGLEPTCFEKSNDIGGLWKFSDHAEEGRASIYKSVFSNSSKEMMCFPDFPFPDDFPNFMHNSKIQEYIIAFAKEKNLLKYIQFKTFVSSVNKHPDFATTGQWDVTTERDGKKESAVFDAVMVCSGHHVYPNLPKESFPGLNHFKGKCFHSRDYKEPGVFNGKRVLVVGLGNSGCDIATELSRTAEQVMISSRSGSWVMSRVWDNGYPWDMLLVTRFGTFLKNNLPTAISDWLYVKQMNARFKHENYGLMPLNGVLRKEPVFNDELPASILCGIVSVKPNVKEFTETSAIFEDGTIFEGIDCVIFATGYSFAYPFLDESIIKSRNNEIILFKGVFPPLLEKSTIAVIGFVQSLGAAIPTVDLQSRWAAQVIKGTCTLPSMEDMMNDINEKMEKKRKWFGKSETIQTDYIVYMDELSSFIGAKPNIPWLFLTDPKLAMEVYFGPCSPYQFRLVGPGQWPGARNAILTQWDRSLKPMQTRVVGRLQKPCFFFHWLKLFAIPILLIAVFLVLT

SEQ ID NO: 74 - DNA, 831 bp, Halothece sp.
  source 1..831: mol_type = unassigned DNA
atgacaaaagcggacgccgttgctaaacaagcccaagattactatgattcgggcagcgctgatggattctactatagaatctggggcggcgaggatctgcacataggtatatacaatacaccagatgaaccaatttacgatgcaagcgttcgaacggtgtctcgtatctgcgacaaaatcaaaaactggcccgcaggcacaaaggtgttagacttaggtgcggggtatggaggcagtgcccgctacatggcgaaacatcatggattcgacgtagattgcttgaacatttctttagtacaaaacgaaagaaatcgtcaaatgaatcaagaacagggtcttgcagataaaattagggtctttgacggatcatttgaggaattgccgttcgagaataagtcatatgatgtgctatggtcccaagactccattctgcactcagggaatcgcagaaaagttatggaagaagccgatcgtgtacttaaatctgggggcgattttgtctttactgacccgatgcaaaccgataactgtcctgaaggtgtcttggagccggtgctggcgcggattcatctcgatagtctgggttcagttggcttttatagacaagtggcagaggaattagggtgggaattcgtcgaatttgacgaacagacgcatcagttggtcaatcattatagccgggtacttcaggagttagaagctcattatgatcagcttcagcctgaatgttcgcaggaatatcttgatcgcatgaaagttggactcaatcactggattaacgcaggcaaaagcggatatatggcttggggaatcctgaagtttcataagccg

SEQ ID NO: 75 - protein, 277 aa, Halothece sp.
MTKADAVAKQAQDYYDSGSADGFYYRIWGGEDLHIGIYNTPDEPIYDASVRTVSRICDKIKNWPAGTKVLDLGAGYGGSARYMAKHHGFDVDCLNISLVQNERNRQMNQEQGLADKIRVFDGSFEELPFENKSYDVLWSQDSILHSGNRRKVMEEADRVLKSGGDFVFTDPMQTDNCPEGVLEPVLARIHLDSLGSVGFYRQVAEELGWEFVEFDEQTHQLVNHYSRVLQELEAHYDQLQPECSQEYLDRMKVGLNHWINAGKSGYMAWGILKFHKP

SEQ ID NO: 76 - DNA, 837 bp, Halorhodospira halochloris
  source 1..837: mol_type = unassigned DNA
atggccacacgttacgacgatcaagcgattgagacagcacgccagtactataatagtgaggacgcggataatttctatgccattatctggggaggggaggacattcatatcggcttatataacgatgacgaagaacctatagccgatgctagtcggagaactgttgaacgcatgtcttcgttgtccaggcaattaggtccagactcttatgtactcgatatgggagcaggatacgggggctcagctcgttatcttgcacataaatatggttgtaaggtagcagctttgaacttgtccgaaagagaaaatgaacgagaccgtcaaatgaacaaagaacaaggtgtcgatcatttaattgaagtcgttgatgccgcgtttgaagacgtgccgtatgatgatggcgtgtttgatctcgtctggtcacaagattcattcttacatagccctgatcgcgaacgtgtactgagagaagcgagccgtgttctgcggtctggaggagagttcatatttacagatccgatgcaagctgacgattgcccggagggagttattcagccaatccttgatagaattcaccttgaaacgatgggaaccccgaatttttatagacagaccctgcgagacctaggatttgaagagattacgttcgaagatcatacacaccagcttcccaggcactatgggcgcgtccggcgcgaactggatagacgagagggcgagctgcagggccatgtgagcgcagaatacatcgaacggatgaaaaacggtttagaccattgggtgaatggcggcaataaagggtaccttacgtggggtatcttttattttaggaagggc

SEQ ID NO: 77 - protein, 279 aa, Halorhodospira halochloris
MATRYDDQAIETARQYYNSEDADNFYAIIWGGEDIHIGLYNDDEEPIADASRRTVERMSSLSRQLGPDSYVLDMGAGYGGSARYLAHKYGCKVAALNLSERENERDRQMNKEQGVDHLIEVVDAAFEDVPYDDGVFDLVWSQDSFLHSPDRERVLREASRVLRSGGEFIFTDPMQADDCPEGVIQPILDRIHLETMGTPNFYRQTLRDLGFEEITFEDHTHQLPRHYGRVRRELDRREGELQGHVSAEYIERMKNGLDHWVNGGNKGYLTWGIFYFRKG

SEQ ID NO: 78 - DNA, 804 bp, Halorhodospira halochloris
  source 1..804: mol_type = unassigned DNA
atgaatacaacgacggaacaagattttggagcggaccctaccaaagtaagagacacagatcattacactgaagaatacgtggatggattcgttgacaaatgggatgacttaattgattgggatagccgggctaagtccgaaggggatttttttattcaggaacttaaaaagcgcggggccacgagaattctagacgccgcaacaggcacgggctttcattctgtgagacttctcgaagccggttttgatgtcgtctccgcggatggctctgctgagatgcttgcgaaagcctttgagaatggccgtaaacgtggacacatcctcaggaccgtccaggtggactggagatggttgaaccgcgatatacacggtcggtatgatgcaatcatttgtctgggcaattcatttactcatctgtttaatgaaaaggatagacgtaaaactcttgcagagttttacagcgcattgaacccggaaggcgtattaatcctggatcaacgcaactatgatggtatactggatcatggctatgatagcagtcattcgtattactattgcggagagggagtctcagtttatccggaacacgttgacgacggattagcgcgatttaaatatgaatttaacgacggatcaacctacttcctgaatatgttcccattacgtaaagactatacacgaaggttgatgcatgaagtagggttccaaaagatcgacacatatggtgatttcaaagcaacataccgcgatgctgaccccgatttctttattcatgttgctgaaaaagaatatcgggaggaggat

SEQ ID NO: 79 - protein, 268 aa, Halorhodospira halochloris
MNTTTEQDFGADPTKVRDTDHYTEEYVDGFVDKWDDLIDWDSRAKSEGDFFIQELKKRGATRILDAATGTGFHSVRLLEAGFDVVSADGSAEMLAKAFENGRKRGHILRTVQVDWRWLNRDIHGRYDAIICLGNSFTHLFNEKDRRKTLAEFYSALNPEGVLILDQRNYDGILDHGYDSSHSYYYCGEGVSVYPEHVDDGLARFKYEFNDGSTYFLNMFPLRKDYTRRLMHEVGFQKIDTYGDFKATYRDADPDFFIHVAEKEYREED

SEQ ID NO: 80 - protein, 360 aa, Lactococcus lactis
IIELNNLSVQFHQKGRLVTAVKNATLHIEKGDIYGVIGYSGAGKSTLVRTINLLQKPTEGQIVINGEKIFDSENPVKFTGAKLREFRQKIGMIFQHFNLLSEKTVFNNVAFALQHSQIEDKNGKKRYLTKKEKNDKVTELLKLVDLADLSDKYPAQLSGGQKQRVAIARALANDPEILISDEGTSALDPKTTNQILDLLKSLHEKLGITVVLITHEMQVVKEIANKVAVMQNGEIIEQNSLIDIFAQPKEALTKQFIETTSSVNRFIASLSKTELLAQLADDEELIHLDYSGSELEDPVVSDITKKFDVTTNIFYGNVELLQGQPFGSLVLTLKGSSEHRAAAKAYFVERHLKFEVLGKI

SEQ ID NO: 81 - protein, 360 aa, synthetic construct
IIELNNLSVQFHQKGRLVTAVKNATLHIEKGDIYGVIGYSGAGKSTLVRTINLLQKPTEGQIVINGEKIFDSENPVKFTGAKLREFRQKIGMIFQHFNLLSEKTVFNNVAFALQHSQIEDKNGKKRYLTKKEKNDKVTELLKLVDLADLSDKYPAQLSGGQKQRVAIARALANDPEILISDEGTSALDPKTTNQILDLLKSLHEKLGITVVLITHEMQVVKEIANKVAVMQNGEIIEQNSLIDIFAQPKEALTKQFIETTSSVNRFIASLSKTELLAQLADDEELIHLDYSGSELEDPVVSDITKKFDVTTAIFYGNVELLQGQPFGSLVLTLKGSSEHRAAAKAYFVERHLKFEVLGKI

SEQ ID NO: 82 - protein, 355 aa, Lactiplantibacillus plantarum
MTEAVIDLTKIGVTFKDGQQTIQAVQDVDLKIEAGDIYGIIGYSGAGKSTLVRVINLLQVPTTGRVVVNGQTLQELSPVALRQARKRVGMIFQHFNLMQSRTVLGNVVYPLLGQKISKSERRAKALRLLKLVGLEDYAESYPDKLSGGQKQRVAIARALVTDPQILISDEATSALDPKTTAAILSLLQQVNRNLGVTIVLITHEMQVIKSVCHHVAVMENGRIIERGPVAQVFTAPQAPLTVDFVETSTNVRAAIERITRTIKLSELADDQELIAFKFVGQSTKQGIVSHLSQTLGVDVNILFANIDQIDGQNVGDMIAIITGNLPAFNQAIAQLADQGVQTHVINEQDVKELVD

SEQ ID NO: 83 - protein, 355 aa, synthetic construct
MTEAVIDLTKIGVTFKDGQQTIQAVQDVDLKIEAGDIYGIIGYSGAGKSTLVRVINLLQVPTTGRVVVNGQTLQELSPVALRQARKRVGMIFQHFNLMQSRTVLGNVVYPLLGQKISKSERRAKALRLLKLVGLEDYAESYPDKLSGGQKQRVAIARALVTDPQILISDEATSALDPKTTAAILSLLQQVNRNLGVTIVLITHEMQVIKSVCHHVAVMENGRIIERGPVAQVFTAPQAPLTVDFVETSTNVRAAIERITRTIKLSELADDQELIAFKFVGQSTKQGIVSHLSQTLGVDVAILFANIDQIDGQNVGDMIAIITGNLPAFNQAIAQLADQGVQTHVINEQDVKELVD

SEQ ID NO: 84 - protein, 231 aa, Lactococcus lactis
MAEWFAHTFPNVVYLGWTGETGWWTAIVQTLYMTFISALIGGLLGLIFGIGVVVTAEDGITPNRPLFWILDKIVSIGRAFPFIILLAAIAPLTKILVGTQIGVTAALVPLALGVAPFYARQVQASLESVDHGKVEAAQTVGADFLDIVFTVYLREELASLVRVSTVTLISLIGLTAMAGAIGAGGLGNTAISYGYNRFANDVTWFATILILIFVLLVQLVGDFLARRVSHR

SEQ ID NO: 85 - protein, 225 aa, Lactiplantibacillus plantarum
MLQKLIPNVLQMKGEFVQATWETLYMTFGSAIIAAVLGLLLGVCLVITQPGGILEDVLTYSVLDKITNLLRSIPFIILLAVISPLTQFLIGTTVGTTASLVPLIVGIFPFYARQVQNALLTVEHGVIEAAQAMGSSPTEIVFRVYLREGLADILRVSIVTVISLIGLTTMAGAIGSGGLGDVAISIGYARFENDVTFVAMIIILILVFAVQIIGDLIVKAVEHNE

SEQ ID NO: 86 - protein, 286 aa, Lactococcus lactis
MNPRNRNIIIGIIVVVIVAIAAFIGFGQKSQANKTVNKTVKIGIMTGTKEDDSIWQTVSKTAKDKYGITLKFTHFTDYTQPNTALKNGDIDLNAFQHYAFLKAWNKANNGNLVAIGDTVISPISVYSKQLKNISDIKEGGTIAVPNDASNESRALYVLKSAGLIKLDVSGQTLATVKDITSNPKNLVIKELDASQTARALDSVDAAVINNNYAVTAGLKKSDAIFTEPVNKDSQQWINIIVANKKDENNTVYKDVVKAYETEATKETIAKAYPDKSTIPAWGLKLK

SEQ ID NO: 87 - protein, 286 aa, synthetic construct
MNPRNRNIIIGIIVVVIVAIAAFIGFGQKSQANKTVNKTVKIGIMTGTKEDDSIWQTVSKTAKDKYGITLKFTHFTDYTQPNTALKNGDIDLNAFQHYAFLKAWNKANNGNLVAIGDTVISPISVYSKQLKNISDIKEGGTIAVPNDASNESRALYVLKSAGLIKLDVSGQTLATVKDITSNPKNLVIKELDASQTARALDSVDAAVINNNYAVTAGLKKSDAIFTEPVNKDSQQWIAIIVANKKDENNTVYKDVVKAYETEATKETIAKAYPDKSTIPAWGLKLK

SEQ ID NO: 88 - protein, 275 aa, Lactiplantibacillus plantarum
MKKKGILGLLAVAATAFLLVGCGKSSSATKTTTITVGASSVPHAQVLKHVQPELKKEGVNLKIKAFQDYVLPNKALASKELDANYFQHIPFLDNWNKENNGTLVSAGKVHLEPIGVYSKKVKSLKDLKDGATVLVSSNVADYGRVLTLFKDAGLITLKPGTKLTSATFNDIKTNKRHLKFKHSYEAKLMPTFYKNNEGDAVVINANYAVQAGLSPKKDAIALEKSDSPYANIVAVRKGDKNKPAIKKLMKALRSKSTQQWIEKKYKGAILPVSAD

SEQ ID NO: 89 - protein, 275 aa, synthetic construct
MKKKGILGLLAVAATAFLLVGCGKSSSATKTTTITVGASSVPHAQVLKHVQPELKKEGVNLKIKAFQDYVLPNKALASKELDANYFQHIPFLDNWNKENNGTLVSAGKVHLEPIGVYSKKVKSLKDLKDGATVLVSSNVADYGRVLTLFKDAGLITLKPGTKLTSATFNDIKTNKRHLKFKHSYEAKLMPTFYKNNEGDAVVINANYAVQAGLSPKKDAIALEKSDSPYAAIVAVRKGDKNKPAIKKLMKALRSKSTQQWIEKKYKGAILPVSAD

SEQ ID NO: 90 - protein, 32 aa, synthetic construct
MKKKKTWKRFLHFSSAALAAGLIFTSAAPAEA

SEQ ID NO: 91 - protein, 33 aa, synthetic construct
MFAKRFKTSLLPLFAGFLLLFHLVLAGPAAASA

SEQ ID NO: 92 - protein, 29 aa, synthetic construct
MRSKKLWISLLFALTLIFTMAFSNMSVQA

SEQ ID NO: 93 - protein, 22 aa, synthetic construct
MKLAKRVSALTPSTTLAITAKA

SEQ ID NO: 94 - protein, 29 aa, synthetic construct
MKRSISIFITCLLITLLTMGGMIASPASA

SEQ ID NO: 95 - protein, 28 aa, synthetic construct
MPYLKRVLLLLVTGLFMSLFAVTATASA

SEQ ID NO: 96 - protein, 30 aa, synthetic construct
MRKKTKNRLISSVLSTVVISSLLFPGAAGA

SEQ ID NO: 97 - protein, 27 aa, synthetic construct
MKWNPLIPFLLIAVLGIGLTFFLSVKG

SEQ ID NO: 98 - protein, 32 aa, synthetic construct
MGNTRKKVSVIGAGFTGATTAFLIAQKELADV

SEQ ID NO: 99 - protein, 37 aa, synthetic construct
MKNRLFILICFCVICLFLSFGQPFFPSMILTVQAAKS

SEQ ID NO: 100 - protein, 35 aa, synthetic construct
MKISMQKADFWKKAAISLLVFTMFFTLMMSETVFA

SEQ ID NO: 101 - protein, 27 aa, synthetic construct
MRKKLKWLSFLLGFIILLFLFKYQFSN

SEQ ID NO: 102 - protein, 27 aa, synthetic construct
MRIFKKAVFVIMISFLIATVNVNTAHA

SEQ ID NO: 103 - protein, 23 aa, synthetic construct
MKRLLSTLLIGIMLLTFAPSAFA

SEQ ID NO: 104 - protein, 21 aa, synthetic construct
MKKRFFGPIILAFILFAGAIA

SEQ ID NO: 105 - protein, 27 aa, synthetic construct
MKNMSCKLVVSVTLFFSFLTIGPLAHA

SEQ ID NO: 106 - protein, 23 aa, synthetic construct
MKKKLMIILLIILIVIGALGAAA

SEQ ID NO: 107 - protein, 27 aa, synthetic construct
MKKSQYFIVFICFFVLFSVHPIAAAAA

SEQ ID NO: 108 - protein, 26 aa, synthetic construct
MRKNRILALFVLSLGLLSFMVTPVSA

SEQ ID NO: 109 - protein, 31 aa, synthetic construct
MKFVKRRIIALVTILMLSVTSLFALQPSAKA

SEQ ID NO: 110 - protein, 28 aa, synthetic construct
MKKVLMAFIICLSLILSVLAAPPSGAKA

SEQ ID NO: 111 - protein, 25 aa, synthetic construct
MKSCKQLIVCSLAAILLLIPSVSFA

SEQ ID NO: 112 - protein, 24 aa, synthetic construct
MRSYIKVLTMCFLGLILFVPTALA

SEQ ID NO: 113 - protein, 27 aa, synthetic construct
MKKRLIAPMLLSAASLAFFAMSGSAQA

SEQ ID NO: 114 - protein, 19 aa, synthetic construct
MKKQIITATTAVVLGALFA

SEQ ID NO: 115 - protein, 26 aa, synthetic construct
MKKKLAAGLTASAIVGTTLVVTPAEA

SEQ ID NO: 116 - protein, 39 aa, synthetic construct
MRNERRKKKKTLLLTILTIIGLLVLGTGGYAYYLWHKAA

SEQ ID NO: 117 - protein, 35 aa, synthetic construct
MDTTTAKQASTKFVVLGLLLGILMSAMDNTIVATA

SEQ ID NO: 118 - protein, 40 aa, synthetic construct
MARKKKKKHEDEHVDESWLVPYADILTLLLALFIVLYASS

SEQ ID NO: 119 - protein, 34 aa, synthetic construct
MKLVPRFRKQWFAYLTVLCLALAAAVSFGVPAKA

SEQ ID NO: 120 - protein, 23 aa, synthetic construct
MPNKRLMLLLLCIIILVAMIGFS

SEQ ID NO: 121 - protein, 28 aa, synthetic construct
MRNLTKTSLLLAGLCTAAQMVFVTHASA

SEQ ID NO: 122 - protein, 27 aa, synthetic construct
MGLGKKLSVAVAASFMSLSISLPGVQA

SEQ ID NO: 123 - protein, 29 aa, synthetic construct
MKKWMAGLFLAAAVLLCLMVPQQIQGASS

SEQ ID NO: 124 - protein, 29 aa, synthetic construct
MKKSIKLYVAVLLLFVVASVPYMHQAALA

SEQ ID NO: 125 - protein, 34 aa, synthetic construct
MIQMPKKNKFMNRGAAILSICFALFFFVILGRMA

SEQ ID NO: 126 - protein, 26 aa, synthetic construct
MTMLRKIIGWILLLCIIPLFAFTVIA

SEQ ID NO: 127 - protein, 39 aa, synthetic construct
MTSPTRRRTAKRRRRKLNKRGKLLFGLLAVMVCITIWNA

SEQ ID NO: 128 - protein, 21 aa, synthetic construct
MKKVMLATALFLGLTPAGANA

SEQ ID NO: 129 - protein, 24 aa, synthetic construct
MKRLCLWFTVFSLFLVLLPGKALG

SEQ ID NO: 130 - protein, 36 aa, synthetic construct
MKLKTKASIKFGICVGLLCLSITGFTPFFNSTHAEA

SEQ ID NO: 131 - protein, 30 aa, synthetic construct
KKMSLFQNMKSKLLPIAAVSVLTAGIFAGA

SEQ ID NO: 132 - protein, 32 aa, synthetic construct
MKKFPKKLLPIAVLSSIAFSSLASGSVPEASA

SEQ ID NO: 133 - protein, 25 aa, synthetic construct
MKSKWMSGLLLVAVGFSFTQVMVHA

SEQ ID NO: 134 - protein, 26 aa, synthetic construct
MLKSKLFVICLAAAAIFTAAGVSANA

SEQ ID NO: 135 - protein, 24 aa, synthetic construct
MKLKSKLLLSCLALSTVFVATTIA

SEQ ID NO: 136 - protein, 21 aa, synthetic construct
MKRFLIGAGVAAVILSGWFIA

SEQ ID NO: 137 - protein, 19 aa, synthetic construct
MKKLVLCVSILAVILSGVA

SEQ ID NO: 138 - protein, 26 aa, synthetic construct
MRKKITLACKTCGNRNYTTMKSSASA

SEQ ID NO: 139 - protein, 29 aa, synthetic construct
MNIKKFAKQATVLTFTTALLAGGATQAFA

SEQ ID NO: 140 - protein, 24 aa, synthetic construct
MKKRLIQVMIMFTLLLTMAFSADA

SEQ ID NO: 141 - protein, 31 aa, synthetic construct
MKSKGSIMACLILFSFTITTFINTETISAFS

SEQ ID NO: 142 - protein, 34 aa, synthetic construct
MKQFAITLSVLCALILLVPTLLVIPFQHNKEAGA

SEQ ID NO: 143 - protein, 39 aa, synthetic construct
MRNKRRNRQIVVAVNGGKAVKAIFLFIVSLIVIFVLSGV

SEQ ID NO: 144 - protein, 40 aa, synthetic construct
MREEEKKTSQVKKLQQFFRKRWVFPAIYLVSAAVILTAVL

SEQ ID NO: 145 - protein, 20 aa, synthetic construct
MKKTVIICIYIFLLLSGALV

SEQ ID NO: 146 - protein, 27 aa, synthetic construct
MGMKKKLSLGVASAALGLALVGGGTWA

SEQ ID NO: 147 - protein, 23 aa, synthetic construct
MNQMKDTILLAGLGLIGGSIALA

SEQ ID NO: 148 - protein, 31 aa, synthetic construct
MKKGIIRFLLVSFVLFFALSTGITGVQAAPA

SEQ ID NO: 149 - protein, 32 aa, synthetic construct
MKKRKRRNFKRFIAAFLVLALMISLVPADVLA

SEQ ID NO: 150 - protein, 31 aa, synthetic construct
MKRRKFSSVVAAVLIFALIFSLFSPGTKAAA

SEQ ID NO: 151 - protein, 28 aa, synthetic construct
MFKFKKNFLVGLSAALMSISLFSATASA

SEQ ID NO: 152 - protein, 23 aa, synthetic construct
MRKTIFAFLTGLMMFGTITAASA

SEQ ID NO: 153 - protein, 25 aa, synthetic construct
MKTKTLFIFSAILTLSIFAPNETFA

SEQ ID NO: 154 - protein, 28 aa, synthetic construct
MDKFLNNRWAVKIIALLFALLLYVAVNS

SEQ ID NO: 155 - protein, 25 aa, synthetic construct
MKTLWKVLKIVFVSLAALVLLVSVS

SEQ ID NO: 156 - protein, 25 aa, synthetic construct
MVKKWLIQFAVMLSVLSTFTYSASA

SEQ ID NO: 157 - protein, 28 aa, synthetic construct
MKRMIVRMTLPLLIVCLAFSSFSASARA

SEQ ID NO: 158 - protein, 23 aa, synthetic construct
MKKWIYVVLVLSIAGIGGFSVHA

SEQ ID NO: 159 - protein, 26 aa, synthetic construct
MKRITINIITMFIAAAVISLTGTAEA

SEQ ID NO: 160 - protein, 33 aa, synthetic construct
MKLFNRKVTLVSLILMAVFQFFMALIIKRIVIS

SEQ ID NO: 161 - protein, 28 aa, synthetic construct
MRKKRVITCVMAASLTLGSLLPAGYASA

SEQ ID NO: 162 - protein, 21 aa, synthetic construct
MFKKHTISLLIIFLLASAVLA

SEQ ID NO: 163 - protein, 28 aa, synthetic construct
MLKKVILAAFILVGSTLGAFSFSSDASA

SEQ ID NO: 164 - protein, 21 aa, synthetic construct
MKKRIILLLAVIIAAAAAGVA

SEQ ID NO: 165 - protein, 29 aa, synthetic construct
MKKKQVMLALTAAAGLGLTALHSAPAAKA

SEQ ID NO: 166 - protein, 21 aa, synthetic construct
MKWMCSICCAAVLLAGGAAQA

SEQ ID NO: 167 - protein, 26 aa, synthetic construct
MMKKLFHSTLIVLLFFSFFGVQPIHA

SEQ ID NO: 168 - protein, 35 aa, synthetic construct
MRIQKRRTHVENILRILLPPIMILSLILPTPPIHA

SEQ ID NO: 169 - protein, 33 aa, synthetic construct
MRTWKRIPKTTMLISLVSPFLLITPVLFYAALA

SEQ ID NO: 170 - protein, 46 aa, synthetic construct
MLSVEMISRQNRCHYVYKGGNMMRRILHIVLITALMFLNVMYTFEA

SEQ ID NO: 171 - protein, 21 aa, synthetic construct
MKSLPYTIALLFCGLIIVSMA

SEQ ID NO: 172 - protein, 34 aa, synthetic construct
MKKIVAAIVVIGLVFIAFFYLYSRSGDVYQSVDA

SEQ ID NO: 173 - protein, 27 aa, synthetic construct
MKKAAAVLLSLGLVFGFSYGAGHVAEA

SEQ ID NO: 174 - protein, 26 aa, synthetic construct
MKKELLASLVLCLSLSPLVSTNEVFA

SEQ ID NO: 175 - protein, 34 aa, synthetic construct
MKKKTKIILSLLAALIVILIVLPVLSPVVFTASS

SEQ ID NO: 176 - protein, 26 aa, synthetic construct
MNFKKTVVSALSISALALSVSGVASA

SEQ ID NO: 177 - protein, 28 aa, synthetic construct
MKRLFMKASLVLFAVVFVFAVKGAPAKA

SEQ ID NO: 178 - protein, 26 aa, synthetic construct
MAAQTDYKKQVVGILLSLAFVLFVFS

SEQ ID NO: 179 - protein, 33 aa, synthetic construct
MKKVWIGIGIAVIVALFVGINIYRSAAPTSGSA

SEQ ID NO: 180 - protein, 26 aa, synthetic construct
MLKKKWMVGLLAGCLAAGGFSYNAFA

SEQ ID NO: 181 - protein, 21 aa, synthetic construct
MTTKFTALAVFLLCFMPAAKI

SEQ ID NO: 182 - protein, 26 aa, synthetic construct
MLTKRLLTIYIMLLGLIAWFPGAAQA

SEQ ID NO: 183 - protein, 26 aa, synthetic construct
MKKAFILSAAAAVGLFTFGGVQQASA

SEQ ID NO: 184 - protein, 31 aa, synthetic construct
MKKTFVKKAMLTTAAMTSAALLTFGPDAASA

SEQ ID NO: 185 - protein, 35 aa, synthetic construct
MLRKKHFSWMLVILILIAVLSFIKLPYYITKPGEA

SEQ ID NO: 186 - protein, 27 aa, synthetic construct
MKKIGLLFMLCLAALFTIGFPAQQADA

SEQ ID NO: 187 - protein, 37 aa, synthetic construct
MSGKKKESGKFRSVLLIIILPLMFLLIAGGIVLWAAG

SEQ ID NO: 188 - protein, 35 aa, synthetic construct
MRGKSAVLLSLIMLIAGFLISFSFQMTKENNKSAA

SEQ ID NO: 189 - protein, 33 aa, synthetic construct
MYKKFVPFAVFLFLFFVSFEMMENPHALDYIGA

SEQ ID NO: 190 - protein, 42 aa, synthetic construct
MAKPLSKGGILVKKVLIAGAVGTAVLFGTLSSGIPGLPAADA

SEQ ID NO: 191 - protein, 25 aa, synthetic construct
MRFTKVVGFLSVLGLAAVFPLTAQA

SEQ ID NO: 192 - protein, 32 aa, synthetic construct
MIPRIKKTICVLLVCFTMLSVMLGPGATEVLA

SEQ ID NO: 193 - protein, 30 aa, synthetic construct
MKVCQKSIVRFLVSLIIGTFVISVPFMANA

SEQ ID NO: 194 - protein, 28 aa, synthetic construct
MELSFTKILVILFVGFLVFGPDKLPALG

SEQ ID NO: 195 - protein, 24 aa, synthetic construct
MKKMLMLAFTFLLALTIHVGEASA

SEQ ID NO: 196 - protein, 25 aa, synthetic construct
MKIRKILLSSALSFGMLISAVPALA

SEQ ID NO: 197 - protein, 21 aa, synthetic construct
MKLERLLAMVVLLISKKQVQA

SEQ ID NO: 198 - protein, 23 aa, synthetic construct
MKKKRKGCFAAAGFMMIFVFVIA

SEQ ID NO: 199 - protein, 23 aa, synthetic construct
MKKTIMSFVAVAALSTTAFGAHA

SEQ ID NO: 200 - protein, 26 aa, synthetic construct
MKVPKTMLLSTAAGLLLSLTATSVSA

SEQ ID NO: 201 - protein, 26 aa, synthetic construct
MKKKIVAGLAVSAVVGSSMAAAPAEA

SEQ ID NO: 202 - protein, 28 aa, synthetic construct
MKKRITYSLLALLAVVAFAFTDSSKAKA

SEQ ID NO: 203 - protein, 25 aa, synthetic construct
MKKRLIGFLVLVPALIMSGITLIEA

SEQ ID NO: 204 - protein, 27 aa, synthetic construct
MKKWIVLFLVLIAAAISIFVYVSTGSE

SEQ ID NO: 205 - protein, 28 aa, synthetic construct
MRKKRVITCVMAASLTLGSLLPAGYATA

SEQ ID NO: 206 - protein, 21 aa, synthetic construct
MKKLIMALVILGALGTSYISA

SEQ ID NO: 207 - protein, 23 aa, synthetic construct
MKRFILVLSFLSIIVAYPIQTNA

SEQ ID NO: 208 - protein, 25 aa, synthetic construct
MKLRKVLTGSVLSLGLLVSASPAFA

SEQ ID NO: 209 - protein, 25 aa, synthetic construct
KLSVKIAGVLTVAAAAMTAKMYATA

SEQ ID NO: 210 - protein, 23 aa, synthetic construct
MNNNKLLLVDGMALLFRAFFATA

SEQ ID NO: 211 - protein, 29 aa, synthetic construct
MKLWMRKTLVVLFTIVTFGLVSPPAALMA

SEQ ID NO: 212 - protein, 32 aa, synthetic construct
MRKKALIFTVIFGIIFLAVLLVSASIYKSAMA

SEQ ID NO: 213 - protein, 34 aa, synthetic construct
NKWKRLFFILLAINFILAAGFVALVLLPGEQAQV

SEQ ID NO: 214 - protein, 26 aa, synthetic construct
MKKIWIGMLAAAVLLLMVPKVSLADA

SEQ ID NO: 215 - protein, 38 aa, synthetic construct
MGRIKTKITILLVLLLLLAGGYMYINDIELKDVPTAIG

SEQ ID NO: 216 - protein, 38 aa, synthetic construct
MKRLTLVCSIVFILFILFYDLKIGTIPIQDLPVYEASA

SEQ ID NO: 217 - protein, 28 aa, synthetic construct
MKQGKFSVFLILLLMLTLVVAPKGKAEA

SEQ ID NO: 218 - protein, 28 aa, synthetic construct
MFKKLLLATSALTFSLSLVLPLDGHAKA

SEQ ID NO: 219 - protein, 43 aa, synthetic construct
MFRLFHNQQKAKTKLKVLLIFQLSVIFSLTAAICLQFSDDTSA

SEQ ID NO: 220 - protein, 34 aa, synthetic construct
MTKRGIQAFAGGIILATAVLAAVFYLTDEDQAAA

SEQ ID NO: 221 - protein, 22 aa, synthetic construct
MMIKQCVICLSLLVFGTTAAHA

SEQ ID NO: 222 - protein, 28 aa, synthetic construct
MTLTKLKMLSMLTVMIASLFIFSSQALA

SEQ ID NO: 223 - protein, 33 aa, synthetic construct
MYINQQKKSFFNKKRIILSSIVVLFLIIGGAFL

SEQ ID NO: 224 - protein, 28 aa, synthetic construct
MKISKRMKLAVIAFLIVFFLLLLRLAEI

SEQ ID NO: 225 - protein, 37 aa, synthetic construct
MSNNQSRYENRDKRRKANLVLNILIAIVSILIVVVAA

SEQ ID NO: 226 - protein, 27 aa, synthetic construct
MNKKYFVLIVCIIFTSALFPTFSSVTA

SEQ ID NO: 227 - protein, 30 aa, synthetic construct
MKRKLLSSLAISALSLGLLVSAPTASFAAE

SEQ ID NO: 228 - protein, 37 aa, synthetic construct
MITDIFKPGCRKLCVFNMKGDYFVKVLLSALLLLLFA

SEQ ID NO: 229 - protein, 35 aa, synthetic construct
MPRYRGPFRKRGPLPFRYVMLLSVVFFILSTTVSL

SEQ ID NO: 230 - protein, 28 aa, synthetic construct
MTKKAWFLPLVCVLLISGWLAPAASASA

SEQ ID NO: 231 - protein, 31 aa, synthetic construct
MHLIRAAGAVCLAVVLIAGCRFNEDQHQAEG

SEQ ID NO: 232 - protein, 27 aa, synthetic construct
MKKWLIIAVSLAIAIVLFMYTKGEAKA

SEQ ID NO: 233 - protein, 30 aa, synthetic construct
MRKSLITLGLASVIGTSSFLIPFTSKTASA

SEQ ID NO: 234 - protein, 25 aa, synthetic construct
MNYIKAGKWLTVFLTFLGILLFIDL

SEQ ID NO: 235 - protein, 26 aa, synthetic construct
MKRIRIPMTLALGAALTIAPLSFASA

SEQ ID NO: 236 - protein, 27 aa, synthetic construct
MKKKQQSSAKFAVILTVVVVVLLAAIV

SEQ ID NO: 237 - protein, 21 aa, synthetic construct
MRKYTVIASILLSFLSVLSGG

SEQ ID NO: 238 - protein, 27 aa, synthetic construct
MKKIVSILFMFGLVMGFSQFQPSTVFA

SEQ ID NO: 239 - protein, 25 aa, synthetic construct
MKTLRTLCVLMILSGVIFFGLKIDA

SEQ ID NO: 240 - protein, 31 aa, synthetic construct
MKKLLTVMTMAVLTAGTLLLPAQSVTPAAHA

SEQ ID NO: 241 - protein, 28 aa, synthetic construct
MKRLLVSLRVWMVFLMNWVTPDRKTARA

SEQ ID NO: 242 - protein, 22 aa, synthetic construct
MKVFIILGAINALLAVGLGAFG

SEQ ID NO: 243 - protein, 30 aa, synthetic construct
MLKRTSFVSSLFISSAVLLSILLPSGQAHA

SEQ ID NO: 244 - protein, 25 aa, synthetic construct
MKGNIYSLFVLIAAFFWGTTGTVQA

SEQ ID NO: 245 - protein, 24 aa, synthetic construct
MKMKSGMEQAVSVLLLLSRLPVQA

SEQ ID NO: 246 - protein, 27 aa, synthetic construct
MKVFIVIMIIVVIFFALILLDIFMGRA

SEQ ID NO: 247 - protein, 28 aa, synthetic construct
MKKKQVSHAIIISVMLSFVIAVFHTIHA

SEQ ID NO: 248 - protein, 23 aa, synthetic construct
MKKRFSLIMMTGLLFGLTSPAFA

SEQ ID NO: 249 - protein, 23 aa, synthetic construct
MKKLLAAGIIGLLTVSIASPSFA

SEQ ID NO: 250 - protein, 27 aa, synthetic construct
MRKWYFILLAGVLTSVILAFVYDKTKA

SEQ ID NO: 251 - protein, 30 aa, synthetic construct
MGESTSLKEILSTLTKRILLIMIVTAAATA

SEQ ID NO: 252 - protein, 28 aa, synthetic construct
MKFLLSVIAGLLILALYLFWKVQPPVWI

SEQ ID NO: 253 - protein, 30 aa, synthetic construct
MNKPTKLFSTLALAAGMTAAAAGGAGTIHA

SEQ ID NO: 254 - protein, 22 aa, synthetic construct
MKFVKAIWPFVAVAIVFMFMSA

SEQ ID NO: 255 - protein, 32 aa, synthetic construct
MNTLANWKKFLLVAVIICFLVPIMTKAEIAEA

SEQ ID NO: 256 - protein, 39 aa, synthetic construct
MEERSQRRKKKRKLKKWVKVVAGLMAFLVIAAGSVGAYA

SEQ ID NO: 257 - protein, 41 aa, synthetic construct
MVKSFRMKALIAGAAVAAAVSAGAVSDVPAAKVLQPTAAYA

SEQ ID NO: 258 - protein, 26 aa, synthetic construct
MFNRLFRVCFLAALIMAFTLPNSVYA

SEQ ID NO: 259 - protein, 32 aa, synthetic construct
MKWNNMLKAAGIAVLLFSVFAYAAPSLKAVQA

SEQ ID NO: 260 - protein, 26 aa, synthetic construct
MVYQTKRDVPVTLMIVFLILLIQADA

SEQ ID NO: 261 - protein, 30 aa, synthetic construct
MNKFLKSNFRFLLAAALGISLLASSNFIKA

SEQ ID NO: 262 - protein, 27 aa, synthetic construct
MKKWMITIAMLILAGIALFVFISPLKS

SEQ ID NO: 263 - DNA, 96 bp, synthetic construct
  source 1..96: mol_type = other DNA
atgaagaagaagaaaacctggaagcgtttccttcatttttcaagcgcggctctggctgctggtctcattttcacttccgcggctcctgccgaagca

SEQ ID NO: 264 - DNA, 99 bp, synthetic construct
  source 1..99: mol_type = other DNA
atgtttgccaagcgttttaaaacatctctgcttccgttgtttgcgggatttctcttactgttccatttggtgctggccggaccggccgccgcaagcgca

SEQ ID NO: 265 - DNA, 87 bp, synthetic construct
  source 1..87: mol_type = other DNA
atgagatcaaaaaaactctggatttccctgttatttgccctcacgttaatcttcacaatggctttctctaatatgagtgttcaagcg

SEQ ID NO: 266 - DNA, 66 bp, synthetic construct
  source 1..66: mol_type = other DNA
atgaagttggcaaagagggtgagcgcgcttactccaagcaccacacttgcaatcacagcaaaagca

SEQ ID NO: 267 - DNA, 87 bp, synthetic construct
  source 1..87: mol_type = other DNA
atgaaacgtagcatcagcatttttattacatgtttgctgatcacattgttgaccatgggagggatgatcgcctccccggcctctgcc

SEQ ID NO: 268 - DNA, 84 bp, synthetic construct
  source 1..84: mol_type = other DNA
atgccttatctgaaaagggttcttctcctgcttgtaacgggcctgtttatgtcacttttcgcggtgacagcaacggcgtccgcg

SEQ ID NO: 269 - DNA, 90 bp, synthetic construct
  source 1..90: mol_type = other DNA
atgagaaagaagaccaaaaaccgccttatttcgtcagtcctgagcacagtagttatctcttccttactgtttccaggtgctgcgggcgca

SEQ ID NO: 270 - DNA, 81 bp, synthetic construct
  source 1..81: mol_type = other DNA
atgaaatggaatccgttgattccgtttcttctcatagcagtactgggaataggattaacttttttcttgtctgtcaaggga

SEQ ID NO: 271 - DNA, 96 bp, synthetic construct
  source 1..96: mol_type = other DNA
atgggaaacacgcgcaaaaaggtttcagtcatcggcgcagggttcacaggagctacaactgcctttctgattgcccaaaaagagttagcagacgta

SEQ ID NO: 272 - DNA, 111 bp, synthetic construct
  source 1..111: mol_type = other DNA
atgaagaacaggttatttatcttgatctgcttttgtgtcatatgcctcttcttatcgtttggccaaccgttttttccttctatgattcttacggtacaagcagcaaaaagc

SEQ ID NO: 273 - DNA, 105 bp, synthetic construct
  source 1..105: mol_type = other DNA
atgaagattagcatgcagaaagccgacttctggaagaaagcggctatttcattattagtttttacgatgttctttactctgatgatgtctgagactgtgtttgct

SEQ ID NO: 274 - DNA, 81 bp, synthetic construct
  source 1..81: mol_type = other DNA
atgcggaaaaaactgaaatggcttagttttctgttgggcttcataattctgctttttttatttaagtatcaattttcaaat

SEQ ID NO: 275 - DNA, 81 bp, synthetic construct
  source 1..81: mol_type = other DNA
atgagaatttttaaaaaagcggtttttgtcattatgatctcctttctgattgccactgttaacgtcaatactgcacacgcc

SEQ ID NO: 276 - DNA, 69 bp, synthetic construct
  source 1..69: mol_type = other DNA
atgaagagattattgtcaaccttattaattggcattatgttactcacatttgcaccgagcgcgtttgca

SEQ ID NO: 277 - DNA, 63 bp, synthetic construct
  source 1..63: mol_type = other DNA
atgaaaaaaagattcttcgggccaatcattcttgcctttattctgtttgcgggggcgatcgca

SEQ ID NO: 278 - DNA, 81 bp, synthetic construct
  source 1..81: mol_type = other DNA
atgaaaaatatgtcgtgcaaacttgtggtatccgttaccttattttttagctttctcactatcggccctttagcgcacgcg

SEQ ID NO: 279 - DNA, 69 bp, synthetic construct
  source 1..69: mol_type = other DNA
atgaaaaaaaaacttatgatcattcttcttattatcttaatcgtgattggggcactgggagcagctgcg

SEQ ID NO: 280 - DNA, 81 bp, synthetic construct
  source 1..81: mol_type = other DNA
atgaaaaagagccagtattttattgttttcatctgcttttttgtacttttttcagttcatcctatcgcagcggcagctgca

SEQ ID NO: 281 - DNA, 78 bp, synthetic construct
  source 1..78: mol_type = other DNA
atgcggaaaaaccggattctggccctgtttgtgcttagcttgggcctgctgtctttcatggttaccccggtgtcagct

SEQ ID NO: 282 - DNA, 93 bp, synthetic construct
  source 1..93: mol_type = other DNA
atgaaatttgtgaaaagacggattatagctctggtcactattcttatgctctcggttacgtcgctgttcgctcttcaaccatctgcaaaggca

SEQ ID NO: 283 - DNA, 84 bp, synthetic construct
  source 1..84: mol_type = other DNA
atgaagaaggtgttgatggccttcatcatctgcttatcactgatactctccgtactcgccgcacctccaagcggagccaaagcg

SEQ ID NO: 284 - DNA, 75 bp, synthetic construct
  source 1..75: mol_type = other DNA
atgaagtcatgcaaacagttgattgtgtgctctctggcagctattctcctgttgattccttcagtttccttcgcg

SEQ ID NO: 285 - DNA, 72 bp, synthetic construct
  source 1..72: mol_type = other DNA
atgcgtagctacattaaagtactcactatgtgcttcttaggccttattctgtttgtgccgaccgcactggcc

SEQ ID NO: 286 - DNA, 81 bp, synthetic construct
  source 1..81: mol_type = other DNA
atgaagaaaagactgattgcacctatgttgttaagtgccgcttcattggcgttcttcgccatgtcaggatcagcccaggcc

SEQ ID NO: 287 - DNA, 57 bp, synthetic construct
  source 1..57: mol_type = other DNA
atgaaaaaacaaatcattacggcaactacggcagttgtgctgggcgctctttttgcc

SEQ ID NO: 288 - DNA, 78 bp, synthetic construct
  source 1..78: mol_type = other DNA
atgaaaaaaaaactcgcggccggcctgaccgcttctgcaatcgtcggaacgacattggttgtaacaccggccgaagct

SEQ ID NO: 289 - DNA, 117 bp, synthetic construct
  source 1..117: mol_type = other DNA
atgcgtaacgaacggcgaaaaaaaaaaaaaactctccttctgacaattcttacgattattggacttttggttcttgggacgggcggctacgcgtactacctgtggcataaagcagcg

SEQ ID NO: 290 - DNA, 105 bp, synthetic construct
  source 1..105: mol_type = other DNA
atggataccaccaccgccaaacaagcgtccacaaaatttgtcgtattgggacttcttctcggaattttgatgagcgcgatggataacacaattgtcgcgacagca

SEQ ID NO: 291 - DNA, 120 bp, synthetic construct
  source 1..120: mol_type = other DNA
atggctcggaagaagaaaaaaaaacatgaagatgagcatgtcgatgaatcttggttggttccatatgcggacatcttaaccctgcttctggcactttttattgtcctgtatgcgtctagc

SEQ ID NO: 292 - DNA, 102 bp, synthetic construct
  source 1..102: mol_type = other DNA
atgaaattagttccgaggttccgcaaacaatggtttgcatatttaacagtgctgtgcttggcactcgccgcagctgtcagcttcggcgttccggccaaagcg

SEQ ID NO: 293 - DNA, 69 bp, synthetic construct
  source 1..69: mol_type = other DNA
atgcctaataaacggttaatgctgttgcttttatgcatcattattttagtggcgatgattgggttctca

SEQ ID NO: 294 - DNA, 84 bp, synthetic construct
  source 1..84: mol_type = other DNA
atgcgcaatttgacgaaaacatcattacttctggcgggattgtgcacagccgcacaaatggtgttcgtcacccacgccagtgcg

SEQ ID NO: 295 - DNA, 81 bp, synthetic construct
  source 1..81: mol_type = other DNA
atgggactcggtaagaaactctccgtagcagttgcggcaagctttatgtcactttcaatctctttaccgggtgttcaagcg

SEQ ID NO: 296 - DNA, 87 bp, synthetic construct
  source 1..87: mol_type = other DNA
atgaaaaagtggatggcgggcctgtttctggcagcggcagttctgctgtgtctgatggtcccgcaacagattcaaggcgcttcctca

SEQ ID NO: 297 - DNA, 87 bp, synthetic construct
  source 1..87: mol_type = other DNA
atgaagaaatcaattaaactctatgtagctgttctgctcctttttgtggttgccagtgtgccgtatatgcaccaagctgccctcgca

SEQ ID NO: 298 - DNA, 102 bp, synthetic construct
  source 1..102: mol_type = other DNA
atgatacagatgccgaagaagaacaaatttatgaacagaggtgcagccatcttgagtatttgttttgctctctttttttttgtgattcttggacgtatggca

SEQ ID NO: 299 - DNA, 78 bp, synthetic construct
  source 1..78: mol_type = other DNA
atgacgatgctccgaaagataattgggtggatcttacttctgtgtattattccccttttcgcttttacagtcatcgct

SEQ ID NO: 300 - DNA, 117 bp, synthetic construct
  source 1..117: mol_type = other DNA
atgacaagcccaacgcgaagaagaacggcaaaaaggaggcgtagaaaacttaacaaaagaggcaaattgttatttggacttttagcggttatggtctgcataacgatctggaacgcc

SEQ ID NO: 301 - DNA, 63 bp, synthetic construct
  source 1..63: mol_type = other DNA
atgaaaaaagtaatgttagcgacagcactttttctggggcttacccctgcaggagcgaatgca

SEQ ID NO: 302 - DNA, 72 bp, synthetic construct
  source 1..72: mol_type = other DNA
atgaagaggttatgtctgtggttcaccgtgttctcactttttcttgtattacttcccggaaaggctcttggg

SEQ ID NO: 303 - DNA, 108 bp, synthetic construct
  source 1..108: mol_type = other DNA
atgaaactgaaaacgaaagcgagtataaaatttgggatatgtgtgggacttctttgtctgtccattaccggttttacaccatttttcaattctacacacgcggaggca

SEQ ID NO: 304 - DNA, 90 bp, synthetic construct
  source 1..90: mol_type = other DNA
aaaaaaatgtctctgttccaaaacatgaagagcaagctgctgccgatcgcggcggtatcagtattaactgcgggcatctttgcaggcgcg

SEQ ID NO: 305 - DNA, 96 bp, synthetic construct
  source 1..96: mol_type = other DNA
atgaaaaaatttccaaagaaacttttgccaatcgcggtgctgagcagtattgcattctcttccttagcctcaggttctgttcctgaagcaagcgca

SEQ ID NO: 306 - DNA, 75 bp, synthetic construct
  source 1..75: mol_type = other DNA
atgaaatctaaatggatgagtggtcttttacttgtagcagttggttttagttttacacaagtaatggttcatgct

SEQ ID NO: 307 - DNA, 78 bp, synthetic construct
  source 1..78: mol_type = other DNA
atgctgaaatcgaagttatttgttatttgcctggcagccgccgcaatttttacagcggcgggtgtctcagcaaatgcc

SEQ ID NO: 308 - DNA, 72 bp, synthetic construct
  source 1..72: mol_type = other DNA
atgaaactgaaatctaaactgctgctgagttgtctggccttgtccacagtttttgtagctacgaccatcgct

SEQ ID NO: 309 - DNA, 63 bp, synthetic construct
  source 1..63: mol_type = other DNA
atgaaaaggttccttattggagctggtgtggcggctgtgatactgtcaggttggtttatcgca

SEQ ID NO: 310 - DNA, 57 bp, synthetic construct
  source 1..57: mol_type = other DNA
atgaaaaaactggtcttatgtgtaagcattctcgcagtcattctctctggagttgcc

SEQ ID NO: 311 - DNA, 78 bp, synthetic construct
  source 1..78: mol_type = other DNA
atgcgtaaaaaaattacattagcatgtaaaacatgtggaaaccgcaattacacaacaatgaaaagctccgcttcggcc

SEQ ID NO: 312 - DNA, 87 bp, synthetic construct
  source 1..87: mol_type = other DNA
atgaatatcaaaaaattcgctaagcaagcgactgtgttaacatttaccacggcactcctggcaggaggtgctacacaagcttttgca

SEQ ID NO: 313 - DNA, 72 bp, synthetic construct
  source 1..72: mol_type = other DNA
atgaaaaagagattgattcaagtaatgatcatgtttacgttgcttctgactatggccttcagtgccgatgct

SEQ ID NO: 314 - DNA, 93 bp, synthetic construct
  source 1..93: mol_type = other DNA
atgaaatctaaaggaagtatcatggcatgcttgattttgtttagctttacgataacgacctttattaacacagagactattagcgccttcagt

SEQ ID NO: 315 - DNA, 102 bp, synthetic construct
  source 1..102: mol_type = other DNA
atgaagcaatttgctatcaccttaagtgttctgtgtgcgttaatattacttgttcctacgctcctcgtgatcccgtttcaacataataaagaagcaggagca

SEQ ID NO: 316 - DNA, 117 bp, synthetic construct
  source 1..117: mol_type = other DNA
atgcgtaataaacgtagaaaccggcagattgtcgtagctgtcaacggaggtaaagcagtaaaagctatctttctgtttatcgtaagtttaattgttatttttgttttgtccggggtg

SEQ ID NO: 317 - DNA, 120 bp, synthetic construct
  source 1..120: mol_type = other DNA
atgagggaagaagaaaaaaagacgagtcaggtgaaaaaactccaacagttttttagaaaacgttgggtgtttccagcgatctatcttgtctctgccgctgttatcctgacagcagtgttg

SEQ ID NO: 318 - DNA, 60 bp, synthetic construct
  source 1..60: mol_type = other DNA
atgaagaaaacagttattatctgcatttacatttttttgttgttatcaggggcgctggtt

SEQ ID NO: 319 - DNA, 81 bp, synthetic construct
  source 1..81: mol_type = other DNA
atgggaatgaaaaaaaaactttctctcggcgttgcgtcagccgcgttggggttggcacttgtaggaggtggaacatgggct

SEQ ID NO: 320 - DNA, 69 bp, synthetic construct
  source 1..69: mol_type = other DNA
atgaatcagatgaaagacacgatcctgctcgcgggtttaggcttaattggcggcagcatcgcgttagct

SEQ ID NO: 321 - DNA, 93 bp, synthetic construct
  source 1..93: mol_type = other DNA
atgaaaaaaggcattatccggttcttgctcgttagcttcgtattgttttttgccctgagtactggtattacaggagtacaagcggcccccgct

SEQ ID NO: 322 - DNA, 96 bp, synthetic construct
  source 1..96: mol_type = other DNA
atgaaaaaacggaagagacggaattttaaacgttttattgcggcttttcttgttcttgccttaatgatttctcttgtgcctgcagatgtgttagcc

SEQ ID NO: 323 - DNA, 93 bp, synthetic construct
  source 1..93: mol_type = other DNA
atgaaaagacgtaaattctccagcgttgtggctgcggttttaatatttgctttaattttcagcttgttttcgccaggaactaaagcggccgcg

SEQ ID NO: 324 - DNA, 84 bp, synthetic construct
  source 1..84: mol_type = other DNA
atgtttaagtttaaaaaaaatttcttggtaggcttgtcagccgcactgatgtcaatctcgctgttttctgccaccgcgtccgcc

SEQ ID NO: 325 - DNA, 69 bp, synthetic construct
  source 1..69: mol_type = other DNA
atgcgcaagactatctttgcattcttaaccggtttaatgatgtttggcacgattaccgctgcatcggcg

SEQ ID NO: 326 - DNA, 75 bp, synthetic construct
  source 1..75: mol_type = other DNA
atgaaaactaagaccctgtttatatttagcgctattctgacattatctattttcgcgcccaatgaaacattcgct

SEQ ID NO: 327 - DNA, 84 bp, synthetic construct
  source 1..84: mol_type = other DNA
atggataagttcttaaataaccgctgggcagtgaaaatcatcgcattgctttttgcgctgcttctgtatgtagccgttaatagc

SEQ ID NO: 328 - DNA, 75 bp, synthetic construct
  source 1..75: mol_type = other DNA
atgaagacgctttggaaggtgcttaaaatcgttttcgtgagtcttgcggccttggttttacttgtatctgtaagc

SEQ ID NO: 329 - DNA, 75 bp, synthetic construct
  source 1..75: mol_type = other DNA
atggttaagaaatggcttatacagttcgcagttatgttgagcgtgctgagcaccttcacatatagcgcgagcgca

SEQ ID NO: 330 - DNA, 84 bp, synthetic construct
  source 1..84: mol_type = other DNA
atgaaacgcatgattgtccgaatgacactgccgttgctgatagtttgtctggctttttcaagctttagcgctagtgcccgcgcg

SEQ ID NO: 331 - DNA, 69 bp, synthetic construct
  source 1..69: mol_type = other DNA
atgaagaaatggatatatgttgtacttgttttgtcgatcgcgggaatcggcggcttttcggttcacgcc

SEQ ID NO: 332 - DNA, 78 bp, synthetic construct
  source 1..78: mol_type = other DNA
atgaaaagaattacgattaacattattacaatgtttattgctgcagccgtcatttctctcacgggcaccgccgaggcc

SEQ ID NO: 333 - DNA, 99 bp, synthetic construct
  source 1..99: mol_type = other DNA
atgaaactgttcaatcggaaagtcacgttagtgagcttaattcttatggccgtgttccaattttttatggccttaatcatcaaacgaatagtaatttcc

SEQ ID NO: 334 - DNA, 84 bp, synthetic construct
  source 1..84: mol_type = other DNA
atgagaaagaaacgtgtgatcacgtgcgtgatggcggcgagcctcacattaggaagcttactccctgcaggctacgcctccgct

SEQ ID NO: 335 - DNA, 63 bp, synthetic construct
  source 1..63: mol_type = other DNA
atgtttaagaaacacactatatctttactgatcatttttcttctggcgtcagcagtacttgca

SEQ ID NO: 336 - DNA, 84 bp, synthetic construct
  source 1..84: mol_type = other DNA
atgctgaaaaaggtaatactggcagctttcatcctggtgggaagcacgctgggggcattttctttcagctctgacgcctcggct

SEQ ID NO: 337 - DNA, 63 bp, synthetic construct
  source 1..63: mol_type = other DNA
atgaagaagcggattatcctccttctggcggttattatcgccgccgctgcagcgggggttgcc

SEQ ID NO: 338 - DNA, 87 bp, synthetic construct
  source 1..87: mol_type = other DNA
atgaaaaaaaagcaggtgatgctggctcttaccgccgctgctgggctgggacttaccgctctccattctgctcctgccgcgaaagca

SEQ ID NO: 339 - DNA, 63 bp, synthetic construct
  source 1..63: mol_type = other DNA
atgaaatggatgtgctcaatctgctgtgctgcagtattacttgcaggaggagctgctcaagct

SEQ ID NO: 340 - DNA, 78 bp, synthetic construct
  source 1..78: mol_type = other DNA
atgatgaaaaagctgttccatagcacactgatcgtcctcctgtttttttctttttttggggtacaacccatacatgca

SEQ ID NO: 341 - DNA, 105 bp, synthetic construct
  source 1..105: mol_type = other DNA
atgcgaattcagaaacggaggactcatgtagaaaatatcctccgtattttgttaccgccgataatgatcctctcgctgattttgcctactccgccgatccatgca

SEQ ID NO: 342 - DNA, 99 bp, synthetic construct
  source 1..99: mol_type = other DNA
atgcgcacgtggaaacgtattccgaaaacgactatgctcatctcactcgtgtcaccgttccttttgattacaccggttcttttttatgcggcattagct

SEQ ID NO: 343 - DNA, 138 bp, synthetic construct
  source 1..138: mol_type = other DNA
atgctttctgtcgaaatgatttccaggcagaatcgttgtcactacgtctacaaaggcggcaacatgatgagacggattttacacatcgtactcataaccgcgttgatgtttcttaatgttatgtatacttttgaagcg

SEQ ID NO: 344 - DNA, 63 bp, synthetic construct
  source 1..63: mol_type = other DNA
atgaaatctctcccgtatacgatcgccctgctgttttgtggcttaattattgtctccatggca

SEQ ID NO: 345 - DNA, 102 bp, synthetic construct
  source 1..102: mol_type = other DNA
atgaaaaaaattgtcgcagcgatcgtagtgatcggattggttttcattgcttttttttatctgtattcacgatctggagacgtttatcaatccgttgatgca

SEQ ID NO: 346 - DNA, 81 bp, synthetic construct
  source 1..81: mol_type = other DNA
atgaaaaaggccgcggcagtgctgctttctctgggactggtgttcgggttcagctacggagcaggtcacgtggctgaagcc

SEQ ID NO: 347 - DNA, 78 bp, synthetic construct
  source 1..78: mol_type = other DNA
atgaaaaaagagctgctcgcgagtctggtcctgtgcttatccctgagccctttggtatctacaaacgaggtgttcgca

SEQ ID NO: 348 - DNA, 102 bp, synthetic construct
  source 1..102: mol_type = other DNA
atgaaaaaaaaaacaaagattatcctctctcttcttgcggccttgatcgtcattttgatcgtgttgccggtcctttccccggttgtatttacagcaagttcg

SEQ ID NO: 349 - DNA, 78 bp, synthetic construct
  source 1..78: mol_type = other DNA
atgaattttaaaaaaacagttgtatcagcgttgtcaatctctgcacttgcgctttcagtgtcaggtgtcgcaagcgct

SEQ ID NO: 350 - DNA, 84 bp, synthetic construct
  source 1..84: mol_type = other DNA
atgaaaagattgtttatgaaagcaagtttagttttgtttgcggttgttttcgtgttcgcggtaaaaggcgcgcctgccaaagcc

SEQ ID NO: 351 - DNA, 78 bp, synthetic construct
  source 1..78: mol_type = other DNA
atggctgctcagaccgattataaaaagcaagtcgtcggaatacttctcagccttgcctttgtgttgtttgtgttttct

SEQ ID NO: 352 - DNA, 99 bp, synthetic construct
  source 1..99: mol_type = other DNA
atgaaaaaagtgtggatcggaatcggcattgccgtaatcgttgctttatttgtaggcattaatatttataggtcggccgcaccgacatctgggtcggca

SEQ ID NO: 353 - DNA, 78 bp, synthetic construct
  source 1..78: mol_type = other DNA
atgctgaaaaaaaagtggatggttggacttctggctggctgcctggcggctggaggattttcttacaatgcatttgcg

SEQ ID NO: 354 - DNA, 63 bp, synthetic construct
  source 1..63: mol_type = other DNA
atgaccacaaaattcaccgctctggcggtgttcttgttatgtttcatgccggctgcaaaaatc

SEQ ID NO: 355 - DNA, 78 bp, synthetic construct
  source 1..78: mol_type = other DNA
atgctcacaaaacgtcttttaacgatctatatcatgcttttaggtctgatagcgtggtttcctggggctgcccaagcg

SEQ ID NO: 356 - DNA, 78 bp, synthetic construct
  source 1..78: mol_type = other DNA
atgaaaaaggcatttatcttatcggcagcggcggcagtaggattgtttacatttggtggggtccaacaggcctccgca

SEQ ID NO: 357 - DNA, 93 bp, synthetic construct
  source 1..93: mol_type = other DNA
atgaagaaaacctttgtgaaaaaggcaatgctgacaacggcggcgatgacctcggccgcactgcttacattcggcccggacgcagcttcagcc

SEQ ID NO: 358 - DNA, 105 bp, synthetic construct
  source 1..105: mol_type = other DNA
atgttgagaaaaaaacatttttcgtggatgcttgtgattcttatcttaattgcagtgctttcattcattaaattgccgtactatatcacaaaacccggagaagca

SEQ ID NO: 359 - DNA, 81 bp, synthetic construct
  source 1..81: mol_type = other DNA
atgaagaagatcggcttgctttttatgttgtgtctggctgctttgttcacaattggctttccagctcagcaagctgatgcg

SEQ ID NO: 360 - DNA, 111 bp, synthetic construct
  source 1..111: mol_type = other DNA
atgtctggaaaaaagaaagagtccgggaaatttcgctcagttttgctgatcattattttgccgcttatgtttctgttgattgctgggggtatcgtcttatgggcggcggga

SEQ ID NO: 361 - DNA, 105 bp, synthetic construct
  source 1..105: mol_type = other DNA
atgcggggcaaaagtgctgtattacttagtctcatcatgttaatcgcaggatttctcattagcttctcgtttcagatgaccaaggagaataataaatcggcagca

SEQ ID NO: 362 - DNA, 99 bp, synthetic construct
  source 1..99: mol_type = other DNA
atgtacaaaaaatttgtcccatttgcggtgtttttgtttcttttttttgtgtcttttgaaatgatggagaatccacatgcacttgattacatcggcgca

SEQ ID NO: 363 - DNA, 126 bp, synthetic construct
  source 1..126: mol_type = other DNA
atggcaaaaccgctttccaaaggaggaatattggtgaaaaaagttctgatcgccggtgcggtaggtacagcggtattatttgggactttatcatccggaatccctggtttaccagcggcagatgct

SEQ ID NO: 364 - DNA, 75 bp, synthetic construct
  source 1..75: mol_type = other DNA
atgagatttacaaaggtagtcggctttttatctgtccttggattggcggcggttttcccgctgacggcgcaggcg

SEQ ID NO: 365 - DNA, 96 bp, synthetic construct
  source 1..96: mol_type = other DNA
atgataccgaggattaaaaagacgatttgcgtgctgctggtatgtttcacgatgttgagtgtcatgcttggtccaggagcgacagaagtattggcg

SEQ ID NO: 366 - DNA, 90 bp, synthetic construct
  source 1..90: mol_type = other DNA
atgaaagtctgtcaaaagagtatagtcagatttcttgtatctcttattattggtacctttgttatttcagtcccatttatggcaaacgcg

SEQ ID NO: 367 - DNA, 84 bp, synthetic construct
  source 1..84: mol_type = other DNA
atggaattatcctttacgaaaatcctggttattctttttgtcgggtttttggtttttggcccggataaacttccagctcttgga

SEQ ID NO: 368 - DNA, 72 bp, synthetic construct
  source 1..72: mol_type = other DNA
atgaaaaaaatgttgatgctggccttcactttcctgcttgccctcacaatccatgttggtgaagcgtcggca

SEQ ID NO: 369 - DNA, 75 bp, synthetic construct
  source 1..75: mol_type = other DNA
atgaaaatcagaaaaattctgttgagctcagctctttcatttggtatgttgatcagcgctgtacctgcacttgca

SEQ ID NO: 370 - DNA, 63 bp, synthetic construct
  source 1..63: mol_type = other DNA
atgaaactcgaacggttgttagcaatggtagtcctcttaataagtaaaaaacaggttcaagcg

SEQ ID NO: 371 - DNA, 69 bp, synthetic construct
  source 1..69: mol_type = other DNA
atgaaaaaaaaaagaaaggggtgctttgcggcagccgggttcatgatgatctttgttttcgtcattgcg

SEQ ID NO: 372 - DNA, 69 bp, synthetic construct
  source 1..69: mol_type = other DNA
atgaaaaagacaatcatgtcatttgtggcagttgcagcacttagtacaacagcgttcggcgcacatgca

SEQ ID NO: 373 - DNA, 78 bp, synthetic construct
  source 1..78: mol_type = other DNA
atgaaggtccccaaaacgatgctgttgtctacagcggcgggactcttactctcactgacggcgacgtccgtgtcagcc

SEQ ID NO: 374 - DNA, 78 bp, synthetic construct
  source 1..78: mol_type = other DNA
atgaaaaagaaaattgtcgcggggctggccgtttcagcggtagtcggttctagtatggccgcagctccggctgaagcc

SEQ ID NO: 375 - DNA, 84 bp, synthetic construct
  source 1..84: mol_type = other DNA
atgaagaagcgcattacttattctcttttggcactgcttgcagttgttgcattcgcttttaccgactcgtctaaggcgaaagca

SEQ ID NO: 376 - DNA, 75 bp, synthetic construct
  source 1..75: mol_type = other DNA
atgaaaaagcgtctgattggctttctggtgttagtaccggcattaattatgtctggaattaccttaattgaagca

SEQ ID NO: 377 - DNA, 81 bp, synthetic construct
  source 1..81: mol_type = other DNA
atgaaaaaatggattgtcctgtttttagtacttatcgcagctgcgatttctatatttgtctatgtgtccacagggtcagag

SEQ ID NO: 378 - DNA, 84 bp, synthetic construct
  source 1..84: mol_type = other DNA
atgaggaagaaacgggtgattacctgcgtaatggcagctagtctgactctgggctcgcttcttccagcaggatatgccacagct

SEQ ID NO: 379 - DNA, 63 bp, synthetic construct
  source 1..63: mol_type = other DNA
atgaaaaaactgattatggcgcttgttatcttgggggcacttggaacctcttacatttcagcc

SEQ ID NO: 380 - DNA, 69 bp, synthetic construct
  source 1..69: mol_type = other DNA
atgaaacgctttatactcgtgttatcttttctctcgattatagtagcgtatccgattcaaactaacgca

SEQ ID NO: 381 - DNA, 75 bp, synthetic construct
  source 1..75: mol_type = other DNA
atgaaattacgcaaagtactcaccggatccgtcttaagcttaggattgttagtcagcgcgagtccggctttcgcc

SEQ ID NO: 382 - DNA, 75 bp, synthetic construct
  source 1..75: mol_type = other DNA
aaattgtctgtaaagattgcaggcgtgctcaccgttgcagctgctgccatgacggcgaaaatgtacgcaacagcc

SEQ ID NO: 383 - DNA, 69 bp, synthetic construct
  source 1..69: mol_type = other DNA
atgaacaataacaagcttctccttgtcgatggcatggcactgctgtttcgtgccttttttgcgaccgca

SEQ ID NO: 384 - DNA, 87 bp, synthetic construct
  source 1..87: mol_type = other DNA
atgaagctgtggatgcgtaaaacgcttgtcgtgctttttaccatcgtaacttttggcctggtatctcctccagctgctttgatggcg

SEQ ID NO: 385 - DNA, 96 bp, synthetic construct
  source 1..96: mol_type = other DNA
atgcggaagaaggcattaatttttacggttatttttggtatcattttcctggcagtgttgttggtctctgcaagcatatataaatcagcaatggcc

SEQ ID NO: 386 - DNA, 102 bp, synthetic construct
  source 1..102: mol_type = other DNA
aacaaatggaagcgtctgttttttattctgcttgctatcaattttatcctggctgccggctttgtcgcgttagtgcttttaccgggagaacaagctcaagtc

SEQ ID NO: 387 - DNA, 78 bp, synthetic construct
  source 1..78: mol_type = other DNA
atgaagaagatctggatcggcatgcttgccgcggcagtattgttgcttatggtgccaaaggttagtctggccgatgct

SEQ ID NO: 388 - DNA, 114 bp, synthetic construct
  source 1..114: mol_type = other DNA
atgggacgtatcaagacgaagattacgatattattggttctgttactcttgttagcgggaggttacatgtatatcaatgatatcgaacttaaagatgtccctacagctatagga

SEQ ID NO: 389 - DNA, 114 bp, synthetic construct
  source 1..114: mol_type = other DNA
atgaaacgcctgacactggtctgttctatcgtttttatcctctttattttgttttatgatcttaaaatcggtacaattcccatccaagaccttccggtatacgaggcctcggct

SEQ ID NO: 390 - DNA, 84 bp, synthetic construct
  source 1..84: mol_type = other DNA
atgaaacaaggtaaattttcggtctttttgattcttctgcttatgctcacgcttgttgtggcaccaaaaggaaaagcggaagcc

SEQ ID NO: 391 - DNA, 84 bp, synthetic construct
  source 1..84: mol_type = other DNA
atgttcaagaaacttctgctcgctacaagcgcccttacattttctctttcactggtattgcctttggacggacacgccaaagcg

SEQ ID NO: 392 - DNA, 129 bp, synthetic construct
  source 1..129: mol_type = other DNA
atgttcaggctgtttcataaccagcagaaagctaaaacaaaattaaaggttttattgatatttcaactgagcgtcatttttagtttaaccgccgccatttgtttacaatttagcgatgatacatccgca

SEQ ID NO: 393 - DNA, 102 bp, synthetic construct
  source 1..102: mol_type = other DNA
atgacgaagagaggcatccaggccttcgcggggggcatcattttggcgacagcagtgcttgccgctgtattctatcttacagacgaggatcaagcagctgct

SEQ ID NO: 394 - DNA, 66 bp, synthetic construct
  source 1..66: mol_type = other DNA
atgatgatcaaacagtgtgttatttgccttagcttacttgtcttcggaacaacagccgcccacgcc

SEQ ID NO: 395 - DNA, 84 bp, synthetic construct
  source 1..84: mol_type = other DNA
atgacactcacgaaattgaaaatgctcagtatgctgacagtaatgattgcatcattgtttatctttagctcacaggctcttgcg

SEQ ID NO: 396 - DNA, 99 bp, synthetic construct
  source 1..99: mol_type = other DNA
atgtatattaaccagcaaaaaaaatcattttttaacaaaaaacgaattatcttgtcaagcatcgtcgtattgtttttaatcatcggaggcgctttcctc

SEQ ID NO: 397 - DNA, 84 bp, synthetic construct
  source 1..84: mol_type = other DNA
atgaaaatcagcaaacgcatgaaacttgccgttattgcttttttgattgtcttttttctgcttttgctcagacttgctgagatc

SEQ ID NO: 398 - DNA, 111 bp, synthetic construct
  source 1..111: mol_type = other DNA
atgtctaataaccaatcccggtacgaaaatcgcgacaaacgcagaaaagcaaacctggtattaaacattttaattgcaatcgtcagcatcctcattgtggtggtggctgct

SEQ ID NO: 399 - DNA, 81 bp, synthetic construct
  source 1..81: mol_type = other DNA
atgaataaaaagtactttgtcctgatcgtatgtattattttcacatccgccttatttccgacgttctcgtccgtgacagct

SEQ ID NO: 400 - DNA, 90 bp, synthetic construct
  source 1..90: mol_type = other DNA
atgaaacggaaactgctgtcttctcttgccatttcagctttgtcattaggattacttgttagcgcgccgacggcgtcttttgcagctgag

SEQ ID NO: 401 - DNA, 111 bp, synthetic construct
  source 1..111: mol_type = other DNA
atgatcacagacattttcaaaccaggttgcagaaaattatgcgtatttaatatgaagggcgattactttgttaaagtcctcctgtctgctttacttctgctcctttttgca

SEQ ID NO: 402 - DNA, 105 bp, synthetic construct
  source 1..105: mol_type = other DNA
atgcccaggtaccggggaccgtttcgcaaaagaggacctttaccttttcggtatgtcatgttacttagcgtagtttttttcattttaagcacaacagtttcactt

SEQ ID NO: 403 - DNA, 84 bp, synthetic construct
  source 1..84: mol_type = other DNA
atgacgaaaaaagcatggtttctcccgctcgtttgtgttctcttaatcagtggatggttagcccctgcagcgtctgccagtgcg

SEQ ID NO: 404 - DNA, 93 bp, synthetic construct
  source 1..93: mol_type = other DNA
atgcatttaataagggcggctggcgcggtgtgccttgcagttgttttaatcgctggatgtagatttaatgaggatcaacaccaagcagaaggt

SEQ ID NO: 405 - DNA, 81 bp, synthetic construct
  source 1..81: mol_type = other DNA
atgaagaagtggctgatcattgcggtcagtctcgccattgcgattgtgctgtttatgtatacaaaaggcgaggcgaaagct

SEQ ID NO: 406 - DNA, 90 bp, synthetic construct
  source 1..90: mol_type = other DNA
atgcggaaaagcctgatcacgctgggcctggcatctgtcattggcacaagctcttttctcattccgtttacttcaaagacagcgtctgct

SEQ ID NO: 407 - DNA, 75 bp, synthetic construct
  source 1..75: mol_type = other DNA
atgaattatattaaggccggcaaatggctcacggtcttcttaacatttctgggaattttactgttcattgatctg

SEQ ID NO: 408 - DNA, 78 bp, synthetic construct
  source 1..78: mol_type = other DNA
atgaagcgcatcaggatccctatgacgctcgcactgggtgcggcacttaccatcgcgccgttgtcctttgcaagcgcg

SEQ ID NO: 409 - DNA, 81 bp, synthetic construct
  source 1..81: mol_type = other DNA
atgaagaaaaagcaacaaagttcagctaaatttgctgttatcttaacagtagttgtggtcgtgttgttggcagcgattgtc

SEQ ID NO: 410 - DNA, 63 bp, synthetic construct
  source 1..63: mol_type = other DNA
atgcgtaaatacacagtaattgcctcgatcctgctttcatttctgtcggtgctgtctggtggc

SEQ ID NO: 411 - DNA, 81 bp, synthetic construct
  source 1..81: mol_type = other DNA
atgaaaaaaatcgtatcaattctgtttatgtttggattggtcatgggatttagtcagttccagccgtcgacagtatttgca

SEQ ID NO: 412 - DNA, 75 bp, synthetic construct
  source 1..75: mol_type = other DNA
atgaaaacattgcgcacgctttgtgtacttatgatcttgagcggtgttatattttttggcctgaaaattgacgcc

SEQ ID NO: 413 - DNA, 93 bp, synthetic construct
  source 1..93: mol_type = other DNA
atgaagaagctgctcacagtaatgacaatggccgttttgacggcaggaaccttgctcttgccggcgcagtctgtgaccccggccgctcacgca

SEQ ID NO: 414 - DNA, 84 bp, synthetic construct
  source 1..84: mol_type = other DNA
atgaaacgcttgctcgtaagcttgagagtttggatggtgttcttgatgaactgggttacaccggaccgcaaaacggctcgtgcc

SEQ ID NO: 415 - DNA, 66 bp, synthetic construct
  source 1..66: mol_type = other DNA
atgaaagtttttatcatacttggcgctatcaatgcgttgttagcagtgggactcggggcctttggt

SEQ ID NO: 416 - DNA, 90 bp, synthetic construct
  source 1..90: mol_type = other DNA
atgttgaagcgcacttcattcgtaagctctcttttcatatcaagtgcggttctgcttagcattcttctgccgagcggacaggcgcacgcc

SEQ ID NO: 417 - DNA, 75 bp, synthetic construct
  source 1..75: mol_type = other DNA
atgaaaggcaacatttattcattgtttgtgttaattgctgccttcttctggggtacaacaggcacagtccaagcg

SEQ ID NO: 418 - DNA, 72 bp, synthetic construct
  source 1..72: mol_type = other DNA
atgaaaatgaaatcaggtatggagcaggcggtatcagtccttcttcttttgtcgcgtttgccagttcaggcc

SEQ ID NO: 419 - DNA, 81 bp, synthetic construct
  source 1..81: mol_type = other DNA
atgaaagtttttatcgtcataatgattatcgtggttatttttttcgcgcttattcttttagatatcttcatgggacgtgcg

SEQ ID NO: 420 - DNA, 84 bp, synthetic construct
  source 1..84: mol_type = other DNA
atgaagaaaaagcaggtctcacacgccattattattagcgttatgttatcttttgtaatcgcagttttccacacgattcatgcg

SEQ ID NO: 421 - DNA, 69 bp, synthetic construct
  source 1..69: mol_type = other DNA
atgaaaaagcggttttctttaatcatgatgaccggattgttattcggtcttacctctcccgcattcgct

SEQ ID NO: 422 - DNA, 69 bp, synthetic construct
  source 1..69: mol_type = other DNA
atgaaaaaattacttgcagccggcattattgggttgcttacggtgtccattgccagcccgtcttttgcc

SEQ ID NO: 423 - DNA, 81 bp, synthetic construct
  source 1..81: mol_type = other DNA
atgcgtaaatggtattttatcttattagctggcgtccttacctccgttatccttgcgtttgtctatgataaaacgaaagcg

SEQ ID NO: 424 - DNA, 90 bp, synthetic construct
  source 1..90: mol_type = other DNA
atgggcgaatctacatccctcaaagaaattttaagcaccttgactaaacgcatccttctgatcatgatagtgacggccgctgccaccgca

SEQ ID NO: 425 - DNA, 84 bp, synthetic construct
  source 1..84: mol_type = other DNA
atgaaatttttgctgtcggtgattgctggccttttaatcctggcattatatttgttttggaaagtccaaccacctgtctggatt

SEQ ID NO: 426 - DNA, 90 bp, synthetic construct
  source 1..90: mol_type = other DNA
atgaacaaacctacaaagctcttttcaacgcttgcattagctgctggaatgacagcagcagccgccggaggagccgggacaatccatgcc

SEQ ID NO: 427 - DNA, 66 bp, synthetic construct
  source 1..66: mol_type = other DNA
atgaaattcgttaaagctatttggccgtttgttgcagtagcaatcgtctttatgtttatgtcggca

SEQ ID NO: 428 - DNA, 96 bp, synthetic construct
  source 1..96: mol_type = other DNA
atgaatacacttgcaaactggaagaaatttctgttagtcgctgtgattatttgctttctggtacctattatgacaaaggccgaaattgcagaagca

SEQ ID NO: 429 - DNA, 117 bp, synthetic construct
  source 1..117: mol_type = other DNA
atggaagaaagaagccagcgccggaaaaaaaagagaaagctcaaaaaatgggtaaaagttgtagctggacttatggcatttcttgtcattgcggccggatcagttggcgcctatgct

SEQ ID NO: 430 - DNA, 123 bp, synthetic construct
  source 1..123: mol_type = other DNA
atggtcaaatcgttcagaatgaaggcattgatcgcaggcgcagccgtagcggcggccgtgtccgcaggcgccgtgtccgatgtcccggctgctaaagtactccaacccacagcggcctacgct

SEQ ID NO: 431 - DNA, 78 bp, synthetic construct
  source 1..78: mol_type = other DNA
atgttcaaccgtttgtttcgggtgtgttttcttgcggcgctgattatggcattcacgttgcctaactctgtttatgcc

SEQ ID NO: 432 - DNA, 96 bp, synthetic construct
  source 1..96: mol_type = other DNA
atgaagtggaacaatatgctgaaagcggcgggcattgctgtcttgttatttagcgtattcgcatatgccgcaccgtcattaaaagcagtacaggcc

SEQ ID NO: 433 - DNA, 78 bp, synthetic construct
  source 1..78: mol_type = other DNA
atggtatatcagacaaaaagagatgttcctgtcacacttatgattgtcttcttaatcctgctgattcaggccgatgca

SEQ ID NO: 434 - DNA, 90 bp, synthetic construct
  source 1..90: mol_type = other DNA
atgaataagtttttaaaatcgaacttccgtttcctgttagctgccgctcttggaatatctcttcttgcgtcatcaaacttcatcaaagcg

SEQ ID NO: 435 - DNA, 81 bp, synthetic construct
  source 1..81: mol_type = other DNA
atgaaaaaatggatgattacaatcgccatgctcattttggccggaatcgcattatttgttttcatttctcctctgaaaagc